(19) United States
(12) Patent Application Publication
(10) Pub. No.: US 2025/0161436 A1
(43) Pub. Date: May 22, 2025

(54) MULTIVALENT VACCINE FOR PARAMYXOVIRUSES AND USES THEREOF

(71) Applicant: Icosavax, Inc., Seattle, WA (US)

(72) Inventors: ANDREW L. FELDHAUS, SEATTLE, WA (US); DOUGLAS A. HOLTZMAN, SEATTLE, WA (US); MAX CIARLET, SEATTLE, WA (US); NIRANJAN KANESA-THASAN, SEATTLE, WA (US)

(21) Appl. No.: 18/940,825

(22) Filed: Nov. 7, 2024

Related U.S. Application Data
(63) Continuation of application No. PCT/US23/67127, filed on May 17, 2023.
(60) Provisional application No. 63/342,953, filed on May 17, 2022.
(60) Provisional application No. 63/367,109, filed on Jun. 27, 2022.
(60) Provisional application No. 63/378,151, filed on Oct. 3, 2022.
(60) Provisional application No. 63/387,092, filed on Dec. 12, 2022.

(51) Int. Cl. A61K 39/295; A61K 39/00; A61P 31/14; C12N 7/00

(52) U.S. Cl. CPC A61K 39/295; A61P 31/14; C12N 7/00; A61K 2039/5258; A61K 2039/70; C12N 2760/18323; C12N 2760/18334; C12N 2760/18362; C12N 2760/18371; C12N 2760/18523; C12N 2760/18534; C12N 2760/18562; C12N 2760/18571

(57) ABSTRACT

Provided are compositions pharmaceutical compositions, comprising two or more virus-like particles (VLPs), wherein a first virus-like particle (VLP) comprises a first component comprising a respiratory syntactical virus (RSV) F protein ectodomain or antigenic variant thereof; and a second virus-like particle (VLP) comprises a first component comprising a comprises a human metapneumovirus (hMPV) F protein ectodomain or antigenic variant thereof. Further provided are methods of using said compositions for vaccination.

### CROSS-REFERENCE TO RELATED APPLICATION

This application is a continuation of International Patent Application No. PCT/US23/67127, filed May 17, 2023, which claims the benefit of U.S. Provisional Patent Application No. 63/342,953, filed May 17, 2022, U.S. Provisional Patent Application No. 63/367,109, filed Jun. 27, 2022, U.S. Provisional Patent Application No. 63/378,151, filed Oct. 3, 2022, and U.S. Provisional Patent Application No. 63/387,092, filed Dec. 12, 2022, each of which is incorporated by reference herein in its entirety.

### INCORPORATION BY REFERENCE OF SEQUENCE LISTING

This application contains a Sequence Listing which has been submitted in .XML format via EFS-WEB and is hereby incorporated by reference in its entirety. Said .XML copy, created on Feb. 6, 2025, is named 062191-508C01US_SeqList_ST26.xml and is 315,392 bytes in size.

### BACKGROUND

Respiratory syncytial virus (RSV) is a single-strand, negative sense RNA virus of the family Pneumoviridae. There are two major genetic lineages, A and B (subgroups A and B, respectively), which are antigenically related and cross-neutralizing antibodies are induced upon infection with either subgroup. RSV circulates seasonally and is a major cause of lower respiratory tract infection (LRTI) worldwide in all age groups. Accumulating data has identified a substantial disease burden in adults, comparable to influenza, with most of the hospitalization and mortality occurring in older adults over 65 years of age. Epidemiological data suggest that in the US alone RSV may cause >170,000 hospitalizations and ˜14,000 deaths annually. Similarly, RSV is an important cause of respiratory disease in Europe. The incidence and severity of RSV disease is particularly high in the frail elderly and in older adults with cardiopulmonary conditions, who are considered at high risk for complications and hospitalization. RSV spreads by respiratory droplets and close contact with infected persons or fomites. In temperate climates there is an annual winter epidemic, while in tropical areas seasonality is less distinct, but infection is most common during the rainy season. The severity of RSV disease is largely determined by the extent of viral replication following infection. Induction of neutralizing antibodies (NAbs) should be the goal of vaccination as it is associated with protection against disease. However, seropositivity only confers partial protection against infection.

Human Metapneumovirus (hMPV) is a single-strand, negative sense RNA virus of the Metapneumovirus genus in the Pneumoviridae family. The most closely related human pathogen is RSV. As with RSV, there are two major genetic lineages, A and B (subgroups A and B, respectively), which are antigenically related, and cross-neutralizing antibodies are induced upon infection with either subgroup. In mouse models, infection with hMPV protects against near term re-infection; macaques infected with hMPV demonstrated seroconversion and temporary protection from subsequent infection that waned over several months. Similar to RSV, NAbs alone can protect against hMPV disease. Despite the presence of protective antibody titers in adults, reinfections with hMPV occur in both healthy and immunocompromised humans. In older adults, hMPV is responsible for a significant proportion of serious respiratory infections, with similar rates of infection as RSV. In the Etiology of Pneumonia in the Community (EPIC) trial, hMPV was confirmed in 4% of adults hospitalized with community-acquired pneumonia whereas RSV was confirmed in 3% of adults. There are currently no approved vaccines for RSV or hMPV.

Accordingly, there is a long-felt and unmet need for multivalent vaccines for paramyxoviruses, such as a bivalent RSV-hMPV vaccine.

### SUMMARY

The present disclosure relates to multivalent vaccines for paramyxoviruses and uses thereof In one aspect, the disclosure provides a composition or pharmaceutical composition, comprising two or more virus-like particles (VLPs). Each VLP comprises a first component and optionally one or more further components, which components collectively are assembled to form the VLP. The paramyxoviruses may be respiratory syntactical virus (RSV) and a metapneumovirus (hMPV), respectively. However, other combinations of paramyxoviruses are contemplated. The first virus-like particle (VLP) may comprise a first component (of this first VLP) comprising a respiratory syntactical virus (RSV) F protein ectodomain or antigenic variant thereof The second virus-like particle (VLP) may comprise a first component (of this second VLP) comprising a human metapneumovirus (hMPV) F protein ectodomain or antigenic variant thereof. The two VLPs may be mixed together to form the composition or pharmaceutical composition, and then used for vaccination or other purposes.

In another aspect, the disclosure provides a composition or pharmaceutical composition, a virus-like particle (VLP) comprising a plurality of first components. Some first components may comprise a respiratory syntactical virus (RSV) F protein ectodomain or antigenic variant thereof, while other first components comprising a human metapneumovirus (hMPV) F protein ectodomain or antigenic variant thereof The VLP is assembled from a mixture of the first components and optionally one or more further components. These “mosaic” VLPs may be used for vaccination or other purposes.

The present disclosure provides a composition or a pharmaceutical composition comprising two or more virus-like particles (VLPs), wherein a first virus-like particle (VLP) comprises a first component comprising a respiratory syntactical virus (RSV) F protein ectodomain or antigenic variant thereof, and a second virus-like particle (VLP) comprises a first component comprising a human metapneumovirus (hMPV) F protein ectodomain or antigenic variant thereof; and/or a virus-like particle (VLP) comprising a plurality of first components, some first components comprising a respiratory syntactical virus (RSV) F protein ectodomain or antigenic variant thereof and some first components comprising a human metapneumovirus (hMPV) F protein ectodomain or antigenic variant thereof.

In some embodiments, the VLPs each independently comprise a first component comprising a first multimerization domain; and a second component comprising a second multimerization domain.

In some embodiments, the first multimerization domain is selected from SEQ ID NOS: 1, 4, 5, 7, 9, 18, 19, 21, 24, 25, 26, 29, 30, 31, 34, 36, 37, 39, 42, 43, 44, 45, 46, 47, 48, 49, 50, 51, 52, 53, 144, 145, or functional variants thereof.

In some embodiments, the first multimerization domain shares at least 70%, at least 80%, at least 85%, at least 90%, at least 91%, at least 92%, at least 93%, at least 94%, at least 95%, at least 96%, at least 97%, at least 98%, at least 99%, or 100% identity to I53-50A (SEQ ID NO: 144) or I53-50A ΔCys (SEQ ID NO: 145).

In some embodiments, the first multimerization domain comprises the amino acid substitutions C74A and C98A; the amino acid substitutions C163A and C201A; or the amino acid substitutions C74A, C98A, C163A, and C201A relative to SEQ ID NO: 144.

In some embodiments, second multimerization domain is selected from SEQ ID NOs: 2, 3, 6, 8, 10, 11, 12, 13, 14, 15, 16, 17, 20, 22, 23, 27, 28, 32, 33, 35, 38, 40, and 41 or functional variants and fragments thereof.

In some embodiments, the second multimerization domain shares at least 70%, at least 80%, at least 85%, at least 90%, at least 91%, at least 92%, at least 93%, at least 94%, at least 95%, at least 96%, at least 97%, at least 98%, at least 99%, or 100% identity to I53-50B (SEQ ID NO: 8) or I53-50B.4PosT1 (SEQ ID NO: 34).

In some embodiments, the RSV F protein ectodomain shares at least 70%, at least 80%, at least 85%, at least 90%, at least 91%, at least 92%, at least 93%, at least 94%, at least 95%, at least 96%, at least 97%, at least 98%, at least 99%, or 100% identity to RSV F DS-Cav1 (SEQ ID NO: 173).

In some embodiments, the RSV F protein ectodomain comprises amino acid substitutions S155C and S290C; and/or amino acid substitutions S190F and V207L.

In some embodiments, the first component of the first VLP comprises a polypeptide that shares at least 70%, at least 80%, at least 90%, at least 91%, at least 92%, at least 93%, at least 94%, at least 95%, at least 96%, at least 97%, at least 98%, at least 99%, or 100% identity to DS-Cav1-I53-50A (SEQ ID NO: 148).

In some embodiments, the hMPV F protein ectodomain shares at least 70%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 174 or SEQ ID NO: 175.

In some embodiments, the hMPV F protein ectodomain comprises amino acid substitutions A63C, A140C, A147C, K188C, K450C, S470C, N97G, P98G, R99G, Q100G, S101G, and/or R102G; or wherein the hMPV F protein ectodomain comprises amino acid substitutions T127C, N153C, T365C, V463C, A185P, L219K, V231I, G294E, N97G, P98G, R99G, Q100G, H386N, S101G and/or R102G relative to relative to reference hMPV F protein sequence (SEQ ID NO: 56).

In some embodiments, the first component of the second VLP comprises a polypeptide that shares at least 70%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 176.

In some embodiments, the first component of the second VLP comprises a polypeptide that shares at least 70%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 177.

In some embodiments, the composition comprises one or more pharmaceutically acceptable diluents, adjuvants, or excipients.

In some embodiments, the composition comprises a stable emulsion. In some embodiments, the vaccine comprises one or more adjuvants. In some embodiments, the one or more adjuvants is squalene, alum, SLA, GLA, R848, IMQ, 3M-052, CpG, saponin (QS21), or combinations thereof. In some embodiments, the adjuvant is alum. In some embodiments, the adjuvant is an oil-in-water emulsion containing squalene (4.3%) in citric acid buffer with stabilizing nonionic surfactants Tween 80 (0.5%) and Span 85 (0.5%). In some embodiments, the adjuvant is a squalene-based emulsion. In some embodiments, the adjuvant is a squalene-based emulsion and a TLR4 agonist.

The present disclosure provides a unit dose of a composition or a pharmaceutical composition as disclosed herein, wherein the unit dose comprises: about 0.5 μg, about 1 μg, about 20 μg, about 25 μg, about 50 μg, about 70 μg, about 75 μg, about 100 μg, about 125 μg, about 150 μg, about 175 μg, about 200 μg, about 225 μg, about 250 μg, about 275 μg, about 300 μg, about 325 μg, about 350 μg, about 375 μg, about 400 μg, about 425 μg, about 450 μg, about 475 μg, or about 500 μg of the first VLP; and about 0.5 μg, about 1 μg, about 20 μg, about 25 μg, about 50 μg, about 70 μg, about 75 μg, about 100 μg, about 125 μg, about 150 μg, about 175 μg, about 200 μg, about 225 μg, about 250 μg, about 275 μg, about 300 μg, about 325 μg, about 350 μg, about 375 μg, about 400 μg, about 425 μg, about 450 μg, about 475 μg, or about 500 μg of the second VLP.

In some embodiments, the unit dose comprises about 75 μg of the first VLP comprising a RSV F protein ectodomain or antigenic variant thereof, and about 25 μg, about 50 μg, about 70 μg, about 75 μg, about 100 μg, about 125 μg, about 150 μg, about 175 μg, about 200 μg, about 225 μg, about 250 μg, about 275 μg, about 300 μg, about 325 μg, about 350 μg, about 375 μg, about 400 μg, about 425 μg, about 450 μg, about 475 μg, or about 500 μg of the second VLP comprising a hMPV F protein ectodomain or antigenic variant thereof.

In some embodiments, the unit dose comprises about 100 μg of the first VLP comprising a RSV F protein ectodomain or antigenic variant thereof, and about 25 μg, about 50 μg, about 70 μg, about 75 μg, about 100 μg, about 125 μg, about 150 μg, about 175 μg, about 200 μg, about 225 μg, about 250 μg, about 275 μg, about 300 μg, about 325 μg, about 350 μg, about 375 μg, about 400 μg, about 425 μg, about 450 μg, about 475 μg, or about 500 μg of the second VLP comprising a hMPV F protein ectodomain or antigenic variant thereof.

In some embodiments, the unit dose comprises about 125 μg of the first VLP comprising a RSV F protein ectodomain or antigenic variant thereof, and about 25 μg, about 50 μg, about 70 μg, about 75 μg, about 100 μg, about 125 μg, about 150 μg, about 175 μg, about 200 μg, about 225 μg, about 250 μg, about 275 μg, about 300 μg, about 325 μg, about 350 μg, about 375 μg, about 400 μg, about 425 μg, about 450 μg, about 475 μg, or about 500 μg of the second VLP comprising a hMPV F protein ectodomain or antigenic variant thereof.

In some embodiments, the unit dose comprises about 150 μg of the first VLP comprising a RSV F protein ectodomain or antigenic variant thereof, and about 25 μg, about 50 μg, about 70 μg, about 75 μg, about 100 μg, about 125 μg, about 150 μg, about 175 μg, about 200 μg, about 225 μg, about 250 μg, about 275 μg, about 300 μg, about 325 μg, about 350 μg, about 375 μg, about 400 μg, about 425 μg, about 450 μg, about 475 μg, or about 500 μg of the second VLP comprising a hMPV F protein ectodomain or antigenic variant thereof.

In some embodiments, the unit dose comprises about 175 μg of the first VLP comprising a RSV F protein ectodomain or antigenic variant thereof, and about 25 μg, about 50 μg, about 70 μg, about 75 μg, about 100 μg, about 125 μg, about 150 μg, about 175 μg, about 200 μg, about 225 μg, about 250 μg, about 275 μg, about 300 μg, about 325 μg, about 350 μg, about 375 μg, about 400 μg, about 425 μg, about 450 μg, about 475 μg, or about 500 μg of the second VLP comprising a hMPV F protein ectodomain or antigenic variant thereof.

In some embodiments, the unit dose comprises about 200 μg of the first VLP comprising a RSV F protein ectodomain or antigenic variant thereof, and about 25 μg, about 50 μg, about 70 μg, about 75 μg, about 100 μg, about 125 μg, about 150 μg, about 175 μg, about 200 μg, about 225 μg, about 250 μg, about 275 μg, about 300 μg, about 325 μg, about 350 μg, about 375 μg, about 400 μg, about 425 μg, about 450 μg, about 475 μg, or about 500 μg of the second VLP comprising a hMPV F protein ectodomain or antigenic variant thereof.

In some embodiments, the unit dose comprises about 225 μg of the first VLP comprising a RSV F protein ectodomain or antigenic variant thereof, and about 25 μg, about 50 μg, about 70 μg, about 75 μg, about 100 μg, about 125 μg, about 150 μg, about 175 μg, about 200 μg, about 225 μg, about 250 μg, about 275 μg, about 300 μg, about 325 μg, about 350 μg, about 375 μg, about 400 μg, about 425 μg, about 450 μg, about 475 μg, or about 500 μg of the second VLP comprising a hMPV F protein ectodomain or antigenic variant thereof.

In some embodiments, the unit dose comprises about 250 μg of the first VLP comprising a RSV F protein ectodomain or antigenic variant thereof, and about 25 μg, about 50 μg, about 70 μg, about 75 μg, about 100 μg, about 125 μg, about 150 μg, about 175 μg, about 200 μg, about 225 μg, about 250 μg, about 275 μg, about 300 μg, about 325 μg, about 350 μg, about 375 μg, about 400 μg, about 425 μg, about 450 μg, about 475 μg, or about 500 μg of the second VLP comprising a hMPV F protein ectodomain or antigenic variant thereof.

In some embodiments, the unit dose comprises about 75 μg of the first VLP comprising a RSV F protein ectodomain or antigenic variant thereof and about 75 μg of the second VLP comprising an hMPV F protein ectodomain or antigenic variant thereof.

In some embodiments, the unit dose comprises about 75 μg of the first VLP comprising a RSV F protein ectodomain or antigenic variant thereof and about 150 μg of the second VLP comprising an hMPV F protein ectodomain or antigenic variant thereof.

In some embodiments, the unit dose comprises about 75 μg of the first VLP comprising a RSV F protein ectodomain or antigenic variant thereof and about 225 μg of the second VLP comprising an hMPV F protein ectodomain or antigenic variant thereof.

In some embodiments, the unit dose comprises about 150 μg of the first VLP comprising a RSV F protein ectodomain or antigenic variant thereof and about 150 μg of the second VLP comprising an hMPV F protein ectodomain or antigenic variant thereof.

In some embodiments, the unit dose comprises about 225 μg of the first VLP comprising a RSV F protein ectodomain or antigenic variant thereof and about 75 μg of the second VLP comprising an hMPV F protein ectodomain or antigenic variant thereof.

In some embodiments, the unit dose comprises about 150 μg of the first VLP comprising a RSV F protein ectodomain or antigenic variant thereof and about 75 μg of the second VLP comprising an hMPV F protein ectodomain or antigenic variant thereof.

In some embodiments, the unit dose comprises about 75 μg of the first VLP comprising a RSV F protein ectodomain or antigenic variant thereof, about 75 μg of the second VLP comprising an hMPV F protein ectodomain or antigenic variant thereof, and an adjuvant comprising MF59.

In some embodiments, the unit dose comprises about 75 μg of the first VLP comprising a RSV F protein ectodomain or antigenic variant thereof, about 150 μg of the second VLP comprising an hMPV F protein ectodomain or antigenic variant thereof, and an adjuvant comprising MF59.

In some embodiments, the unit dose comprises about 75 μg of the first VLP comprising a RSV F protein ectodomain or antigenic variant thereof, about 225 μg of the second VLP comprising an hMPV F protein ectodomain or antigenic variant thereof, and an adjuvant comprising MF59.

In some embodiments, the unit dose comprises about 150 μg of the first VLP comprising a RSV F protein ectodomain or antigenic variant thereof, about 150 μg of the second VLP comprising an hMPV F protein ectodomain or antigenic variant thereof, and an adjuvant comprising MF59.

In some embodiments, the unit dose comprises about 225 μg of the first VLP comprising a RSV F protein ectodomain or antigenic variant thereof, about 75 μg of the second VLP comprising an hMPV F protein ectodomain or antigenic variant thereof, and an adjuvant comprising MF59.

In some embodiments, the unit dose comprises about 150 μg of the first VLP comprising a RSV F protein ectodomain or antigenic variant thereof, about 75 μg of the second VLP comprising an hMPV F protein ectodomain or antigenic variant thereof, and an adjuvant comprising MF59.

The present disclosure provides a method of vaccinating a subject, comprising administering to the subject an effective amount a composition according to the present disclosure.

In some embodiments, a method of generating an immune response or strengthening an existing immune response in a subject comprises administering to the subject an effective amount of a composition according to the present disclosure.

In some embodiments, a method of preventing infection by a paramyxovirus comprises administering to the subject an effective amount a composition according to the present disclosure.

In some embodiments, a method of immunizing a subject against a paramyxovirus comprises administering to the subject an effective amount of a composition according to the present disclosure.

In some embodiments, the paramyxovirus is respiratory syncytial virus (RSV), human metapneumovirus (hMPV), or both RSV and hMPV.

In some embodiments, the present method generates a protective immunity to respiratory syncytial virus (RSV), human metapneumovirus (hMPV), or both RSV and hMPV.

In some embodiments, the present method generates neutralizing antibodies to respiratory syncytial virus (RSV), human metapneumovirus (hMPV), or both RSV and hMPV.

In some embodiments, the subject is at risk of severe RSV disease and/or at risk of severe hMPV disease.

In some embodiments, the vaccine is administered by subcutaneous injection. In some embodiments, the vaccine is administered by intramuscular injection. In some embodiments, the vaccine is administered by intradermal injection. In some embodiments, the vaccine is administered intranasally. In some embodiments, the vaccine is administered orally. In some embodiments, the vaccine is administered sublingually. In some embodiments, the vaccine is administered buccally. In some embodiments, the vaccine is administered by one or more of the following methods: subcutaneous injection, intramuscular injection, intradermal injection, intranasally, orally, sublingually or buccally.

In some embodiments, the subject is an adult of over 60 years of age. In some embodiments, the subject is a healthy adult of 18-45 years of age.

In some embodiments, the effective amount comprises about 0.5 μg, about 1 μg, about 2 μg, about 20 μg, about 25 μg, about 40 μg, about 50 μg, about 70 μg, about 75 μg, about 100 μg, about 125 μg, about 140 μg, about 150 μg, about 175 μg, about 200 μg, about 225 μg, about 250 μg, about 275 μg, about 280 μg, about 300 μg, about 325 μg, about 350 μg, about 375 μg, about 400 μg, about 425 μg, about 450 μg, about 475 μg, about 500 μg, or about 600 μg of the first VLP and/or of the second VLP.

In some embodiments, the effective amount comprises about 75 μg of the first VLP comprising a RSV F protein ectodomain or antigenic variant thereof, and about 25 μg, about 50 μg, about 70 μg, about 75 μg, about 100 μg, about 125 μg, about 150 μg, about 175 μg, about 200 μg, about 225 μg, about 250 μg, about 275 μg, about 300 μg, about 325 μg, about 350 μg, about 375 μg, about 400 μg, about 425 μg, about 450 μg, about 475 μg, or about 500 μg of the second VLP comprising a hMPV F protein ectodomain or antigenic variant thereof.

In some embodiments, the effective amount comprises about 100 μg of the first VLP comprising a RSV F protein ectodomain or antigenic variant thereof, and about 25 μg, about 50 μg, about 70 μg, about 75 μg, about 100 μg, about 125 μg, about 150 μg, about 175 μg, about 200 μg, about 225 μg, about 250 μg, about 275 μg, about 300 μg, about 325 μg, about 350 μg, about 375 μg, about 400 μg, about 425 μg, about 450 μg, about 475 μg, or about 500 μg of the second VLP comprising a hMPV F protein ectodomain or antigenic variant thereof.

In some embodiments, the effective amount comprises about 125 μg of the first VLP comprising a RSV F protein ectodomain or antigenic variant thereof, and about 25 μg, about 50 μg, about 70 μg, about 75 μg, about 100 μg, about 125 μg, about 150 μg, about 175 μg, about 200 μg, about 225 μg, about 250 μg, about 275 μg, about 300 μg, about 325 μg, about 350 μg, about 375 μg, about 400 μg, about 425 μg, about 450 μg, about 475 μg, or about 500 μg of the second VLP comprising a hMPV F protein ectodomain or antigenic variant thereof.

In some embodiments, the effective amount comprises about 150 μg of the first VLP comprising a RSV F protein ectodomain or antigenic variant thereof, and about 25 μg, about 50 μg, about 70 μg, about 75 μg, about 100 μg, about 125 μg, about 150 μg, about 175 μg, about 200 μg, about 225 μg, about 250 μg, about 275 μg, about 300 μg, about 325 μg, about 350 μg, about 375 μg, about 400 μg, about 425 μg, about 450 μg, about 475 μg, or about 500 μg of the second VLP comprising a hMPV F protein ectodomain or antigenic variant thereof.

In some embodiments, the effective amount comprises about 175 μg of the first VLP comprising a RSV F protein ectodomain or antigenic variant thereof, and about 25 μg, about 50 μg, about 70 μg, about 75 μg, about 100 μg, about 125 μg, about 150 μg, about 175 μg, about 200 μg, about 225 μg, about 250 μg, about 275 μg, about 300 μg, about 325 μg, about 350 μg, about 375 μg, about 400 μg, about 425 μg, about 450 μg, about 475 μg, or about 500 μg of the second VLP comprising a hMPV F protein ectodomain or antigenic variant thereof.

In some embodiments, the effective amount comprises about 200 μg of the first VLP comprising a RSV F protein ectodomain or antigenic variant thereof, and about 25 μg, about 50 μg, about 70 μg, about 75 μg, about 100 μg, about 125 μg, about 150 μg, about 175 μg, about 200 μg, about 225 μg, about 250 μg, about 275 μg, about 300 μg, about 325 μg, about 350 μg, about 375 μg, about 400 μg, about 425 μg, about 450 μg, about 475 μg, or about 500 μg of the second VLP comprising a hMPV F protein ectodomain or antigenic variant thereof.

In some embodiments, the effective amount comprises about 225 μg of the first VLP comprising a RSV F protein ectodomain or antigenic variant thereof, and about 25 μg, about 50 μg, about 70 μg, about 75 μg, about 100 μg, about 125 μg, about 150 μg, about 175 μg, about 200 μg, about 225 μg, about 250 μg, about 275 μg, about 300 μg, about 325 μg, about 350 μg, about 375 μg, about 400 μg, about 425 μg, about 450 μg, about 475 μg, or about 500 μg of the second VLP comprising a hMPV F protein ectodomain or antigenic variant thereof.

In some embodiments, the effective amount comprises about 250 μg of the first VLP comprising a RSV F protein ectodomain or antigenic variant thereof, and about 25 μg, about 50 μg, about 70 μg, about 75 μg, about 100 μg, about 125 μg, about 150 μg, about 175 μg, about 200 μg, about 225 μg, about 250 μg, about 275 μg, about 300 μg, about 325 μg, about 350 μg, about 375 μg, about 400 μg, about 425 μg, about 450 μg, about 475 μg, or about 500 μg of the second VLP comprising a hMPV F protein ectodomain or antigenic variant thereof.

In some embodiments, the effective amount comprises about 0.5 μg, about 1 μg, about 20 μg, about 25 μg, about 70 μg, about 75 μg, about 100 μg, about 125 μg, about 150 μg, about 200 μg, about 225 μg, about 250 μg, or about 500 μg of the first VLP and/or of the second VLP.

In some embodiments, the effective amount comprises about 75 μg of the first VLP comprising a RSV F protein ectodomain or antigenic variant thereof, and about 25 μg, about 70 μg, about 75 μg, about 100 μg, about 125 μg, about 150 μg, about 200 μg, about 225 μg, about 250 μg, or about 500 μg of the second VLP comprising a hMPV F protein ectodomain or antigenic variant thereof.

In some embodiments, the effective amount comprises about 150 μg of the first VLP comprising a RSV F protein ectodomain or antigenic variant thereof, and about 25 μg, about 70 μg, about 75 μg, about 100 μg, about 125 μg, about 150 μg, about 200 μg, about 225 μg, about 250 μg, or about 500 μg of the second VLP comprising an hMPV F protein ectodomain or antigenic variant thereof.

In some embodiments, the effective amount comprises about 150 μg of the first VLP comprising a RSV F protein ectodomain or antigenic variant thereof, and about 150 μg of the second VLP comprising an hMPV F protein ectodomain or antigenic variant thereof.

In some embodiments, the method comprises administering a second dose of the pharmaceutical composition.

In some embodiments, the second dose is administered within about 2, 3, 4, 5, 6, 7, 8, 9, 10, 11, or 12 weeks, about 1 month, about 2 months, about 3 months, about 4 months, about 5 months, about 6 months, about 9 months, or about 12 months of the first dose.

In some embodiments, the method comprises administering a third dose of the pharmaceutical composition. In some embodiments, the third dose is administered within about 1 month, about 2 months, about 3 months, about 4 months, about 5 months, about 6 months, about 9 months, about 1 year, about 2 years, about 3 years, about 4 years, or about 5 years after the second dose.

In some embodiments, the method comprises administering subsequent doses at regular intervals of about 12 weeks, about 1 month, about 2 months, about 3 months, about 4 months, about 5 months, about 6 months, about 9 months, about 1 year, about 2 years, about 3 years, about 4 years, or about 5 years.

In some embodiments, the method limits the development of an RSV infection in a subject and/or the method limits the development of an hMPV infection in a subject.

In some embodiments, the method results in the production of RSV-A-specific neutralizing antibodies in the subject.

In some embodiments, the method results in an increase in RSV-A-specific neutralizing antibodies in the subject of at least about 2-fold, about 3-fold, at least about 4-fold, at least about 5-fold, at least about 10-fold, at least about 15-fold, at least about 20-fold, or at least about 25-fold compared to baseline.

In some embodiments, the increase in RSV-A-specific neutralizing antibodies is detectable within about one day, about 2 days, about 3 days, about 4 days, about 5 days, about 6 days, about 1 week, about 2 weeks, about 3 weeks, about 4 weeks, 5 weeks, about 6 weeks, about 7 weeks, about 8 weeks, about 9 weeks, about 10 weeks, about 11 weeks, or about 12 weeks of administration of the pharmaceutical composition.

In some embodiments, the method results in the production of RSV-B-specific neutralizing antibodies in the subject.

In some embodiments, the method results in an increase in RSV-B-specific neutralizing antibodies in the subject of at least about 2-fold, about 3-fold, at least about 4-fold, at least about 5-fold, at least about 10-fold, at least about 15-fold, at least about 20-fold, or at least about 25-fold compared to baseline.

In some embodiments, the increase in RSV-B-specific neutralizing antibodies is detectable within about one week, within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the pharmaceutical composition.

In some embodiments, the method results in the production of hMPV-A-specific neutralizing antibodies in the subject.

In some embodiments, the method results in an increase in hMPV-A-specific neutralizing antibodies in the subject of at least about 2-fold, about 3-fold, at least about 4-fold, at least about 5-fold, at least about 10-fold, at least about 15-fold, at least about 20-fold, or at least about 25-fold compared to baseline.

In some embodiments, the increase in hMPV-A-specific neutralizing antibodies is detectable within about one week, within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the pharmaceutical composition.

In some embodiments, the method results in the production of hMPV-B-specific neutralizing antibodies in the subject.

In some embodiments, the method results in an increase in hMPV-B-specific neutralizing antibodies in the subject of at least about 2-fold, about 3-fold, at least about 4-fold, at least about 5-fold, at least about 10-fold, at least about 15-fold, at least about 20-fold, or at least about 25-fold compared to baseline.

In some embodiments, the increase in hMPV-B-specific neutralizing antibodies is detectable within about one week, within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the pharmaceutical composition.

In some embodiments, the method prevents a severe Lower Respiratory Tract Infection (LRTI).

A pre-filled syringe is provided comprising a composition according to the present disclosure.

A kit is provided comprising a composition according to the present disclosure or comprising the pre-filled syringe of the present disclosure.

The present disclosure provides a kit, comprising one or more of:
a composition comprising a virus-like particle (VLP) comprising a first component comprising a respiratory syntactical virus (RSV) F protein ectodomain or antigenic variant thereof,
a composition comprising a second virus-like particle (VLP) comprising a first component comprising a human metapneumovirus (hMPV) F protein ectodomain or antigenic variant thereof, and
a composition comprising a virus-like particle (VLP) comprising a plurality of first components, some first components comprising a respiratory syntactical virus (RSV) F protein ectodomain or antigenic variant thereof and some first components comprising a human metapneumovirus (hMPV) F protein ectodomain or antigenic variant thereof.

In some embodiments, the kit comprises a composition comprising an adjuvant to be combined with the one or more VLP compositions prior to administration to a subject.

### BRIEF DESCRIPTION OF THE DRAWINGS

FIG. 1A shows an illustrative embodiment of a protein-based virus-like particle (VLP) according to the present disclosure.

FIG. 1B shows further illustrative embodiments of VLPs and VLP components (with F protein not shown).

FIG. 2 shows a secondary structure map of hMPV F protein in the pre-fusion configuration (PDB ID: 5WB0; diagram generated at www.rcsb.org).

FIGS. 3A-3C is a series of plots showing antibody binding profiles to illustrative VLPs of the disclosure. Antibodies are specific for various forms of the hMPV F protein. MF14 binds pre- and postfusion forms of hMPV F protein at the site II epitope (FIG. 3A). MF16 binds pre- and postfusion forms of hMPV F protein at the site IV epitope (FIG. 3B). MPE8 binds the pre-fusion form of hMPV F protein at the site III epitope (FIG. 3C).

FIGS. 4A and 4B show the immunogenicity of hMPV F protein mutants on a 2-component virus-like particle (VLP) or expressed as a soluble protein (sol). Graphs show neutralizing antibody titers against hMPV-A (FIG. 4A) and hMPV-B (FIG. 4B) in serum samples collected on day 35 after two immunizations.

FIGS. 5A and 5B show the immunogenicity of hMPV008 VLPs and the corresponding soluble protein, CompA-hMPV008 under different dose and adjuvant conditions. Also shown is hMPV033 when adjuvanted with squalene emulsion (SE). Graphs show neutralizing antibody titers against hMPV-A (FIG. 5A) and hMPV-B (FIG. 5B) in serum samples collected on day 35 after two immunizations.

FIG. 6 shows the RSV neutralization titers of sera from mice immunized with monovalent, bivalent, or mosaic vaccine compositions. Mice immunized with only the hMPV monovalent vaccine (IVX-241) show low immune response to RSV. Monovalent RSV, bivalent RSV/hMPV, and mosaic RSV/hMPV groups exhibited high anti-RSV immune responses.

FIG. 7 shows the hMPV neutralization titers of sera from mice immunized with monovalent or bivalent vaccine compositions. Mice immunized with only the RSV monovalent vaccine (IVX-121) show low immune response to hMPV. Monovalent hMPV, bivalent RSV/hMPV, and mosaic RSV/hMPV groups exhibited high anti-hMPV immune responses.

FIGS. 8A-8F show the induction of immunogenicity and protective effect of bivalent RSV-hMPV VLP vaccine compared with monovalent RSV or hMPV VLP vaccines in cotton rat live-virus challenge model.

FIG. 8A shows titers of RSV-A neutralizing antibodies in sera from animals immunized with monovalent RSV (“IVX-121”) or bivalent (“IVX-A12”) VLPs.

FIG. 8B shows titers of hMPV-A neutralizing antibodies in sera from animals immunized with monovalent hMPV (“IVX-241”) or bivalent (“IVX-A12”) VLPs.

FIG. 8C shows titers of RSV-A virus in the lungs of animals immunized with monovalent RSV (“IVX-121”) or bivalent (“IVX-A12”) VLPs and then challenged with RSV virus.

FIG. 8D shows titers of hMPV-A virus in the lungs of animals immunized with monovalent hMPV (“IVX-241”) or bivalent (“IVX-A12”) VLPs and then challenged with hMPV virus.

FIG. 8E shows nasal titers of RSV-A virus in animals immunized with monovalent RSV (“IVX-121”) or bivalent (“IVX-A12”) VLPs and then challenged with RSV virus.

FIG. 8F shows nasal titers of hMPV-A virus in animals immunized with monovalent hMPV (“IVX-241”) or bivalent (“IVX-A12”) VLPs and then challenged with hMPV virus.

FIG. 9 is a series of plots showing the time-course induction of neutralizing antibody titers in mice in response to dosing with hMPV F protein VLPs formulated with adjuvants.

FIG. 10 is the study design for a Phase 1/1b randomized, observer-blinded, placebo-controlled study to evaluate IVX-121 administration in young and older adult subjects.

FIG. 11 shows a summary of safety data. There were no serious adverse events (SAEs), AEs of special interest (AESIs) or adverse events (AEs) leading to study withdrawal.

FIG. 12 shows graphs of solicited local and systemic adverse events within 7 days of a single dose, maximal severity (“alum”=500 μg/mL aluminum hydroxide). Unadjuvanted IVX-121 reactogenicity is mild in older adults with similar tolerability to placebo.

FIG. 13 shows a graph of RSV-A neutralizing antibodies (nAB). Geometric mean titer (GMT) is expressed in international units per milliliter (IU/mL). GMT of unadjuvanted IVX-121 are comparable in young and older adults.

FIG. 14 shows a graph of RSV-A neutralizing antibodies (nAB), unadjuvanted versus adjuvanted. Geometric mean titer (GMT) is expressed in international units per milliliter (IU/mL). Alum adjuvant had no beneficial effect in young and older adults.

FIG. 15 shows tables summarizing neutralizing and binding antibody data.

FIG. 16 shows a graph of RSV-A neutralizing antibodies (nAB), unadjuvanted, in young adult subjects (left panel) and older adult subjects (right panel) through Day 180. Geometric mean titer (GMT) is expressed in international units per milliliter (IU/mL). lower limit of quantitation (LloQ)=9.9; Geometric Mean Fold Rise (Day 28) from Baseline. Data reflect all subjects available at the time of this six-month analysis; individual figures may differ immaterially from initial interim readout.

FIG. 17 shows a graph of RSV-A neutralizing antibodies (nAB), unadjuvanted, in young adult subjects through Day 180. μg of total VLP and μg of RSV-A antigen are shown for each treatment dose. Percentage of retained titers between Study Day 180 and Study Day 28 shown for each treatment dose. Geometric mean titer (GMT) is expressed in international units per milliliter (IU/mL).

FIG. 18 shows a graph of RSV-A neutralizing antibodies (nAB), unadjuvanted, in older adult subjects through Day 180. μg of total VLP and μg of RSV-A antigen are shown for each treatment dose. Percentage of retained titers between Study Day 180 (M6) and Study Day 28 (M1) shown for each treatment dose Geometric mean titer (GMT) is expressed in international units per milliliter (IU/mL).

FIG. 19 shows a summary of safety data. There were no serious adverse events (SAEs), AEs of special interest (AESIs) or adverse events (AEs) leading to study withdrawal.

FIG. 20 shows a graph of RSV-A neutralizing antibodies (nAB), unadjuvanted (left panel), and RSV-B neutralizing antibodies (nAB), unadjuvanted (right panel), in older adult subjects comparing Day 28 and Day 180. Geometric mean titer (GMT) is expressed in international units per milliliter (IU/mL).

FIG. 21 is the study design for a Phase 1 randomized, observer-blinded, placebo-controlled study to evaluate a single administration of IVX-A12 in older adult subjects (aged 60-75). Dosage groups with total VLP content (top left panel). Each dose included both RSV and hMPV VLPs. VLP composition of low, medium, and high hMPV doses combined with consistent RSV VLP dosage (top right panel). Interim readout included safety and immunogenicity readouts through D28 (bottom panel).

FIG. 22A shows the demographics of trial participants. Demographic characteristics were similar between IVX-A12 and placebo recipients. A total of 141 subjects were enrolled, 140 dosed (safety analysis set) with 17 subjects excluded for protocol violations, leaving 123 subjects in per protocol analysis set. FIG. 22B shows a summary of safety data. As of D28, there were no serious adverse events (SAEs), AEs of special interest (AESIs) or adverse events (AEs) leading to study withdrawal. Severe unsolicited AE (unrelated to vaccine) of hypertension on Day 28 was onserved in subject with prior medical history; AE was resolved. Unsolicited AEs were collected to Day 28. Other AEs will be collected to Day 180.

FIG. 23A shows graphs of solicited local adverse events within 7 days of a single dose, maxmal severity. Unadjuvanted IVX-A12 reactogenicity is mild in older adults. Dosage group VLPs: 150 μg=75 μg RSV+75 μg hMPV; 225 μg=75 μg RSV+150 μg hMPV; 300 μg=75 μg RSV+225 μg hMPV.

FIG. 23B shows graphs of solicited systemic adverse events within 7 days of a single dose. Unadjuvanted and adjuvanted (MF59®) IVX-A12 shows similar tolerability to placebo. Dosage group VLPs: 150 μg=75 μg RSV+75 μg hMPV; 225 μg=75 μg RSV+150 μg hMPV; 300 μg=75 μg RSV+225 μg hMPV.

FIG. 24A shows a graph of RSV-A neutralizing antibodies (nAB). Per-protocol analysis set of participants. Geometric mean titer (GMT) is expressed in international units per milliliter (IU/mL). RSV-A LLoQ=9.4. MF59®: Seqirus Inc.'s proprietary adjuvant. GMFR: Geometric Mean Fold Rise from Baseline. VLPs: 150 μg=75 μg RSV+75 μg hMPV; 225 μg=75 μg RSV+150 μg hMPV; 300 μg=75 μg RSV+225 μg hMPV.

FIG. 24B shows a graph of RSV-B neutralizing antibodies (nAB). Per-protocol analysis set of participants. Geometric mean titer (GMT) is expressed in international units per milliliter (IU/mL).

RSV-B LLoQ=8.0. MF59@: Seqirus Inc.'s proprietary adjuvant. GMFR: Geometric Mean Fold Rise from Baseline. VLPs: 150 μg=75 μg RSV+75 μg hMPV; 225 μg=75 μg RSV+150 μg hMPV; 300 μg=75 μg RSV+225 μg hMPV.

FIG. 25A shows a graph of hMPV-A neutralizing antibodies (nAB). Geometric mean titer (GMT) is expressed in assay units. Per-protocol analysis set of participants. hMPV-A and hMPV-B LLoQ=4.0 log 2. GMFR: Geometric Mean Fold Rise from Baseline. VLPs: 150 μg=75 μg RSV+75 μg hMPV; 225 μg=75 μg RSV+150 μg hMPV; 300 μg=75 μg RSV+225 μg hMPV.

FIG. 25B shows a graph of hMPV-B neutralizing antibodies (nAB). Geometric mean titer (GMT) is expressed in assay units. Per-protocol analysis set of participants. hMPV-A and hMPV-B LLoQ=4.0 log 2. GMFR: Geometric Mean Fold Rise from Baseline. VLPs: 150 μg=75 μg RSV+75 μg hMPV; 225 μg=75 μg RSV+150 μg hMPV; 300 μg=75 μg RSV+225 μg hMPV.

FIG. 26 shows a pre-specified sub-analysis assessing subjects with lowest tertile baseline RSV-A and RSV-B titers. Data shown for RSV-A (FIG. 26A) and RSV-B (FIG. 26B) neutralizing antibodies (nAB) in subjects with lowest tertile baseline nAB titers. Data for adjuvanted and non-adjuvanted subjects is pooled by dose. Geometric mean titer (GMT) is expressed in international units per milliliter (IU/mL). Per-protocol analysis set of participants. RSV-A LLoQ=9.4. RSV-B LLoQ=8.0. GMFR: Geometric Mean Fold Rise from Baseline. Dosage group VLPs: 150 μg=75 μg RSV+75 μg hMPV; 225 μg=75 μg RSV+150 μg hMPV; 300 μg=75 μg RSV+225 μg hMPV. *Data pooled across adjuvanted and non-adjuvanted groups within a dose.

### DETAILED DESCRIPTION

Provided herein are compositions or pharmaceutical compositions comprising two or more VLPs displaying the ectodomains of the F proteins of diverse paramyxoviruses. For example, the disclosure provides compositions comprising two VLPs: a VLP having a component comprising a respiratory syntactical virus (RSV) F protein ectodomain or antigenic variant thereof, and a VLP having a component comprising a human metapneumovirus (hMPV) F protein ectodomain or antigenic variant thereof. These compositions may be used to vaccinate against RSV (e.g., RSV-A subtype and/or RSV-B subtype) and human metapneumovirus (hMPV; e.g., hMPV-A subtype and or hMPV-B subtype).

In particular, provided herein are protein-based virus-like particle (VLP) vaccines for respiratory syntactical virus (RSV) and human metapneumovirus (hMPV) in which the RSV F protein ectodomain and hMPV F protein ectodomain are each independently linked to, and thereby displayed on, a protein-based VLP, e.g., a designed VLP, e.g., a symmetric VLP. For example, the vaccine antigen may be a N-terminal fusion of the ectodomain of the RSV F protein or hMPV F protein to a protein having a multimerization domain for a one- or two-component de novo designed VLP, such as a two-component icosahedral VLP. Further provided are vaccine compositions, methods of manufacturing, and methods of use, e.g., immunizing a subject to generate a protective immune response or strengthen an existing immune response to RSV virus and/or hMPV virus.

In variations, the disclosure provides a “mosaic” VLP generated by mixing the components of the RSV and hMPV vaccine before assembly into VLPs, so that each VLP contains some components displaying the ectodomain of the RSV F proteins and some components displaying the ectodomain of the hMPV F proteins. Accordingly, the disclosure further provides a virus-like particle (VLP) comprising a plurality of first components, some first components comprising a respiratory syntactical virus (RSV) F protein ectodomain or antigenic variant thereof and some first components comprising a human metapneumovirus (hMPV) F protein ectodomain or antigenic variant thereof.

### Definitions

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

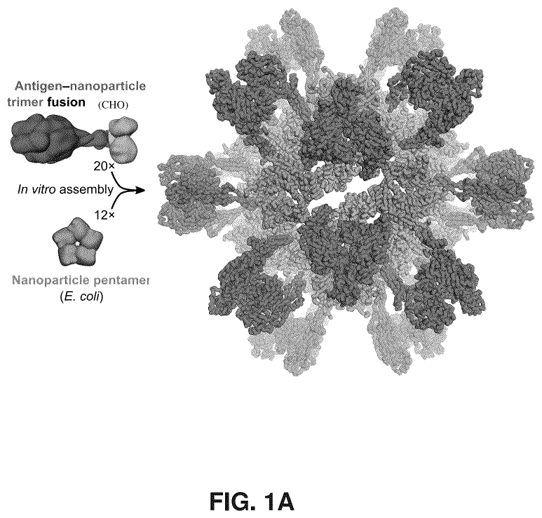

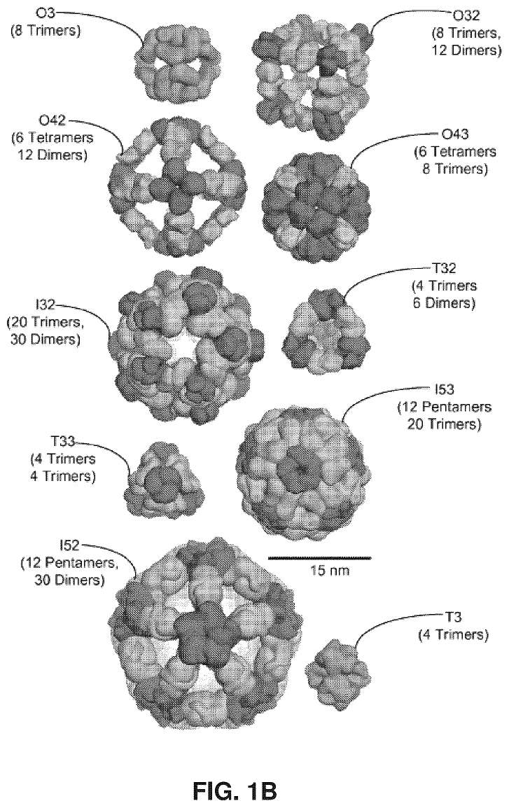

The term “virus-like particle” or “VLP” refers to a molecular assembly that resembles a virus but is non-infectious that displays an antigenic protein, or antigenic fragment thereof, of a viral protein or glycoprotein. A “protein-based VLP” refers to a VLP formed from proteins or glycoproteins and substantially free of other components (e.g., lipids). Protein-based VLPs may include post-translation modification and chemical modification, but are to be distinguished from micellar VLPs and VLPs formed by extraction of viral proteins from live or live inactivated virus preparations. The term “designed VLP” refers to a VLP comprising one or more polypeptides generated by computational protein design. Illustrative designed VLP are VLPs that comprise nanostructures depicted in FIG. 1B. The term “symmetric VLP” refers to a protein-based VLP with a symmetric core, such as shown in FIG. 1B. These include but are not limited to designed VLPs. For example, the protein ferritin has been used to generate a symmetric, protein-based VLP using naturally occurring ferritin sequences. Ferritin-based VLPs are distinguished from designed VLPs in that no protein engineering is necessary to form a symmetric VLP from ferritin, other than fusing the viral protein to the ferritin molecule. Protein design methods can be used to generate similar one- and two-component nanostructures based on template structures (e.g., structures deposited in the Protein Data Bank) or de novo (i.e., by computational design of new proteins having a desired structure but little or no homology to naturally occurring proteins). Such one- and two-component nanostructures can then be used as the core of a designed VLP.

The VLP of the present disclosure display antigens capable of eliciting immune responses to paramyxoviruses, including respiratory syncicial virus (RSV) and human metapneumovirus (hMPV) or other metapneumoviruses. As used herein and throughout the present disclosure, immune response(s) include generating an immune response or strengthening an existing immune response. The vaccines of the present disclosure are useful for preventing and/or decreasing the severity of infection with respiratory syncicial virus and/or human metapneumovirus.

The term “icosahedral particle” refers to a designed VLP having a core with icosahedral symmetry (e.g., the particles labeled 153 and 152 in Table 1). 153 refers to an icosahedral particle constructed from pentamers and trimers. 152 refers to an icosahedral particle constructed from pentamers and dimers. T33 refers to a tetrahedral particle constructed from two sets of trimers. T32 refers to a tetrahedral particle constructed from trimers and dimers.

The antigens may be attached to the core of the protein-based VLP either non-covalently or covalently, including as a fusion protein or by other means disclosed herein. Multimeric antigens may optionally be displayed along a symmetry axis of the VLP. Also provided are proteins and nucleic acid molecules encoding such proteins, formulations, and methods of use.

The term “antigen” refers to a polypeptide or polypeptide complex including at least one component designed to elicit an immune response. The term antigen, as used herein, is not limited to the portion of the polypeptide or polypeptide complex that contains antigenic epitopes.

The term “polypeptide” refers to a series of amino acid residues joined by peptide bonds and optionally one or more post-translational modifications (e.g., glycosylation) and/or other modifications (including but not limited to conjugation of the polypeptide moiety used as a marker-such as a fluorescent tag-or an adjuvant).

The term “infection” refers to both symptomatic and asymptomatic infections.

The term “ectodomain” refers to the portion of a transmembrane protein or glycoprotein that, in the native state of the protein, is on the outside of the cellular or viral membrane.

The term “variant” refers to a polypeptide having one or more insertions, deletions, replacements, or amino acid substitutions relative to a reference polypeptide, but retains one or more properties of the reference protein.

The term “antigenic variant” refers to a variant that has one or more epitopes in common with a reference polypeptide and/or generates the same or similar immune response when administered to a subject as a reference polypeptide.

The term “functional variant” refers to a variant that exhibits the same or similar functional effect(s) as a reference polypeptide. For example, a functional variant of a multimerization domain is able to promote multimerization to the same extent, or to similar extent, as a reference multimerization domain and/or is able to multimerize with the same cognate multimerization domains as a reference multimerization domain.

The term “linker” refers to either chemical linkage (i.e., a covalent bond or series of covalent bonds with intervening chemical moieties) or to a polypeptide that is N-terminally and C-terminally joined by peptide bonds to product a fusion protein.

The term “domain” refers to refers to any portion of a polypeptide that adopts a tertiary structure.

The terms “multimerization domain” and “multimerize” refer to the ability of a polypeptide, or domain of a polypeptide, to form dimers, trimers, tetramers, pentamers, or hexamers and/or to form heteromers with other multimerization domains.

The term “trimerization domain” refers to a multimerization domain that forms trimers.

The term “VLP-forming domain” refers to a multimerization domain that, alone or with other multimerization domains, forms a symmetric protein complex.

The term “fragment” refers to a polypeptide having one or more N-terminal or C-terminal truncations compared to a reference polypeptide.

The term “functional fragment” refers to a functional variant of a fragment.

The term “substitution” or “replacement” refers to replacing a single amino acid in a sequence with another amino acid residue. The standard form of abbreviations for amino acid substitution are used. For example, V94R refers to substitution of valine (V) in a reference sequence with arginine (R). The abbreviation Arg94 refers to any sequence in which the 94th residue, relative to a reference sequence, is arginine (Arg).

The terms “helix” or “helical” refer to an α-helical secondary structure in a polypeptide that is known to occur, or predicted to occur. For example, a sequence may be described as helical when computational modeling suggests the sequence is likely to adopt a helical conformation.

The term “component” refers to a protein, or protein complex, capable of assembly into a virus-like particle under appropriate conditions (e.g., an antigen or polypeptide comprising a multimerization domain).

The term “vaccine” refers to a pharmaceutical composition capable of use in producing an immune response or strengthening an existing immune response in a subject.

The term “pharmaceutically acceptable excipients” means excipients biologically or pharmacologically compatible for in vivo use in animals or humans, and can mean excipients approved by a regulatory agency of the Federal or a state government or listed in the U.S. Pharmacopeia or other generally recognized pharmacopeia for use in animals, and more particularly in humans.

The term “adjuvants” refers to a pharmaceutically acceptable substance that enhances the immune response to an antigen when co-administered with the antigen or administered before, during, or after administration of the antigen to a subject.

The term “TLR4 immunostimulant” refers to an adjuvant that stimulates Toll-like Receptor 4 (TLR4) in the immune cells of a subject to modulate an immune response e.g., Monophosphoryl Lipid A (MPL), Glucopyranosyl Lipid A (GLA), and/or Soluble Leishmania Antigen (SLA).

The term “effective amount” refers to the amount of a formulation according to the invention that, when administered to a patient for treating a state, disorder or condition is sufficient to effect such treatment or when administered to a patient for generating an immune response is sufficient to generate such an immune response, such as antibody response. The “effective amount” will vary depending on the active ingredient, the state, disorder, or condition to be treated and its severity, and the age, weight, physical condition and responsiveness of the subject to be treated.

This amount can vary depending upon the health and physical condition of the individual to be treated, their age, the capacity of the individual's immune system to generate antibodies, the degree of protection desired, the formulation of the vaccine, and other relevant factors.

As used herein, an “effective amount” refers to an amount of the immunogenic composition that is effective for treating and/or limiting RSV infection (e.g., RSV-A infection and/or RSV-B infection) and/or hMPV infection (e.g., hMPV-A infection and/or hMPV-B infection).

The term “immune response” refers to elicitation of activity of one or more immune cell types in the subject. Immune responses include, for example, T cell and B cell responses.

The term “humoral immune response” refers to an immune response that generates plasma or serum antibodies (e.g., IgG).

The term “protective immune response” refers to an immune response that prevents and/or reduces the severity of infection with a pathogen when the subject is later challenged with the pathogen, or to an immune response that generates a level of immune response that correlates with protection. For example, vaccination may generate a protective immune response if it results in production, in the plasma or serum, of the subject (e.g., human, pet, or agricultural animal), of neutralizing antibodies that protect the subject against subsequent infection and/or are present in a quantity observed to confer protection upon test subjects (e.g., New Zealand White (NZW) rabbits).

The term “polyclonal antibody response” refers to an antibody response comprising antibodies having more than one specificities and/or variation in their antibody sequences.

The term “neutralizing” (e.g., “neutralizing antibody response”) refers to antibodies that prevent infection and/or reduce the level of infection by a pathogen. A neutralizing antibody response can be measured either in in vitro assays (e.g., infection of cells in culture by a pathogen in the presence of the antibody) or in an in vivo assay (e.g., by determining a protective dose of an antibody through administering the antibody to a subject prior to challenge with an infective dose of a pathogen).

An antibody “binds to” or is “specific to” or “specifically binds” (used interchangeably herein) to a target (e.g., an RSV or hMPV F protein) are terms well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. For example, an immunoglobulin that specifically or preferentially binds to thymocyte is an immunoglobulin that binds thymocytes with greater affinity, avidity, more readily, and/or with greater duration than it binds to other cells. An immunoglobulin that specifically binds to a first cell or substance may or may not specifically or preferentially bind to a second cell or substance. As such, “specific binding” does not necessarily require (although it can include) exclusive binding. Generally, but not necessarily, reference to binding means specific binding.

The term “predetermined time” refers to an interval of time selected as appropriate for observing a particular effect. A predetermined time may be selected before, or during, an experiment or procedure.

The term “post-exposure prophylaxis” refers to administering an antigenic composition (e.g., a vaccine) to a subject previously exposed to and/or infected with a pathogen in order to elicit an immune response to protect against infection by the pathogen and/or decrease the severity of one or more symptoms of infection by the pathogen.

The term “administering” refers to providing a composition to a subject in a manner that permits the composition to have its intended effect. Administration for vaccination or post-exposure prophylaxis may be performed by intramuscular injection, intravenous injection, intraperitoneal injection, or any other suitable route.

The terms “immunization” and “immunizing” refer to administering a composition to a subject (e.g., a virus-like particle) in an amount sufficient to elicit, after one or more administering steps, a desired immune response (e.g., a humoral immune response to the VLP). Immunization may comprise between one and ten, or more administrations (e.g., injections) of the composition, such as 1, 2, 3, 4, 5, 6, 7, 8, 9, 10, or more administrations. The first administration may elicit no detectable immune response as generally each subsequent administration will boost the immune response generated by prior administrations. The term “immunizing” as used herein includes post-exposure prophylaxis.

The term “subject” refers to a human or non-human animal to which a composition may be administered for vaccination, treatment, or other purpose. In some embodiments, the non-human animal is a non-human primate including, but not limited to, rabbit, hamster, gerbil, pig, cow, sheep, goat, guinea pig, rat, mouse, squirrel, wolf, fox, horse, zebra, giraffe, elephant, cat, dog, llama, or ferret.

The term “manufacturing” refers to production of a recombinant polypeptide or virus-like particle at any scale, including but not limited to at least 25-mL, 50-mL, 1-L, 1,000-L, 50,000-L, or greater scale.

The terms “culturing” and “culture medium” refers to standard cell culture and recombinant protein expression techniques.

The term “host cell” refers to any cell capable of use in expression of a recombinant polypeptide.

The term “secretes” refers to the ability of host cells to secrete polypeptides into the media in which they are cultured.

The term “signal sequence” refers to a polypeptide sequence, typically at the N terminus of a polypeptide expressed in a host cell that directs the polypeptide to a particular cellular compartment. A signal sequence may be a secretion signal to cause the host cell to secrete the polypeptide into the media in which with host cell is cultured. Various signal sequences are known and it is within the skill of an ordinary artisan to select an appropriate signal sequence.

The term “mixing” refers to placing two solutions into contact to permit the solutions to mix.

The term “purify” refers to separating a molecule from other substances present in a composition. Polypeptides may be purified by affinity (e.g., to an antibody or to a tag, e.g., using a His-tag capture resin), by charge (e.g., ion-exchange chromatography), by size (e.g., preparative ultracentrifugation, size exclusion chromatography), or otherwise.

The terms “polynucleotide” and “nucleic acid,” used interchangeably herein, refer to a polymeric form of nucleotides of more than about 100 nucleotides, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” and “nucleic acid” should be understood to include, as applicable to the embodiments being described, single-stranded (such as sense or antisense) and double-stranded polynucleotides.

The terms “identical,” “identity,” “percent identity,” “sequence identity,” or “percent sequence identity,” in the context of two or more nucleic acid or polypeptide sequences, refers to two or more sequences or subsequences that are the same or have a specified percentage of amino acid residues or nucleotides that are the same, when compared and aligned for maximum correspondence. Methods of alignment of sequences for comparison are well known in the art. Once aligned, the number of matches is determined by counting the number of positions where an identical nucleotide or amino acid residue is present in both sequences. The percent sequence identity is determined by dividing the number of matches in the alignment by the length of the reference sequence, followed by multiplying the resulting value by 100. For example, a peptide sequence that has 1166 matches when aligned with a test sequence having 1554 amino acids is 75.0 percent identical to the test sequence (1166÷1554*100=75.0). As the terms are used herein, gaps in the alignment do not decrease the percent sequence identity.

Unless otherwise specified, optimal alignment of sequences for comparison is conducted by the global alignment algorithm of Needleman and Wunsch, Mol. Biol. 48:443 (1970) as implemented by EMBOSS Needle (on the World Wide Web at ebi.ac.uk/Tools/psa/emboss_needle/) (Madeira et al. Nucleic Acids Res. 50(W1):W276-W279 (2022)). In embodiments, other alignment methods may be used, including without limitation those described in Devereux, et al, Nucleic Acids Res. 12:387-95 (1984); Atschul et al. J. Mo. Biol. 215:403-10 (1990) (BLAST); Carrillo and Lipman Siam J. Appl. Math. 48(5) (1988); Computational Molecular Biology (Lesk, A M, ed., 1989); Biocomputing Informatics and Genome Projects, (Smith, D W, ed., 1993); Computer Analysis of Sequence Data, Part I, (Griffin and Griffin, eds., 1994); Sequence Analysis in Molecular Biology (von Heinje, 2012); Sequence Analysis Primer (Gribskov and Devereux, J., eds. 1993). In embodiments, sequence identity is calculated using the implementation of the Needleman-Wunsch algorithm provided by the National Library of Medicine (on the World Wide Web at blast.ncbi.nlm.nih.gov/Blast.cgi?PAGE_TYPE=BlastSearch&BLAST_SPEC=GlobalAln).

For example, sequence identity can be determined by standard methods that are commonly used to compare the similarity of two polypeptide or two polynucleotide sequences. Using a computer program such as EMBOSS Needle or BLAST, two polypeptide or two polynucleotide sequences are aligned for optimal matching of their respective residues (either along the full length of one or both sequences, or along a pre-determined portion of one or both sequences). The programs provide a default opening penalty and a default gap penalty, and a scoring matrix such as PAM 250 (a standard scoring matrix; see Dayhoff et al., in Atlas of Protein Sequence and Structure, vol. 5, supp. 3 (1978)) that can be used in conjunction with the computer program.

The term “treating” means one or more of relieving, alleviating, delaying, reducing, reversing, improving, or managing at least one symptom of a condition in a subject. The term “treating” may also mean one or more of arresting, delaying the onset (i.e., the period prior to clinical manifestation of the condition) or reducing the risk of developing or worsening a condition.

The term “a” or “an” refers to one or more of that entity, i.e. can refer to plural referents. As such, the terms “a,” “an,” “one or more,” and “at least one” are used interchangeably herein. In addition, reference to “an element” by the indefinite article “a” or “an” does not exclude the possibility that more than one of the elements is present, unless the context clearly requires that there is one and only one of the elements.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation. Alternatively, “about” can mean plus or minus a range of, for example, up to 20%, up to 10%, or up to 5%. When used in conjunction with a range or series of values, the term “about” applies to the endpoints of the range or each of the values enumerated in the series, unless otherwise indicated. As used in this application, the terms “about” and “approximately” are used as equivalents.

All weight percentages (i.e., “% by weight” and “wt. %” and w/w) referenced herein, unless otherwise indicated, are measured relative to the total weight of the pharmaceutical composition.

As used herein, “substantially” or “substantial” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking, the nearness of completion will be so as to have the same overall result as if absolute and total completion were obtained. The use of “substantially” is equally applicable when used in a negative connotation to refer to the complete or near complete lack of action, characteristic, property, state, structure, item, or result. For example, a composition that is “substantially free of” other active agents would either completely lack other active agents, or so nearly completely lack other active agents that the effect would be the same as if it completely lacked other active agents. In other words, a composition that is “substantially free of” an ingredient or element or another active agent may still contain such an item as long as there is no measurable effect thereof.

The following description includes information that may be useful in understanding the present invention. It is not an admission that any of the information provided herein is prior art or relevant to the presently claimed inventions, or that any publication specifically or implicitly referenced is prior art.

### Overview

Vaccination is a treatment modality used to prevent or decrease the severity of infection with various infectious agents, including bacteria, viruses, and parasites. Development of new vaccines has important commercial and public health implications. In particular, lyme disease, pertussis, herpes virus, orthomyxovirus, paramyxovirus, pneumovirus, filovirus, flavivirus, reovirus, retrovirus, coronavirus, and malaria are infectious agents for which vaccines already exist, are being developed, or would be desirable.

Subunit vaccines are vaccines made from isolated antigens, usually proteins expressed recombinantly in bacterial, insect, or mammalian cell hosts. Typically, the antigenic component of a subunit vaccine is selected from among the proteins of an infectious agent observed to elicit a natural immune response upon infection, although in some cases other components of the infectious agent can be used. Typical antigens for use in subunit vaccines include protein expressed on the surface of the target infectious agent, as such surface-expressed envelope glycoproteins of viruses. Preferably, the antigen is a target for neutralizing antibodies. More preferably, the antigen is a target for broadly neutralizing antibodies, such that the immune response to the antigen covers immunity against multiple strains of the infectious agent. In some cases, glycans that are N-linked or O-linked to the subunit vaccine may also be important in vaccination, either by contributing to the epitope of the antigen or by guiding the immune response to particular epitopes on the antigen by steric hindrance. The immune response that occurs in response to vaccination may be direct to the protein itself, to the glycan, or to both the protein and linked glycans. Subunit vaccines have various advantages including that they contain no live pathogen, which eliminates concerns about infection of the patient by the vaccine; they may be designed using standard genetic engineering techniques; they are more homogenous than other forms of vaccine; and they can be manufactured in standardized recombinant protein expression production systems using well-characterized expression systems. In some cases, the antigen may be genetically engineered to favor generation of desirable antibodies, such as neutralizing or broadly neutralizing antibodies. In particular, structural information about an antigen of interest, obtained by X-ray crystallography, electron microscopy, or nuclear magnetic resonance experiments, can be used to guide rational design of subunit vaccines.

A known limitation of subunit vaccines is that the immune response elicited may sometimes be weaker than the immune response to other types of vaccines, such as whole virus, live, or live-attenuated vaccines. The present inventors have recognized and herein disclose that designed and/or protein-based VLP vaccines have the potential to harness the advantages of subunit vaccines while increasing the potency and breadth of the vaccine-induced immune response through multivalent display of the antigen in symmetrically ordered arrays. In the present disclosure, protein-based VLPs are distinguished from nanoparticle vaccines, because the term nanoparticle vaccine has been used in the art to refer to protein-based or glycoprotein-based vaccines (see, e.g. Patent No. U.S. Pat. No. 9,441,019), polymerized liposomes (see, e.g., U.S. Pat. No. 7,285,289), surfactant micelles (see, e.g., US Patent Pub. No. US 2004/0038406 A1), and synthetic biodegradable particles (see, e.g., U.S. Pat. No. 8,323,696).

The protein-based VLPs of the present disclosure are distinguishable from VLPs that display an antigen on the surface of a micelle particle containing a surfactant (e.g., NP-9); or alternatively, made by extracting antigenic proteins from live virus while retaining lipid constituents of the viral envelope. By contrast, the protein-based VLPs described herein are free of, or substantially free of, lipid and surfactants. Furthermore, the symmetric display of the RSV and/or hMPV F protein on some embodiments of the protein-based VLPs of this disclosure may generate superior immune response to the F protein compared to other VLPs.

The disclosure relates, in part, to combination vaccines for paramyxovirus, such as RSV and hMPV, such as bivalent (or multivalent) vaccines having two (or more) VLPs that display paramyxovirus antigens, such as the ectodomain of the F protein of each paramyxovirus. In some variations, the VLP is a mosaic VLP displaying multiple antigens. In further variations, the bivalent displays antigens from two strains of the same paramyxovirus (such as RSV-A nad RSV-B, or hMPV-A and hMPV-B). In further variations, the multivalent vaccine includes VLPs displaying antigens from three or more paramyxoviruses, such as RSV-A, RSV-B, hMPV-A, and hMPV-B.

In some embodiments, the F protein(s) of the respective paramyxovirus(es) may be engineered to preferentially adopt a pre-fusion conformation. Accordingly, the disclosure provides pre-fusion-stabilized hMPV F protein ectodomains and pre-fusion-stabilized RSV F protein ectodomains for use in VLPs.

### Metapneumovirus Virus-Like Particle

Further provided are protein-based virus-like particle (VLP) vaccines for human metapneumovirus in which the hMPV F protein ectodomain is linked to, and thereby displayed on, a protein-based VLP.

In one aspect, the disclosure provides a virus-like particle (VLP), comprising a first component and optionally a second component, wherein the first component is a fusion protein, comprising a human metapneumovirus (hMPV) F protein ectodomain or antigenic variant thereof, and a first multimerization domain; and wherein the second component, if present, is a protein comprising a second multimerization domain.

In some embodiments, first multimerization domain is a trimerization domain. In some embodiments, the multimerization domain is selected from SEQ ID NOS: 1, 4, 5, 7, 9, 18, 19, 21, 24, 25, 26, 29, 30, 31, 34, 36, 37, 39, 42, 43, 44, 45, 46, 47, 48, 49, 50, 51, 52, 53, 144, or 145, or functional variants thereof. In some embodiments, the multimerization domain is I53-50A (SEQ ID NO: 7 or SEQ ID NO: 144) or a functional variant or variant thereof. In some embodiments, the first multimerization domain shares at least 70%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 7, SEQ ID NO: 53, SEQ ID NO: 144, or SEQ ID NO: 145. In some embodiments, the first multimerization domain comprises: the amino acid substitutions C74A and C98A; the amino acid substitutions C163A and C201A; or the amino acid substitutions C74A, C98A, C163A, and C201A relative to SEQ ID NO: 144. In some embodiments, the first multimerization domain has a polypeptide sequence identical to SEQ ID NO: 7 or SEQ ID NO: 144. In some embodiments, the first multimerization domain has a polypeptide sequence identical to SEQ ID NO: 53 or SEQ ID NO: 145. In some embodiments, the first multimerization domain is a VLP-forming domain. In some embodiments, the first multimerization domain is adapted to drive assembly of an icosahedral particle or tetrahedral particle, optionally with a second multimerization domain.

In some embodiments, second multimerization domain is selected from SEQ ID NOs: 2, 3, 6, 8, 10, 11, 12, 13, 14, 15, 16, 17, 20, 22, 23, 27, 28, 32, 33, 35, 38, 40, and 41 or functional variants and fragments thereof. In some embodiments, the second multimerization domain is I53-50B (SEQ ID NO: 8), I53-50B.4PosT1 (SEQ ID NO: 34), or a functional variant thereof.

In some embodiments, the first component and the second component are joined by a linker sequence. In some embodiments, the linker sequence comprises a foldon, wherein the foldon sequence is EKAAKAEEAARK (SEQ ID NO: 125). In some embodiments, the linker sequence comprises GSGGSGSGSGGS (SEQ ID NO: 126).

In some embodiments, the hMPV F protein ectodomain contains one or more substitutions or deletions selected from A185P, Q100R, S101R, T127C, N153C, V84C, A140C, A147C, A249C, N97G, P98G, R99G, Q100G, S101G, R102G, A63C, K188C, K450C, S470C, G106 deletion, A113C, A120C, A339C, Q426C, T160F, Q100K, S101A, I177L, K450A, S470A, G294E, T365C, V463C, L219K, H368N, and/or V231I. In some embodiments, the hMPV F protein ectodomain contains one or more substitutions or deletions selected from V84C, A140C, A147C, N97G, P98G, Q100G, S101G, R102G, A63C, K188C, K450C, S470C, R99G, A113C, A120C, A339C, Q426C, T160F, Q100K, S101A, Q100R, S101R, G106 deletion, S101R, A185P, I177L, and/or G294E.

In some embodiments, the hMPV F protein ectodomain contains two or more substitutions or deletions selected from V84C, A140C, A147C, N97G, P98G, Q100G, S101G, R102G, A63C, K188C, K450C, S470C, R99G, A113C, A120C, A339C, Q426C, T160F, Q100K, S101A, Q100R, S101R, G106 deletion, S101R, A185P, I177L, and/or G294E. In some embodiments, the hMPV F protein ectodomain contains one or more substitutions selected from A185P, Q100R, S101R, T127C, N153C, T365C, V463C, L219K, V231I, G294E, N153C, N97G, P98G, R99G, Q100G, S101G, H368N, and/or R102G.

In some embodiments, the hMPV F protein ectodomain contains two or more of A185P, Q100R, S101R, T127C, N153C, T365C, V463C, L219K, V231I, G294E, N153C, N97G, P98G, R99G, Q100G, S101G, H368N, and/orR102G. In some embodiments, the hMPV F protein ectodomain comprises: the substitutions Q100R and S101R; the substitutions A185P, Q100R, and S101R; the substitutions A185P, T127C, N153C, Q100R, and S101R; the substitutions V84C, A140C, A147C, A249C, N97G, P98G, R99G, Q100G, S101G, and R102G; the substitutions A63C, A140C, A147C, K188C, K450C, S470C, N97G, P98G, R99G, Q100G, S101G, and R102G; the substitutions and deletion A63C, A140C, A147C, K188C, G106 deletion, N97G, P98G, R99G, Q100G, S101G, and R102G; the substitutions A113C, A120C, A339C, Q426C, T160F, I177L, Q100K, and S101A; the substitutions V84C, A140C, A147C, A249C, Q100R, and S101R; the substitutions A63C, A140C, A147C, K188C, K450C, S470C, Q100R, and S101R; the substitutions and deletion A63C, A140C, A147C, K188C, G106 deletion, Q100R, and S101R; the substitutions A113C, A120C, A339C, Q426C, T160F, I177L, Q100R, and S101R; the substitutions A185P, A113C, A339C, Q100R, and S101R; the substitutions A185P, T160F, I177L, Q100R, and S101R; the substitutions A185P, A113C, A339C, T160F, I177L, Q100R, and S101R; the substitutions A63C, K188C, N97G, P98G, R99G, Q100G, S101G, and R102G; the substitutions A63C, K188C, K450A, S470A, N97G, P98G, R99G, Q100G, S101G, and R102G; the substitutions A63C, A140C, A147C, K188C, G294E, N97G, P98G, R99G, Q100G, S101G, and R102G; the substitutions A63C, A140C, A147C, K188C, K450C, S470C, N97G, P98G, R99G, Q100G, S101G, R102G, and G294E; the substitutions A63C, K188C, N97G, P98G, R99G, Q100G, S101G, R102G, and G294E; the substitutions T127C, N153C, T365C, V463C, A185P, L219K, V231I, G294E, H368N, Q100R, and S101R; the substitutions A63C, A140C, A147C, K188C, K450C, S470C, N97G, P98G, R99G, Q100G, S101G, and R102G; the substitutions V84C, A140C, A147C, A249C, N97G, P98G, R99G, Q100G, S101G, and R102G; the substitutions T127C, N153C, T365C, V463C, A185P, L219K, V231I, G294E, H368N, N97G, P98G, R99G, Q100G, S101G, and R102G.

In some embodiments, the hMPV F protein ectodomain comprises a sequence that shares at least 85%, at least 90%, at least 91%, at least 92%, at least 93%, at least 94%, at least 95%, at least 96%, at least 97%, at least 98%, at least 99%, or 100% identity to SEQ ID NOs: 58. In some embodiments, the hMPV F protein ectodomain comprises a sequence that shares at least 85%, at least 90%, at least 91%, at least 92%, at least 93%, at least 94%, at least 95%, at least 96%, at least 97%, at least 98%, at least 99%, or 100% identity to SEQ ID NO: 59. In some embodiments, the hMPV F protein ectodomain comprises a sequence that shares at least 85%, at least 90%, at least 91%, at least 92%, at least 93%, at least 94%, at least 95%, at least 96%, at least 97%, at least 98%, at least 99%, or 100% identity to SEQ ID NO: 60. In some embodiments, the hMPV F protein ectodomain comprises a sequence that shares at least 85%, at least 90%, at least 91%, at least 92%, at least 93%, at least 94%, at least 95%, at least 96%, at least 97%, at least 98%, at least 99%, or 100% identity to SEQ ID NO: 61. In some embodiments, the hMPV F protein ectodomain comprises a sequence that shares at least 85%, at least 90%, at least 91%, at least 92%, at least 93%, at least 94%, at least 95%, at least 96%, at least 97%, at least 98%, at least 99%, or 100% identity to SEQ ID NO: 62. In some embodiments, the hMPV F protein ectodomain comprises a sequence that shares at least 85%, at least 90%, at least 91%, at least 92%, at least 93%, at least 94%, at least 95%, at least 96%, at least 97%, at least 98%, at least 99%, or 100% identity to SEQ ID NO: 63. In some embodiments, the hMPV F protein ectodomain comprises a sequence that shares at least 85%, at least 90%, at least 91%, at least 92%, at least 93%, at least 94%, at least 95%, at least 96%, at least 97%, at least 98%, at least 99%, or 100% identity to SEQ ID NO: 64.

In some embodiments, the hMPV F protein ectodomain comprises a sequence that shares at least 85%, at least 90%, at least 91%, at least 92%, at least 93%, at least 94%, at least 95%, at least 96%, at least 97%, at least 98%, at least 99%, or 100% identity to SEQ ID NO: 65. In some embodiments, the hMPV F protein ectodomain comprises a sequence that shares at least 85%, at least 90%, at least 91%, at least 92%, at least 93%, at least 94%, at least 95%, at least 96%, at least 97%, at least 98%, at least 99%, or 100% identity to SEQ ID NO: 66. In some embodiments, the hMPV F protein ectodomain comprises a sequence that shares at least 85%, at least 90%, at least 91%, at least 92%, at least 93%, at least 94%, at least 95%, at least 96%, at least 97%, at least 98%, at least 99%, or 100% identity to SEQ ID NO: 67. In some embodiments, the hMPV F protein ectodomain comprises a sequence that shares at least 85%, at least 90%, at least 91%, at least 92%, at least 93%, at least 94%, at least 95%, at least 96%, at least 97%, at least 98%, at least 99%, or 100% identity to SEQ ID NO: 68. In some embodiments, the hMPV F protein ectodomain comprises a sequence that shares at least 85%, at least 90%, at least 91%, at least 92%, at least 93%, at least 94%, at least 95%, at least 96%, at least 97%, at least 98%, at least 99%, or 100% identity to SEQ ID NO: 69. In some embodiments, the hMPV F protein ectodomain comprises a sequence that shares at least 85%, at least 90%, at least 91%, at least 92%, at least 93%, at least 94%, at least 95%, at least 96%, at least 97%, at least 98%, at least 99%, or 100% identity to SEQ ID NO: 70. In some embodiments, the hMPV F protein ectodomain comprises a sequence that shares at least 85%, at least 90%, at least 91%, at least 92%, at least 93%, at least 94%, at least 95%, at least 96%, at least 97%, at least 98%, at least 99%, or 100% identity to SEQ ID NO: 71. In some embodiments, the hMPV F protein ectodomain comprises a sequence that shares at least 85%, at least 90%, at least 91%, at least 92%, at least 93%, at least 94%, at least 95%, at least 96%, at least 97%, at least 98%, at least 99%, or 100% identity to SEQ ID NO: 72. In some embodiments, the hMPV F protein ectodomain comprises a sequence that shares at least 85%, at least 90%, at least 91%, at least 92%, at least 93%, at least 94%, at least 95%, at least 96%, at least 97%, at least 98%, at least 99%, or 100% identity to SEQ ID NO: 73. In some embodiments, the hMPV F protein ectodomain comprises a sequence that shares at least 85%, at least 90%, at least 91%, at least 92%, at least 93%, at least 94%, at least 95%, at least 96%, at least 97%, at least 98%, at least 99%, or 100% identity to SEQ ID NO: 74. In some embodiments, the hMPV F protein ectodomain comprises a sequence that shares at least 85%, at least 90%, at least 91%, at least 92%, at least 93%, at least 94%, at least 95%, at least 96%, at least 97%, at least 98%, at least 99%, or 100% identity to SEQ ID NO: 75. In some embodiments, the hMPV F protein ectodomain comprises a sequence that shares at least 85%, at least 90%, at least 91%, at least 92%, at least 93%, at least 94%, at least 95%, at least 96%, at least 97%, at least 98%, at least 99%, or 100% identity to SEQ ID NO: 76. In some embodiments, the hMPV F protein ectodomain comprises a sequence that shares at least 85%, at least 90%, at least 91%, at least 92%, at least 93%, at least 94%, at least 95%, at least 96%, at least 97%, at least 98%, at least 99%, or 100% identity to SEQ ID NO: 77. In some embodiments, the hMPV F protein ectodomain comprises a sequence that shares at least 85%, at least 90%, at least 91%, at least 92%, at least 93%, at least 94%, at least 95%, at least 96%, at least 97%, at least 98%, at least 99%, or 100% identity to SEQ ID NO: 78. In some embodiments, the hMPV F protein ectodomain comprises a sequence that shares at least 85%, at least 90%, at least 91%, at least 92%, at least 93%, at least 94%, at least 95%, at least 96%, at least 97%, at least 98%, at least 99%, or 100% identity to SEQ ID NO: 79. In some embodiments, the hMPV F protein ectodomain comprises a sequence that shares at least 85%, at least 90%, at least 91%, at least 92%, at least 93%, at least 94%, at least 95%, at least 96%, at least 97%, at least 98%, at least 99%, or 100% identity to SEQ ID NO: 80. In some embodiments, the hMPV F protein ectodomain comprises a sequence that shares at least 85%, at least 90%, at least 91%, at least 92%, at least 93%, at least 94%, at least 95%, at least 96%, at least 97%, at least 98%, at least 99%, or 100% identity to SEQ ID NO: 81. In some embodiments, the hMPV F protein ectodomain comprises a sequence that shares at least 85%, at least 90%, at least 91%, at least 92%, at least 93%, at least 94%, at least 95%, at least 96%, at least 97%, at least 98%, at least 99%, or 100% identity to SEQ ID NO: 82. In some embodiments, the hMPV F protein ectodomain comprises a sequence that shares at least 85%, at least 90%, at least 91%, at least 92%, at least 93%, at least 94%, at least 95%, at least 96%, at least 97%, at least 98%, at least 99%, or 100% identity to SEQ ID NO: 83. In some embodiments, the hMPV F protein ectodomain comprises a sequence that shares at least 85%, at least 90%, at least 91%, at least 92%, at least 93%, at least 94%, at least 95%, at least 96%, at least 97%, at least 98%, at least 99%, or 100% identity to SEQ ID NO: 84. In some embodiments, the hMPV F protein ectodomain comprises a sequence that shares at least 85%, at least 90%, at least 91%, at least 92%, at least 93%, at least 94%, at least 95%, at least 96%, at least 97%, at least 98%, at least 99%, or 100% identity to SEQ ID NO: 85. In some embodiments, the hMPV F protein ectodomain comprises a sequence that shares at least 85%, at least 90%, at least 91%, at least 92%, at least 93%, at least 94%, at least 95%, at least 96%, at least 97%, at least 98%, at least 99%, or 100% identity to SEQ ID NO: 86. In some embodiments, the hMPV F protein ectodomain comprises a sequence that shares at least 85%, at least 90%, at least 91%, at least 92%, at least 93%, at least 94%, at least 95%, at least 96%, at least 97%, at least 98%, at least 99%, or 100% identity to SEQ ID NO: 87. In some embodiments, the hMPV F protein ectodomain comprises a sequence that shares at least 85%, at least 90%, at least 91%, at least 92%, at least 93%, at least 94%, at least 95%, at least 96%, at least 97%, at least 98%, at least 99%, or 100% identity to SEQ ID NO: 88. In some embodiments, the hMPV F protein ectodomain comprises a sequence that shares at least 85%, at least 90%, at least 91%, at least 92%, at least 93%, at least 94%, at least 95%, at least 96%, at least 97%, at least 98%, at least 99%, or 100% identity to SEQ ID NO: 89. In some embodiments, the hMPV F protein ectodomain comprises a sequence that shares at least 85%, at least 90%, at least 91%, at least 92%, at least 93%, at least 94%, at least 95%, at least 96%, at least 97%, at least 98%, at least 99%, or 100% identity to SEQ ID NO: 90. In some embodiments, the hMPV F protein ectodomain shares at least 85%, at least 90%, at least 91%, at least 92%, at least 93%, at least 94%, at least 95%, at least 96%, at least 97%, at least 98%, at least 99%, or 100% identity to SEQ ID NOs: 58, 60, 61, 77, 78, 86, 87, 88, or 89. In some embodiments, the hMPV F protein ectodomain shares at least 85%, at least 90%, at least 91%, at least 92%, at least 93%, at least 94%, at least 95%, at least 96%, at least 97%, at least 98%, at least 99%, or 100% identity to SEQ ID NOs: 59, 62, 63, 64, 71, 76, 81, or 83. In some embodiments, the hMPV F protein ectodomain shares at least 85%, at least 90%, at least 91%, at least 92%, at least 93%, at least 94%, at least 95%, at least 96%, at least 97%, at least 98%, at least 99%, or 100% identity to SEQ ID NOs: 72, 79, or 80. In some embodiments, the hMPV F protein ectodomain shares at least 85%, at least 90%, at least 91%, at least 92%, at least 93%, at least 94%, at least 95%, at least 96%, at least 97%, at least 98%, at least 99%, or 100% identity to SEQ ID NOs: 65, 66, 82, 84, 85, 90, or 91. In some embodiments, the hMPV F protein ectodomain shares at least 85%, at least 90%, at least 91%, at least 92%, at least 93%, at least 94%, at least 95%, at least 96%, at least 97%, at least 98%, at least 99%, or 100% identity to SEQ ID NOs: 65, 79, or 80. In some embodiments, the first component shares at least 85%, at least 90%, at least 91%, at least 92%, at least 93%, at least 94%, at least 95%, at least 96%, at least 97%, at least 98%, at least 99%, or 100% identity to SEQ ID NOs: 92-124. In some embodiments, the first component shares at least 85%, at least 90%, at least 91%, at least 92%, at least 93%, at least 94%, at least 95%, at least 96%, at least 97%, at least 98%, at least 99%, or 100% identity to SEQ ID NOs: 97-116, 119, or 122. In some embodiments, the first component shares at least 85%, at least 90%, at least 91%, at least 92%, at least 93%, at least 94%, at least 95%, at least 96%, at least 97%, at least 98%, at least 99%, or 100% identity to SEQ ID NOs: 92-94, 96, 117, 118, 120, 121, 123, or 124. In some embodiments, the first component shares at least 85%, at least 90%, at least 91%, at least 92%, at least 93%, at least 94%, at least 95%, at least 96%, at least 97%, at least 98%, at least 99%, or 100% identity to SEQ ID NOs: 99, 112, 117, or 118.

In some embodiments, the VLP binds one or more hMPV F protein antibodies selected from MF1, MF2, MF3, MF9, MF12, MF14, MF15, MF11, MF16, MF20, MF17, MF18, MF19, MF10, or MPE8. In some embodiments, the VLP binds two or more antibodies selected from MF1, MF2, MF3, MF9, MF12, MF14, MF15, MF11, MF16, MF20, MF17, MF18, MF19, MF10, MPE8. In some embodiments, the VLP binds three or more antibodies selected from MF1, MF2, MF3, MF9, MF12, MF14, MF15, MF11, MF16, MF20, MF17, MF18, MF19, MF10, MPE8.

In some embodiments, the hMPV F protein ectodomain binds an antibody that preferentially binds the prefusion form of the hMPV F protein ectodomain. In some embodiments, the first component shares at least 85%, at least 90%, at least 91%, at least 92%, at least 93%, at least 94%, at least 95%, at least 96%, at least 97%, at least 98%, at least 99%, or 100% identity to SEQ ID NOs: 42, 45, 58, 61, 63, or 64. In some embodiments, the hMPV F protein ectodomain has no or low binding to an antibody that preferentially binds the postfusion form of the hMPV F protein ectodomain. In some embodiments, the VLP sequence shares at least 85%, at least 90%, at least 91%, at least 92%, at least 93%, at least 94%, at least 95%, at least 96%, at least 97%, at least 98%, at least 99%, or 100% identity to SEQ ID NOs: 99, 115, 117, or 118.

In some embodiments, the first component shares at least 85%, at least 90%, at least 91%, at least 92%, at least 93%, at least 94%, at least 95%, at least 96%, at least 97%, at least 98%, at least 99%, or 100% identity with SEQ ID NO: 99.

In some embodiments, the first component is a single chain. In some embodiments, the single chain shares at least 85%, at least 90%, at least 91%, at least 92%, at least 93%, at least 94%, at least 95%, at least 96%, at least 97%, at least 98%, at least 99%, or 100% identity with SEQ ID NO: 123.

In some embodiments, the first component shares at least 85%, at least 90%, at least 91%, at least 92%, at least 93%, at least 94%, at least 95%, at least 96%, at least 97%, at least 98%, at least 99%, or 100% identity with SEQ ID NO: 117.

In one aspect, the disclosure provides a polynucleotide encoding the VLP described herein.

In one aspect, the disclosure provides a host cell, comprising the polynucleotide described herein.

### Respiratory Syntactical Virus Virus-Like Particle

In one aspect, provided herein is a pharmaceutical composition, comprising a Virus-like Particle (VLP) comprising a first component comprising an RSV F protein and a first multimerization domain, and a second component comprising a second multimerization domain; and one or more pharmaceutically acceptable diluents or excipients. In some embodiments, the VLP is an icosahedral VLP. In some embodiments, the VLP comprises 20 copies of the first component and 12 copies of the second component.

In some embodiments, the RSV F protein comprises an amino acid sequence which is at least 75%, 80%, 85%, 90%, 91%, 92%, 93%, 94%, 95%, 96%, 97%, 98%, 99%, or 100% identical to an amino acid sequence of any one of SEQ ID NOs: 14, 34, and 35. In some embodiments, the first multimerization domain comprises an amino acid sequence which is at least 75%, 80%, 85%, 90%, 91%, 92%, 93%, 94%, 95%, 96%, 97%, 98%, 99%, or 100% identical to an amino acid sequence of any one of SEQ ID NOs: 24 and 30-31; and/or the second multimerization domain comprises an amino acid sequence which is at least 95%, at least 96%, at least 97%, at least 98%, at least 99%, or at least 100% identical to an amino acid sequence selected from any one of SEQ ID NOS: 22-23, 25-29, and 32. In some embodiments, the first component comprises an amino acid sequence which is at least 75%, 80%, 85%, 90%, 91%, 92%, 93%, 94%, 95%, 96%, 97%, 98%, 99%, or 100% identical to SEQ ID NO: 6; and the second component comprises an amino acid sequence which is at least 75%, 80%, 85%, 90%, 91%, 92%, 93%, 94%, 95%, 96%, 97%, 98%, 99%, or 100% identical to SEQ ID NO: 26.

### EMBODIMENTS

The present disclosure relates to protein-based VLPs and protein-based VLP vaccines. The VLP of the present disclosure display an antigen capable of eliciting immune responses to paramyxoviruses (e.g. RSV and hMPV). Some vaccines of the present disclosure are useful for preventing or decreasing the severity of infection with paramyxoviruses (e.g. RSV and hMPV). In particular, the antigens of the disclosure display the ectodomain of the paramyxoviruses (e.g. RSV and hMPV) F protein. The ectodomain may be attached to the core of the VLP either non-covalently or covalently, including as a fusion protein or by other means disclosed herein. In some embodiments, a linker connects the ectodomain to a first polypeptide comprising a multimerization domain. The linker may be any chemical linkage including but not limited to a polypeptide used to form N-terminal or C-terminal fusion of the ectodomain to the first polypeptide. The RSV and/or hMPV F protein may optionally be displayed along a symmetry axis of the VLP. In some embodiments, the F protein is C-terminally linked to the first polypeptide comprising a trimerization domain and optionally an additional trimerization tag (e.g., FoldOn tag). Also provided are proteins and nucleic acid molecules encoding such proteins, formulations, and methods of use.

### Protein-Based Virus-Like Particles

The present disclosure relates, in part, to vaccination of a subject with a protein-based Virus-like Particle (VLP) comprising a first component comprising an RSV F protein and a first multimerization domain, along with a (VLP) comprising a first component comprising an hMPV F protein and a first multimerization domain. The protein complex may comprise the F protein of two or more of RSV-A, RSV-B, hMPV-A and hMPV-B. The F protein portion and the first multimerization domain may be linked by any suitable means, including co-expression as a fusion protein. The protein complex may optionally comprise a second component comprising a second multimerization domain. The pharmaceutical composition typically comprises one or more pharmaceutically acceptable diluents or excipients.

The VLPs of the present invention may comprise multimeric protein assemblies adapted for display of the ectodomain of RSV or hMPV F protein, or an antigenic fragment thereof The VLPs of the present invention comprise at least a first plurality of polypeptides. The first plurality of polypeptides (also referred to a “first component”) may be derived from a naturally-occurring protein sequence by substitution of at least one amino acid residue or by additional at the N- or C-terminus of one or more residues. In some cases, the first component comprises a protein sequence determined by computational methods. This first component may form the entire core of the VLP; or the core of the VLP may comprise one or more additional polypeptides (also referred to a “second component” or third, fourth, fifth component and so on), such that the VLP comprises two, three, four, five, six, seven, or more pluralities of polypeptides. In some cases, the first plurality will form trimers related by 3-fold rotational symmetry and the second plurality will form pentamers related by 5-fold rotational symmetry. In such cases, the VLP forms an “icosahedral particle” having 153 symmetry. Together these one or more pluralities of polypeptides may be arranged such that the members of each plurality of polypeptides are related to one another by symmetry operators. A general computational method for designing self-assembling protein materials, involving symmetrical docking of protein building blocks in a target symmetric architecture, is disclosed in Patent Pub. No. US 2015/0356240 A1.

In some cases, the VLP is adapted to display the F protein from two or more diverse strains of RSV. In non-limiting examples, the same VLP displays mixed populations of protein antigens or mixed heterotrimers of protein antigens from different strains of RSV. The sequences of the F protein of various RSV strains are known in the art, see, e.g., NCBI Accession Nos.: QFX69124.1, QFX69112.1, APW78900.1, APW78889.1, APW78878.1, APW78867.1, APW78856.1, APW78845.1, APW78834.1, APW78823.1, APW78812.1, APW78801.1, APW78790.1, APW78779.1, APW78768.1, APW78757.1, APW78746.1, APW78735.1, APW78724.1, APW78713.1, APW78702.1, APW78691.1, APW78680.1, APW78669.1, APW78658.1, APW78647.1, APW78636.1, APW78625.1, APW78614.1, and AAR14266.1.

The “core” of the VLP is used herein to describe the central portion of the VLP that links together the several copies of the RSV and/or hMPV F protein ectodomain, or antigenic fragments thereof, displayed by the VLP. In an embodiment, the first component comprises a first polypeptide comprising an RSV and/or hMPV F protein ectodomain or antigenic variant thereof, a linker, and a first polypeptide comprising a multimerization domain. Thereby, the “antigen” is a single polypeptide, capable of self-assembly into a VLP either alone or this a second component (third, fourth, or fifth, etc., components). An advantage of designing the antigens themselves to self-assemble is that the entire VLP then acts as the antigenic component of the vaccine. In other embodiments, the ectodomain, or antigenic fragment thereof, is non-covalently or covalently linked to the first polypeptide comprising a multimerization domain. For example, an antibody or antigenic fragment thereof may be fused to the first polypeptide and configured to bind a portion of the first polypeptide, or a chemical tag on the first polypeptide. A streptavidin-biotin (or neuravidin-biotin) linker can be employed. Or various chemical linkers may be used. An advantage of designing a core to be a generic platform is that the one or more pluralities of polypeptides that comprise the core can be designed and optimized in advance and then applied to the ectodomain serially to identify VLP(s) having superior efficacy as vaccines. It will be understood that in some cases, the same polypeptide may form a portion of the “core” and then extend outward as either an adaptor for attachment of the RSV and/or hMPV F protein ectodomain and to include the ectodomain (i.e., a fusion protein). In embodiments of the present disclosure, the antigen comprises further polypeptide sequences in addition to the RSV and/or hMPV F protein ectodomain. In certain embodiments, the ectodomain is glycosylated either natively or using alternative oligosaccharides (e.g., oligosaccharides specific to the host cell used to express the antigen).

In some cases, self-assembly may be further promoted by multimerization of the ectodomain even though the core would, in absence of the ectodomain, be independently capable of self-assembly. The RSV and/or hMPV F protein is detected as a trimer in its native state (Cseke et al. J. Virol. 21(2):698-707 (2007)). Display of the ectodomain on a VLP, at least in some embodiments, decreases the thermodynamic barrier for assembly and/or the equilibrium ratio of assembled to non-assembled VLP components. In some cases, the trimeric ectodomain placed along a 3-fold axis of the VLP promotes proper folding and conformation stability of the ectodomain and makes self-assembly of the VLP a cooperative process, in that the ectodomain is trimerized properly in part due to its display on a 3-fold axis of the core of the VLP, and the VLP is stabilized in its assembled form, at least in part, by non-covalent or covalent interactions amongst the trimer units. In some cases, introduction of mutations to the antigen or to the VLP components may optionally further stabilize assembly, in particular if cysteine residues are positioned to create intramolecular disulfide bonds. In some examples, a dimeric, trimeric, tetrameric, pentameric, or hexameric antigen is displayed upon a core designed to have a matching 2-fold, 3-fold, 4-fold, 5-fold, or 6-fold symmetry axis such that the core accommodates the arrangement of the multimeric antigen with the native symmetry of the antigen.

### Various Non-Limiting Examples of VLPs

A non-limiting example of an embodiment is shown in FIG. 1A, which depicts an ectodomain antigen genetically fused to a component (a first plurality of polypeptides) of the VLP, which optionally is expressed recombinantly in a host cell (e.g., 293F cells or CHO cells); along with a pentameric protein assembly (a second plurality of polypeptides), which is optionally expressed recombinantly in the same or a different host cell (e.g., E. coli cells), these two pluralities of polypeptides self-assembling into a VLP displaying 20 antigen trimers around an icosahedral core. In this embodiment, the core has a generic design. As explained below, in other embodiments, an antigen of the disclosure is mixed with another antigen protein in the same VLP, such as an ectodomain of a protein from a second virus, or such as a trimeric glycoprotein from another virus. In some embodiments, the VLP comprises, in addition to an RSV and/or hMPV F protein ectodomain, the trimeric glycoproteins of HIV-1, HIV-2, EBV, CMV, RSV, influenza, Ebola, Marburg, Dengue, SARS, MERS, Hanta, or Zika virus. In some embodiments, the VLP comprises the trimeric glycoproteins of viruses that are related evolutionarily or in sequence identity to any of these illustrative viruses, including without limitation, a herpes virus, orthomyxovirus, paramyxovirus, pneumovirus, filovirus, flavivirus, reovirus, or retrovirus. In an embodiment, the VLP comprises the extracellular domain or domains of a transmembrane protein or glycoprotein, or an antigenic fragment thereof. In some embodiments, the VLP is further linked to polypeptides or other agents capable of acting as an adjuvant. In some embodiments, the antigen (first component) and/or the second component comprises one or more T cell epitopes, optionally a T cell epitope of heterologous origin.

Trimeric antigens that may be used with VLPs of the disclosure form mixed VLPs are in some cases, without limitation, SARS-CoV-2, respiratory syncytial virus, HIV gp140, influenza HA, dengue E protein, or Ebola sGP. Mixed VLPs of the disclosure include those that comprise both an RSV and/or hMPV F protein ectodomain and antigenic protein(s) from one or more of Vesicular stomatitis virus, Herpes simplex virus, Baculovirus, Thogotovirus, and Bornaviridae. When other trimeric antigens are used, they may optionally be placed on the 3-fold symmetry axis of the VLP. In some cases, the antigen chosen is monomeric and nevertheless placed on a 3-fold axis. Thus, the VLP depicted in FIG. 1A is capable of displaying 20 trimeric antigens or 60 monomeric antigens. Additionally or alternatively the pentameric complexes of the VLP is used to display a 12 pentameric antigens or 70 monomeric antigens. In an embodiment, the VLP comprises 20 copies of a trimeric antigen and 12 copies of a pentameric antigen.

### VLP Cores

Other potential arrangements of polypeptides of the present disclosure are shown in FIG. 1B. In some embodiments, the VLP is adapted for display of up to 8 trimers; 8 trimers and 12 dimers; 6 tetramers and 12 dimers; 6 tetramers and 8 trimers; 20 trimers and 30 dimers; 4 trimers and 6 dimers; 4 first trimers and 4 second trimers, or 8 trimers; 12 pentamers and 20 trimers; or 12 pentamers and 30 dimers; or 4 trimers. In some cases, one of the symmetric axes is not used for antigen display, thus, in some embodiments the VLP is adapted for display of up to 8 trimers; 12 dimers; 6 tetramers; 20 trimers; 30 dimers; 4 trimers; 6 dimers; 8 trimers; or 12 pentamers. In some cases, monomeric antigens are displayed and thus, the VLP is adapted for display of up to 12, 24, 60, or 70 monomeric antigens. In some cases, the VLP comprises mixed pluralities of polypeptides such that otherwise identical polypeptides of the core of the VLP display different antigens or no antigen. Thus, depending on the ratio of polypeptides, the VLP is in some cases adapted for display of between 1 and 130 antigens (e.g., on the 152 particle) where each of the antigens displayed may be the same or may be different members of mixed population in proportion to any ratio chosen. The antigens may be co-expressed in a recombinant expression system and self-assembled before purification. Alternatively, the antigens may be expressed separately and then mixed together, either before or after purification from expression host and associated contaminants. In various embodiments, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more antigens are displayed. Non-limiting illustrative VLPs are provided in Bale et al. Science 353:389-94 (2016); Heinze et al. J. Phys. Chem B. 120:5945-5952 (2016); King et al. Nature 510:103-108 (2014); and King et al. Science 336:1171-71 (2012).

### Mixed VLPs

In some cases, the VLP is adapted to display the same antigen from two or more diverse strains of hMPV. In non-limiting examples, the same VLP displays mixed populations of homotrimeric protein antigens or mixed heterotrimers of protein antigens from different strains of hMPV. In an embodiment, the VLP displays the F proteins, or antigenic fragment thereof, disclosed in any sequence of GenBank found by searching the Protein database with the keyword “human metapneumovirus F,” individually or in mixed VLPs.

When mixed VLPs are made, it may be advantageous to ensure formation of homomultimers in a strain-specific manner rather than permit heteromultimerization, such that, for example all strain 1 F proteins are displayed on one 3-fold axis of a T33 particle whereas all strain 2 F proteins are displayed on the other 3-fold axis of the T33 particle. This may be achieved by use a VLP comprising two or more pluralities of polypeptides as the core of the VLP with each plurality of polypeptides attached to a different antigen. Alternatively, a VLP may be engineered with one or more symmetry-breaking mutations, such as knob-in-hole mutations or intramolecular disulfide mutations, which have the effect of preventing trimer formation between the different antigens. In that case, the VLP displays multimeric antigens from different strains at symmetrically equivalent positions on the VLP, but each position on the VLP is occupied by homomultimers from the same strain, with only an insignificant proportion of inter-strain heteromeric antigens. In some cases, the antigen itself may be genetically engineered to prevent inter-strain heteromultimerization. In an embodiment, the VLP is engineered to prevent heteromultimerization of two antigenic proteins with conserved structure but divergent antigenicity, such as for example, a strain 1 F protein and strain 2 F protein, or a hMPV F protein and a non-hMPV antigenic protein. Furthermore, when mixed VLPs are made and the antigens are displayed as fusion proteins, the VLP will comprise three or more different proteins, as the fusion proteins will share identical (or equivalent) domains used to form the core of the VLP with different antigenic domains, one for each antigen displayed on the VLP.

### Attachment Modalities (Linkers)

The VLPs of the present disclosure display antigenic proteins in various ways including as gene fusion or by other means disclosed herein. As used herein, “linked to” or “attached to” denotes any means known in the art for causing two polypeptides to associate. The association may be direct or indirect, reversible or irreversible, weak or strong, covalent or non-covalent, and selective or nonselective.

In some embodiments, attachment is achieved by genetic engineering to create an N- or C-terminus fusion of an antigen to one of the pluralities of polypeptides composing the VLP. Thus, the VLP may consist of, or consist essentially of, one, two, three, four, five, six, seven, eight, nine, or ten pluralities of polypeptides displaying one, two, three, four, five, six, seven, eight, nine, or ten pluralities of antigens, where at least one of the pluralities of antigen is genetically fused to at least one of the plurality of polypeptides. In some cases, the VLP consists essentially of one plurality of polypeptides capable of self-assembly and comprising the plurality of antigenic proteins genetically fused thereto. In some cases, the VLP consists essentially of a first plurality of polypeptides comprising a plurality of antigens; and a second plurality of polypeptides capable of co-assembling into two-component VLP, one plurality of polypeptides linking the antigenic protein to the VLP and the other plurality of polypeptides promoting self-assembly of the VLP.

In some embodiments, attachment is achieved by post-translational covalent attachment between one or more pluralities of polypeptides and one or more pluralities of antigenic protein. In some cases, chemical cross-linking is used to non-specifically attach the antigen to a VLP polypeptide. In some cases, chemical cross-linking is used to specifically attach the antigenic protein to a VLP polypeptide (e.g. to the first polypeptide or the second polypeptide). Various specific and non-specific cross-linking chemistries are known in the art, such as Click chemistry and other methods. In general, any cross-linking chemistry used to link two proteins may be adapted for use in the presently disclosed VLPs. In particular, chemistries used in creation of immunoconjugates or antibody drug conjugates may be used. In some cases, an VLP is created using a cleavable or non-cleavable linker. Processes and methods for conjugation of antigens to carriers are provided by, e.g., Patent Pub. No. US 2008/0145373 A1.

In an embodiment, attachment is achieved by non-covalent attachment between one or more pluralities of polypeptides and one or more pluralities of antigen. In some cases, the antigenic protein is engineered to be negatively charged on at least one surface and the core polypeptide is engineered to be positively charged on at least one surface, or positively and negatively charged, respectively. This promotes intermolecular association between the antigenic protein and the core polypeptide by electrostatic force. In some cases, shape complementarity is employed to cause linkage of antigen protein to core. Shape complementarity can be pre-existing or rationally designed. In some cases, computational designed of protein-protein interfaces is used to achieve attachment. In an embodiment, the antigen is biotin-labeled and the polypeptide comprises a streptavidin, or vice versa. In an embodiment, streptavidin is displayed by gene fusion or otherwise as a tetramer on a 4-fold axis of the core and the biotin-labeled antigen is monomeric, dimeric, or tetrameric, permitting association to the core in a configuration appropriate for native multimerization of the antigen. In some cases, a protein-based adaptor is used to capture the antigenic protein. In some cases, the polypeptide is fused to a protein capable of binding a complementary protein, which is fused to the antigenic protein.

The immune reaction to RSV and/or hMPV F may be controlled by altering the orientation of the ectodomain relative to the core. Depending on how the antigenic protein is attached to the core of the VLP, the antigenic protein may be displayed in various orientations. In some embodiments, the antigenic protein is displayed so that one or more known epitopes are oriented at or towards the distal end of the antigenic protein, such that these epitope(s) are preferentially accessible to the immune system. In some cases, the orientation will recapitulate the orientation of a viral protein with respect to the virus. The choice of orientation may direct the immune system to one or the other epitope.

In some embodiments, epitope preference is control by other means, such as positioning of glycans on the VLP by addition or subtraction of the N-linked glycan sequence motif N-X-[T/S] at predetermined positions in the amino acid sequence of any of the polypeptides of the VLP including in the amino acid sequence of the antigenic protein.

In some cases, the epitopes found at intermediate distances from the proximal to the distal end will be the preferred over epitopes more distally located depending on various considerations including but not limited to the overall geometry of the VLP, surface hydrophobicity, surface charge, and competitive binding of proteins endogenously present in the subject or proteins exogenously provided in the vaccine composition. The present disclosure encompasses all known methods of rational design of protein structure and the foregoing is not intended to be limiting.

### VLP Polypeptide Sequences

The polypeptides of the present disclosure may have any of various amino acids sequences. Patent Pub No. US 2015/0356240 A1 describes various methods for designing protein assemblies. As described in US Patent Pub No. US 2016/0122392 A1 and in International Patent Pub. No. WO 2014/124301 A1, the isolated polypeptides of SEQ ID NOS:1-51 were designed for their ability to self-assemble in pairs to form VLPs, such as icosahedral particles. The design involved design of suitable interface residues for each member of the polypeptide pair that can be assembled to form the VLP. The VLPs so formed include symmetrically repeated, non-natural, non-covalent polypeptide-polypeptide interfaces that orient a first assembly and a second assembly into a VLP, such as one with an icosahedral symmetry. Thus, in one embodiment the first polypeptide and second polypeptides (that is, the two polypeptides of the core of the VLP) are selected from the group consisting of SEQ ID NOS:1-51. In each case, an N-terminal methionine residue present in the full length protein but may be removed to make a fusion is not included in the sequence. The identified residues in Table 1 are numbered beginning with an N-terminal methionine (not shown). In various embodiments, one or more additional residues are deleted from the N-terminus and/or additional residues are added to the N-terminus (e.g. to form a helical extension).

TABLE 1

| | | | Identified |
|---|---|---|---|
| | Component | | interface |
| Name | Multimer | Amino Acid Sequence | residues |
| I53-34A | trimer | EGMDPLAVLAESRLLPLLTVRGGEDLAGLATVLELMGVGA | I53-34A: |
| SEQ ID | | LEITLRTEKGLEALKALRKSGLLLGAGTVRSPKEAEAALE | 28, 32, 36, 37, |
| NO: 1 | | AGAAFLVSPGLLEEVAALAQARGVPYLPGVLTPTEVERAL | 186, 188, 191, |
| | | ALGLSALKFFPAEPFQGVRVLRAYAEVFPEVRELPTGGIK | 192, 195 |
| | | EEHLPHYAALPNLLAVGGSWLLQGDLAAVMKKVKAAKALL | |
| | | SPQAPG | |
| I53-34B | pentamer | TKKVGIVDTTFARVDMAEAAIRTLKALSPNIKIIRKTVPG | I53-34B: |
| SEQ ID | | IKDLPVACKKLLEEEGCDIVMALGMPGKAEKDKVCAHEAS | 19, 20, 23, 24, |
| NO: 2 | | LGLMLAQLMTNKHIIEVFVHEDEAKDDDELDILALVRAIE | 27, 109, 113, |
| | | HAANVYYLLFKPEYLTRMAGKGLRQGREDAGPARE | 116, 117, 120, |
| | | | 124, 148 |
| I53-40A | pentamer | TKKVGIVDTTFARVDMASAAILTLKMESPNIKIIRKTVPG | I53-40A: |
| SEQ ID | | IKDLPVACKKLLEEEGCDIVMALGMPGKAEKDKVCAHEAS | 20, 23, 24, 27, |
| NO: 3 | | LGLMLAQLMTNKHIIEVFVHEDEAKDDAELKILAARRAIE | 28, 109, 112, |
| | | HALNVYYLLFKPEYLTRMAGKGLRQGFEDAGPARE | 113, 116, 120, |
| | | | 124 |
| I53-40B | trimer | STINNQLKALKVIPVIAIDNAEDIIPLGKVLAENGLPAAE | I53-40B: |
| SEQ ID | | ITFRSSAAVKAIMLLRSAQPEMLIGAGTILNGVQALAAKE | 47, 51, 54, 58, |
| NO: 4 | | AGATFVVSPGFNPNTVRACQIIGIDIVPGVNNPSTVEAAL | 74, 102 |
| | | EMGLTTLKFFPAEASGGISMVKSLVGPYGDIRLMPTGGIT | |
| | | PSNIDNYLAIPQVLACGGTWMVDKKLVTNGEWDEIARLTR | |
| | | EIVEQVNP | |
| I53-47A | trimer | PIFTLNTNIKATDVPSDFLSLTSRLVGLILSKPGSYVAVH | I53-47A: |
| SEQ ID | | INTDQQLSFGGSTNPAAFGTLMSIGGIEPSKNRDHSAVLF | 22, 25, 29, 72, |
| NO: 5 | | DHLNAMLGIPKNRMYIHFVNLNGDDVGWNGTTF | 79, 86, 87 |
| I53-47B | pentamer | NQHSHKDYETVRIAVVRARWHADIVDACVEAFEIAMAAIG | I53-47B: |
| SEQ ID | | GDRFAVDVEDVPGAYEIPLHARTLAETGRYGAVLGTAFVV | 28, 31, 35, 36, |
| NO: 6 | | NGGIYRHEFVASAVIDGMMNVQLSTGVPVLSAVLTPHRYR | 39, 131, 132, |
| | | DSAEHHRFFAAHFAVKGVEAARACIEILAAREKIAA | 135, 139, 146 |
| I53-50A | trimer | EELFKKHKIVAVLRANSVEEAIEKAVAVFAGGVHLIEITE | I53-50A: |
| SEQ ID | | TVPDADTVIKALSVLKEKGAIIGAGTVTSVEQCRKAVESG | 25, 29, 33, 54, |
| NO: 7 | | AEFIVSPHLDEEISQFCKEKGVFYMPGVMTPTELVKAMKL | 57 |
| | | GHTILKLFPGEVVGPQFVKAMKGPFPNVKFVPTGGVNLDN | |
| | | VCEWFKAGVLAVGVGSALVKGTPDEVREKAKAFVEKIRGC | |
| | | TE | |
| I53-50B | pentamer | NQHSHKDYETVRIAVVRARWHAEIVDACVSAFEAAMADIG | I53-50B: |
| SEQ ID | | GDRFAVDVEDVPGAYEIPLHARTLAETGRYGAVLGTAFVV | 24, 28, 36, 124, |
| NO: 8 | | NGGIYRHEFVASAVIDGMMNVQLSTGVPVLSAVLTPHRYR | 125, 127, 128, |
| | | DSDAHTLLFLALFAVKGMEAARACVEILAAREKIAA | 129, 131, 132, |
| | | | 133, 135, 139 |
| I53-51A | trimer | FTKSGDDGNTNVINKRVGKDSPLVNFLGDLDELNSFIGFA | I53-51A: |
| SEQ ID | | ISKIPWEDMKKDLERVQVELFEIGEDLSTQSSKKKIDESY | 80, 83, 86, 87, |
| NO: 9 | | VLWLLAATAIYRIESGPVKLFVIPGGSEEASVLHVTRSVA | 88, 90, 91, |
| | | RRVERNAVKYTKELPEINRMIIVYLNRLSSLLFAMALVAN | 94, 166, 172, |
| | | KRRNQSEKIYEIGKSW | 176 |
| I53-51B | pentamer | NQHSHKDYETVRIAVVRARWHADIVDQCVRAFEEAMADAG | I53-51B: |
| SEQ ID | | GDRFAVDVFDVPGAYEIPLHARTLAETGRYGAVLGTAFVV | 31, 35, 36, 40, |
| NO: 10 | | NGGIYRHEFVASAVIDGMMNVQLSTGVPVLSAVLTPHRYR | 122, 124, 120, |
| | | SSREHHEFFREHFMVKGVEAAAACITILAAREKIAA | 131, 135, |
| | | | 139, 143, 146, |
| | | | 147 |
| I52-03A | pentamer | GHTKGPTPQQHDGSALRIGIVHARWNKTIIMPLLIGTIAK | I52-03A: |
| SEQ ID | | LLECGVKASNIVVQSVPGSWELPIAVQRLYSASQLQTPSS | 28, 32, 36, 39, |
| NO: 11 | | GPSLSAGDLLGSSTTDLTALPTTTASSTGPFDALIAIGVL | 44, 49 |
| | | IKGETMHFEYIADSVSHGLMRVQLDTGVPVIFGVLTVLTD | |
| | | DQAKARAGVIEGSHNHGEDWGLAAVEMGVRRRDWAAGKTE | |
| I52-03B | dimer | YEVDHADVYDLFYLGRGKDYAAEASDIADLVRSRTPEASS | I52-03B: |
| SEQ ID | | LLDVACGTGTHLEHFTKEFGDTAGLELSEDMLTHARKRLP | 94, 115, 116, |
| NO: 12 | | DATLHQGDMRDFQLGRKFSAVVSMFSSVGYLKTVAELGAA | 206, 213 |
| | | VASFAEHLEPGGVVVVEPWWFPETFADGWVSADVVRRDGR | |
| | | TVARVSHSVREGNATRMEVHFTVADPGKGVRHFSDVHLIT | |
| | | LFHQREYEAAFMAAGLRVEYLEGGPSGRGLFVGVPA | |
| I52-32A | dimer | GMKEKFVLIITHGDFGKGLLSGAEVIIGKQENVHTVGLNL | I52-32A: |
| SEQ ID | | GDNIEKVAKEVMRIIIAKLAEDKEIIIVVDLFGGSPFNIA | 47, 49, 53, 54, |
| NO: 13 | | LEMMKTFDVKVITGINMPMLVELLTSINVYDTTELLENIS | 57, 58, 61, 83, |
| | | KIGKDGIKVIEKSSLKM | 87, 88 |
| I52-32B | pentamer | KYDGSKLRIGILHARWNLEIIAALVAGAIKRLQEFGVKAE | I52-32B: |
| SEQ ID | | NIIIETVPGSFELPYGSKLFVEKQKRLGKPLDAIIPIGVL | 19, 20, 23, 30, |
| NO: 14 | | IKGSTMHFEYICDSTTHQLMKLNFELGIPVIFGVLTCLTD | 40 |
| | | EQAEARAGLIEGKMHNHGEDWGAAAVEMATKFN | |
| I52-33A | pentamer | AVKGLGEVDQKYDGSKLRIGILHARWNRKIILALVAGAVL | I52-33A: |
| SEQ ID | | RLLEFGVKAENIIIETVPGSFELPYGSKLFVEKQKRLGKP | 33, 41, 44, 50 |
| NO: 15 | | LDAIIPIGVLIKGSTMHFEYICDSTTHQLMKLNFELGIPV | |
| | | IFGVLTCLTDEQAEARAGLIEGKMHNHGEDWGAAAVEMAT | |
| | | KFN | |
| I52-33B | dimer | GANWYLDNESSRLSFTSTKNADIAEVHRFLVLHGKVDPKG | I52-33B: |
| SEQ ID | | LAEVEVETESISTGIPLRDMLLRVLVFQVSKFPVAQINAQ | 61, 63, 66, 67, |
| NO: 16 | | LDMRPINNLAPGAQLELRLPLTVSLRGKSHSYNAELLATR | 72, 147, 148, |
| | | LDERRFQVVTLEPLVIHAQDEDMVRAFNALRLVAGLSAVS | 154, 155 |
| | | LSVPVGAVLIFTAR | |
| I32-06A | dimer | TDYIRDGSAIKALSFAIILAEADLRHIPQDLQRLAVRVIH | I32-06A: |
| SEQ ID | | ACGMVDVANDLAFSEGAGKAGRNALLAGAPILCDARMVAE | 9, 12, 13, 14, |
| NO: 17 | | GITRSRLPADNRVIYTLSDPSVPELAKKIGNTRSAAALDL | 20, 30, 33, 34 |
| | | WLPHIEGSIVAIGNAPTALFRLFELLDAGAPKPALIIGMP | |
| | | VGFVGAAESKDELAANSRGVPYVIVRGRRGGSAMTAAAVN | |
| | | ALASERE | |
| I32-06B | trimer | ITVFGLKSKLAPRREKLAEVIYSSLHLGLDIPKGKHAIRF | I32-06B: |
| SEQ ID | | LCLEKEDFYYPFDRSDDYTVIEINLMAGRSEETKMLLIFL | 24, 71, 73, 76, |
| NO: 18 | | LFIALERKLGIRAHDVEITIKEQPAHCWGFRGRTGDSARD | 77, 80, 81, 84, |
| | | LDYDIYV | 85, 88, 114, |
| | | | 118 |
| I32-19A | trimer | GSDLQKLQRFSTCDISDGLLNVYNIPTGGYFPNLTAISPP | I32-19A: |
| SEQ ID | | QNSSIVGTAYTVLFAPIDDPRPAVNYIDSVPPNSILVLAL | 208, 213, 218, |
| NO: 19 | | EPHLQSQFHPFIKITQAMYGGLMSTRAQYLKSNGTVVFGR | 222, 225, 226, |
| | | IRDVDEHRTLNHPVFAYGVGSCAPKAVVKAVGTNVQLKIL | 229, 233 |
| | | TSDGVTQTICPGDYIAGDNNGIVRIPVQETDISKLVTYIE | |
| | | KSIEVDRLVSEAIKNGLPAKAAQTARRMVLKDYI | |
| I32-19B | dimer | SGMRVYLGADHAGYELKQAIIAFLKMTGHEPIDCGALRYD | I32-19B: |
| SEQ ID | | ADDDYPAFCIAAATRTVADPGSLGIVLGGSGNGEQIAANK | 20, 23, 24, 27, |
| NO: 20 | | VPGARCALAWSVQTAALAREHNNAQLIGIGGRMHTLEEAL | 117, 118, 122, |
| | | RIVKAFVTTPWSKAQRHQRRIDILAEYERTHEAPPVPGAP | 125 |
| | | A | |
| I32-28A | trimer | GDDARIAAIGDVDELNSQIGVLLAEPLPDDVRAALSAIQH | I32-28A: |
| SEQ ID | | DLFDLGGELCIPGHAAITEDHLLRLALWLVHYNGQLPPLE | 60, 61, 64, 67, |
| NO: 21 | | EFILPGGARGAALAHVCRTVCRRAERSIKALGASEPLNIA | 68, 71, 110, |
| | | PAAYVNLLSDLLFVLARVLNRAAGGADVLWDRTRAH | 120, 123, 124, |
| | | | 128 |
| I32-28B | dimer | ILSAEQSFTLRHPHGQAAALAFVREPAAALAGVQRLRGLD | I32-28B: |
| SEQ ID | | SDGEQVWGELLVRVPLLGEVDLPFRSEIVRTPQGAELRPL | 35, 36, 54, 122, |
| NO: 22 | | TLTGERAWVAVSGQATAAEGGEMAFAFQFQAHLATPEAEG | 129, 137, 140, |
| | | EGGAAFEVMVQAAAGVTLLLVAMALPQGLAAGLPPA | 141, 144, |
| | | | 148 |
| I53- | pentamer | TKKVGIVDTTFARVDMASAAILTLKMESPNIKIIRKTVPG | I53-40A: |
| 40A.1 | | IKDLPVACKKLLEEEGCDIVMALGMPGKKEKDKVCAHEAS | 20, 23, 24, 27, |
| SEQ ID | | LGLMLAQLMTNKHIIEVFVHEDEAKDDAELKILAARRAIE | 28, 109, 112, |
| NO: 23 | | HALNVYYLLFKPEYLTRMAGKGLRQGFEDAGPARE | 113, 116, 120, |
| | | | 124 |
| I53- | trimer | DDINNQLKRIKVIPVIAIDNAEDIIPLGKVLAENGLPAAE | I53-40B: |
| 40B.1 | | ITFRSSAAVKAIMLLRSAQPEMLIGAGTILNGVQALAAKE | 47, 51, 54, 58, |
| SEQ ID | | AGADFVVSPGFNPNTVRACQIIGIDIVPGVNNPSTVEQAL | 74, 102 |
| NO: 24 | | EMGLTTLKFFPAEASGGISMVKSLVGPYGDIRLMPTGGIT | |
| | | PDNIDNYLAIPQVLACGGTWMVDKKLVRNGEWDEIARLTR | |
| | | EIVEQVNP | |
| I53- | trimer | PIFTLNTNIKADDVPSDFLSLTSRLVGLILSKPGSYVAVH | I53-47A: |
| 47A.1 | | INTDQQLSFGGSTNPAAFGTLMSIGGIEPDKNRDHSAVLF | 22, 25, 29, 72, |
| SEQ ID | | DHLNAMLGIPKNRMYIHFVNLNGDDVGWNGTTF | 79, 86,87 |
| NO: 25 | | | |
| I53- | trimer | PIFTLNTNIKADDVPSDFLSLTSRLVGLILSEPGSYVAVH | I53-47A: |
| 47A.1Neg | | INTDQQLSFGGSTNPAAFGTLMSIGGIEPDKNEDHSAVLF | 22, 25, 29, 72, |
| T2 | | DHLNAMLGIPKNRMYIHFVDLDGDDVGWNGTTE | 79, 86, 87 |
| SEQ ID | | | |
| NO: 26 | | | |
| I53- | pentamer | NQHSHKDHETVRIAVVRARWHADIVDACVEAFEIAMAAIG | I53-47B: |
| 47B.1 | | GDRFAVDVFDVPGAYEIPLHARTLAETGRYGAVLGTAFVV | 28, 31, 35, 36, |
| SEQ ID | | NGGIYRHEFVASAVIDGMMNVQLDTGVPVLSAVLTPHRYR | 39, 131, 132, |
| NO: 27 | | DSDEHHRFFAAHFAVKGVEAARACIEILNAREKIAA | 135, 139, 146 |
| I53- | pentamer | NQHSHKDHETVRIAVVRARWHADIVDACVEAFEIAMAAIG | I53-47B: |
| 47B.1Neg | | GDRFAVDVFDVPGAYEIPLHARTLAETGRYGAVLGTAFVV | 28, 31, 35, 36, |
| T2 | | DGGIYDHEFVASAVIDGMMNVQLDTGVPVLSAVLTPHEYE | 39, 131, 132, |
| SEQ ID | | DSDEDHEFFAAHFAVKGVEAARACIEILNAREKIAA | 135, 139, 146 |
| NO: 28 | | | |
| I53- | trimer | EELFKKHKIVAVLRANSVEEAIEKAVAVFAGGVHLIEITF | I53-50A: |
| 50A.1 | | TVPDADTVIKALSVLKEKGAIIGAGTVTSVEQCRKAVESG | 25, 29, 33, 54, |
| SEQ ID | | AEFIVSPHLDEEISQFCKEKGVFYMPGVMTPTELVKAMKL | 57 |
| NO: 29 | | GHDILKLFPGEVVGPQFVKAMKGPFPNVKFVPTGGVNLDN | |
| | | VCEWFKAGVLAVGVGDALVKGDPDEVREKAKKFVEKIRGC | |
| | | TE | |
| I53- | trimer | EELFKKHKIVAVLRANSVEEAIEKAVAVFAGGVHLIEITF | I53-50A: |
| 50A.1Neg | | TVPDADTVIKALSVLKEKGAIIGAGTVTSVEQCRKAVESG | 25, 29, 33, 54, |
| T2 | | AEFIVSPHLDEEISQFCKEKGVFYMPGVMTPTELVKAMKL | 57 |
| SEQ ID | | GHDILKLFPGEVVGPEFVEAMKGPFPNVKFVPTGGVDLDD | |
| NO: 30 | | VCEWFDAGVLAVGVGDALVEGDPDEVREDAKEFVEEIRGC | |
| | | TE | |
| I53- | trimer | EELFKKHKIVAVLRANSVEEAIEKAVAVFAGGVHLIEITF | I53-50A: |
| 50A.1Pos | | TVPDADTVIKALSVLKEKGAIIGAGTVTSVEQCRKAVESG | 25, 29, 33, 54, |
| T1 | | AEFIVSPHLDEEISQFCKEKGVFYMPGVMTPTELVKAMKL | 57 |
| SEQ ID | | GHDILKLFPGEVVGPQFVKAMKGPFPNVKFVPTGGVNLDN | |
| NO: 31 | | VCKWFKAGVLAVGVGKALVKGKPDEVREKAKKFVKKIRGC | |
| | | TE | |
| I53- | pentamer | NQHSHKDHETVRIAVVRARWHAEIVDACVSAFEAAMRDIG | I53-50B: |
| 50B.1 | | GDRFAVDVFDVPGAYEIPLHARTLAETGRYGAVLGTAFVV | 24, 28, 36, 124, |
| SEQ ID | | NGGIYRHEFVASAVIDGMMNVQLDTGVPVLSAVLTPHRYR | 125, 127, |
| NO: 32 | | DSDAHTLLELALFAVKGMEAARACVEILAAREKIAA | 128, 129, 131, |
| | | | 132, 133, 135, |
| | | | 139 |
| I53- | pentamer | NQHSHKDHETVRIAVVRARWHAEIVDACVSAFEAAMRDIG | I53-50B: |
| 50B.1Neg | | GDRFAVDVFDVPGAYEIPLHARTLAETGRYGAVLGTAFVV | 24, 28, 36, 124, |
| T2 | | DGGIYDHEFVASAVIDGMMNVQLDTGVPVLSAVLTPHEYE | 125, 127, |
| SEQ ID | | DSDADTLLFLALFAVKGMEAARACVEILAAREKIAA | 128, 129, 131, |
| NO: 33 | | | 132, 133, 135, |
| | | | 139 |
| I53- | trimer | NQHSHKDHETVRIAVVRARWHAEIVDACVSAFEAAMRDIG | I53-50B: |
| 50B.4Pos | | GDRFAVDVFDVPGAYEIPLHARTLAETGRYGAVLGTAFVV | 24, 28, 36, 124, |
| T1 | | NGGIYRHEFVASAVINGMMNVQLNTGVPVLSAVLTPHNYD | 125, 127, |
| SEQ ID | | KSKAHTLLFLALFAVKGMEAARACVEILAAREKIAA | 128, 129, 131, |
| NO: 34 | | | 132, 133, 135, |
| | | | 139 |
| I53-40A | pentamer | TKKVGIVDTTFARVDMASAAILTLKMESPNIKIIRKTVPG | |
| genus | | IKDLPVACKKLLEEEGCDIVMALGMPGK(A/K)EKDKVCA | |
| SEQ ID | | HEASLGLMLAQLMTNKHIIEVFVHEDEAKDDAELKILAAR | |
| NO: 35 | | RAIEHALNVYYLLFKPEYLTRMAGKGLRQGFEDAGPARE | |
| I53-40B | trimer | (S/D)(T/D)INNQLK(A/R)LKVIPVIAIDNAEDIIPLG | |
| genus | | KVLAENGLPAAEITFRSSAAVKAIMLLRSAQPEMLIGAGT | |
| SEQ ID | | ILNGVQALAAKEAGA(T/D)FVVSPGFNPNTVRACQIIGI | |
| NO: 36 | | DIVPGVNNPSTVE(A/Q)ALEMGLTTLKFFPAEASGGISM | |
| | | VKSLVGPYGDIRLMPTGGITP(S/D)NIDNYLAIPQVLAC | |
| | | GGTWMVDKKLV(T/R)NGEWDEIARLTREIVEQVNP | |
| I53-47A | trimer | PIFTLNTNIKA(T/D)DVPSDFLSLTSRLVGLILS(K/E) | |
| genus | | PGSYVAVHINTDQQLSFGGSTNPAAFGTLMSIGGIEP(S/ | |
| SEQ ID | | D)KN(R/E)DHSAVLFDHLNAMLGIPKNRMYIHFV(N/D) | |
| NO: 37 | | L(N/D)GDDVGWNGTTF | |
| I53-47B | pentamer | NQHSHKD(Y/H)ETVRIAVVRARWHADIVDACVEAFEIAM | |
| genus | | AAIGGDRFAVDVFDVPGAYEIPLHARTLAETGRYGAVLGT | |
| SEQ ID | | AFVV(N/D)GGIY(R/D)HEFVASAVIDGMMNVQL(S/D) | |
| NO: 38 | | TGVPVLSAVLTPH(R/E)Y(R/E)DS(A/D)E(H/D)H | |
| | | (R/E)FFAAHFAVKGVEAARACIEIL(A/N)AREKIAA | |
| I53-50A | trimer | EELFKKHKIVAVLRANSVEEAIEKAVAVFAGGVHLIEITF | |
| genus | | TVPDADTVIKALSVLKEKGAIIGAGTVTSVEQCRKAVESG | |
| SEQ ID | | AEFIVSPHLDEEISQFCKEKGVFYMPGVMTPTELVKAMKL | |
| NO: 39 | | GH(T/D)ILKLFPGEVVGP(Q/E)FV(K/E)AMKGPFPNV | |
| | | KFVPTGGV(N/D)LD(N/D)VC(E/K)WF(K/D)AGVLAV | |
| | | GVG(S/K/D)ALV(K/E)G(T/D/K)PDEVRE(K/D)AK | |
| | | (A/E/K)FV(E/K)(K/E)IRGCTE | |
| I53-50B | pentamer | NQHSHKD(Y/H)ETVRIAVVRARWHAEIVDACVSAFEAAM | |
| genus | | (A/R)DIGGDRFAVDVFDVPGAYEIPLHARTLAETGRYGA | |
| SEQ ID | | VLGTAFVV(N/D)GGIY(R/D)HEFVASAVI(D/N)GMMN | |
| NO: 40 | | VQL(S/D/N)TGVPVLSAVLTPH(R/E/N)Y(R/D/E) | |
| | | (D/K)S(D/K)A(H/D)TLLFLALFAVKGMEAARACVEIL | |
| | | AAREKIAA | |
| T32-28A | dimer | GEVPIGDPKELNGMEIAAVYLQPIEMEPRGIDLAASLADI | |
| SEQ ID | | HLEADIHALKNNPNGFPEGFWMPYLTIAYALANADTGAIK | |
| NO: 41 | | TGTLMPMVADDGPHYGANIAMEKDKKGGFGVGTYALTFLI | |
| | | SNPEKQGFGRHVDEETGVGKWFEPFVVTYFFKYTGTPK | |
| T32-28B | trimer | SQAIGILELTSIAKGMELGDAMLKSANVDLLVSKTISPGK | |
| SEQ ID | | FLLMLGGDIGAIQQAIETGTSQAGEMLVDSLVLANIHPSV | |
| NO: 42 | | LPAISGLNSVDKRQAVGIVETWSVAACISAADLAVKGSNV | |
| | | TLVRVHMAFGIGGKCYMVVAGDVLDVAAAVATASLAAGAK | |
| | | GLLVYASIIPRPHEAMWRQMVEG | |
| T33-09A | trimer | EEVVLITVPSALVAVKIAHALVEERLAACVNIVPGLTSIY | |
| SEQ ID | | RWQGSVVSDHELLLLVKTTTHAFPKLKERVKALHPYTVPE | |
| NO: 43 | | IVALPIAEGNREYLDWLRENTG | |
| T33-09B | trimer | VRGIRGAITVEEDTPAAILAATIELLLKMLEANGIQSYEE | |
| SEQ ID | | LAAVIFTVTEDLTSAFPAEAARLIGMHRVPLLSAREVPVP | |
| NO: 44 | | GSLPRVIRVLALWNTDTPQDRVRHVYLNEAVRLRPDLESA | |
| | | Q | |
| T33-15A | trimer | SKAKIGIVTVSDRASAGITADISGKAIILALNLYLTSEWE | |
| SEQ ID | | PIYQVIPDEQDVIETTLIKMADEQDCCLIVTTGGTGPAKR | |
| NO: 45 | | DVTPEATEAVCDRMMPGFGELMRAESLKEVPTAILSRQTA | |
| | | GLRGDSLIVNLPGDPASISDCLLAVFPAIPYCIDLMEGPY | |
| | | LECNEAMIKPFRPKAK | |
| T33-15B | trimer | VRGIRGAITVNSDTPTSIIIATILLLEKMLEANGIQSYEE | |
| SEQ ID | | LAAVIFTVTEDLTSAFPAEAARQIGMHRVPLLSAREVPVP | |
| NO: 46 | | GSLPRVIRVLALWNTDTPQDRVRHVYLSEAVRLRPDLESA | |
| | | Q | |
| T33-21A | trimer | RITTKVGDKGSTRLFGGEEVWKDSPIIEANGTLDELTSFI | |
| SEQ ID | | GEAKHYVDEEMKGILEEIQNDIYKIMGEIGSKGKIEGISE | |
| NO: 47 | | ERIAWLLKLILRYMEMVNLKSFVLPGGTLESAKLDVCRTI | |
| | | ARRALRKVLTVTREFGIGAEAAAYLLALSDLLFLLARVIE | |
| | | IEKNKLKEVRS | |
| T33-21B | trimer | PHLVIEATANLRLETSPGELLEQANKALFASGQFGEADIK | |
| SEQ ID | | SRFVTLEAYRQGTAAVERAYLHACLSILDGRDIATRTLLG | |
| NO: 48 | | ASLCAVLAEAVAGGGEEGVQVSVEVREMERLSYAKRVVAR | |
| | | QR | |
| T33-28A | trimer | ESVNTSFLSPSLVTIRDFDNGQFAVLRIGRTGFPADKGDI | |
| SEQ ID | | DLCLDKMIGVRAAQIFLGDDTEDGFKGPHIRIRCVDIDDK | |
| NO: 49 | | HTYNAMVYVDLIVGTGASEVERETAEEEAKLALRVALQVD | |
| | | IADEHSCVTQFEMKLREELLSSDSFHPDKDEYYKDFL | |
| T33-28B | trimer | PVIQTFVSTPLDHHKRLLLAIIYRIVTRVVLGKPEDLVMM | |
| SEQ ID | | TFHDSTPMHFFGSTDPVACVRVEALGGYGPSEPEKVTSIV | |
| NO: 50 | | TAAITAVCGIVADRIFVLYFSPLHCGWNGTNF | |
| T33-31A | trimer | EEVVLITVPSALVAVKIAHALVEERLAACVNIVPGLTSIY | |
| SEQ ID | | REEGSVVSDHELLLLVKTTTDAFPKLKERVKELHPYEVPE | |
| NO: 51 | | IVALPIAEGNREYLDWLRENTG | |
| I53-50A | trimer | EELFKKHKIVAVLRANSVEEAIEKAVAVFAGGVHLIEITF | |
| ACys | | TVPDADTVIKALSVLKEKGAIIGAGTVTSVEQARKAVESG | |
| SEQ ID | | AEFIVSPHLDEEISQFAKEKGVFYMPGVMTPTELVKAMKL | |
| NO: 53 | | GHTILKLFPGEVVGPQFVKAMKGPFPNVKFVPTGGVNLDN | |
| | | VAEWFKAGVLAVGVGSALVKGTPDEVREKAKAFVEKIRGA | |
| | | TE | |
| T33_dn2A | | NLAEKMYKAGNAMYRKGQYTIAIIAYTLALLKDPNNAEAW | |
| SEQ ID | | YNLGNAAYKKGEYDEAIEAYQKALELDPNNAEAWYNLGNA | |
| NO: 135 | | YYKQGDYDEAIEYYKKALRLDPRNVDAIENLIEAEEKQG | |
| T33_dn2B | | EEAELAYLLGELAYKLGEYRIAIRAYRIALKRDPNNAEAW | |
| SEQ ID | | YNLGNAYYKQGDYREAIRYYLRALKLDPENAEAWYNLGNA | |
| NO: 136 | | LYKQGKYDLAIIAYQAALEEDPNNAEAKQNLGNAKQKQG | |
| T33_dn5A | | NSAEAMYKMGNAAYKQGDYILAIIAYLLALEKDPNNAEAW | |
| SEQ ID | | YNLGNAAYKQGDYDEAIEYYQKALELDPNNAEAWYNLGNA | |
| NO: 137 | | YYKQGDYDEAIEYYEKALELDPNNAEALKNLLEAIAEQD | |
| T33_dn5A | | TDPLAVILYIAILKAEKSIARAKAAEALGKIGDERAVEPL | |
| SEQ ID | | IKALKDEDALVRAAAADALGQIGDERAVEPLIKALKDEEG | |
| NO: 138 | | LVRASAAIALGQIGDERAVQPLIKALTDERDLVRVAAAVA | |
| | | LGRIGDEKAVRPLIIVLKDEEGEVREAAAIALGSIGGERV | |
| | | RAAMEKLAERGTGFARKVAVNYLETHK | |
| T33_dn10A | | EEAELAYLLGELAYKLGEYRIAIRAYRIALKRDPNNAEAW | |
| SEQ ID | | YNLGNAYYKQGDYDEAIEYYQKALELDPNNAEAWYNLGNA | |
| NO: 139 | | YYKQGDYDEAIEYYEKALELDPENLEALQNLLNAMDKQG | |
| T33_dn10B | | IEEVVAEMIDILAESSKKSIEELARAADNKTTEKAVAEAI | |
| SEQ ID | | EEIARLATAAIQLIEALAKNLASEEFMARAISAIAELAKK | |
| NO: 140 | | AIEAIYRLADNHTTDTFMARAIAAIANLAVTAILAIAALA | |
| | | SNHTTEEFMARAISAIAELAKKAIEAIYRLADNHTTDKFM | |
| | | AAAIEAIALLATLAILAIALLASNHTTEKFMARAIMAIAI | |
| | | LAAKAIEAIYRLADNHTSPTYIEKAIEAIEKIARKAIKAI | |
| | | EMLAKNITTEEYKEKAKKIIDIIRKLAKMAIKKLEDNRT | |
| I53_dn5A | | KYDGSKLRIGILHARWNAEIILALVLGALKRLQEFGVKRE | |
| SEQ ID | | NIIIETVPGSFELPYGSKLFVEKQKRLGKPLDAIIPIGVL | |
| NO: 141 | | IKGSTMHFEYICDSTTHQLMKLNFELGIPVIFGVLTCLTD | |
| | | EQAEARAGLIEGKMHNHGEDWGAAAVEMATKFN | |
| I53_dn5B | | EEAELAYLLGELAYKLGEYRIAIRAYRIALKRDPNNAEAW | |
| SEQ ID | | YNLGNAYYKQGRYREAIEYYQKALELDPNNAEAWYNLGNA | |
| NO: 142 | | YYERGEYEEAIEYYRKALRLDPNNADAMQNLLNAKMREE | |

Table 1 provides the amino acid sequence of the first polypeptide and second polypeptides of embodiments of the present disclosure. In each case, the pairs of sequences together form an 153 multimer with icosahedral symmetry. The right hand column in Table 1 identifies the residue numbers in each illustrative polypeptide that were identified as present at the interface of resulting assembled virus-like particles (i. e.: “identified interface residues”). As can be seen, the number of interface residues for the illustrative polypeptides of SEQ ID NO:1-34 range from 4-13. In various embodiments, the first polypeptide and second polypeptides comprise an amino acid sequence that is at least 75%, 80%, 85%, 90%, 91%, 92%, 93%, 94%, 95%, 96%, 97%, 98%, or 99% identical over its length, and identical at least at 1, 2, 3, 4, 5, 6, 7, 8, 9, 10, 11, 12, or 13 identified interface positions (depending on the number of interface residues for a given polypeptide), to the amino acid sequence of a polypeptide selected from the group consisting of SEQ ID NOS: 1-34. SEQ ID NOs: 35-51 represent other amino acid sequences of the first polypeptide and second polypeptides from embodiments of the present disclosure. In other embodiments, the first polypeptide and/or second polypeptides comprise an amino acid sequence that is at least 75%, 80%, 85%, 90%, 91%, 92%, 93%, 94%, 95%, 96%, 97%, 98%, or 99% identical over its length, and identical at least at 20%, 25%, 30%, 33%, 35%, 40%, 45%, 50%, 60%, 70%, 75%, 80%, 85%, 90%, 95%, or 100% of the identified interface positions, to the amino acid sequence of a polypeptide selected from the group consisting of SEQ ID NOS:1-51.

As is the case with proteins in general, the polypeptides are expected to tolerate some variation in the designed sequences without disrupting subsequent assembly into virus-like particles: particularly when such variation comprises conservative amino acid substitutions. As used here, “conservative amino acid substitution” means that: hydrophobic amino acids (Ala, Cys, Gly, Pro, Met, Val, Ile, Leu) can only be substituted with other hydrophobic amino acids; hydrophobic amino acids with bulky side chains (Phe, Tyr, Trp) can only be substituted with other hydrophobic amino acids with bulky side chains; amino acids with positively charged side chains (Arg, His, Lys) can only be substituted with other amino acids with positively charged side chains; amino acids with negatively charged side chains (Asp, Glu) can only be substituted with other amino acids with negatively charged side chains; and amino acids with polar uncharged side chains (Ser, Thr, Asn, Gln) can only be substituted with other amino acids with polar uncharged side chains.

In some cases, a non-conservative amino acid substitution may be preferred, for example, for eradication of a flexible portion of the native RSV F protein secondary structure is desired, for example, by adding a cysteine residue (or vice versa). “Non-conservative substitution” refers to the substitution of an amino acid in one class with an amino acid from another class; for example, substitution of an Ala with Asp, Asn, Glu, or Gln. Additional non-limiting examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue. Substitutions of D-Cys for D-Ala, D-Ser, or D-Tyr (or another residue) may be used to remove intramolecular disulfide bonds, which may, in some cases improve protein stability or expression. Substitutions to D-Cys may be used to generate disulfide bonds that stability a protein or lock a protein into a desired conformation.

In various embodiments of the VLPs of the invention, the first polypeptide and second polypeptides, or the vice versa, comprise polypeptides with the amino acid sequence selected from the following pairs, or modified versions thereof (i.e., permissible modifications as disclosed for the polypeptides of the invention: isolated polypeptides comprising an amino acid sequence that is at least 75%, at least 80%, at least 85%, at least 90%, at least 91%, at least 92%, at least 93%, at least 94%, at least 95%, at least 96%, at least 97%, at least 98%, at least 99%, or 100% over its length, and/or identical at least at one identified interface position, to the amino acid sequence indicated by the SEQ ID NO):
SEQ ID NO:1 and SEQ ID NO:2 (I53-34A and I53-34B);
SEQ ID NO:3 and SEQ ID NO:4 (I53-40A and I53-40B);
SEQ ID NO:3 and SEQ ID NO:24 (I53-40A and I53-40B.1);
SEQ ID NO:23 and SEQ ID NO:4 (I53-40A.1 and I53-40B);
SEQ ID NO:35 and SEQ ID NO:36 (I53-40A genus and I53-40B genus);
SEQ ID NO:5 and SEQ ID NO:6 (I53-47A and I53-47B);
SEQ ID NO:5 and SEQ ID NO:27 (I53-47A and I53-47B.1);
SEQ ID NO:5 and SEQ ID NO:28 (I53-47A and I53-47B.1NegT2);
SEQ ID NO:25 and SEQ ID NO:6 (I53-47A.1 and I53-47B);
SEQ ID NO:25 and SEQ ID NO:27 (I53-47A.1 and I53-47B.1);
SEQ ID NO:25 and SEQ ID NO:28 (I53-47A.1 and I53-47B.1NegT2);
SEQ ID NO:26 and SEQ ID NO:6 (I53-47A.1NegT2 and I53-47B);
SEQ ID NO:26 and SEQ ID NO:27 (I53-47A.1NegT2 and I53-47B.1);
SEQ ID NO:26 and SEQ ID NO:28 (I53-47A.1NegT2 and I53-47B.1NegT2);
SEQ ID NO:37 and SEQ ID NO:38 (I53-47A genus and I53-47B genus);
SEQ ID NO:7 and SEQ ID NO:8 (I53-50A and I53-50B);
SEQ ID NO:7 and SEQ ID NO:32 (I53-50A and I53-50B.1);
SEQ ID NO:7 and SEQ ID NO:33 (I53-50A and I53-50B.1NegT2);
SEQ ID NO:7 and SEQ ID NO:34 (I53-50A and I53-50B.4PosT1);
SEQ ID NO:29 and SEQ ID NO:8 (I53-50A.1 and I53-50B);
SEQ ID NO:29 and SEQ ID NO:32 (I53-50A.1 and I53-50B.1);
SEQ ID NO:29 and SEQ ID NO:33 (I53-50A.1 and I53-50B.1NegT2);
SEQ ID NO:29 and SEQ ID NO:34 (I53-50A.1 and I53-50B.4PosT1);
SEQ ID NO:30 and SEQ ID NO:8 (I53-50A.1NegT2 and I53-50B);
SEQ ID NO:30 and SEQ ID NO:32 (I53-50A.1NegT2 and I53-50B.1);
SEQ ID NO:30 and SEQ ID NO:33 (I53-50A.1NegT2 and I53-50B.1NegT2);
SEQ ID NO:30 and SEQ ID NO:34 (I53-50A.1NegT2 and I53-50B.4PosT1);
SEQ ID NO:31 and SEQ ID NO:8 (I53-50A.1PosT1 and I53-50B);
SEQ ID NO:31 and SEQ ID NO:32 (I53-50A.1PosT1 and I53-50B.1);
SEQ ID NO:31 and SEQ ID NO:33 (I53-50A.1PosT1 and I53-50B.1NegT2);
SEQ ID NO:31 and SEQ ID NO:34 (I53-50A.1PosT1 and I53-50B.4PosT1);
SEQ ID NO:39 and SEQ ID NO:40 (I53-50A genus and I53-50B genus);
SEQ ID NO:9 and SEQ ID NO:10 (I53-51A and I53-51B);
SEQ ID NO:11 and SEQ ID NO:12 (I52-03A and I52-03B);
SEQ ID NO:13 and SEQ ID NO:14 (I52-32A and I52-32B);
SEQ ID NO:15 and SEQ ID NO:16 (I52-33A and I52-33B)
SEQ ID NO:17 and SEQ ID NO:18 (I32-06A and I32-06B);
SEQ ID NO:19 and SEQ ID NO:20 (I32-19A and I32-19B);
SEQ ID NO:21 and SEQ ID NO:22 (I32-28A and I32-28B);
SEQ ID NO:23 and SEQ ID NO:24 (I53-40A.1 and I53-40B.1);
SEQ ID NO:41 and SEQ ID NO:42 (T32-28A and T32-28B);
SEQ ID NO:43 and SEQ ID NO:44 (T33-09A and T33-09B);
SEQ ID NO:45 and SEQ ID NO:46 (T33-15A and T33-15B);
SEQ ID NO:47 and SEQ ID NO:48 (T33-21A and T33-21B);
SEQ ID NO:49 and SEQ ID NO:50 (T33-28A and T32-28B); and
SEQ ID NO:51 and SEQ ID NO:44 (T33-31A and T33-09B (also referred to as T33-31B))

In some embodiments, the one or more RSV and/or hMPV F proteins, or antigenic fragments thereof, are expressed as a fusion protein with the first multimerization domain. In some embodiments, the first multimerization domain and the RSV and/or hMPV F protein ectodomain are joined by a linker sequence. In some embodiments, the linker sequence comprises a foldon, wherein the foldon sequence is EKAAKAEEAARK (SEQ ID NO: 125).

Non-limiting examples of designed protein complexes useful in protein-based VLPs of the present disclosure include those disclosed in U.S. Pat. No. 9,630,994; Int'l Pat. Pub No. WO2018187325A1; U.S. Pat. Pub. No. 2018/0137234 A1; U.S. Pat. Pub. No. 2019/0155988 A2, each of which is incorporated herein in its entirety.

In various embodiments of the VLPs of the disclosure, the multimerization domains are polypeptides with the amino acid sequence selected from the following pairs, or modified versions thereof (i.e., permissible modifications as disclosed for the polypeptides of the invention: isolated polypeptides comprising an amino acid sequence that is at least 75%, at least 80%, at least 85%, at least 90%, at least 91%, at least 92%, at least 93%, at least 94%, at least 95%, at least 96%, at least 97%, at least 98%, at least 99%, or 100% over its length, and/or identical at least at one identified interface position, to the amino acid sequence indicated by the SEQ ID NO):
SEQ ID NO: 135 and SEQ ID NO: 136 (T33_dn2A and T33_dn2B);
SEQ ID NO: 137 and SEQ ID NO: 138 (T33_dn5A and T33_dn5B);
SEQ ID NO: 139 and SEQ ID NO: 140 (T33_dn10A and T33_dn10B); or
SEQ ID NO: 141 and SEQ ID NO: 142 (I53_dn5A and I53_dn5B).

### Antigenic Proteins

The present disclosure provides protein-based VLP vaccines for RSV and/or hMPV in humans or animals (in particular pets, agricultural animals, and any animal linked to spread of the disease). The present disclosure relates to incorporation of any of antigenic fragment of hMPV F protein—e.g., the ectodomain or an antigenic fragment thereof-into VLP vaccines. Guidance is particularly available from studies of the immune response to infection or vaccination, such as isolation of binding or neutralizing antibodies, genetic analysis of F protein sequence, structural studies of antigenic proteins and antibodies, and most particularly clinical and veterinary experience with subunit vaccines. Subunit vaccine for RSV or hMPV can be adapted for use with the VLPs of the present disclosure by employing the display modalities provided above. The disclosure refers to the ectodomain of RSV or hMPV F but it will be understood that portions of the transmembrane domain may be included.

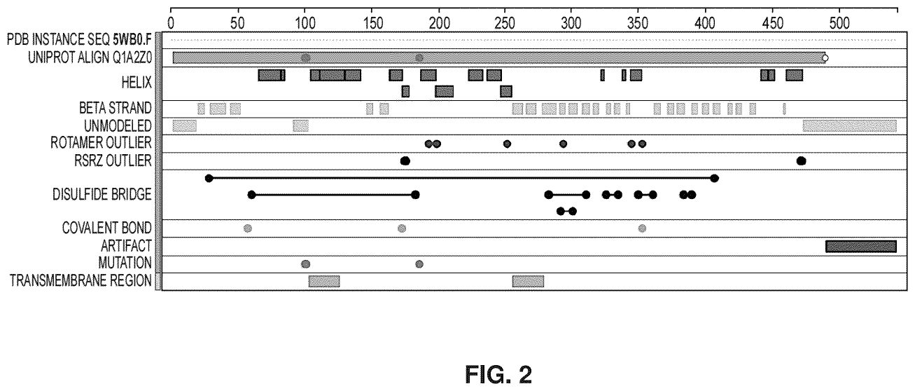

The term “antigenic fragment” refers to any fragment of a protein that generates an immune response (humoral or T cell response) to the protein in vivo. The antigenic fragment may be a linear epitope, discontinuous epitope, or a conformation epitope (e.g., a folded domain). The antigenic fragment may preserve the secondary, tertiary, and/or quaternary structure of the full-length protein. In some embodiments, the antigenic fragment comprises a neutralizing epitope. In such cases, the VLP may generate a neutralizing antibody response. Antigenic fragments may be designed computationally, such as by predicting the secondary structure and rationally removing N- or C-terminal unstructured regions or internally loops, or entire structural elements (alpha helices and/or beta sheets). FIG. 2 provides an illustrative secondary structure map. In some embodiments, the ectodomain comprises a truncation or deletion of some or all of the N-terminal signal peptide (e.g., MSWKVVIIFSLLITPQHGL (SEQ ID NO: 133) or MSWKVMIIISLLITPQHGL (SEQ ID NO: 134).

In some embodiments, the hMPV F protein ectodomain is a C-terminal truncation. In some embodiments, the hMPV F protein ectodomain sequence terminates at residue 470, 471, 472, 473, 474, 475, 476, 477, 478, 479, 480, 481, 482, 483, 484, 485, 486, 487, 488, 489, or 490 of the native sequence.

The native sequence of the A strain hMPV F protein (GenBank AY145297) is shown below with the signal sequence underlined and italicized and the transmembrane and intracellular portion underlined (SEQ ID NO: 56):

(SEQ ID NO: 56)

| | |
|---|---|
| 1 | MSWKVVIIFS LLITPQHGLK ESYLEESCST ITEGYLSVLR |
| 41 | TGWYTNVFTL EVGDVENLTC SDGPSLIKTE LDLTKSALRE |
| 81 | LKTVSADQLA REEQIENPRQ SRFVLGAIAL GVATAAAVTA |
| 121 | GVAIAKTIRL ESEVTAIKNA LKTTNEAVST LGNGVRVLAT |
| 161 | AVRELKDFVS KNLTRAINKN KCDIDDLKMA VSFSQFNRRF |
| 201 | LNVVRQFSDN AGITPAISLD LMTDAELARA VSNMPTSAGQ |
| 241 | IKLMLENRAM VRRKGFGILI GVYGSSVIYM VQLPIFGVID |
| 281 | TPCWIVKAAP SCSGKKGNYA CLLREDQGWY CQNAGSTVYY |
| 321 | PNEKDCETRG DHVFCDTAAG INVAEQSKEC NINISTTNYP |
| 361 | CKVSTGRHPI SMVALSPLGA LVACYKGVSC SIGSNRVGII |
| 401 | KQLNKGCSYI TNQDADTVTI DNTVYQLSKV EGEQHVIKGR |
| 441 | PVSSSFDPIK FPEDQFNVAL DQVFENIENS QALVDQSNRI |
| 481 | LSSAEKGNTG FIIVIILIAV LGSSMILVSI FIIIKKTKKP |
| 521 | TGAPPELSGV TNNGFIPHS |

The native signal sequence is post-translationally cleaved when the protein is expressed. The native signal sequence may be replaced with another signal sequence for expression of the ectodomain, or in some embodiments no signal sequence is used. Thus, in some embodiments, the hMPV F protein ectodomain is SEQ ID NO: 57, or a variant thereof:

(SEQ ID NO: 57)

| | |
|---|---|
| 1 | K ESYLEESCST ITEGYLSVLR |
| 41 | TGWYTNVFTL EVGDVENLTC SDGPSLIKTE LDLTKSALRE |
| 81 | LKTVSADQLA REEQIENPRQ SRFVLGAIAL GVATAAAVTA |
| 121 | GVAIAKTIRL ESEVTAIKNA LKTTNEAVST LGNGVRVLAT |
| 161 | AVRELKDFVS KNLTRAINKN KCDIDDLKMA VSFSQFNRRF |
| 201 | LNVVRQFSDN AGITPAISLD LMTDAELARA VSNMPTSAGQ |
| 241 | IKLMLENRAM VRRKGFGILI GVYGSSVIYM VQLPIFGVID |
| 281 | TPCWIVKAAP SCSGKKGNYA CLLREDQGWY CQNAGSTVYY |
| 321 | PNEKDCETRG DHVFCDTAAG INVAEQSKEC NINISTINYP |
| 361 | CKVSTGRHPI SMVALSPLGA LVACYKGVSC SIGSNRVGII |
| 401 | KQLNKGCSYI TNQDADTVTI DNTVYQLSKV EGEQHVIKGR |
| 441 | PVSSSFDPIK FPEDQFNVAL DQVFENIENS QALVDQSNRI |
| 481 | LSSAEKGNTG F |
| 521 | |

In some embodiments, the hMPV F protein ectodomain shares at least 90%, at least 91%, at least 92%, at least 93%, at least 94%, at least 95%, at least 96%, at least 97%, at least 98%, at least 99%, or 100% sequence identity to SEQ ID NO: 57, or an antigenic fragment thereof.

Further hMPV F protein sequences are provided in Table 2. In some embodiments, the hMPV F protein ectodomain shares at least 90%, at least 91%, at least 92%, at least 93%, at least 94%, at least 95%, at least 96%, at least 97%, at least 98%, at least 99%, or 100% sequence identity to an ectodomain in Table 2, or an antigenic fragment thereof.

Various signal sequences can be used, paired with the native ectodomain sequence or swapped between different hMPV sequences. Thus, in some embodiments, the antigen comprises a sequence that shares at least 90%, at least 91%, at least 92%, at least 93%, at least 94%, at least 95%, at least 96%, at least 97%, at least 98%, at least 99%, or 100% sequence identity to a signal sequence in Table 2.

The hMPV F protein may comprise one or more substitutions.

Illustrative substitutions include A185P, Q100R, S101R, T127C, N153C, V84C, A140C, A147C, A249C, N97G, P98G, R99G, Q100G, S101G, R102G, A63C, K188C, K450C, S470C, G106 deletion, A113C, A120C, A339C, Q426C, T160F, Q100K, S101A, I177L, K450A, S470A, G294E, T365C, V463C, L219K, H368N, and/or V231I relative to SEQ ID NO: 57. Thus, in various embodiments, the hMPV F protein ectodomain comprises one or more substitutions selected from this list.

Illustrative substitutions include V84C, A140C, A147C, N97G, P98G, Q100G, S101G, R102G, A63C, K188C, K450C, S470C, R99G, A113C, A120C, A339C, Q426C, T160F, Q100K, S101A, Q100R, S101R, G106 deletion, S101R, A185P, 1177L, and G294E relative to SEQ ID NO: 57. Thus, in various embodiments, the hMPV F protein ectodomain comprises one or more substitutions selected from this list.

Illustrative substitutions include V84C, A140C, A147C, N97G, P98G, Q100G, S101G, R102G, A63C, K188C, K450C, S470C, R99G, A113C, A120C, A339C, Q426C, T160F, Q100K, S101A, Q100R, S101R, G106 deletion, S101R, A185P, 1177L, and G294E. Thus, in various embodiments, the hMPV F protein ectodomain comprises two or more substitutions selected from this list.

Illustrative substitutions include A185P, Q100R, S101R, T127C, N153C, T365C, V463C, L219K, V231I, G294E, N153C, N97G, P98G, R99G, Q100G, S101G, and R102G. Thus, in various embodiments, the hMPV F protein ectodomain comprises one or more substitutions selected from this list.

Illustrative substitutions include A185P, Q100R, S101R, T127C, N153C, T365C, V463C, L219K, V231I, G294E, N153C, N97G, P98G, R99G, Q100G, S101G, and R102G. Thus, in various embodiments, the hMPV F protein ectodomain comprises two or more substitutions selected from this list.

In some embodiments, the hMPV F protein ectodomain comprises the substitutions Q100R and S101R. In some embodiments, the hMPV F protein ectodomain comprises the substitutions A185P, Q100R, and S101R. In some embodiments, the hMPV F protein ectodomain comprises the substitutions A185P, T127C, N153C, Q100R, and S101R. In some embodiments, the hMPV F protein ectodomain comprises the substitutions V84C, A140C, A147C, A249C, N97G, P98G, R99G, Q100G, S101G, and R102G. In some embodiments, the hMPV F protein ectodomain comprises the substitutions A63C, A140C, A147C, K188C, K450C, S470C, N97G, P98G, R99G, Q100G, S101G, and R102G. In some embodiments, the hMPV F protein ectodomain comprises the substitutions and deletion A63C, A140C, A147C, K188C, G106 deletion, N97G, P98G, R99G, Q100G, S101G, and R102G. In some embodiments, the hMPV F protein ectodomain comprises the substitutions A113C, A120C, A339C, Q426C, T160F, I177L, Q100K, and S101A. In some embodiments, the hMPV F protein ectodomain comprises the substitutions V84C, A140C, A147C, A249C, Q100R, and S101R. In some embodiments, the hMPV F protein ectodomain comprises the substitutions A63C, A140C, A147C, K188C, K450C, S470C, Q100R, and S101R. In some embodiments, the hMPV F protein ectodomain comprises the substitutions and deletion A63C, A140C, A147C, K188C, G106 deletion, Q100R, and S101R. In some embodiments, the hMPV F protein ectodomain comprises the substitutions A113C, A120C, A339C, Q426C, T160F, I177L, Q100R, and S101R. In some embodiments, the hMPV F protein ectodomain comprises the substitutions A185P, A113C, A339C, Q100R, and S101R. In some embodiments, the hMPV F protein ectodomain comprises the substitutions A185P, T160F, I177L, Q100R, and S101R. In some embodiments, the hMPV F protein ectodomain comprises the substitutions A185P, A113C, A339C, T160F, I177L, Q100R, and S101R. In some embodiments, the hMPV F protein ectodomain comprises the substitutions A63C, K188C, N97G, P98G, R99G, Q100G, S101G, and R102G. In some embodiments, the hMPV F protein ectodomain comprises the substitutions A63C, K188C, K450A, S470A, N97G, P98G, R99G, Q100G, S101G, and R102G. In some embodiments, the hMPV F protein ectodomain comprises the substitutions A63C, A140C, A147C, K188C, G294E, N97G, P98G, R99G, Q100G, S101G, and R102G. In some embodiments, the hMPV F protein ectodomain comprises the substitutions A63C, A140C, A147C, K188C, K450C, S470C, N97G, P98G, R99G, Q100G, S101G, R102G, and G294E. In some embodiments, the hMPV F protein ectodomain comprises the substitutions A63C, K188C, N97G, P98G, R99G, Q100G, S101G, R102G, and G294E. In some embodiments, the hMPV F protein ectodomain comprises the substitutions T127C, N153C, T365C, V463C, A185P, L219K, V231I, G294E, H368N, Q100R, and S101R. In some embodiments, the hMPV F protein ectodomain comprises the substitutions A63C, A140C, A147C, K188C, K450C, S470C, N97G, P98G, R99G, Q100G, S101G, and R102G. In some embodiments, the hMPV F protein ectodomain comprises the substitutions V84C, A140C, A147C, A249C, N97G, P98G, R99G, Q100G, S101G, and R102G. In some embodiments, the hMPV F protein ectodomain comprises the substitutions T127C, N153C, T365C, V463C, A185P, L219K, V231I, G294E, N97G, P98G, R99G, Q100G, S101G, H368N, and R102G.

In some embodiments, the substitutions have the intended effect on the VLP, it being understood that the intended effect is not achieved in every embodiment of the VLP having that substitution. In some embodiments, the intended effect is increased expression in a production cell line (Mas et al. PLoS Pathog. 12: e1005859 (2016)). In some embodiments, the intended effect is to stabilize a post-fusion or pre-fusion form of the hMIPV F protein ectodomain (Battles et al. Nat Commun. 8:1528 (2017)). In some embodiments, the intended effect is binding neutralizing antibodies against hMIPV F protein. The antigen may further comprise a Leucine at the N-terminus, as set forth in SEQ ID NOs: 146-178.

TABLE 2
Illustrative hMPV Protein F Ectodomain Antigenic Variants

| | | | C-term F |
|---|---|---|---|
| SEQ | | hMPV | protein |
| ID | | Antigen | Termination |
| NO: | Ectodomain | Mutations | Residue |
| 58 | KESYLEESCSTITEGYLSVLRTGWYTNVFTLEVGDVENLTCAD | A185P, | 481 |
| | GPSLIKTELDLTKSALRELRTVSADQLAREEQIENPRRRRFVL | Q100R, | |
| | GAIALGVATAAAVTAGVAIAKTIRLESEVTAIKNALKKTNEAV | S101R | |
| | STLGNGVRVLATAVRELKDFVSKNLTRAINKNKCDIPDLKMAV | | |
| | SFSQFNRRFLNVVRQFSDNAGITPAISLDLMTDAELARAVSNM | | |
| | PTSAGQIKLMLENRAMVRRKGFGFLIGVYGSSVIYMVQLPIFG | | |
| | VIDTPCWIVKAAPSCSGKKGNYACLLREDQGWYCQNAGSTVYY | | |
| | PNEKDCETRGDHVFCDTAAGINVAEQSKECNINISTTNYPCKV | | |
| | STGRHPISMVALSPLGALVACYKGVSCSIGSNRVGIIKQLNKG | | |
| | CSYITNQDADTVTIDNTVYQLSKVEGEQHVIKGRPVSSSFDPV | | |
| | KFPEDQFNVALDQVFESIENSQALVDQSNRIL | | |
| 59 | KESYLEESCSTITEGYLSVLRTGWYTNVFTLEVGDVENLTCAD | A185P, | 472 |
| | GPSLIKTELDLTKSALRELRTVSADQLAREEQIENPRRRRFVL | Q100R, | |
| | GAIALGVATAAAVTAGVAIAKTIRLESEVTAIKNALKKTNEAV | S101R | |
| | STLGNGVRVLATAVRELKDFVSKNLTRAINKNKCDIPDLKMAV | | |
| | SFSQFNRRFLNVVRQFSDNAGITPAISLDLMTDAELARAVSNM | | |
| | PTSAGQIKLMLENRAMVRRKGFGFLIGVYGSSVIYMVQLPIFG | | |
| | VIDTPCWIVKAAPSCSGKKGNYACLLREDQGWYCQNAGSTVYY | | |
| | PNEKDCETRGDHVFCDTAAGINVAEQSKECNINISTTNYPCKV | | |
| | STGRHPISMVALSPLGALVACYKGVSCSIGSNRVGIIKQLNKG | | |
| | CSYITNQDADTVTIDNTVYQLSKVEGEQHVIKGRPVSSSFDPV | | |
| | KFPEDQFNVALDQVFESIENSQA | | |
| 60 | KESYLEESCSTITEGYLSVLRTGWYTNVFTLEVGDVENLTCAD | A185P, | 489 |
| | GPSLIKTELDLTKSALRELRTVSADQLAREEQIENPRRRRFVL | Q100R, | |
| | GAIALGVATAAAVTAGVAIAKTIRLESEVTAIKNALKKTNEAV | S101R | |
| | STLGNGVRVLATAVRELKDFVSKNLTRAINKNKCDIPDLKMAV | | |
| | SFSQFNRRFLNVVRQFSDNAGITPAISLDLMTDAELARAVSNM | | |
| | PTSAGQIKLMLENRAMVRRKGFGFLIGVYGSSVIYMVQLPIFG | | |
| | VIDTPCWIVKAAPSCSGKKGNYACLLREDQGWYCQNAGSTVYY | | |
| | PNEKDCETRGDHVFCDTAAGINVAEQSKECNINISTTNYPCKV | | |
| | STGRHPISMVALSPLGALVACYKGVSCSIGSNRVGIIKQLNKG | | |
| | CSYITNQDADTVTIDNTVYQLSKVEGEQHVIKGRPVSSSFDPV | | |
| | KFPEDQFNVALDQVFESIENSQALVDQSNRILSSAEKGNT | | |
| 61 | KESYLEESCSTITEGYLSVLRTGWYTNVFTLEVGDVENLTCAD | Q100R, | 472 |
| | GPSLIKTELDLTKSALRELRTVSADQLAREEQIENPRRRRFVL | S101R | |
| | GAIALGVATAAAVTAGVAIAKTIRLESEVTAIKNALKKTNEAV | | |
| | STLGNGVRVLATAVRELKDFVSKNLTRAINKNKCDIADLKMAV | | |
| | SFSQFNRRFLNVVRQFSDNAGITPAISLDLMTDAELARAVSNM | | |
| | PTSAGQIKLMLENRAMVRRKGFGFLIGVYGSSVIYMVQLPIFG | | |
| | VIDTPCWIVKAAPSCSGKKGNYACLLREDQGWYCQNAGSTVYY | | |
| | PNEKDCETRGDHVFCDTAAGINVAEQSKECNINISTTNYPCKV | | |
| | STGRHPISMVALSPLGALVACYKGVSCSIGSNRVGIIKQLNKG | | |
| | CSYITNQDADTVTIDNTVYQLSKVEGEQHVIKGRPVSSSFDPV | | |
| | KFPEDQFNVALDQVFESIENSQA | | |
| 62 | KESYLEESCSTITEGYLSVLRTGWYTNVFTLEVGDVENLTCAD | A185P, | 472 |
| | GPSLIKTELDLTKSALRELRTVSADQLAREEQIENPRRRRFVL | T127C, | |
| | GAIALGVATAAAVTAGVAIAKCIRLESEVTAIKNALKKTNEAV | N153C, | |
| | STLGCGVRVLATAVRELKDFVSKNLTRAINKNKCDIPDLKMAV | Q100R, | |
| | SFSQFNRRFLNVVRQFSDNAGITPAISLDLMTDAELARAVSNM | S101R | |
| | PTSAGQIKLMLENRAMVRRKGFGILIGVYGSSVIYMVQLPIFG | | |
| | VIDTPCWIVKAAPSCSGKKGNYACLLREDQGWYCQNAGSTVYY | | |
| | PNEKDCETRGDHVFCDTAAGINVAEQSKECNINISTTNYPCKV | | |
| | STGRNPISMVALSPLGALVACYKGVSCSIGSNRVGIIKQLNKG | | |
| | CSYITNQDADTVTIDNTVYQLSKVEGEQHVIKGRPVSSSFDPV | | |
| | KFPEDQFNVALDQVFESIENSQA | | |
| 63 | KESYLEESCSTITEGYLSVLRTGWYTNVFTLEVGDVENLTCTD | V84C, | 472 |
| | GPSLIKTELDLTKSALRELKTCSADQLAREEQIEGGGGGGFVL | A140C, | |
| | GAIALGVATAAAVTAGIAIAKTIRLESEVNAIKGCLKTTNECV | A147C, | |
| | STLGNGVRVLATAVRELKEFVSKNLTSAINKNKCDIADLKMAV | A249C, | |
| | SFSQFNRRFLNVVRQFSDNAGITPAISLDLMTDAELARAVSYM | N97G, | |
| | PTSAGQIKLMLENRCMVRRKGFGILIGVYGSSVIYMVQLPIFG | P98G, | |
| | VIDTPCWIIKAAPSCSEKDGNYACLLREDQGWYCKNAGSTVYY | R99G, | |
| | PNDKDCETRGDHVFCDTAAGINVAEQSRECNINISTTNYPCKV | Q100G, | |
| | STGRHPISMVALSPLGALVACYKGVSCSIGSNRVGIIKQLPKG | S101G, | |
| | CSYITNQDADTVTIDNTVYQLSKVEGEQHVIKGRPVSSSFDPI | R102G | |
| | KFPEDQFNVALDQVFESIENSQA | | |
| 64 | KESYLEESCSTITEGYLSVLRTGWYTNVFTLEVGDVENLTCTD | V84C, | 488 |
| | GPSLIKTELDLTKSALRELKTCSADQLAREEQIEGGGGGGFVL | A140C, | |
| | GAIALGVATAAAVTAGIAIAKTIRLESEVNAIKGCLKTTNECV | A147C, | |
| | STLGNGVRVLATAVRELKEFVSKNLTSAINKNKCDIADLKMAV | A249C, | |
| | SFSQFNRRFLNVVRQFSDNAGITPAISLDLMTDAELARAVSYM | N97G, | |
| | PTSAGQIKLMLENRCMVRRKGFGILIGVYGSSVIYMVQLPIFG | P98G, | |
| | VIDTPCWIIKAAPSCSEKDGNYACLLREDQGWYCKNAGSTVYY | R99G, | |
| | PNDKDCETRGDHVFCDTAAGINVAEQSRECNINISTTNYPCKV | Q100G, | |
| | STGRHPISMVALSPLGALVACYKGVSCSIGSNRVGIIKQLPKG | S101G, | |
| | CSYITNQDADTVTIDNTVYQLSKVEGEQHVIKGRPVSSSFDPI | R102G | |
| | KFPEDQFNVALDQVFESIENSQALVDQSNKILNSAESAI | | |
| 65 | KESYLEESCSTITEGYLSVLRTGWYTNVFTLEVGDVENLTCTD | A63C, | 472 |
| | CPSLIKTELDLTKSALRELKTVSADQLAREEQIEGGGGGGFVL | A140C, | |
| | GAIALGVATAAAVTAGIAIAKTIRLESEVNAIKGCLKTTNECV | A147C, | |
| | STLGNGVRVLATAVRELKEFVSKNLTSAINKNKCDIADLCMAV | K188C, | |
| | SFSQFNRRFLNVVRQFSDNAGITPAISLDLMTDAELARAVSYM | K450C, | |
| | PTSAGQIKLMLENRAMVRRKGFGILIGVYGSSVIYMVQLPIFG | S470C, | |
| | VIDTPCWIIKAAPSCSEKDGNYACLLREDQGWYCKNAGSTVYY | N97G, | |
| | PNDKDCETRGDHVFCDTAAGINVAEQSRECNINISTTNYPCKV | P98G, | |
| | STGRHPISMVALSPLGALVACYKGVSCSIGSNRVGIIKQLPKG | R99G, | |
| | CSYITNQDADTVTIDNTVYQLSKVEGEQHVIKGRPVSSSFDPI | Q100G, | |
| | CFPEDQFNVALDQVFESIENCQA | S101G, | |
| | | R102G | |
| 66 | KESYLEESCSTITEGYLSVLRTGWYTNVFTLEVGDVENLTCTD | A63C, | 488 |
| | CPSLIKTELDLTKSALRELKTVSADQLAREEQIEGGGGGGFVL | A140C, | |
| | GAIALGVATAAAVTAGIAIAKTIRLESEVNAIKGCLKTTNECV | A147C, | |
| | STLGNGVRVLATAVRELKEFVSKNLTSAINKNKCDIADLCMAV | K188C, | |
| | SFSQFNRRFLNVVRQFSDNAGITPAISLDLMTDAELARAVSYM | K450C, | |
| | PTSAGQIKLMLENRAMVRRKGFGILIGVYGSSVIYMVQLPIFG | S470C, | |
| | VIDTPCWIIKAAPSCSEKDGNYACLLREDQGWYCKNAGSTVYY | N97G, | |
| | PNDKDCETRGDHVFCDTAAGINVAEQSRECNINISTTNYPCKV | P98G, | |
| | STGRHPISMVALSPLGALVACYKGVSCSIGSNRVGIIKQLPKG | R99G, | |
| | CSYITNQDADTVTIDNTVYQLSKVEGEQHVIKGRPVSSSFDPI | Q100G, | |
| | CFPEDQFNVALDQVFESIENCQALVDQSNKILNSAESAI | S101G, | |
| | | R102G | |
| 67 | KESYLEESCSTITEGYLSVLRTGWYTNVFTLEVGDVENLTCSD | A63C, | 472 |
| | CPSLIKTELDLTKSALRELKTVSADQLAREEQIEGGGGGGFVL | A140C, | |
| | AIALGVATAAAVTAGVAIAKTIRLESEVTAIKNCLKTTNECVS | A147C, | |
| | TLGNGVRVLATAVRELKDFVSKNLTSAINKNKCDIDDLCMAVS | K188C, | |
| | FSQFNRRFLNVVRQFSDNAGITPAISLDLMTDAELARAVSNMP | G106 | |
| | TSAGQIKLMLENRAMVRRKGFGILIGVYGSSVIYMVQLPIFGV | deleted, | |
| | IDTPCWIIKAAPSCSGKKGNYACLLREDQGWYCQNAGSTVYYP | N97G, | |
| | NEKDCETRGDHVFCDTAAGINVAEQSKECNINISTTNYPCKVS | P98G, | |
| | TGRHPISMVALSPLGALVACYKGVSCSIGSNRVGIIKQLNKGC | R99G, | |
| | SYITNQDADTVTIDNTVYQLSKVEGEQHVIKGRPVSSSFDPIK | Q100G, | |
| | FPEDQFNVALDQVFENIENSQA | S101G, | |
| | | R102G | |
| 68 | KESYLEESCSTITEGYLSVLRTGWYTNVFTLEVGDVENLTCSD | A63C, | 488 |
| | CPSLIKTELDLTKSALRELKTVSADQLAREEQIEGGGGGGFVL | A140C, | |
| | AIALGVATAAAVTAGVAIAKTIRLESEVTAIKNCLKTTNECVS | A147C, | |
| | TLGNGVRVLATAVRELKDFVSKNLTSAINKNKCDIDDLCMAVS | K188C, | |
| | FSQFNRRFLNVVRQFSDNAGITPAISLDLMTDAELARAVSNMP | G106 | |
| | TSAGQIKLMLENRAMVRRKGFGILIGVYGSSVIYMVQLPIFGV | deleted, | |
| | IDTPCWIIKAAPSCSGKKGNYACLLREDQGWYCQNAGSTVYYP | N97G, | |
| | NEKDCETRGDHVFCDTAAGINVAEQSKECNINISTTNYPCKVS | P98G, | |
| | TGRHPISMVALSPLGALVACYKGVSCSIGSNRVGIIKQLNKGC | R99G, | |
| | SYITNQDADTVTIDNTVYQLSKVEGEQHVIKGRPVSSSFDPIK | Q100G, | |
| | FPEDQFNVALDQVFENIENSQALVDQSNRILSSAESAI | S101G, | |
| | | R102G | |
| 69 | KESYLEESCSTITEGYLSVLRTGWYTNVFTLEVGDVENLTCTD | A113C, | 472 |
| | GPSLIKTELDLTKSALRELKTVSADQLAREEQIENPRKARFVL | A120C, | |
| | GAIALGVCTAAAVTCGIAIAKTIRLESEVNAIKGALKTTNEAV | A339C, | |
| | STLGNGVRVLAFAVRELKEFVSKNLTSALNKNKCDIADLKMAV | Q426C, | |
| | SFSQFNRRFLNVVRQFSDNAGITPAISLDLMTDAELARAVSYM | T160F, | |
| | PTSAGQIKLMLENRAMVRRKGFGILIGVYGSSVIYMVQLPIFG | I17L, | |
| | VIDTPCWIIKAAPSCSEKDGNYACLLREDQGWYCKNAGSTVYY | Q100K, | |
| | PNDKDCETRGDHVFCDTACGINVAEQSRECNINISTTNYPCKV | S101A | |
| | STGRHPISMVALSPLGALVACYKGVSCSIGSNRVGIIKQLPKG | | |
| | CSYITNQDADTVTIDNTVYCLSKVEGEQHVIKGRPVSSSFDPI | | |
| | KFPEDQFNVALDQVFESIENSQA | | |
| 70 | KESYLEESCSTITEGYLSVLRTGWYTNVFTLEVGDVENLTCTD | A113C, | 488 |
| | GPSLIKTELDLTKSALRELKTVSADQLAREEQIENPRKARFVL | A120C, | |
| | GAIALGVCTAAAVTCGIAIAKTIRLESEVNAIKGALKTTNEAV | A339C, | |
| | STLGNGVRVLAFAVRELKEFVSKNLTSALNKNKCDIADLKMAV | Q426C, | |
| | SFSQFNRRFLNVVRQFSDNAGITPAISLDLMTDAELARAVSYM | T160F, | |
| | PTSAGQIKLMLENRAMVRRKGFGILIGVYGSSVIYMVQLPIFG | I177L, | |
| | VIDTPCWIIKAAPSCSEKDGNYACLLREDQGWYCKNAGSTVYY | Q100K, | |
| | PNDKDCETRGDHVFCDTACGINVAEQSRECNINISTTNYPCKV | S101A | |
| | STGRHPISMVALSPLGALVACYKGVSCSIGSNRVGIIKQLPKG | | |
| | CSYITNQDADTVTIDNTVYCLSKVEGEQHVIKGRPVSSSFDPI | | |
| | KFPEDQFNVALDQVFESIENSQALVDQSNKILNSAESAI | | |
| 71 | MSWKVMIIISLLITPQHGLKESYLEESCSTITEGYLSVLRTGW | V84C, | 472 |
| | YTNVFTLEVGDVENLTCTDGPSLIKTELDLTKSALRELKTCSA | A140C, | |
| | DQLAREEQIENPRRRRFVLGAIALGVATAAAVTAGIAIAKTIR | A147C, | |
| | LESEVNAIKGCLKTTNECVSTLGNGVRVLATAVRELKEFVSKN | A249C, | |
| | LTSAINKNKCDIADLKMAVSFSQFNRRFLNVVRQFSDNAGITP | Q100R, | |
| | AISLDLMTDAELARAVSYMPTSAGQIKLMLENRCMVRRKGFGI | S101R | |
| | LIGVYGSSVIYMVQLPIFGVIDTPCWIIKAAPSCSEKDGNYAC | | |
| | LLREDQGWYCKNAGSTVYYPNDKDCETRGDHVFCDTAAGINVA | | |
| | EQSRECNINISTTNYPCKVSTGRHPISMVALSPLGALVACYKG | | |
| | VSCSIGSNRVGIIKQLPKGCSYITNQDADTVTIDNTVYQLSKV | | |
| | EGEQHVIKGRPVSSSFDPIKFPEDQFNVALDQVFESIENSQA | | |
| 72 | KESYLEESCSTITEGYLSVLRTGWYTNVFTLEVGDVENLTCTD | A63C, | 472 |
| | CPSLIKTELDLTKSALRELKTVSADQLAREEQIENPRRRRFVL | A140C, | |
| | GAIALGVATAAAVTAGIAIAKTIRLESEVNAIKGCLKTTNECV | A147C, | |
| | STLGNGVRVLATAVRELKEFVSKNLTSAINKNKCDIADLCMAV | K188C, | |
| | SFSQFNRRFLNVVRQFSDNAGITPAISLDLMTDAELARAVSYM | K450C, | |
| | PTSAGQIKLMLENRAMVRRKGFGILIGVYGSSVIYMVQLPIFG | S470C, | |
| | VIDTPCWIIKAAPSCSEKDGNYACLLREDQGWYCKNAGSTVYY | Q100R, | |
| | PNDKDCETRGDHVFCDTAAGINVAEQSRECNINISTTNYPCKV | S101R | |
| | STGRHPISMVALSPLGALVACYKGVSCSIGSNRVGIIKQLPKG | | |
| | CSYITNQDADTVTIDNTVYQLSKVEGEQHVIKGRPVSSSFDPI | | |
| | CFPEDQFNVALDQVFESIENCQA | | |
| 73 | KESYLEESCSTITEGYLSVLRTGWYTNVFTLEVGDVENLTCSD | A63C, | 472 |
| | CPSLIKTELDLTKSALRELKTVSADQLAREEQIENPRRRRFVL | A140C, | |
| | AIALGVATAAAVTAGVAIAKTIRLESEVTAIKNCLKTTNECVS | A147C, | |
| | TLGNGVRVLATAVRELKDFVSKNLTSAINKNKCDIDDLCMAVS | K188C, | |
| | FSQFNRRFLNVVRQFSDNAGITPAISLDLMTDAELARAVSNMP | G106 | |
| | TSAGQIKLMLENRAMVRRKGFGILIGVYGSSVIYMVQLPIFGV | deleted, | |
| | IDTPCWIIKAAPSCSGKKGNYACLLREDQGWYCQNAGSTVYYP | Q100R, | |
| | NEKDCETRGDHVFCDTAAGINVAEQSKECNINISTTNYPCKVS | S101R | |
| | TGRHPISMVALSPLGALVACYKGVSCSIGSNRVGIIKQLNKGC | | |
| | SYITNQDADTVTIDNTVYQLSKVEGEQHVIKGRPVSSSFDPIK | | |
| | FPEDQFNVALDQVFENIENSQA | | |
| 74 | KESYLEESCSTITEGYLSVLRTGWYTNVFTLEVGDVENLTCTD | A113C, | 472 |
| | GPSLIKTELDLTKSALRELKTVSADQLAREEQIENPRRRRFVL | A120C, | |
| | GAIALGVCTAAAVTCGIAIAKTIRLESEVNAIKGALKTTNEAV | A339C, | |
| | STLGNGVRVLAFAVRELKEFVSKNLTSALNKNKCDIADLKMAV | Q426C, | |
| | SFSQFNRRFLNVVRQFSDNAGITPAISLDLMTDAELARAVSYM | T160F, | |
| | PTSAGQIKLMLENRAMVRRKGFGILIGVYGSSVIYMVQLPIFG | I177L, | |
| | VIDTPCWIIKAAPSCSEKDGNYACLLREDQGWYCKNAGSTVYY | Q100R, | |
| | PNDKDCETRGDHVFCDTACGINVAEQSRECNINISTTNYPCKV | S101R | |
| | STGRHPISMVALSPLGALVACYKGVSCSIGSNRVGIIKQLPKG | | |
| | CSYITNQDADTVTIDNTVYCLSKVEGEQHVIKGRPVSSSFDPI | | |
| | KFPEDQFNVALDQVFESIENSQA | | |
| 75 | KESYLEESCSTITEGYLSVLRTGWYTNVFTLEVGDVENLTCAD | A185P, | 472 |
| | GPSLIKTELDLTKSALRELRTVSADQLAREEQIENPRRRRFVL | A113C, | |
| | GAIALGVCTAAAVTAGVAIAKTIRLESEVTAIKNALKKTNEAV | A339C, | |
| | STLGNGVRVLATAVRELKDFVSKNLTRAINKNKCDIPDLKMAV | Q100R, | |
| | SFSQFNRRFLNVVRQFSDNAGITPAISLDLMTDAELARAVSNM | S101R | |
| | PTSAGQIKLMLENRAMVRRKGFGFLIGVYGSSVIYMVQLPIFG | | |
| | VIDTPCWIVKAAPSCSGKKGNYACLLREDQGWYCQNAGSTVYY | | |
| | PNEKDCETRGDHVFCDTACGINVAEQSKECNINISTTNYPCKV | | |
| | STGRHPISMVALSPLGALVACYKGVSCSIGSNRVGIIKQLNKG | | |
| | CSYITNQDADTVTIDNTVYQLSKVEGEQHVIKGRPVSSSFDPV | | |
| | KFPEDQFNVALDQVFESIENSQA | | |
| 76 | KESYLEESCSTITEGYLSVLRTGWYTNVFTLEVGDVENLTCAD | A185P, | 472 |
| | GPSLIKTELDLTKSALRELRTVSADQLAREEQIENPRRRRFVL | T160F, | |
| | GAIALGVATAAAVTAGVAIAKTIRLESEVTAIKNALKKTNEAV | I177L, | |
| | STLGNGVRVLAFAVRELKDFVSKNLTRALNKNKCDIPDLKMAV | Q100R, | |
| | SFSQFNRRFLNVVRQFSDNAGITPAISLDLMTDAELARAVSNM | S101R | |
| | PTSAGQIKLMLENRAMVRRKGFGFLIGVYGSSVIYMVQLPIFG | | |
| | VIDTPCWIVKAAPSCSGKKGNYACLLREDQGWYCQNAGSTVYY | | |
| | PNEKDCETRGDHVFCDTAAGINVAEQSKECNINISTTNYPCKV | | |
| | STGRHPISMVALSPLGALVACYKGVSCSIGSNRVGIIKQLNKG | | |
| | CSYITNQDADTVTIDNTVYQLSKVEGEQHVIKGRPVSSSFDPV | | |
| | KFPEDQFNVALDQVFESIENSQA | | |
| 77 | KESYLEESCSTITEGYLSVLRTGWYTNVFTLEVGDVENLTCAD | A185P, | 472 |
| | GPSLIKTELDLTKSALRELRTVSADQLAREEQIENPRRRRFVL | A113C, | |
| | GAIALGVCTAAAVTAGVAIAKTIRLESEVTAIKNALKKTNEAV | A339C, | |
| | STLGNGVRVLAFAVRELKDFVSKNLTRALNKNKCDIPDLKMAV | T160F, | |
| | SFSQFNRRFLNVVRQFSDNAGITPAISLDLMTDAELARAVSNM | I177L, | |
| | PTSAGQIKLMLENRAMVRRKGFGFLIGVYGSSVIYMVQLPIFG | Q100R, | |
| | VIDTPCWIVKAAPSCSGKKGNYACLLREDQGWYCQNAGSTVYY | S101R | |
| | PNEKDCETRGDHVFCDTACGINVAEQSKECNINISTTNYPCKV | | |
| | STGRHPISMVALSPLGALVACYKGVSCSIGSNRVGIIKQLNKG | | |
| | CSYITNQDADTVTIDNTVYQLSKVEGEQHVIKGRPVSSSFDPV | | |
| | KFPEDQFNVALDQVFESIENSQA | | |
| 78 | KESYLEESCSTITEGYLSVLRTGWYTNVFTLEVGDVENLTCTD | A63C, | 472 |
| | CPSLIKTELDLTKSALRELKTVSADQLAREEQIEGGGGGGFVL | K188C, | |
| | GAIALGVATAAAVTAGIAIAKTIRLESEVNAIKGALKTTNEAV | N97G, | |
| | STLGNGVRVLATAVRELKEFVSKNLTSAINKNKCDIADLCMAV | P98G, | |
| | SFSQFNRRFLNVVRQFSDNAGITPAISLDLMTDAELARAVSYM | R99G, | |
| | PTSAGQIKLMLENRAMVRRKGFGILIGVYGSSVIYMVQLPIFG | Q100G, | |
| | VIDTPCWIIKAAPSCSEKDGNYACLLREDQGWYCKNAGSTVYY | S101G, | |
| | PNDKDCETRGDHVFCDTAAGINVAEQSRECNINISTTNYPCKV | R102G | |
| | STGRHPISMVALSPLGALVACYKGVSCSIGSNRVGIIKQLPKG | | |
| | CSYITNQDADTVTIDNTVYQLSKVEGEQHVIKGRPVSSSFDPI | | |
| | KFPEDQFNVALDQVFESIENSQA | | |
| 79 | KESYLEESCSTITEGYLSVLRTGWYTNVFTLEVGDVENLTCTD | A63C, | 472 |
| | CPSLIKTELDLTKSALRELKTVSADQLAREEQIEGGGGGGFVL | K188C, | |
| | GAIALGVATAAAVTAGIAIAKTIRLESEVNAIKGALKTTNEAV | K450A, | |
| | STLGNGVRVLATAVRELKEFVSKNLTSAINKNKCDIADLCMAV | S470A, | |
| | SFSQFNRRFLNVVRQFSDNAGITPAISLDLMTDAELARAVSYM | N97G, | |
| | PTSAGQIKLMLENRAMVRRKGFGILIGVYGSSVIYMVQLPIFG | P98G, | |
| | VIDTPCWIIKAAPSCSEKDGNYACLLREDQGWYCKNAGSTVYY | R99G, | |
| | PNDKDCETRGDHVFCDTAAGINVAEQSRECNINISTTNYPCKV | Q100G, | |
| | STGRHPISMVALSPLGALVACYKGVSCSIGSNRVGIIKQLPKG | S101G, | |
| | CSYITNQDADTVTIDNTVYQLSKVEGEQHVIKGRPVSSSFDPI | R102G | |
| | AFPEDQFNVALDQVFESIENAQA | | |
| 80 | KESYLEESCSTITEGYLSVLRTGWYTNVFTLEVGDVENLTCSD | A63C, | 472 |
| | CPSLIKTELDLTKSALRELKTVSADQLAREEQIEGGGGGGFVL | A140C, | |
| | GAIALGVATAAAVTAGVAIAKTIRLESEVTAIKNCLKTTNECV | A147C, | |
| | STLGNGVRVLATAVRELKDFVSKNLTSAINKNKCDIDDLCMAV | K188C, | |
| | SFSQFNRRFLNVVRQFSDNAGITPAISLDLMTDAELARAVSNM | G294E, | |
| | PTSAGQIKLMLENRAMVRRKGFGILIGVYGSSVIYMVQLPIFG | N97G, | |
| | VIDTPCWIIKAAPSCSEKKGNYACLLREDQGWYCQNAGSTVYY | P98G, | |
| | PNEKDCETRGDHVFCDTAAGINVAEQSKECNINISTTNYPCKV | R99G, | |
| | STGRHPISMVALSPLGALVACYKGVSCSIGSNRVGIIKQLNKG | Q100G, | |
| | CSYITNQDADTVTIDNTVYQLSKVEGEQHVIKGRPVSSSFDPI | S101G, | |
| | KFPEDQFNVALDQVFENIENSQA | R102G | |
| 81 | KESYLEESCSTITEGYLSVLRTGWYTNVFTLEVGDVENLTCAD | A63C, | 472 |
| | CPSLIKTELDLTKSALRELRTVSADQLAREEQIEGGGGGGFVL | A140C, | |
| | GAIALGVATAAAVTAGVAIAKTIRLESEVTAIKNCLKKTNECV | A147C, | |
| | STLGNGVRVLATAVRELKDFVSKNLTRAINKNKCDIADLCMAV | K188C, | |
| | SFSQFNRRFLNVVRQFSDNAGITPAISLDLMTDAELARAVSNM | K450C, | |
| | PTSAGQIKLMLENRAMVRRKGFGFLIGVYGSSVIYMVQLPIFG | S470C, | |
| | VIDTPCWIVKAAPSCSEKKGNYACLLREDQGWYCQNAGSTVYY | N97G, | |
| | PNEKDCETRGDHVFCDTAAGINVAEQSKECNINISTTNYPCKV | P98G, | |
| | STGRHPISMVALSPLGALVACYKGVSCSIGSNRVGIIKQLNKG | R99G, | |
| | CSYITNQDADTVTIDNTVYQLSKVEGEQHVIKGRPVSSSFDPV | Q100G, | |
| | CFPEDQFNVALDQVFESIENCQA | S101G, | |
| | | R102G, | |
| | | G294E | |
| 82 | KESYLEESCSTITEGYLSVLRTGWYTNVFTLEVGDVENLTCAD | A63C, | 472 |
| | CPSLIKTELDLTKSALRELRTVSADQLAREEQIEGGGGGGFVL | K188C, | |
| | GAIALGVATAAAVTAGVAIAKTIRLESEVTAIKNALKKTNEAV | N97G, | |
| | STLGNGVRVLATAVRELKDFVSKNLTRAINKNKCDIADLCMAV | P98G, | |
| | SFSQFNRRFLNVVRQFSDNAGITPAISLDLMTDAELARAVSNM | R99G, | |
| | PTSAGQIKLMLENRAMVRRKGFGFLIGVYGSSVIYMVQLPIFG | Q100G, | |
| | VIDTPCWIVKAAPSCSEKKGNYACLLREDQGWYCQNAGSTVYY | S101G, | |
| | PNEKDCETRGDHVFCDTAAGINVAEQSKECNINISTTNYPCKV | R102G, | |
| | STGRHPISMVALSPLGALVACYKGVSCSIGSNRVGIIKQLNKG | G294E | |
| | CSYITNQDADTVTIDNTVYQLSKVEGEQHVIKGRPVSSSFDPV | | |
| | KFPEDQFNVALDQVFESIENSQA | | |
| 83 | KESYLEESCSTITEGYLSVLRTGWYTNVFTLEVGDVENLTCAD | T127C, | 472 |
| | GPSLIKTELDLTKSALRELRTVSADQLAREEQIENPRRRRFVL | N153C, | |
| | GAIALGVATAAAVTAGVAIAKCIRLESEVTAIKNALKKTNEAV | T365C, | |
| | STLGCGVRVLATAVRELKDFVSKNLTRAINKNKCDIPDLKMAV | V463C, | |
| | SFSQFNRRFLNVVRQFSDNAGITPAISKDLMTDAELARAISNM | A185P, | |
| | PTSAGQIKLMLENRAMVRRKGFGILIGVYGSSVIYMVQLPIFG | L219K, | |
| | VIDTPCWIVKAAPSCSEKKGNYACLLREDQGWYCQNAGSTVYY | V231I, | |
| | PNEKDCETRGDHVFCDTAAGINVAEQSKECNINISTTNYPCKV | G294E, | |
| | SCGRNPISMVALSPLGALVACYKGVSCSIGSNRVGIIKQLNKG | H368N, | |
| | CSYITNQDADTVTIDNTVYQLSKVEGEQHVIKGRPVSSSFDPV | Q100R, | |
| | KFPEDQFNVALDQCFESIENSQA | S101R | |
| 84 | KESYLEESCSTITEGYLSVLRTGWYTNVFTLEVGDVENLTCAD | T127C, | 490 |
| | GPSLIKTELDLTKSALRELRTVSADQLAREEQIENPRRRRFVL | N153C, | |
| | GAIALGVATAAAVTAGVAIAKCIRLESEVTAIKNALKKTNEAV | T365C, | |
| | STLGCGVRVLATAVRELKDFVSKNLTRAINKNKCDIPDLKMAV | V463C, | |
| | SFSQFNRRFLNVVRQFSDNAGITPAISKDLMTDAELARAISNM | A185P, | |
| | PTSAGQIKLMLENRAMVRRKGFGILIGVYGSSVIYMVQLPIFG | L219K, | |
| | VIDTPCWIVKAAPSCSEKKGNYACLLREDQGWYCQNAGSTVYY | V231I, | |
| | PNEKDCETRGDHVFCDTAAGINVAEQSKECNINISTTNYPCKV | G294E, | |
| | SCGRNPISMVALSPLGALVACYKGVSCSIGSNRVGIIKQLNKG | H368N, | |
| | CSYITNQDADTVTIDNTVYQLSKVEGEQHVIKGRPVSSSFDPV | Q100R, | |
| | KFPEDQFNVALDQCFESIENSQALVDQSNRILSSAEKGNTG | S101R | |
| 85 | KESYLEESCSTITEGYLSVLRTGWYTNVFTLEVGDVENLTCTD | A63C, | 472 |
| | CPSLIKTELDLTKSALRELKTVSADQLAREEQIEGGGGGGFVL | A140C, | |
| | GAIALGVATAAAVTAGIAIAKTIRLESEVNAIKGCLKTTNECV | A147C, | |
| | STLGNGVRVLATAVRELKEFVSKNLTSAINKNKCDIADLCMAV | K188C, | |
| | SFSQFNRRFLNVVRQFSDNAGITPAISLDLMTDAELARAVSYM | K450C, | |
| | PTSAGQIKLMLENRAMVRRKGFGILIGVYGSSVIYMVQLPIFG | S470C, | |
| | VIDTPCWIIKAAPSCSEKDGNYACLLREDQGWYCKNAGSTVYY | N97G, | |
| | PNDKDCETRGDHVFCDTAAGINVAEQSRECNINISTTNYPCKV | P98G, | |
| | STGRHPISMVALSPLGALVACYKGVSCSIGSNRVGIIKQLPKG | R99G, | |
| | CSYITNQDADTVTIDNTVYQLSKVEGEQHVIKGRPVSSSFDPI | Q100G, | |
| | CFPEDQFNVALDQVFESIENCQA | S101G, | |
| | | R102G | |
| 86 | KESYLEESCSTITEGYLSVLRTGWYTNVFTLEVGDVENLTCAD | T127C, | 472 |
| | GPSLIKTELDLTKSALRELRTVSADQLAREEQIENPRRRRFVL | N153C, | |
| | GAIALGVATAAAVTAGVAIAKCIRLESEVTAIKNALKKTNEAV | T365C, | |
| | STLGCGVRVLATAVRELKDFVSKNLTRAINKNKCDIPDLKMAV | V463C, | |
| | SFSQFNRRFLNVVRQFSDNAGITPAISKDLMTDAELARAISNM | A185P, | |
| | PTSAGQIKLMLENRAMVRRKGFGILIGVYGSSVIYMVQLPIFG | L219K, | |
| | VIDTPCWIVKAAPSCSEKKGNYACLLREDQGWYCQNAGSTVYY | V231I, | |
| | PNEKDCETRGDHVFCDTAAGINVAEQSKECNINISTTNYPCKV | G294E, | |
| | SCGRNPISMVALSPLGALVACYKGVSCSIGSNRVGIIKQLNKG | Q100R, | |
| | CSYITNQDADTVTIDNTVYQLSKVEGEQHVIKGRPVSSSFDPV | S101R | |
| | KFPEDQFNVALDQCFESIENSQA | | |
| 87 | KESYLEESCSTITEGYLSVLRTGWYTNVFTLEVGDVENLTCAD | T127C, | 490 |
| | GPSLIKTELDLTKSALRELRTVSADQLAREEQIENPRRRRFVL | N153C, | |
| | GAIALGVATAAAVTAGVAIAKCIRLESEVTAIKNALKKTNEAV | T365C, | |
| | STLGCGVRVLATAVRELKDFVSKNLTRAINKNKCDIPDLKMAV | V463C, | |
| | SFSQFNRRFLNVVRQFSDNAGITPAISKDLMTDAELARAISNM | A185P, | |
| | PTSAGQIKLMLENRAMVRRKGFGILIGVYGSSVIYMVQLPIFG | L219K, | |
| | VIDTPCWIVKAAPSCSEKKGNYACLLREDQGWYCQNAGSTVYY | V231I, | |
| | PNEKDCETRGDHVFCDTAAGINVAEQSKECNINISTTNYPCKV | G294E, | |
| | SCGRNPISMVALSPLGALVACYKGVSCSIGSNRVGIIKQLNKG | Q100R, | |
| | CSYITNQDADTVTIDNTVYQLSKVEGEQHVIKGRPVSSSFDPV | S101R | |
| | KFPEDQFNVALDQCFESIENSQALVDQSNRILSSAEKGNTG | | |
| 88 | KESYLEESCSTITEGYLSVLRTGWYTNVFTLEVGDVENLTCTD | V84C, | 472 |
| | GPSLIKTELDLTKSALRELKTCSADQLAREEQIEGGGGGGFVL | A140C, | |
| | GAIALGVATAAAVTAGIAIAKTIRLESEVNAIKGCLKTTNECV | A147C, | |
| | STLGNGVRVLATAVRELKEFVSKNLTSAINKNKCDIADLKMAV | A249C, | |
| | SFSQFNRRFLNVVRQFSDNAGITPAISLDLMTDAELARAVSYM | N97G, | |
| | PTSAGQIKLMLENRCMVRRKGFGILIGVYGSSVIYMVQLPIFG | P98G, | |
| | VIDTPCWIIKAAPSCSEKDGNYACLLREDQGWYCKNAGSTVYY | R99G, | |
| | PNDKDCETRGDHVFCDTAAGINVAEQSRECNINISTTNYPCKV | Q100G, | |
| | STGRHPISMVALSPLGALVACYKGVSCSIGSNRVGIIKQLPKG | S101G, | |
| | CSYITNQDADTVTIDNTVYQLSKVEGEQHVIKGRPVSSSFDPI | R102G | |
| | KFPEDQFNVALDQVFESIENSQA | | |
| 89 | KESYLEESCSTITEGYLSVLRTGWYTNVFTLEVGDVENLTCAD | T127C, | 472 |
| | GPSLIKTELDLTKSALRELRTVSADQLAREEQIEGGGGGGFVL | N153C, | |
| | GAIALGVATAAAVTAGVAIAKCIRLESEVTAIKNALKKTNEAV | T365C, | |
| | STLGCGVRVLATAVRELKDFVSKNLTRAINKNKCDIPDLKMAV | V463C, | |
| | SFSQFNRRFLNVVRQFSDNAGITPAISKDLMTDAELARAISNM | A185P, | |
| | PTSAGQIKLMLENRAMVRRKGFGILIGVYGSSVIYMVQLPIFG | L219K, | |
| | VIDTPCWIVKAAPSCSEKKGNYACLLREDQGWYCQNAGSTVYY | V231I, | |
| | PNEKDCETRGDHVFCDTAAGINVAEQSKECNINISTTNYPCKV | G294E, | |
| | SCGRNPISMVALSPLGALVACYKGVSCSIGSNRVGIIKQLNKG | N97G, | |
| | CSYITNQDADTVTIDNTVYQLSKVEGEQHVIKGRPVSSSFDPV | P98G, | |
| | KFPEDQFNVALDQCFESIENSQA | R99G, | |
| | | Q100G, | |
| | | S101G, | |
| | | H368N, | |
| | | R102G | |
| 90 | KESYLEESCSTITEGYLSVLRTGWYTNVFTLEVGDVENLTCAD | T127C, | 490 |
| | GPSLIKTELDLTKSALRELRTVSADQLAREEQIEGGGGGGFVL | N153C, | |
| | GAIALGVATAAAVTAGVAIAKCIRLESEVTAIKNALKKTNEAV | T365C, | |
| | STLGCGVRVLATAVRELKDFVSKNLTRAINKNKCDIPDLKMAV | V463C, | |
| | SFSQFNRRFLNVVRQFSDNAGITPAISKDLMTDAELARAISNM | A185P, | |
| | PTSAGQIKLMLENRAMVRRKGFGILIGVYGSSVIYMVQLPIFG | L219K, | |
| | VIDTPCWIVKAAPSCSEKKGNYACLLREDQGWYCQNAGSTVYY | V231I, | |
| | PNEKDCETRGDHVFCDTAAGINVAEQSKECNINISTTNYPCKV | G294E, | |
| | SCGRNPISMVALSPLGALVACYKGVSCSIGSNRVGIIKQLNKG | N97G, | |
| | CSYITNQDADTVTIDNTVYQLSKVEGEQHVIKGRPVSSSFDPV | P98G, | |
| | KFPEDQFNVALDQCFESIENSQALVDQSNRILSSAEKGNTG | R99G, | |
| | | Q100G, | |
| | | S101G, | |
| | | H368N, | |
| | | R102G | |

Thus, in some embodiments, the hMIPV F protein ectodomain shares at least 900o, at least 91%, at least 92%, at least 93%, at least 94%, at least 95%, at least 96%, at least 97%, at least 98%, at least 99%, or 100% sequence identity to SEQ ID NOs: 58-90, or an antigenic fragment thereof.

In some embodiments, the hMPV F protein comprises one or more furin cleavage sites, optionally one or more copies of an Arg-X-X-Arg motif (SEQ ID NO: 52), such as the native sequence RQSR (SEQ ID NO: 54). Where a furin cleavage site is present in the hMPV ectodomain, expression yields may, in some cases, be increased by co-expression of furin, or functional variant thereof, from a transfected plasmid or from a stably integrated polynucleotide sequence in the host cell. In some embodiments, the furin cleavage site of hMPV F protein is modified by mutating the RQSR (SEQ ID NO: 54) motif to RRRR (SEQ ID NO: 55), or removed by substituting a Gly linker, or by making other amino acid substitutions.

hMPV neutralizing activity is mediated by antibodies recognizing both pre-fusion and post-fusion F protein conformations and antibodies generated in an in vivo immunological response can either selectively recognize pre-fusion specific or post-fusion specific sites, or can recognize both the pre-fusion and post-fusion forms of F protein. Battles et al. Nat Commun. 8:1528 (2017). The DS7, MPV196, MPV201, and MPV314 neutralizing antibodies bind an antigenic site accessible in both the pre-fusion and post-fusion forms of F protein, and is located on the DI and DII head domains of the F protein. Wen et al. Nat Struct Mol Biol. 19(4):461-463 (2012); Bar-Peled et al. J Virol. 93:e00342-19 (2019)). The MPE8 neutralizing antibody binds an epitope on F protein spanning the DI, DII, and DIII subunits, forming contacts dependent on the prefusion F conformation. Wen et al. Nat Microbiol. 2:16272 (2017). Several antigenic epitopes are shared with the respiratory syncytial virus (RSV) F protein, such as for example, the site III and site IV epitopes Huang et al. Front Immunol. 10:2778 (2019). These are examples and not an exhaustive list of epitopes described for neutralization antibodies against hMPV F protein.

The neutralizing epitopes are generally conformation epitopes so peptides corresponding to the epitopes do not bind the antibodies and thus do not induce neutralizing titers. Neutralizing antibodies can be isolated from healthy donors that are seropositive for hMPV, Battles et al., or can be generated by immunization in animal models, Gabriella et al. J Virol. 81(2):698-707 (2007).

In some embodiments, the VLPs of the disclosure comprising an hMPV F protein ectodomain can bind a neutralizing antibody against the hMPV F protein, also referred to as anti-hMPV F protein antibodies. In some embodiments, the VLPs described herein can bind anti-hMPV F protein antibodies known to selectively bind the prefusion form of hMPV F protein. Examples of antibodies that selectively bind the prefusion form of hMPV F protein include, without limitation, the MF10 and MPE8 antibodies. In some embodiments, the VLPs described herein bind anti-hMPV F protein antibodies known to selectively bind the postfusion form of hMPV F protein. Examples of antibodies that selectively bind the postfusion form of hMPV F protein include, without limitation, the MF1, MF2, MF3, MF 11, MF17, MF18, and MF19 antibodies. In some embodiments, the VLPs described herein can bind antibodies that bind both the prefusion and postfusion forms of hMPV F protein. Examples of antibodies that bind the prefusion and postfusion forms of hMPV F protein include, without limitation, the MF9, MF12, MF14, MF15, MF16, and MF20 antibodies. In some embodiments, the VLPs described herein bind one or more anti-hMPV F protein antibodies. In some embodiments, the VLPs described herein bind two or more anti-hMPV F protein antibodies.

### Multimerization Domains and Linkers

In some embodiments, the VLP comprises a trimeric assembly of antigens comprising a first polypeptide comprising a first multimerization domain and a first polypeptide comprising a second multimerization domain. The first multimerization domain comprises a protein-protein interface that induces three copies of the first polypeptides to self-associate to form trimeric building blocks. In VLPs that have two or more components, each copy of the first multimerization domain further comprises a surface-exposed interface that interacts with a complementary surface-exposed interface on the second multimerization domain. Similarly stated, the second multimerization domain is adapted to multimerize with first multimerization domain (of the first polypeptide of the first component). As described in King et al. (Nature 510, 103-108, 2014), Bale et al. (Science 353, 389-394, 2016), and patent publications WO2014124301 A1 and US20160122392 A1, the complementary protein-protein interface between the first multimerization domains and second multimerization domains drives the assembly of multiple copies of the trimeric assembly domain and second assembly domain into a target VLP. In some embodiments, each copy of the trimeric assembly domains of the VLP bears an antigenic protein, or antigenic fragment thereof, linked thereto (e.g., as a genetic fusion); these VLPs display the proteins at full valency. In other embodiments, the VLPs of the disclosure comprise one or more copies of first multimerization domains bearing antigens proteins, or antigenic fragments thereof (e.g., as genetic fusions) as well as one or more first multimerization domains that do not bear antigenic proteins; these VLPs display the G proteins at partial valency. The first multimerization domains can be any polypeptide sequence that forms a trimer and interacts with a second multimerization domains to drive assembly to a target VLP. In some embodiments, the VLP comprises first polypeptide and a second polypeptide selected from those disclosed in US 20130274441 A1, US 2015/0356240 A1, US 2016/0122392 A1, WO 2018/187325 A1, each of which is incorporated by reference herein in its entirety.

In some embodiments of the VLPs of the present disclosure, the antigenic protein and the core of the VLP may be genetically fused such that they are both present in a single polypeptide, also called a single chain polypeptide. The linkage between the protein and the core allows the antigenic protein, or antigenic fragment thereof, to be displayed on the exterior of the VLP. As such, the point of connection to the core should be on the exterior of the core of the virus-like particle formed. A wide variety of polypeptide sequences can be used to link the proteins, or antigenic fragments thereof and the core of the virus-like particle. In some cases the linker comprises that polypeptide sequence. Any suitable linker polypeptide can be used. In some embodiments, the linker imposes a rigid relative orientation of the antigenic protein (e.g. ectodomain) or antigenic fragment thereof to the core. In some embodiments, the linker flexibly links the antigenic protein (e.g. ectodomain) or antigenic fragment thereof to the core. In some embodiments, the linker includes additional trimerization domains (e.g., the foldon domain of T4 fibritin) to assist in stabilizing the trimeric form of the F protein—e.g., GYIPEAPRDGQAYVRKDGEWVLLSTFL (SEQ ID NO: 91) or a functional variant thereof.

In some embodiments, the linker may comprise a Gly-Ser linker (i.e. a linker consisting of glycine and serine residues) of any suitable length. In some embodiments, the Gly-Ser linker may be 3, 4, 5, 6, 7, 8, 9, 10, 11, 12, 13, 14, 15, 16, 17, 18, 19, 20, or more amino acids in length. In some embodiments, the Gly-Ser linker may comprise or consist of the amino acid sequence of GSGGSGSGSGGSGSG (SEQ ID NO: 127), GGSGGSGS (SEQ ID NO: 128) or GSGGSGSG (SEQ ID NO: 129). In some embodiments, the linker comprises the sequence GSGSGSG (SEQ ID NO: 130). In some embodiments, the linker comprises the sequence GSGSGSGSGSGSGSG (SEQ ID NO: 131). In some embodiments, the linker comprises the sequence GSGGSGSGSGGS (SEQ ID NO: 126).

In some embodiments of the VLPs of the present disclosure, the first component may optionally contain a poly-His tag, MHISHHHq (SEQ TD NO: 143).

Table 3A and Table 3B3 show illustrative examples of the VLP sequences described herein. The sequences comprise a multimerization domain, an RSV or hMIPV F protein ectodomain, a linker, a poly-His tag, and a signal sequence. The signal and poly-His tag sequences are underlined and optionally included in the sequence.

TABLE 3A

| Component | | | |
|---|---|---|---|
| Type | Description | Mutations | Sequence |
| Comp A-RSV | DS-Cav1- | | QNITEEFYQSTCSAVSKGYLSALRTGWYTS |
| fusion | T33-15B | | VITIELSNIKENKCNGTDAKVKLIKQELDKY |
| | SEQ ID NO: | | KNAVTELQLLMQSTPATNNRARRELPRFMN |
| | 146 | | YTLNNAKKTNVTLSKKRKRRFLGFLLGVGS |
| | | | AIASGVAVCKVLHLEGEVNKIKSALLSTNK |
| | | | AVVSLSNGVSVLTFKVLDLKNYIDKQLLPIL |
| | | | NKQSCSISNIETVIEFQQKNNRLLEITREFSV |
| | | | NAGVTTPVSTYMLTNSELLSLINDMPITNDQ |
| | | | KKLMSNNVQIVRQQSYSIMCIIKEEVLAYVV |
| | | | QLPLYGVIDTPCWKLHTSPLCTTNTKEGSNI |
| | | | CLTRTDRGWYCDNAGSVSFFPQAETCKVQS |
| | | | NRVFCDTMNSLTLPSEVNLCNVDIFNPKYD |
| | | | CKIMTSKTDVSSSVITSLGAIVSCYGKTKCT |
| | | | ASNKNRGIIKTFSNGCDYVSNKGVDTVSVG |
| | | | NTLYYVNKQEGKSLYVKGEPIINFYDPLVFP |
| | | | SDEFDASISQVNEKINQSLAFIRKSDELLGGS |
| | | | MVRGIRGAITVNSDTPTSIIIATILLLEKMLEA |
| | | | NGIQSYEELAAVIFTVTEDLTSAFPAEAARQI |
| | | | GMHRVPLLSAREVPVPGSLPRVIRVLALWN |
| | | | TDTPQDRVRHVYLSEAVRLRPDLESAQ |
| Comp A-RSV | DS-Cav1- | | QNITEEFYQSTCSAVSKGYLSALRTGWYTS |
| fusion | foldon-I53- | | VITIELSNIKENKCNGTDAKVKLIKQELDKY |
| | 50A | | KNAVTELQLLMQSTPATNNRARRELPRFMN |
| | SEQ ID NO: | | YTLNNAKKTNVTLSKKRKRRFLGFLLGVGS |
| | 147 | | AIASGVAVCKVLHLEGEVNKIKSALLSTNK |
| | | | AVVSLSNGVSVLTFKVLDLKNYIDKQLLPIL |
| | | | NKQSCSISNIETVIEFQQKNNRLLEITREFSV |
| | | | NAGVTTPVSTYMLTNSELLSLINDMPITNDQ |
| | | | KKLMSNNVQIVRQQSYSIMCIIKEEVLAYVV |
| | | | QLPLYGVIDTPCWKLHTSPLCTTNTKEGSNI |
| | | | CLTRTDRGWYCDNAGSVSFFPQAETCKVQS |
| | | | NRVFCDTMNSLTLPSEVNLCNVDIFNPKYD |
| | | | CKIMTSKTDVSSSVITSLGAIVSCYGKTKCT |
| | | | ASNKNRGIIKTFSNGCDYVSNKGVDTVSVG |
| | | | NTLYYVNKQEGKSLYVKGEPIINFYDPLVFP |
| | | | SDEFDASISQVNEKINQSLAFIRGYIPEAPRD |
| | | | GQAYVRKDGEWVLLSTFLGSGSHHHHHHH |
| | | | HGGSGGSGSEKAAKAEEAARKMEELFKKH |
| | | | KIVAVLRANSVEEAIEKAVAVFAGGVHLIEI |
| | | | TFTVPDADTVIKALSVLKEKGAIIGAGTVTS |
| | | | VEQCRKAVESGAEFIVSPHLDEEISQFCKEK |
| | | | GVFYMPGVMTPTELVKAMKLGHTILKLFPG |
| | | | EVVGPQFVKAMKGPFPNVKFVPTGGVNLD |
| | | | NVCEWFKAGVLAVGVGSALVKGTPDEVRE |
| | | | KAKAFVEKIRGCTE |
| Comp A-RSV | DS-Cav1-I53- | | QNITEEFYQSTCSAVSKGYLSALRTGWYTS |
| fusion | 50A | | VITIELSNIKENKCNGTDAKVKLIKQELDKY |
| | SEQ ID NO: | | KNAVTELQLLMQSTPATNNRARRELPRFMN |
| | 148 | | YTLNNAKKTNVTLSKKRKRRFLGFLLGVGS |
| | | | AIASGVAVCKVLHLEGEVNKIKSALLSTNK |
| | | | AVVSLSNGVSVLTFKVLDLKNYIDKQLLPIL |
| | | | NKQSCSISNIETVIEFQQKNNRLLEITREFSV |
| | | | NAGVTTPVSTYMLTNSELLSLINDMPITNDQ |
| | | | KKLMSNNVQIVRQQSYSIMCIIKEEVLAYVV |
| | | | QLPLYGVIDTPCWKLHTSPLCTTNTKEGSNI |
| | | | CLTRTDRGWYCDNAGSVSFFPQAETCKVQS |
| | | | NRVFCDTMNSLTLPSEVNLCNVDIFNPKYD |
| | | | CKIMTSKTDVSSSVITSLGAIVSCYGKTKCT |
| | | | ASNKNRGIIKTFSNGCDYVSNKGVDTVSVG |
| | | | NTLYYVNKQEGKSLYVKGEPIINFYDPLVFP |
| | | | SDEFDASISQVNEKINQSLAFIRGGSGGSGSE |
| | | | KAAKAEEAARKMEELFKKHKIVAVLRANS |
| | | | VEEAIEKAVAVFAGGVHLIEITFTVPDADTV |
| | | | IKALSVLKEKGAIIGAGTVTSVEQCRKAVES |
| | | | GAEFIVSPHLDEEISQFCKEKGVFYMPGVMT |
| | | | PTELVKAMKLGHTILKLFPGEVVGPQFVKA |
| | | | MKGPFPNVKFVPTGGVNLDNVCEWFKAGV |
| | | | LAVGVGSALVKGTPDEVREKAKAFVEKIRG |
| | | | CTE |
| Comp A-RSV | DS-Cav1-I32- | | QNITEEFYQSTCSAVSKGYLSALRTGWYTS |
| fusion | 28A | | VITIELSNIKENKCNGTDAKVKLIKQELDKY |
| | SEQ ID NO: | | KNAVTELQLLMQSTPATNNRARRELPRFMN |
| | 149 | | YTLNNAKKTNVTLSKKRKRRFLGFLLGVGS |
| | | | AIASGVAVCKVLHLEGEVNKIKSALLSTNK |
| | | | AVVSLSNGVSVLTFKVLDLKNYIDKQLLPIL |
| | | | NKQSCSISNIETVIEFQQKNNRLLEITREFSV |
| | | | NAGVTTPVSTYMLTNSELLSLINDMPITNDQ |
| | | | KKLMSNNVQIVRQQSYSIMCIIKEEVLAYVV |
| | | | QLPLYGVIDTPCWKLHTSPLCTTNTKEGSNI |
| | | | CLTRTDRGWYCDNAGSVSFFPQAETCKVQS |
| | | | NRVFCDTMNSLTLPSEVNLCNVDIFNPKYD |
| | | | CKIMTSKTDVSSSVITSLGAIVSCYGKTKCT |
| | | | ASNKNRGIIKTFSNGCDYVSNKGVDTVSVG |
| | | | NTLYYVNKQEGKSLYVKGEPIINFYDPLVFP |
| | | | SDEFDASISQVNEKINQSLAFIRKSDELLGGS |
| | | | GGSGSDDARIAAIGDVDELNSQIGVLLAEPL |
| | | | PDDVRAALSAIQHDLFDLGGELCIPGHAAIT |
| | | | EDHLLRLALWLVHYNGQLPPLEEFILPGGA |
| | | | RGAALAHVCRTVCRRAERSIKALGASEPLNI |
| | | | APAAYVNLLSDLLFVLARVLNRAAGGADV |
| | | | LWDRTRAH |
| Comp A-RSV | DS-Cav1- | | QNITEEFYQSTCSAVSKGYLSALRTGWYTS |
| fusion | foldon-15GS- | | VITIELSNIKENKCNGTDAKVKLIKQELDKY |
| | HelExt-I53- | | KNAVTELQLLMQSTPATNNRARRELPRFMN |
| | 50A (F14) | | YTLNNAKKTNVTLSKKRKRRFLGFLLGVGS |
| | SEQ ID NO: | | AIASGVAVCKVLHLEGEVNKIKSALLSTNK |
| | 150 | | AVVSLSNGVSVLTFKVLDLKNYIDKQLLPIL |
| | | | NKQSCSISNIETVIEFQQKNNRLLEITREFSV |
| | | | NAGVTTPVSTYMLTNSELLSLINDMPITNDQ |
| | | | KKLMSNNVQIVRQQSYSIMCIIKEEVLAYVV |
| | | | QLPLYGVIDTPCWKLHTSPLCTTNTKEGSNI |
| | | | CLTRTDRGWYCDNAGSVSFFPQAETCKVQS |
| | | | NRVFCDTMNSLTLPSEVNLCNVDIFNPKYD |
| | | | CKIMTSKTDVSSSVITSLGAIVSCYGKTKCT |
| | | | ASNKNRGIIKTFSNGCDYVSNKGVDTVSVG |
| | | | NTLYYVNKQEGKSLYVKGEPIINFYDPLVFP |
| | | | SDEFDASISQVNEKINQSLAFIRGYIPEAPRD |
| | | | GQAYVRKDGEWVLLSTFLGSGGSGSGSGG |
| | | | SGSGEKAAKAEEAARKMEELFKKHKIVAVL |
| | | | RANSVEEAIEKAVAVFAGGVHLIEITFTVPD |
| | | | ADTVIKALSVLKEKGAIIGAGTVTSVEQCRK |
| | | | AVESGAEFIVSPHLDEEISQFCKEKGVFYMP |
| | | | GVMTPTELVKAMKLGHTILKLFPGEVVGPQ |
| | | | FVKAMKGPFPNVKFVPTGGVNLDNVCEWF |
| | | | KAGVLAVGVGSALVKGTPDEVREKAKAFV |
| | | | EKIRGCTE |
| Comp A-RSV | CompA-RSV- | none | QNITEEFYQSTCSAVSKGYLSALRTGWYTS |
| fusion | 02 | | VITIELSNIKENKCNGTDAKVKLIKQELDKY |
| | SEQ ID NO: | | KNAVTELQLLMQSTPATNNRARRELPRFMN |
| | 151 | | YTLNNAKKTNVTLSKKRKRRFLGFLLGVGS |
| | | | AIASGVAVCKVLHLEGEVNKIKSALLSTNK |
| | | | AVVSLSNGVSVLTFKVLDLKNYIDKQLLPIL |
| | | | NKQSCSISNIETVIEFQQKNNRLLEITREFSV |
| | | | NAGVTTPVSTYMLTNSELLSLINDMPITNDQ |
| | | | KKLMSNNVQIVRQQSYSIMCIIKEEVLAYVV |
| | | | QLPLYGVIDTPCWKLHTSPLCTTNTKEGSNI |
| | | | CLTRTDRGWYCDNAGSVSFFPQAETCKVQS |
| | | | NRVFCDTMNSLTLPSEVNLCNVDIFNPKYD |
| | | | CKIMTSKTDVSSSVITSLGAIVSCYGKTKCT |
| | | | ASNKNRGIIKTFSNGCDYVSNKGVDTVSVG |
| | | | NTLYYVNKQEGKSLYVKGEPIINFYDPLVFP |
| | | | SDEFDASISQVNEKINQSLAFIRKSDELLGSG |
| | | | GSGSGSGGSEKAAKAEEAARKMEELFKKH |
| | | | KIVAVLRANSVEEAIEKAVAVFAGGVHLIEI |
| | | | TFTVPDADTVIKALSVLKEKGAIIGAGTVTS |
| | | | VEQCRKAVESGAEFIVSPHLDEEISQFCKEK |
| | | | GVFYMPGVMTPTELVKAMKLGHTILKLFPG |
| | | | EVVGPQFVKAMKGPFPNVKFVPTGGVNLD |
| | | | NVCEWFKAGVLAVGVGSALVKGTPDEVRE |
| | | | KAKAFVEKIRGCTE |
| RSV F | DS-Cav1- | | QNITEEFYQSTCSAVSKGYLSALRTGWYTS |
| | foldon | | VITIELSNIKENKCNGTDAKVKLIKQELDKY |
| | SEQ ID NO: | | KNAVTELQLLMQSTPATNNRARRELPRFMN |
| | 152 | | YTLNNAKKTNVTLSKKRKRRFLGFLLGVGS |
| | | | AIASGVAVCKVLHLEGEVNKIKSALLSTNK |
| | | | AVVSLSNGVSVLTFKVLDLKNYIDKQLLPIL |
| | | | NKQSCSISNIETVIEFQQKNNRLLEITREFSV |
| | | | NAGVTTPVSTYMLTNSELLSLINDMPITNDQ |
| | | | KKLMSNNVQIVRQQSYSIMCIIKEEVLAYVV |
| | | | QLPLYGVIDTPCWKLHTSPLCTTNTKEGSNI |
| | | | CLTRTDRGWYCDNAGSVSFFPQAETCKVQS |
| | | | NRVFCDTMNSLTLPSEVNLCNVDIFNPKYD |
| | | | CKIMTSKTDVSSSVITSLGAIVSCYGKTKCT |
| | | | ASNKNRGIIKTFSNGCDYVSNKGVDTVSVG |
| | | | NTLYYVNKQEGKSLYVKGEPIINFYDPLVFP |
| | | | SDEFDASISQVNEKINQSLAFIRKSDELLGYI |
| | | | PEAPRDGQAYVRKDGEWVLLSTFL |
| Comp A-RSV | DS-Cav1- | | QNITEEFYQSTCSAVSKGYLSALRTGWYTS |
| fusion | foldon-T33- | | VITIELSNIKENKCNGTDAKVKLIKQELDKY |
| | 31A | | KNAVTELQLLMQSTPATNNRARRELPRFMN |
| | SEQ ID NO: | | YTLNNAKKTNVTLSKKRKRRFLGFLLGVGS |
| | 153 | | AIASGVAVCKVLHLEGEVNKIKSALLSTNK |
| | | | AVVSLSNGVSVLTFKVLDLKNYIDKQLLPIL |
| | | | NKQSCSISNIETVIEFQQKNNRLLEITREFSV |
| | | | NAGVTTPVSTYMLTNSELLSLINDMPITNDQ |
| | | | KKLMSNNVQIVRQQSYSIMCIIKEEVLAYVV |
| | | | QLPLYGVIDTPCWKLHTSPLCTTNTKEGSNI |
| | | | CLTRTDRGWYCDNAGSVSFFPQAETCKVQS |
| | | | NRVFCDTMNSLTLPSEVNLCNVDIFNPKYD |
| | | | CKIMTSKTDVSSSVITSLGAIVSCYGKTKCT |
| | | | ASNKNRGIIKTFSNGCDYVSNKGVDTVSVG |
| | | | NTLYYVNKQEGKSLYVKGEPIINFYDPLVFP |
| | | | SDEFDASISQVNEKINQSLAFIRKSDELLGYI |
| | | | PEAPRDGQAYVRKDGEWVLLSTFLGGSME |
| | | | EVVLITVPSALVAVKIAHALVEERLAACVNI |
| | | | VPGLTSIYREEGSVVSDHELLLLVKTTTDAF |
| | | | PKLKERVKELHPYEVPEIVALPIAEGNREYL |
| | | | DWLRENTG |
| Comp A-RSV | DS-Cav1- | | QNITEEFYQSTCSAVSKGYLSALRTGWYTS |
| fusion | T33-31A | | VITIELSNIKENKCNGTDAKVKLIKQELDKY |
| | SEQ ID NO: | | KNAVTELQLLMQSTPATNNRARRELPRFMN |
| | 154 | | YTLNNAKKTNVTLSKKRKRRFLGFLLGVGS |
| | | | AIASGVAVCKVLHLEGEVNKIKSALLSTNK |
| | | | AVVSLSNGVSVLTFKVLDLKNYIDKQLLPIL |
| | | | NKQSCSISNIETVIEFQQKNNRLLEITREFSV |
| | | | NAGVTTPVSTYMLTNSELLSLINDMPITNDQ |
| | | | KKLMSNNVQIVRQQSYSIMCIIKEEVLAYVV |
| | | | QLPLYGVIDTPCWKLHTSPLCTTNTKEGSNI |
| | | | CLTRTDRGWYCDNAGSVSFFPQAETCKVQS |
| | | | NRVFCDTMNSLTLPSEVNLCNVDIFNPKYD |
| | | | CKIMTSKTDVSSSVITSLGAIVSCYGKTKCT |
| | | | ASNKNRGIIKTFSNGCDYVSNKGVDTVSVG |
| | | | NTLYYVNKQEGKSLYVKGEPIINFYDPLVFP |
| | | | SDEFDASISQVNEKINQSLAFIRKSDELLGGS |
| | | | MEEVVLITVPSALVAVKIAHALVEERLAAC |
| | | | VNIVPGLTSIYREEGSVVSDHELLLLVKTTT |
| | | | DAFPKLKERVKELHPYEVPEIVALPIAEGNR |
| | | | EYLDWLRENTG |
| Comp A-RSV | DS-Cav1- | | QNITEEFYQSTCSAVSKGYLSALRTGWYTS |
| fusion | foldon-T33- | | VITIELSNIKENKCNGTDAKVKLIKQELDKY |
| | 15B | | KNAVTELQLLMQSTPATNNRARRELPRFMN |
| | SEQ ID NO: | | YTLNNAKKTNVTLSKKRKRRFLGFLLGVGS |
| | 155 | | AIASGVAVCKVLHLEGEVNKIKSALLSTNK |
| | | | AVVSLSNGVSVLTFKVLDLKNYIDKQLLPIL |
| | | | NKQSCSISNIETVIEFQQKNNRLLEITREFSV |
| | | | NAGVTTPVSTYMLTNSELLSLINDMPITNDQ |
| | | | KKLMSNNVQIVRQQSYSIMCIIKEEVLAYVV |
| | | | QLPLYGVIDTPCWKLHTSPLCTTNTKEGSNI |
| | | | CLTRTDRGWYCDNAGSVSFFPQAETCKVQS |
| | | | NRVFCDTMNSLTLPSEVNLCNVDIFNPKYD |
| | | | CKIMTSKTDVSSSVITSLGAIVSCYGKTKCT |
| | | | ASNKNRGIIKTFSNGCDYVSNKGVDTVSVG |
| | | | NTLYYVNKQEGKSLYVKGEPIINFYDPLVFP |
| | | | SDEFDASISQVNEKINQSLAFIRKSDELLGYI |
| | | | PEAPRDGQAYVRKDGEWVLLSTFLGGSMV |
| | | | RGIRGAITVNSDTPTSIIIATILLLEKMLEANG |
| | | | IQSYEELAAVIFTVTEDLTSAFPAEAARQIG |
| | | | MHRVPLLSAREVPVPGSLPRVIRVLALWNT |
| | | | DTPQDRVRHVYLSEAVRLRPDLESAQ |
| CompA | I53-50A-Δcys | C74A, C98A, | MEELFKKHKIVAVLRANSVEEAIEKAVAVF |
| | SEQ ID NO: | C163A, C201A | AGGVHLIEITFTVPDADTVIKALSVLKEKGA |
| | 145 | | IIGAGTVTSVEQARKAVESGAEFIVSPHLDE |
| | | | EISQFAKEKGVFYMPGVMTPTELVKAMKL |
| | | | GHTILKLFPGEVVGPQFVKAMKGPFPNVKF |
| | | | VPTGGVNLDNVAEWFKAGVLAVGVGSALV |
| | | | KGTPDEVREKAKAFVEKIRGATE |
| CompA | I53_dn5B | | EEAELAYLLGELAYKLGEYRIAIRAYRIALK |
| | SEQ ID NO: | | RDPNNAEAWYNLGNAYYKQGRYREAIEYY |
| | 156 | | QKALELDPNNAEAWYNLGNAYYERGEYEE |
| | | | AIEYYRKALRLDPNNADAMQNLLNAKMRE |
| | | | E |
| Signal | SEQ ID NO: | | MELLILKANAITTILTAVTFCFASG |
| sequence | 157 | | |
| Signal | SEQ ID NO: | | MELLILKANVIATILTAVTFCFASS |
| sequence | 158 | | |
| Signal | SEQ ID NO: | | MSWKVVIIFSLLITPQHG |
| sequence | 159 | | |
| RSV F | RSV F protein | S155C, S290C, | QNITEEFYQSTCSAVSKGYLSALRTGWYTS |
| (Ectodomain) | DS-Cav1 | S190F, V207L | VITIELSNIKENKCNGTDAKVKLIKQELDKY |
| | SEQ ID NO: | | KNAVTELQLLMQSTPATNNRARRELPRFMN |
| | 160 | | YTLNNAKKTNVTLSKKRKRRFLGFLLGVGS |
| | | | AIASGVAVCKVLHLEGEVNKIKSALLSTNK |
| | | | AVVSLSNGVSVLTFKVLDLKNYIDKQLLPIL |
| | | | NKQSCSISNIETVIEFQQKNNRLLEITREFSV |
| | | | NAGVTTPVSTYMLTNSELLSLINDMPITNDQ |
| | | | KKLMSNNVQIVRQQSYSIMCIIKEEVLAYVV |
| | | | QLPLYGVIDTPCWKLHTSPLCTTNTKEGSNI |
| | | | CLTRTDRGWYCDNAGSVSFFPQAETCKVQS |
| | | | NRVFCDTMNSLTLPSEVNLCNVDIFNPKYD |
| | | | CKIMTSKTDVSSSVITSLGAIVSCYGKTKCT |
| | | | ASNKNRGIIKTFSNGCDYVSNKGVDTVSVG |
| | | | NTLYYVNKQEGKSLYVKGEPIINFYDPLVFP |
| | | | SDEFDASISQVNEKINQSLAFIRKSDELL |
| Ectodomain | RSV F protein | | QNITEEFYQSTCSAVSKGYLSALRTGWYTS |
| | SEQ ID NO: | | VITIELSNIKENKCNGTDAKVKLIKQELDKY |
| | 178 | | KNAVTELQLLMQSTPATNNRARRELPRFMN |
| | | | YTLNNAKKTNVTLSKKRKRRFLGFLLGVGS |
| | | | AIASGVAVSKVLHLEGEVNKIKSALLSTNK |
| | | | AVVSLSNGVSVLTSKVLDLKNYIDKQLLPIV |
| | | | NKQSCSISNIETVIEFQQKNNRLLEITREFSV |
| | | | NAGVTTPVSTYMLTNSELLSLINDMPITNDQ |
| | | | KKLMSNNVQIVRQQSYSIMSIIKEEVLAYVV |
| | | | QLPLYGVIDTPCWKLHTSPLCTTNTKEGSNI |
| | | | CLTRTDRGWYCDNAGSVSFFPQAETCKVQS |
| | | | NRVFCDTMNSLTLPSEVNLCNVDIFNPKYD |
| | | | CKIMTSKTDVSSSVITSLGAIVSCYGKTKCT |
| | | | ASNKNRGIIKTFSNGCDYVSNKGVDTVSVG |
| | | | NTLYYVNKQEGKSLYVKGEPIINFYDPLVFP |
| | | | SDEFDASISQVNEKINQSLAFIRKSDELL |
| Ectodomain | RSV-B F | | QNITEEFYQSTCSAVSRGYFSALRTGWYTS |
| | protein | | VITIELSNITETKCNGTDTKVKLIKQELDKYK |
| | SEQ ID NO: | | NAVTELQLLMQNTPAANNRARREAPQHMN |
| | 161 | | YTINTTKNLNVSISKKRKRRFLGFLLGVGSA |
| | | | IASGIAVSKVLHLEGEVNKIKNALLSTNKAV |
| | | | VSLSNGVSVLTSKVLDLKNYINNQLLPIVNQ |
| | | | QSCRIFNIETVIEFQQKNSRLLEITREFSVNA |
| | | | GVTTPLSTYMLTNSELLSLINDMPITNDQKK |
| | | | LMSSNVQIVRQQSYSIMSIIKEEVLAYVVQL |
| | | | PIYGVIDTPCWKLHTSPLCTTNIKEGSNICLT |
| | | | RTDRGWYCDNAGSVSFFPQADTCKVQSNR |
| | | | VFCDTMNSLTLPSEVSLCNTDIFNSKYDCKI |
| | | | MTSKTDISSSVITSLGAIVSCYGKTKCTASN |
| | | | KNRGIIKTFSNGCDYVSNKGVDTVSVGNTL |
| | | | YYVNKLEGKNLYVKGEPIINYYDPLVFPSDE |
| | | | FDASISQVNEKINQSLAFIRKSDELL |
| Ectodomain | RSV-B F | S155C, S290C, | QNITEEFYQSTCSAVSRGYFSALRTGWYTS |
| | protein | S190F, V207L | VITIELSNITETKCNGTDTKVKLIKQELDKYK |
| | (stabilized) | | NAVTELQLLMQNTPAANNRARREAPQHMN |
| | SEQ ID NO: | | YTINTTKNLNVSISKKRKRRFLGFLLGVGSA |
| | 162 | | IASGIAVCKVLHLEGEVNKIKNALLSTNKAV |
| | | | VSLSNGVSVLTFKVLDLKNYINNQLLPILNQ |
| | | | QSCRIFNIETVIEFQQKNSRLLEITREFSVNA |
| | | | GVTTPLSTYMLTNSELLSLINDMPITNDQKK |
| | | | LMSSNVQIVRQQSYSIMCIIKEEVLAYVVQL |
| | | | PIYGVIDTPCWKLHTSPLCTTNIKEGSNICLT |
| | | | RTDRGWYCDNAGSVSFFPQADTCKVQSNR |
| | | | VFCDTMNSLTLPSEVSLCNTDIFNSKYDCKI |
| | | | MTSKTDISSSVITSLGAIVSCYGKTKCTASN |
| | | | KNRGIIKTFSNGCDYVSNKGVDTVSVGNTL |
| | | | YYVNKLEGKNLYVKGEPIINYYDPLVFPSDE |
| | | | FDASISQVNEKINQSLAFIRKSDELL |
| Linker | Gly-Ser | | GSGGSGSGSGGSGSG |
| (SEQ ID NO: | | | |
| 127) | | | |
| Linker | Gly-Ser | | GSGGSGSGSGGS |
| (SEQ ID NO: | | | |
| 126) | | | |
| Linker | Gly-Ser | | GGSGGSGS |
| (SEQ ID NO: | | | |
| 128) | | | |
| Linker | Gly-Ser | | GSGGSGSG |
| (SEQ ID NO: | | | |
| 129) | | | |
| Linker | Extension | | EKAAKAEEAAR |
| SEQ ID NO: | | | |
| 163 | | | |
| Linker | Extension | | EKAAKAEEAARK |
| (SEQ ID NO: | | | |
| 125) | | | |
| Trimerization | Foldon | | GYIPEAPRDGQAYVRKDGEWVLLSTFL |
| (SEQ ID NO: | | | |
| 91) | | | |
| CompB | I53-50B | | MNQHSHKDYETVRIAVVRARWHAEIVDAC |
| | SEQ ID NO: | | VSAFEAAMADIGGDRFAVDVFDVPGAYEIP |
| | 164 | | LHARTLAETGRYGAVLGTAFVVNGGIYRHE |
| | | | FVASAVIDGMMNVQLSTGVPVLSAVLTPHR |
| | | | YRDSDAHTLLFLALFAVKGMEAARACVEIL |
| | | | AAREKIAA |
| CompB | I53-50B.1 | | MNQHSHKDHETVRIAVVRARWHAEIVDAC |
| | SEQ ID NO: | | VSAFEAAMRDIGGDRFAVDVFDVPGAYEIP |
| | 165 | | LHARTLAETGRYGAVLGTAFVVNGGIYRHE |
| | | | FVASAVIDGMMNVQLDTGVPVLSAVLTPH |
| | | | RYRDSDAHTLLFLALFAVKGMEAARACVEI |
| | | | LAAREKIAA |
| CompB | I53-50A | none | MEELFKKHKIVAVLRANSVEEAIEKAVAVF |
| | SEQ ID NO: | | AGGVHLIEITFTVPDADTVIKALSVLKEKGA |
| | 166 | | IIGAGTVTSVEQCRKAVESGAEFIVSPHLDEE |
| | | | ISQFCKEKGVFYMPGVMTPTELVKAMKLG |
| | | | HTILKLFPGEVVGPQFVKAMKGPFPNVKFV |
| | | | PTGGVNLDNVCEWFKAGVLAVGVGSALVK |
| | | | GTPDEVREKAKAFVEKIRGCTE |
| CompB | I53- | | NQHSHKDHETVRIAVVRARWHAEIVDACV |
| | 50B.1NegT2 | | SAFEAAMRDIGGDRFAVDVFDVPGAYEIPL |
| | SEQ ID NO: | | HARTLAETGRYGAVLGTAFVVDGGIYDHEF |
| | 167 | | VASAVIDGMMNVQLDTGVPVLSAVLTPHE |
| | | | YEDSDADTLLFLALFAVKGMEAARACVEIL |
| | | | AAREKIAA |
| CompB | I53- | H9Y, R38A, | MNQHSHKDHETVRIAVVRARWHAEIVDAC |
| | 50B.4PosT1 | N96D, N105S, | VSAFEAAMRDIGGDRFAVDVFDVPGAYEIP |
| | SEQ ID NO: | N109R, D111R, | LHARTLAETGRYGAVLGTAFVVNGGIYRHE |
| | 168 | K122D, K124D | FVASAVINGMMNVQLNTGVPVLSAVLTPH |
| | | | NYDKSKAHTLLFLALFAVKGMEAARACVEI |
| | | | LAAREKIAA |
| CompB | T33-15B | | MVRGIRGAITVNSDTPTSIIIATILLLEKMLEA |
| | SEQ ID NO: | | NGIQSYEELAAVIFTVTEDLTSAFPAEAARQI |
| | 169 | | GMHRVPLLSAREVPVPGSLPRVIRVLALWN |
| | | | TDTPQDRVRHVYLSEAVRLRPDLESAQ |
| CompB | I32-28A | | MGDDARIAAIGDVDELNSQIGVLLAEPLPD |
| | SEQ ID NO: | | DVRAALSAIQHDLFDLGGELCIPGHAAITED |
| | 170 | | HLLRLALWLVHYNGQLPPLEEFILPGGARG |
| | | | AALAHVCRTVCRRAERSIKALGASEPLNIAP |
| | | | AAYVNLLSDLLFVLARVLNRAAGGADVLW |
| | | | DRTRAH |
| CompB | T33-31A | | MEEVVLITVPSALVAVKIAHALVEERLAAC |
| | SEQ ID NO: | | VNIVPGLTSIYREEGSVVSDHELLLLVKTTT |
| | 171 | | DAFPKLKERVKELHPYEVPEIVALPIAEGNR |
| | | | EYLDWLRENTG |
| CompB | I53_dn5A | | KYDGSKLRIGILHARWNAEIILALVLGALKR |
| | SEQ ID NO: | | LQEFGVKRENIIIETVPGSFELPYGSKLFVEK |
| | 172 | | QKRLGKPLDAIIPIGVLIKGSTMHFEYICDST |
| | | | THQLMKLNFELGIPVIFGVLTCLTDEQAEAR |
| | | | AGLIEGKMHNHGEDWGAAAVEMATKFN |
| Comp A-RSV | DS-Cav1- | | QNITEEFYQSTCSAVSRGYFSALRTGWYTS |
| B fusion | T33-15B | | VITIELSNITETKCNGTDTKVKLIKQELDKYK |
| | SEQ ID NO: | | NAVTELQLLMQNTPAANNRARREAPQHMN |
| | 179 | | YTINTTKNLNVSISKKRKRRFLGFLLGVGSA |
| | | | IASGIAVCKVLHLEGEVNKIKNALLSTNKAV |
| | | | VSLSNGVSVLTFKVLDLKNYINNQLLPILNQ |
| | | | QSCRIFNIETVIEFQQKNSRLLEITREFSVNA |
| | | | GVTTPLSTYMLTNSELLSLINDMPITNDQKK |
| | | | LMSSNVQIVRQQSYSIMCIIKEEVLAYVVQL |
| | | | PIYGVIDTPCWKLHTSPLCTTNIKEGSNICLT |
| | | | RTDRGWYCDNAGSVSFFPQADTCKVQSNR |
| | | | VFCDTMNSLTLPSEVSLCNTDIFNSKYDCKI |
| | | | MTSKTDISSSVITSLGAIVSCYGKTKCTASN |
| | | | KNRGIIKTFSNGCDYVSNKGVDTVSVGNTL |
| | | | YYVNKLEGKNLYVKGEPIINYYDPLVFPSDE |
| | | | FDASISQVNEKINQSLAFIRKSDELLGGSMV |
| | | | RGIRGAITVNSDTPTSIIIATILLLEKMLEANG |
| | | | IQSYEELAAVIFTVTEDLTSAFPAEAARQIG |
| | | | MHRVPLLSAREVPVPGSLPRVIRVLALWNT |
| | | | DTPQDRVRHVYLSEAVRLRPDLESAQ |
| Comp A-RSV | DS-Cav1- | | QNITEEFYQSTCSAVSRGYFSALRTGWYTS |
| B fusion | foldon-I53- | | VITIELSNITETKCNGTDTKVKLIKQELDKYK |
| | 50A | | NAVTELQLLMONTPAANNRARREAPQHMN |
| | SEQ ID NO: | | YTINTTKNLNVSISKKRKRRFLGFLLGVGSA |
| | 180 | | IASGIAVCKVLHLEGEVNKIKNALLSTNKAV |
| | | | VSLSNGVSVLTFKVLDLKNYINNQLLPILNQ |
| | | | QSCRIFNIETVIEFQQKNSRLLEITREFSVNA |
| | | | GVTTPLSTYMLTNSELLSLINDMPITNDQKK |
| | | | LMSSNVQIVRQQSYSIMCIIKEEVLAYVVQL |
| | | | PIYGVIDTPCWKLHTSPLCTTNIKEGSNICLT |
| | | | RTDRGWYCDNAGSVSFFPQADTCKVQSNR |
| | | | VFCDTMNSLTLPSEVSLCNTDIFNSKYDCKI |
| | | | MTSKTDISSSVITSLGAIVSCYGKTKCTASN |
| | | | KNRGIIKTFSNGCDYVSNKGVDTVSVGNTL |
| | | | YYVNKLEGKNLYVKGEPIINYYDPLVFPSDE |
| | | | FDASISQVNEKINQSLAFIRGYIPEAPRDGQA |
| | | | YVRKDGEWVLLSTFLGSGSHHHHHHHHGG |
| | | | SGGSGSEKAAKAEEAARKMEELFKKHKIVA |
| | | | VLRANSVEEAIEKAVAVFAGGVHLIEITFTV |
| | | | PDADTVIKALSVLKEKGAIIGAGTVTSVEQC |
| | | | RKAVESGAEFIVSPHLDEEISQFCKEKGVFY |
| | | | MPGVMTPTELVKAMKLGHTILKLFPGEVVG |
| | | | PQFVKAMKGPFPNVKFVPTGGVNLDNVCE |
| | | | WFKAGVLAVGVGSALVKGTPDEVREKAKA |
| | | | FVEKIRGCTE |
| Comp A-RSV | DS-Cav1-I53- | | QNITEEFYQSTCSAVSRGYFSALRTGWYTS |
| B fusion | 50A | | VITIELSNITETKCNGTDTKVKLIKQELDKYK |
| | SEQ ID NO: | | NAVTELQLLMQNTPAANNRARREAPQHMN |
| | 181 | | YTINTTKNLNVSISKKRKRRFLGFLLGVGSA |
| | | | IASGIAVCKVLHLEGEVNKIKNALLSTNKAV |
| | | | VSLSNGVSVLTFKVLDLKNYINNQLLPILNQ |
| | | | QSCRIFNIETVIEFQQKNSRLLEITREFSVNA |
| | | | GVTTPLSTYMLTNSELLSLINDMPITNDQKK |
| | | | LMSSNVQIVRQQSYSIMCIIKEEVLAYVVQL |
| | | | PIYGVIDTPCWKLHTSPLCTTNIKEGSNICLT |
| | | | RTDRGWYCDNAGSVSFFPQADTCKVQSNR |
| | | | VFCDTMNSLTLPSEVSLCNTDIFNSKYDCKI |
| | | | MTSKTDISSSVITSLGAIVSCYGKTKCTASN |
| | | | KNRGIIKTFSNGCDYVSNKGVDTVSVGNTL |
| | | | YYVNKLEGKNLYVKGEPIINYYDPLVFPSDE |
| | | | FDASISQVNEKINQSLAFIRGGSGGSGSEKA |
| | | | AKAEEAARKMEELFKKHKIVAVLRANSVEE |
| | | | AIEKAVAVFAGGVHLIEITFTVPDADTVIKA |
| | | | LSVLKEKGAIIGAGTVTSVEQCRKAVESGAE |
| | | | FIVSPHLDEEISQFCKEKGVFYMPGVMTPTE |
| | | | LVKAMKLGHTILKLFPGEVVGPQFVKAMK |
| | | | GPFPNVKFVPTGGVNLDNVCEWFKAGVLA |
| | | | VGVGSALVKGTPDEVREKAKAFVEKIRGCT |
| | | | E |
| Comp A-RSV | DS-Cav1-I32- | | QNITEEFYQSTCSAVSRGYFSALRTGWYTS |
| B fusion | 28A | | VITIELSNITETKCNGTDTKVKLIKQELDKYK |
| | SEQ ID NO: | | NAVTELQLLMQNTPAANNRARREAPQHMN |
| | 182 | | YTINTTKNLNVSISKKRKRRFLGFLLGVGSA |
| | | | IASGIAVCKVLHLEGEVNKIKNALLSTNKAV |
| | | | VSLSNGVSVLTFKVLDLKNYINNQLLPILNQ |
| | | | QSCRIFNIETVIEFQQKNSRLLEITREFSVNA |
| | | | GVTTPLSTYMLTNSELLSLINDMPITNDQKK |
| | | | LMSSNVQIVRQQSYSIMCIIKEEVLAYVVQL |
| | | | PIYGVIDTPCWKLHTSPLCTTNIKEGSNICLT |
| | | | RTDRGWYCDNAGSVSFFPQADTCKVQSNR |
| | | | VFCDTMNSLTLPSEVSLCNTDIFNSKYDCKI |
| | | | MTSKTDISSSVITSLGAIVSCYGKTKCTASN |
| | | | KNRGIIKTFSNGCDYVSNKGVDTVSVGNTL |
| | | | YYVNKLEGKNLYVKGEPIINYYDPLVFPSDE |
| | | | FDASISQVNEKINQSLAFIRKSDELLGGSGGS |
| | | | GSDDARIAAIGDVDELNSQIGVLLAEPLPDD |
| | | | VRAALSAIQHDLFDLGGELCIPGHAAITEDH |
| | | | LLRLALWLVHYNGQLPPLEEFILPGGARGA |
| | | | ALAHVCRTVCRRAERSIKALGASEPLNIAPA |
| | | | AYVNLLSDLLFVLARVLNRAAGGADVLWD |
| | | | RTRAH |
| Comp A-RSV | DS-Cav1- | | QNITEEFYQSTCSAVSRGYFSALRTGWYTS |
| B fusion | foldon-15GS- | | VITIELSNITETKCNGTDTKVKLIKQELDKYK |
| | HelExt-I53- | | NAVTELQLLMQNTPAANNRARREAPQHMN |
| | 50A (F14) | | YTINTTKNLNVSISKKRKRRFLGFLLGVGSA |
| | SEQ ID NO: | | IASGIAVCKVLHLEGEVNKIKNALLSTNKAV |
| | 183 | | VSLSNGVSVLTFKVLDLKNYINNQLLPILNQ |
| | | | QSCRIFNIETVIEFQQKNSRLLEITREFSVNA |
| | | | GVTTPLSTYMLTNSELLSLINDMPITNDQKK |
| | | | LMSSNVQIVRQQSYSIMCIIKEEVLAYVVQL |
| | | | PIYGVIDTPCWKLHTSPLCTTNIKEGSNICLT |
| | | | RTDRGWYCDNAGSVSFFPQADTCKVQSNR |
| | | | VFCDTMNSLTLPSEVSLCNTDIFNSKYDCKI |
| | | | MTSKTDISSSVITSLGAIVSCYGKTKCTASN |
| | | | KNRGIIKTFSNGCDYVSNKGVDTVSVGNTL |
| | | | YYVNKLEGKNLYVKGEPIINYYDPLVFPSDE |
| | | | FDASISQVNEKINQSLAFIRGYIPEAPRDGQA |
| | | | YVRKDGEWVLLSTFLGSGGSGSGSGGSGSG |
| | | | EKAAKAEEAARKMEELFKKHKIVAVLRAN |
| | | | SVEEAIEKAVAVFAGGVHLIEITFTVPDADT |
| | | | VIKALSVLKEKGAIIGAGTVTSVEQCRKAVE |
| | | | SGAEFIVSPHLDEEISQFCKEKGVFYMPGVM |
| | | | TPTELVKAMKLGHTILKLFPGEVVGPQFVK |
| | | | AMKGPFPNVKFVPTGGVNLDNVCEWFKAG |
| | | | VLAVGVGSALVKGTPDEVREKAKAFVEKIR |
| | | | GCTE |
| Comp A-RSV | CompA-RSV- | none | QNITEEFYQSTCSAVSRGYFSALRTGWYTS |
| B fusion | 02 | | VITIELSNITETKCNGTDTKVKLIKQELDKYK |
| | SEQ ID NO: | | NAVTELQLLMQNTPAANNRARREAPQHMN |
| | 184 | | YTINTTKNLNVSISKKRKRRFLGFLLGVGSA |
| | | | IASGIAVCKVLHLEGEVNKIKNALLSTNKAV |
| | | | VSLSNGVSVLTFKVLDLKNYINNQLLPILNQ |
| | | | QSCRIFNIETVIEFQQKNSRLLEITREFSVNA |
| | | | GVTTPLSTYMLTNSELLSLINDMPITNDQKK |
| | | | LMSSNVQIVRQQSYSIMCIIKEEVLAYVVQL |
| | | | PIYGVIDTPCWKLHTSPLCTTNIKEGSNICLT |
| | | | RTDRGWYCDNAGSVSFFPQADTCKVQSNR |
| | | | VFCDTMNSLTLPSEVSLCNTDIFNSKYDCKI |
| | | | MTSKTDISSSVITSLGAIVSCYGKTKCTASN |
| | | | KNRGIIKTFSNGCDYVSNKGVDTVSVGNTL |
| | | | YYVNKLEGKNLYVKGEPIINYYDPLVFPSDE |
| | | | FDASISQVNEKINQSLAFIRKSDELLGSGGSG |
| | | | SGSGGSEKAAKAEEAARKMEELFKKHKIVA |
| | | | VLRANSVEEAIEKAVAVFAGGVHLIEITFTV |
| | | | PDADTVIKALSVLKEKGAIIGAGTVTSVEQC |
| | | | RKAVESGAEFIVSPHLDEEISQFCKEKGVFY |
| | | | MPGVMTPTELVKAMKLGHTILKLFPGEVVG |
| | | | PQFVKAMKGPFPNVKFVPTGGVNLDNVCE |
| | | | WFKAGVLAVGVGSALVKGTPDEVREKAKA |
| | | | FVEKIRGCTE |
| RSV BF | DS-Cav1- | | QNITEEFYQSTCSAVSRGYFSALRTGWYTS |
| | foldon | | VITIELSNITETKCNGTDTKVKLIKQELDKYK |
| | SEQ ID NO: | | NAVTELQLLMQNTPAANNRARREAPQHMN |
| | 185 | | YTINTTKNLNVSISKKRKRRFLGFLLGVGSA |
| | | | IASGIAVCKVLHLEGEVNKIKNALLSTNKAV |
| | | | VSLSNGVSVLTFKVLDLKNYINNQLLPILNQ |
| | | | QSCRIFNIETVIEFQQKNSRLLEITREFSVNA |
| | | | GVTTPLSTYMLTNSELLSLINDMPITNDQKK |
| | | | LMSSNVQIVRQQSYSIMCIIKEEVLAYVVQL |
| | | | PIYGVIDTPCWKLHTSPLCTTNIKEGSNICLT |
| | | | RTDRGWYCDNAGSVSFFPQADTCKVQSNR |
| | | | VFCDTMNSLTLPSEVSLCNTDIFNSKYDCKI |
| | | | MTSKTDISSSVITSLGAIVSCYGKTKCTASN |
| | | | KNRGIIKTFSNGCDYVSNKGVDTVSVGNTL |
| | | | YYVNKLEGKNLYVKGEPIINYYDPLVFPSDE |
| | | | FDASISQVNEKINQSLAFIRKSDELLGYIPEA |
| | | | PRDGQAYVRKDGEWVLLSTFL |
| Comp A-RSV | DS-Cav1- | | QNITEEFYQSTCSAVSRGYFSALRTGWYTS |
| B fusion | foldon-T33- | | VITIELSNITETKCNGTDTKVKLIKQELDKYK |
| | 31A | | NAVTELQLLMQNTPAANNRARREAPQHMN |
| | SEQ ID NO: | | YTINTTKNLNVSISKKRKRRFLGFLLGVGSA |
| | 186 | | IASGIAVCKVLHLEGEVNKIKNALLSTNKAV |
| | | | VSLSNGVSVLTFKVLDLKNYINNQLLPILNQ |
| | | | QSCRIFNIETVIEFQQKNSRLLEITREFSVNA |
| | | | GVTTPLSTYMLTNSELLSLINDMPITNDQKK |
| | | | LMSSNVQIVRQQSYSIMCIIKEEVLAYVVQL |
| | | | PIYGVIDTPCWKLHTSPLCTTNIKEGSNICLT |
| | | | RTDRGWYCDNAGSVSFFPQADTCKVQSNR |
| | | | VFCDTMNSLTLPSEVSLCNTDIFNSKYDCKI |
| | | | MTSKTDISSSVITSLGAIVSCYGKTKCTASN |
| | | | KNRGIIKTFSNGCDYVSNKGVDTVSVGNTL |
| | | | YYVNKLEGKNLYVKGEPIINYYDPLVFPSDE |
| | | | FDASISQVNEKINQSLAFIRKSDELLGYIPEA |
| | | | PRDGQAYVRKDGEWVLLSTFLGGSMEEVV |
| | | | LITVPSALVAVKIAHALVEERLAACVNIVPG |
| | | | LTSIYREEGSVVSDHELLLLVKTTTDAFPKL |
| | | | KERVKELHPYEVPEIVALPIAEGNREYLDWL |
| | | | RENTG |
| Comp A-RSV | DS-Cav1- | | QNITEEFYQSTCSAVSRGYFSALRTGWYTS |
| B fusion | T33-31A | | VITIELSNITETKCNGTDTKVKLIKQELDKYK |
| | SEQ ID NO: | | NAVTELQLLMQNTPAANNRARREAPQHMN |
| | 187 | | YTINTTKNLNVSISKKRKRRFLGFLLGVGSA |
| | | | IASGIAVCKVLHLEGEVNKIKNALLSTNKAV |
| | | | VSLSNGVSVLTFKVLDLKNYINNQLLPILNQ |
| | | | QSCRIFNIETVIEFQQKNSRLLEITREFSVNA |
| | | | GVTTPLSTYMLTNSELLSLINDMPITNDQKK |
| | | | LMSSNVQIVRQQSYSIMCIIKEEVLAYVVQL |
| | | | PIYGVIDTPCWKLHTSPLCTTNIKEGSNICLT |
| | | | RTDRGWYCDNAGSVSFFPQADTCKVQSNR |
| | | | VFCDTMNSLTLPSEVSLCNTDIFNSKYDCKI |
| | | | MTSKTDISSSVITSLGAIVSCYGKTKCTASN |
| | | | KNRGIIKTFSNGCDYVSNKGVDTVSVGNTL |
| | | | YYVNKLEGKNLYVKGEPIINYYDPLVFPSDE |
| | | | FDASISQVNEKINQSLAFIRKSDELLGGSMEE |
| | | | VVLITVPSALVAVKIAHALVEERLAACVNIV |
| | | | PGLTSIYREEGSVVSDHELLLLVKTTTDAFP |
| | | | KLKERVKELHPYEVPEIVALPIAEGNREYLD |
| | | | WLRENTG |
| Comp A-RSV | DS-Cav1- | | QNITEEFYQSTCSAVSRGYFSALRTGWYTS |
| B fusion | foldon-T33- | | VITIELSNITETKCNGTDTKVKLIKQELDKYK |
| | 15B | | NAVTELQLLMQNTPAANNRARREAPQHMN |
| | SEQ ID NO: | | YTINTTKNLNVSISKKRKRRFLGFLLGVGSA |
| | 188 | | IASGIAVCKVLHLEGEVNKIKNALLSTNKAV |
| | | | VSLSNGVSVLTFKVLDLKNYINNQLLPILNQ |
| | | | QSCRIFNIETVIEFQQKNSRLLEITREFSVNA |
| | | | GVTTPLSTYMLTNSELLSLINDMPITNDQKK |
| | | | LMSSNVQIVRQQSYSIMCIIKEEVLAYVVQL |
| | | | PIYGVIDTPCWKLHTSPLCTTNIKEGSNICLT |
| | | | RTDRGWYCDNAGSVSFFPQADTCKVQSNR |
| | | | VFCDTMNSLTLPSEVSLCNTDIFNSKYDCKI |
| | | | MTSKTDISSSVITSLGAIVSCYGKTKCTASN |
| | | | KNRGIIKTFSNGCDYVSNKGVDTVSVGNTL |
| | | | YYVNKLEGKNLYVKGEPIINYYDPLVFPSDE |
| | | | FDASISQVNEKINQSLAFIRKSDELLGYIPEA |
| | | | PRDGQAYVRKDGEWVLLSTFLGGSMVRGI |
| | | | RGAITVNSDTPTSIIIATILLLEKMLEANGIQS |
| | | | YEELAAVIFTVTEDLTSAFPAEAARQIGMHR |
| | | | VPLLSAREVPVPGSLPRVIRVLALWNTDTPQ |
| | | | DRVRHVYLSEAVRLRPDLESAQ |

In some embodiments, the RSV protein ectodomain comprises the substitutions S46G, K66E, 176V, K77E, K80E, N88C, E92C, E92D), Q98C, A149C, E161G, E161P, E161Q, S173P, N175P, S182P, G184N, V185N, K201Q, L2031, K209Q, S215P, S238C, 254C, Q279C, Q361C, K421N, N426S, N428C, K465E, K465Q, Y458C, K508E, or any combination thereof. In some embodiments, the RSV protein ectodomain comprises the substitutions Q98C and Q361C, A149C and Y458C, N183GC and N428C, N88C and N254C, E92C and N254C, and/or S238C and Q279C, or any combination thereof.

TABLE 3B
Illustrative Amino Acid Sequences of VLPs of the Disclosure

| Construct | SEQ ID | |
|---|---|---|
| name | NO: | Amino Acid Sequence |
| hMPV001 | 92 | KESYLEESCSTITEGYLSVLRTGWYTNVFTLEVGDVENLTCADGPSLIKTELDLT |
| | | KSALRELRTVSADQLAREEQIENPRRRRFVLGAIALGVATAAAVTAGVAIAKTIR |
| | | LESEVTAIKNALKKTNEAVSTLGNGVRVLATAVRELKDFVSKNLTRAINKNKCDI |
| | | PDLKMAVSFSQFNRRFLNVVRQFSDNAGITPAISLDLMTDAELARAVSNMPTSAG |
| | | QIKLMLENRAMVRRKGFGFLIGVYGSSVIYMVQLPIFGVIDTPCWIVKAAPSCSG |
| | | KKGNYACLLREDQGWYCQNAGSTVYYPNEKDCETRGDHVFCDTAAGINVAEQSKE |
| | | CNINISTTNYPCKVSTGRHPISMVALSPLGALVACYKGVSCSIGSNRVGIIKQLN |
| | | KGCSYITNQDADTVTIDNTVYQLSKVEGEQHVIKGRPVSSSFDPVKFPEDQENVA |
| | | LDQVFESIENSQALVDQSNRILGSGGSGSGSGGSEKAAKAEEAARKMEELFKKHK |
| | | IVAVLRANSVEEAIEKAVAVFAGGVHLIEITFTVPDADTVIKALSVLKEKGAIIG |
| | | AGTVTSVEQARKAVESGAEFIVSPHLDEEISQFAKEKGVFYMPGVMTPTELVKAM |
| | | KLGHTILKLFPGEVVGPQFVKAMKGPFPNVKFVPTGGVNLDNVAEWFKAGVLAVG |
| | | VGSALVKGTPDEVREKAKAFVEKIRGATELE |
| hMPV002 | 93 | LKESYLEESCSTITEGYLSVLRTGWYTNVFTLEVGDVENLTCADGPSLIKTELDL |
| | | TKSALRELRTVSADQLAREEQIENPRRRRFVLGAIALGVATAAAVTAGVAIAKTI |
| | | RLESEVTAIKNALKKTNEAVSTLGNGVRVLATAVRELKDFVSKNLTRAINKNKCD |
| | | IPDLKMAVSFSQFNRRELNVVRQFSDNAGITPAISLDLMTDAELARAVSNMPTSA |
| | | GQIKLMLENRAMVRRKGFGFLIGVYGSSVIYMVQLPIFGVIDTPCWIVKAAPSCS |
| | | GKKGNYACLLREDQGWYCQNAGSTVYYPNEKDCETRGDHVFCDTAAGINVAEQSK |
| | | ECNINISTTNYPCKVSTGRHPISMVALSPLGALVACYKGVSCSIGSNRVGIIKQL |
| | | NKGCSYITNQDADTVTIDNTVYQLSKVEGEQHVIKGRPVSSSFDPVKFPEDQENV |
| | | ALDQVFESIENSQAGSGGSGSGSGGSEKAAKAEEAARKMEELFKKHKIVAVLRAN |
| | | SVEEAIEKAVAVFAGGVHLIEITFTVPDADTVIKALSVLKEKGAIIGAGTVTSVE |
| | | QARKAVESGAEFIVSPHLDEEISQFAKEKGVFYMPGVMTPTELVKAMKLGHTILK |
| | | LFPGEVVGPQFVKAMKGPFPNVKFVPTGGVNLDNVAEWFKAGVLAVGVGSALVKG |
| | | TPDEVREKAKAFVEKIRGATELE |
| hMPV003 | 94 | LKESYLEESCSTITEGYLSVLRTGWYTNVFTLEVGDVENLTCADGPSLIKTELDL |
| | | TKSALRELRTVSADQLAREEQIENPRRRRFVLGAIALGVATAAAVTAGVAIAKTI |
| | | RLESEVTAIKNALKKTNEAVSTLGNGVRVLATAVRELKDFVSKNLTRAINKNKCD |
| | | IPDLKMAVSFSQFNRRFLNVVRQFSDNAGITPAISLDLMTDAELARAVSNMPTSA |
| | | GQIKLMLENRAMVRRKGFGFLIGVYGSSVIYMVQLPIFGVIDTPCWIVKAAPSCS |
| | | GKKGNYACLLREDQGWYCQNAGSTVYYPNEKDCETRGDHVFCDTAAGINVAEQSK |
| | | ECNINISTTNYPCKVSTGRHPISMVALSPLGALVACYKGVSCSIGSNRVGIIKQL |
| | | NKGCSYITNQDADTVTIDNTVYQLSKVEGEQHVIKGRPVSSSFDPVKFPEDQFNV |
| | | ALDQVFESIENSQALVDQSNRILSSAEKGNTGGSGGSGSGSGGSEKAAKAEEAAR |
| | | KMEELFKKHKIVAVLRANSVEEAIEKAVAVFAGGVHLIEITFTVPDADTVIKALS |
| | | VLKEKGAIIGAGTVTSVEQARKAVESGAEFIVSPHLDEEISQFAKEKGVFYMPGV |
| | | MTPTELVKAMKLGHTILKLFPGEVVGPQFVKAMKGPFPNVKFVPTGGVNLDNVAE |
| | | WFKAGVLAVGVGSALVKGTPDEVREKAKAFVEKIRGATELE |
| hMPV004 | 95 | LKESYLEESCSTITEGYLSVLRTGWYTNVFTLEVGDVENLTCADGPSLIKTELDL |
| | | TKSALRELRTVSADQLAREEQIENPRRRRFVLGAIALGVATAAAVTAGVAIAKTI |
| | | RLESEVTAIKNALKKTNEAVSTLGNGVRVLATAVRELKDFVSKNLTRAINKNKCD |
| | | IADLKMAVSFSQFNRRELNVVRQFSDNAGITPAISLDLMTDAELARAVSNMPTSA |
| | | GQIKLMLENRAMVRRKGFGFLIGVYGSSVIYMVQLPIFGVIDTPCWIVKAAPSCS |
| | | GKKGNYACLLREDQGWYCQNAGSTVYYPNEKDCETRGDHVFCDTAAGINVAEQSK |
| | | ECNINISTTNYPCKVSTGRHPISMVALSPLGALVACYKGVSCSIGSNRVGIIKQL |
| | | NKGCSYITNQDADTVTIDNTVYQLSKVEGEQHVIKGRPVSSSFDPVKFPEDQFNV |
| | | ALDQVFESIENSQAGSGGSGSGSGGSEKAAKAEEAARKMEELFKKHKIVAVLRAN |
| | | SVEEAIEKAVAVFAGGVHLIEITFTVPDADTVIKALSVLKEKGAIIGAGTVTSVE |
| | | QARKAVESGAEFIVSPHLDEEISQFAKEKGVFYMPGVMTPTELVKAMKLGHTILK |
| | | LFPGEVVGPQFVKAMKGPFPNVKFVPTGGVNLDNVAEWFKAGVLAVGVGSALVKG |
| | | TPDEVREKAKAFVEKIRGATELE |
| hMPV005 | 96 | LKESYLEESCSTITEGYLSVLRTGWYTNVFTLEVGDVENLTCADGPSLIKTELDL |
| | | TKSALRELRTVSADQLAREEQIENPRRRRFVLGAIALGVATAAAVTAGVAIAKCI |
| | | RLESEVTAIKNALKKTNEAVSTLGCGVRVLATAVRELKDFVSKNLTRAINKNKCD |
| | | IPDLKMAVSFSQFNRRFLNVVRQFSDNAGITPAISLDLMTDAELARAVSNMPTSA |
| | | GQIKLMLENRAMVRRKGFGILIGVYGSSVIYMVQLPIFGVIDTPCWIVKAAPSCS |
| | | GKKGNYACLLREDQGWYCQNAGSTVYYPNEKDCETRGDHVFCDTAAGINVAEQSK |
| | | ECNINISTTNYPCKVSTGRNPISMVALSPLGALVACYKGVSCSIGSNRVGIIKQL |
| | | NKGCSYITNQDADTVTIDNTVYQLSKVEGEQHVIKGRPVSSSFDPVKFPEDQENV |
| | | ALDQVFESIENSQAGSGGSGSGSGGSEKAAKAEEAARKMEELFKKHKIVAVLRAN |
| | | SVEEAIEKAVAVFAGGVHLIEITFTVPDADTVIKALSVLKEKGAIIGAGTVTSVE |
| | | QARKAVESGAEFIVSPHLDEEISQFAKEKGVFYMPGVMTPTELVKAMKLGHTILK |
| | | LFPGEVVGPQFVKAMKGPFPNVKFVPTGGVNLDNVAEWFKAGVLAVGVGSALVKG |
| | | TPDEVREKAKAFVEKIRGATELE |
| hMPV006 | 97 | KESYLEESCSTITEGYLSVLRTGWYTNVFTLEVGDVENLTCTDGPSLIKTELDLT |
| | | KSALRELKTCSADQLAREEQIEGGGGGGFVLGAIALGVATAAAVTAGIAIAKTIR |
| | | LESEVNAIKGCLKTTNECVSTLGNGVRVLATAVRELKEFVSKNLTSAINKNKCDI |
| | | ADLKMAVSFSQFNRRFLNVVRQFSDNAGITPAISLDLMTDAELARAVSYMPTSAG |
| | | QIKLMLENRCMVRRKGFGILIGVYGSSVIYMVQLPIFGVIDTPCWIIKAAPSCSE |
| | | KDGNYACLLREDQGWYCKNAGSTVYYPNDKDCETRGDHVFCDTAAGINVAEQSRE |
| | | CNINISTTNYPCKVSTGRHPISMVALSPLGALVACYKGVSCSIGSNRVGIIKQLP |
| | | KGCSYITNQDADTVTIDNTVYQLSKVEGEQHVIKGRPVSSSFDPIKFPEDQENVA |
| | | LDQVFESIENSQAGSGGSGSGSGGSEKAAKAEEAARKMEELFKKHKIVAVLRANS |
| | | VEEAIEKAVAVFAGGVHLIEITFTVPDADTVIKALSVLKEKGAIIGAGTVTSVEQ |
| | | ARKAVESGAEFIVSPHLDEEISQFAKEKGVFYMPGVMTPTELVKAMKLGHTILKL |
| | | FPGEVVGPQFVKAMKGPFPNVKFVPTGGVNLDNVAEWFKAGVLAVGVGSALVKGT |
| | | PDEVREKAKAFVEKIRGATELE |
| hMPV007 | 98 | KESYLEESCSTITEGYLSVLRTGWYTNVFTLEVGDVENLTCTDGPSLIKTELDLT |
| | | KSALRELKTCSADQLAREEQIEGGGGGGFVLGAIALGVATAAAVTAGIAIAKTIR |
| | | LESEVNAIKGCLKTTNECVSTLGNGVRVLATAVRELKEFVSKNLTSAINKNKCDI |
| | | ADLKMAVSFSQFNRRFLNVVRQFSDNAGITPAISLDLMTDAELARAVSYMPTSAG |
| | | QIKLMLENRCMVRRKGFGILIGVYGSSVIYMVQLPIFGVIDTPCWIIKAAPSCSE |
| | | KDGNYACLLREDQGWYCKNAGSTVYYPNDKDCETRGDHVECDTAAGINVAEQSRE |
| | | CNINISTTNYPCKVSTGRHPISMVALSPLGALVACYKGVSCSIGSNRVGIIKQLP |
| | | KGCSYITNQDADTVTIDNTVYQLSKVEGEQHVIKGRPVSSSFDPIKFPEDQENVA |
| | | LDQVFESIENSQALVDQSNKILNSAESAIGSGGSGSGSGGSEKAAKAEEAARKME |
| | | ELFKKHKIVAVLRANSVEEAIEKAVAVFAGGVHLIEITFTVPDADTVIKALSVLK |
| | | EKGAIIGAGTVTSVEQARKAVESGAEFIVSPHLDEEISQFAKEKGVFYMPGVMTP |
| | | TELVKAMKLGHTILKLFPGEVVGPQFVKAMKGPFPNVKFVPTGGVNLDNVAEWFK |
| | | AGVLAVGVGSALVKGTPDEVREKAKAFVEKIRGATELE |
| hMPV008 | 99 | KESYLEESCSTITEGYLSVLRTGWYTNVFTLEVGDVENLTCTDCPSLIKTELDLT |
| | | KSALRELKTVSADQLAREEQIEGGGGGGFVLGAIALGVATAAAVTAGIAIAKTIR |
| | | LESEVNAIKGCLKTTNECVSTLGNGVRVLATAVRELKEFVSKNLTSAINKNKCDI |
| | | ADLCMAVSFSQFNRRFLNVVRQFSDNAGITPAISLDLMTDAELARAVSYMPTSAG |
| | | QIKLMLENRAMVRRKGFGILIGVYGSSVIYMVQLPIFGVIDTPCWIIKAAPSCSE |
| | | KDGNYACLLREDQGWYCKNAGSTVYYPNDKDCETRGDHVFCDTAAGINVAEQSRE |
| | | CNINISTTNYPCKVSTGRHPISMVALSPLGALVACYKGVSCSIGSNRVGIIKQLP |
| | | KGCSYITNQDADTVTIDNTVYQLSKVEGEQHVIKGRPVSSSFDPICFPEDQENVA |
| | | LDQVFESIENCQAGSGGSGSGSGGSEKAAKAEEAARKMEELFKKHKIVAVLRANS |
| | | VEEAIEKAVAVFAGGVHLIEITFTVPDADTVIKALSVLKEKGAIIGAGTVTSVEQ |
| | | ARKAVESGAEFIVSPHLDEEISQFAKEKGVFYMPGVMTPTELVKAMKLGHTILKL |
| | | FPGEVVGPQFVKAMKGPFPNVKFVPTGGVNLDNVAEWFKAGVLAVGVGSALVKGT |
| | | PDEVREKAKAFVEKIRGATELE |
| hMPV009 | 100 | KESYLEESCSTITEGYLSVLRTGWYTNVFTLEVGDVENLTCTDCPSLIKTELDLT |
| | | KSALRELKTVSADQLAREEQIEGGGGGGFVLGAIALGVATAAAVTAGIAIAKTIR |
| | | LESEVNAIKGCLKTTNECVSTLGNGVRVLATAVRELKEFVSKNLTSAINKNKCDI |
| | | ADLCMAVSFSQFNRRELNVVRQFSDNAGITPAISLDLMTDAELARAVSYMPTSAG |
| | | QIKLMLENRAMVRRKGFGILIGVYGSSVIYMVQLPIFGVIDTPCWIIKAAPSCSE |
| | | KDGNYACLLREDQGWYCKNAGSTVYYPNDKDCETRGDHVFCDTAAGINVAEQSRE |
| | | CNINISTTNYPCKVSTGRHPISMVALSPLGALVACYKGVSCSIGSNRVGIIKQLP |
| | | KGCSYITNQDADTVTIDNTVYQLSKVEGEQHVIKGRPVSSSFDPICFPEDQENVA |
| | | LDQVFESIENCQALVDQSNKILNSAESAIGSGGSGSGSGGSEKAAKAEEAARKME |
| | | ELFKKHKIVAVLRANSVEEAIEKAVAVFAGGVHLIEITFTVPDADTVIKALSVLK |
| | | EKGAIIGAGTVTSVEQARKAVESGAEFIVSPHLDEEISQFAKEKGVFYMPGVMTP |
| | | TELVKAMKLGHTILKLFPGEVVGPQFVKAMKGPFPNVKFVPTGGVNLDNVAEWEK |
| | | AGVLAVGVGSALVKGTPDEVREKAKAFVEKIRGATELE |
| hMPV010 | 101 | LKESYLEESCSTITEGYLSVLRTGWYTNVFTLEVGDVENLTCSDCPSLIKTELDL |
| | | TKSALRELKTVSADQLAREEQIEGGGGGGFVLAIALGVATAAAVTAGVAIAKTIR |
| | | LESEVTAIKNCLKTTNECVSTLGNGVRVLATAVRELKDFVSKNLTSAINKNKCDI |
| | | DDLCMAVSFSQFNRRFLNVVRQFSDNAGITPAISLDLMTDAELARAVSNMPTSAG |
| | | QIKLMLENRAMVRRKGFGILIGVYGSSVIYMVQLPIFGVIDTPCWIIKAAPSCSG |
| | | KKGNYACLLREDQGWYCQNAGSTVYYPNEKDCETRGDHVFCDTAAGINVAEQSKE |
| | | CNINISTTNYPCKVSTGRHPISMVALSPLGALVACYKGVSCSIGSNRVGIIKQLN |
| | | KGCSYITNQDADTVTIDNTVYQLSKVEGEQHVIKGRPVSSSFDPIKFPEDQENVA |
| | | LDQVFENIENSQAGSGGSGSGSGGSEKAAKAEEAARKMEELFKKHKIVAVLRANS |
| | | VEEAIEKAVAVFAGGVHLIEITFTVPDADTVIKALSVLKEKGAIIGAGTVTSVEQ |
| | | ARKAVESGAEFIVSPHLDEEISQFAKEKGVFYMPGVMTPTELVKAMKLGHTILKL |
| | | FPGEVVGPQFVKAMKGPFPNVKFVPTGGVNLDNVAEWFKAGVLAVGVGSALVKGT |
| | | PDEVREKAKAFVEKIRGATELE |
| hMPV011 | 102 | LKESYLEESCSTITEGYLSVLRTGWYTNVFTLEVGDVENLTCSDCPSLIKTELDL |
| | | TKSALRELKTVSADQLAREEQIEGGGGGGFVLAIALGVATAAAVTAGVAIAKTIR |
| | | LESEVTAIKNCLKTTNECVSTLGNGVRVLATAVRELKDFVSKNLTSAINKNKCDI |
| | | DDLCMAVSFSQFNRRFLNVVRQFSDNAGITPAISLDLMTDAELARAVSNMPTSAG |
| | | QIKLMLENRAMVRRKGFGILIGVYGSSVIYMVQLPIFGVIDTPCWIIKAAPSCSG |
| | | KKGNYACLLREDQGWYCQNAGSTVYYPNEKDCETRGDHVFCDTAAGINVAEQSKE |
| | | CNINISTTNYPCKVSTGRHPISMVALSPLGALVACYKGVSCSIGSNRVGIIKQLN |
| | | KGCSYITNQDADTVTIDNTVYQLSKVEGEQHVIKGRPVSSSFDPIKFPEDQENVA |
| | | LDQVFENIENSQALVDQSNRILSSAESAIGSGGSGSGSGGSEKAAKAEEAARKME |
| | | ELFKKHKIVAVLRANSVEEAIEKAVAVFAGGVHLIEITFTVPDADTVIKALSVLK |
| | | EKGAIIGAGTVTSVEQARKAVESGAEFIVSPHLDEEISQFAKEKGVFYMPGVMTP |
| | | TELVKAMKLGHTILKLFPGEVVGPQFVKAMKGPFPNVKFVPTGGVNLDNVAEWEK |
| | | AGVLAVGVGSALVKGTPDEVREKAKAFVEKIRGATELE |
| hMPV012 | 103 | KESYLEESCSTITEGYLSVLRTGWYTNVFTLEVGDVENLTCTDGPSLIKTELDLT |
| | | KSALRELKTVSADQLAREEQIENPRKARFVLGAIALGVCTAAAVTCGIAIAKTIR |
| | | LESEVNAIKGALKTTNEAVSTLGNGVRVLAFAVRELKEFVSKNLTSALNKNKCDI |
| | | ADLKMAVSFSQFNRRELNVVRQFSDNAGITPAISLDLMTDAELARAVSYMPTSAG |
| | | QIKLMLENRAMVRRKGFGILIGVYGSSVIYMVQLPIFGVIDTPCWIIKAAPSCSE |
| | | KDGNYACLLREDQGWYCKNAGSTVYYPNDKDCETRGDHVFCDTACGINVAEQSRE |
| | | CNINISTTNYPCKVSTGRHPISMVALSPLGALVACYKGVSCSIGSNRVGIIKQLP |
| | | KGCSYITNQDADTVTIDNTVYCLSKVEGEQHVIKGRPVSSSFDPIKFPEDQENVA |
| | | LDQVFESIENSQAGSGGSGSGSGGSEKAAKAEEAARKMEELFKKHKIVAVLRANS |
| | | VEEAIEKAVAVFAGGVHLIEITFTVPDADTVIKALSVLKEKGAIIGAGTVTSVEQ |
| | | ARKAVESGAEFIVSPHLDEEISQFAKEKGVFYMPGVMTPTELVKAMKLGHTILKL |
| | | FPGEVVGPQFVKAMKGPFPNVKFVPTGGVNLDNVAEWFKAGVLAVGVGSALVKGT |
| | | PDEVREKAKAFVEKIRGATELE |
| hMPV013 | 104 | KESYLEESCSTITEGYLSVLRTGWYTNVFTLEVGDVENLTCTDGPSLIKTELDLT |
| | | KSALRELKTVSADQLAREEQIENPRKARFVLGAIALGVCTAAAVTCGIAIAKTIR |
| | | LESEVNAIKGALKTTNEAVSTLGNGVRVLAFAVRELKEFVSKNLTSALNKNKCDI |
| | | ADLKMAVSFSQFNRRFLNVVRQFSDNAGITPAISLDLMTDAELARAVSYMPTSAG |
| | | QIKLMLENRAMVRRKGFGILIGVYGSSVIYMVQLPIFGVIDTPCWIIKAAPSCSE |
| | | KDGNYACLLREDQGWYCKNAGSTVYYPNDKDCETRGDHVFCDTACGINVAEQSRE |
| | | CNINISTTNYPCKVSTGRHPISMVALSPLGALVACYKGVSCSIGSNRVGIIKQLP |
| | | KGCSYITNQDADTVTIDNTVYCLSKVEGEQHVIKGRPVSSSFDPIKFPEDQENVA |
| | | LDQVFESIENSQALVDQSNKILNSAESAIGSGGSGSGSGGSEKAAKAEEAARKME |
| | | ELFKKHKIVAVLRANSVEEAIEKAVAVFAGGVHLIEITFTVPDADTVIKALSVLK |
| | | EKGAIIGAGTVTSVEQARKAVESGAEFIVSPHLDEEISQFAKEKGVFYMPGVMTP |
| | | TELVKAMKLGHTILKLFPGEVVGPQFVKAMKGPFPNVKFVPTGGVNLDNVAEWFK |
| | | AGVLAVGVGSALVKGTPDEVREKAKAFVEKIRGATELE |
| hMPV014 | 105 | KESYLEESCSTITEGYLSVLRTGWYTNVFTLEVGDVENLTCTDGPSLIKTELDLT |
| | | KSALRELKTCSADQLAREEQIENPRRRRFVLGAIALGVATAAAVTAGIAIAKTIR |
| | | LESEVNAIKGCLKTTNECVSTLGNGVRVLATAVRELKEFVSKNLTSAINKNKCDI |
| | | ADLKMAVSFSQFNRRFLNVVRQFSDNAGITPAISLDLMTDAELARAVSYMPTSAG |
| | | QIKLMLENRCMVRRKGFGILIGVYGSSVIYMVQLPIFGVIDTPCWIIKAAPSCSE |
| | | KDGNYACLLREDQGWYCKNAGSTVYYPNDKDCETRGDHVFCDTAAGINVAEQSRE |
| | | CNINISTTNYPCKVSTGRHPISMVALSPLGALVACYKGVSCSIGSNRVGIIKQLP |
| | | KGCSYITNQDADTVTIDNTVYQLSKVEGEQHVIKGRPVSSSFDPIKFPEDQENVA |
| | | LDQVFESIENSQAGSGGSGSGSGGSEKAAKAEEAARKMEELFKKHKIVAVLRANS |
| | | VEEAIEKAVAVFAGGVHLIEITFTVPDADTVIKALSVLKEKGAIIGAGTVTSVEQ |
| | | ARKAVESGAEFIVSPHLDEEISQFAKEKGVFYMPGVMTPTELVKAMKLGHTILKL |
| | | FPGEVVGPQFVKAMKGPFPNVKFVPTGGVNLDNVAEWFKAGVLAVGVGSALVKGT |
| | | PDEVREKAKAFVEKIRGATELE |
| hMPV015 | 106 | KESYLEESCSTITEGYLSVLRTGWYTNVFTLEVGDVENLTCTDCPSLIKTELDLT |
| | | KSALRELKTVSADQLAREEQIENPRRRRFVLGAIALGVATAAAVTAGIAIAKTIR |
| | | LESEVNAIKGCLKTTNECVSTLGNGVRVLATAVRELKEFVSKNLTSAINKNKCDI |
| | | ADLCMAVSFSQFNRRELNVVRQFSDNAGITPAISLDLMTDAELARAVSYMPTSAG |
| | | QIKLMLENRAMVRRKGFGILIGVYGSSVIYMVQLPIFGVIDTPCWIIKAAPSCSE |
| | | KDGNYACLLREDQGWYCKNAGSTVYYPNDKDCETRGDHVFCDTAAGINVAEQSRE |
| | | CNINISTTNYPCKVSTGRHPISMVALSPLGALVACYKGVSCSIGSNRVGIIKQLP |
| | | KGCSYITNQDADTVTIDNTVYQLSKVEGEQHVIKGRPVSSSFDPICFPEDQENVA |
| | | LDQVFESIENCQAGSGGSGSGSGGSEKAAKAEEAARKMEELFKKHKIVAVLRANS |
| | | VEEAIEKAVAVFAGGVHLIEITFTVPDADTVIKALSVLKEKGAIIGAGTVTSVEQ |
| | | ARKAVESGAEFIVSPHLDEEISQFAKEKGVFYMPGVMTPTELVKAMKLGHTILKL |
| | | FPGEVVGPQFVKAMKGPFPNVKFVPTGGVNLDNVAEWFKAGVLAVGVGSALVKGT |
| | | PDEVREKAKAFVEKIRGATELE |
| hMPV016 | 107 | LKESYLEESCSTITEGYLSVLRTGWYTNVFTLEVGDVENLTCSDCPSLIKTELDL |
| | | TKSALRELKTVSADQLAREEQIENPRRRRFVLAIALGVATAAAVTAGVAIAKTIR |
| | | LESEVTAIKNCLKTTNECVSTLGNGVRVLATAVRELKDFVSKNLTSAINKNKCDI |
| | | DDLCMAVSFSQFNRRELNVVRQFSDNAGITPAISLDLMTDAELARAVSNMPTSAG |
| | | QIKLMLENRAMVRRKGFGILIGVYGSSVIYMVQLPIFGVIDTPCWIIKAAPSCSG |
| | | KKGNYACLLREDQGWYCQNAGSTVYYPNEKDCETRGDHVFCDTAAGINVAEQSKE |
| | | CNINISTTNYPCKVSTGRHPISMVALSPLGALVACYKGVSCSIGSNRVGIIKQLN |
| | | KGCSYITNQDADTVTIDNTVYQLSKVEGEQHVIKGRPVSSSFDPIKFPEDQENVA |
| | | LDQVFENIENSQAGSGGSGSGSGGSEKAAKAEEAARKMEELFKKHKIVAVLRANS |
| | | VEEAIEKAVAVFAGGVHLIEITFTVPDADTVIKALSVLKEKGAIIGAGTVTSVEQ |
| | | ARKAVESGAEFIVSPHLDEEISQFAKEKGVFYMPGVMTPTELVKAMKLGHTILKL |
| | | FPGEVVGPQFVKAMKGPFPNVKFVPTGGVNLDNVAEWFKAGVLAVGVGSALVKGT |
| | | PDEVREKAKAFVEKIRGATELE |
| hMPV017 | 108 | KESYLEESCSTITEGYLSVLRTGWYTNVFTLEVGDVENLTCTDGPSLIKTELDLT |
| | | KSALRELKTVSADQLAREEQIENPRRRRFVLGAIALGVCTAAAVTCGIAIAKTIR |
| | | LESEVNAIKGALKTTNEAVSTLGNGVRVLAFAVRELKEFVSKNLTSALNKNKCDI |
| | | ADLKMAVSFSQFNRRFLNVVRQFSDNAGITPAISLDLMTDAELARAVSYMPTSAG |
| | | QIKLMLENRAMVRRKGFGILIGVYGSSVIYMVQLPIFGVIDTPCWIIKAAPSCSE |
| | | KDGNYACLLREDQGWYCKNAGSTVYYPNDKDCETRGDHVFCDTACGINVAEQSRE |
| | | CNINISTTNYPCKVSTGRHPISMVALSPLGALVACYKGVSCSIGSNRVGIIKQLP |
| | | KGCSYITNQDADTVTIDNTVYCLSKVEGEQHVIKGRPVSSSEDPIKEPEDQENVA |
| | | LDQVFESIENSQAGSGGSGSGSGGSEKAAKAEEAARKMEELFKKHKIVAVLRANS |
| | | VEEAIEKAVAVFAGGVHLIEITFTVPDADTVIKALSVLKEKGAIIGAGTVTSVEQ |
| | | ARKAVESGAEFIVSPHLDEEISQFAKEKGVFYMPGVMTPTELVKAMKLGHTILKL |
| | | FPGEVVGPQFVKAMKGPFPNVKFVPTGGVNLDNVAEWFKAGVLAVGVGSALVKGT |
| | | PDEVREKAKAFVEKIRGATELE |
| hMPV018 | 109 | LKESYLEESCSTITEGYLSVLRTGWYTNVFTLEVGDVENLTCADGPSLIKTELDL |
| | | TKSALRELRTVSADQLAREEQIENPRRRRFVLGAIALGVCTAAAVTAGVAIAKTI |
| | | RLESEVTAIKNALKKTNEAVSTLGNGVRVLATAVRELKDFVSKNLTRAINKNKCD |
| | | IPDLKMAVSFSQFNRRFLNVVRQFSDNAGITPAISLDLMTDAELARAVSNMPTSA |
| | | GQIKLMLENRAMVRRKGFGFLIGVYGSSVIYMVQLPIFGVIDTPCWIVKAAPSCS |
| | | GKKGNYACLLREDQGWYCQNAGSTVYYPNEKDCETRGDHVFCDTACGINVAEQSK |
| | | ECNINISTTNYPCKVSTGRHPISMVALSPLGALVACYKGVSCSIGSNRVGIIKQL |
| | | NKGCSYITNQDADTVTIDNTVYQLSKVEGEQHVIKGRPVSSSFDPVKFPEDQENV |
| | | ALDQVFESIENSQAGSGGSGSGSGGSEKAAKAEEAARKMEELFKKHKIVAVLRAN |
| | | SVEEAIEKAVAVFAGGVHLIEITFTVPDADTVIKALSVLKEKGAIIGAGTVTSVE |
| | | QARKAVESGAEFIVSPHLDEEISQFAKEKGVFYMPGVMTPTELVKAMKLGHTILK |
| | | LFPGEVVGPQFVKAMKGPFPNVKFVPTGGVNLDNVAEWFKAGVLAVGVGSALVKG |
| | | TPDEVREKAKAFVEKIRGATELE |
| hMPV019 | 110 | LKESYLEESCSTITEGYLSVLRTGWYTNVFTLEVGDVENLTCADGPSLIKTELDL |
| | | TKSALRELRTVSADQLAREEQIENPRRRRFVLGAIALGVATAAAVTAGVAIAKTI |
| | | RLESEVTAIKNALKKTNEAVSTLGNGVRVLAFAVRELKDFVSKNLTRALNKNKCD |
| | | IPDLKMAVSFSQFNRRELNVVRQFSDNAGITPAISLDLMTDAELARAVSNMPTSA |
| | | GQIKLMLENRAMVRRKGFGFLIGVYGSSVIYMVQLPIFGVIDTPCWIVKAAPSCS |
| | | GKKGNYACLLREDQGWYCQNAGSTVYYPNEKDCETRGDHVFCDTAAGINVAEQSK |
| | | ECNINISTTNYPCKVSTGRHPISMVALSPLGALVACYKGVSCSIGSNRVGIIKQL |
| | | NKGCSYITNQDADTVTIDNTVYQLSKVEGEQHVIKGRPVSSSFDPVKFPEDQFNV |
| | | ALDQVFESIENSQAGSGGSGSGSGGSEKAAKAEEAARKMEELFKKHKIVAVLRAN |
| | | SVEEAIEKAVAVFAGGVHLIEITFTVPDADTVIKALSVLKEKGAIIGAGTVTSVE |
| | | QARKAVESGAEFIVSPHLDEEISQFAKEKGVFYMPGVMTPTELVKAMKLGHTILK |
| | | LFPGEVVGPQFVKAMKGPFPNVKFVPTGGVNLDNVAEWFKAGVLAVGVGSALVKG |
| | | TPDEVREKAKAFVEKIRGATELE |
| hMPV020 | 111 | LKESYLEESCSTITEGYLSVLRTGWYTNVFTLEVGDVENLTCADGPSLIKTELDL |
| | | TKSALRELRTVSADQLAREEQIENPRRRRFVLGAIALGVCTAAAVTAGVAIAKTI |
| | | RLESEVTAIKNALKKTNEAVSTLGNGVRVLAFAVRELKDFVSKNLTRALNKNKCD |
| | | IPDLKMAVSFSQFNRRFLNVVRQFSDNAGITPAISLDLMTDAELARAVSNMPTSA |
| | | GQIKLMLENRAMVRRKGFGFLIGVYGSSVIYMVQLPIFGVIDTPCWIVKAAPSCS |
| | | GKKGNYACLLREDQGWYCQNAGSTVYYPNEKDCETRGDHVFCDTACGINVAEQSK |
| | | ECNINISTTNYPCKVSTGRHPISMVALSPLGALVACYKGVSCSIGSNRVGIIKQL |
| | | NKGCSYITNQDADTVTIDNTVYQLSKVEGEQHVIKGRPVSSSFDPVKFPEDQENV |
| | | ALDQVFESIENSQAGSGGSGSGSGGSEKAAKAEEAARKMEELFKKHKIVAVLRAN |
| | | SVEEAIEKAVAVFAGGVHLIEITFTVPDADTVIKALSVLKEKGAIIGAGTVTSVE |
| | | QARKAVESGAEFIVSPHLDEEISQFAKEKGVFYMPGVMTPTELVKAMKLGHTILK |
| | | LFPGEVVGPQFVKAMKGPFPNVKFVPTGGVNLDNVAEWFKAGVLAVGVGSALVKG |
| | | TPDEVREKAKAFVEKIRGATELE |
| hMPV021 | 112 | KESYLEESCSTITEGYLSVLRTGWYTNVFTLEVGDVENLTCTDCPSLIKTELDLT |
| | | KSALRELKTVSADQLAREEQIEGGGGGGFVLGAIALGVATAAAVTAGIAIAKTIR |
| | | LESEVNAIKGALKTTNEAVSTLGNGVRVLATAVRELKEFVSKNLTSAINKNKCDI |
| | | ADLCMAVSFSQFNRRELNVVRQFSDNAGITPAISLDLMTDAELARAVSYMPTSAG |
| | | QIKLMLENRAMVRRKGFGILIGVYGSSVIYMVQLPIFGVIDTPCWIIKAAPSCSE |
| | | KDGNYACLLREDQGWYCKNAGSTVYYPNDKDCETRGDHVFCDTAAGINVAEQSRE |
| | | CNINISTTNYPCKVSTGRHPISMVALSPLGALVACYKGVSCSIGSNRVGIIKQLP |
| | | KGCSYITNQDADTVTIDNTVYQLSKVEGEQHVIKGRPVSSSFDPIKFPEDQENVA |
| | | LDQVFESIENSQAGSGGSGSGSGGSEKAAKAEEAARKMEELFKKHKIVAVLRANS |
| | | VEEAIEKAVAVFAGGVHLIEITFTVPDADTVIKALSVLKEKGAIIGAGTVTSVEQ |
| | | ARKAVESGAEFIVSPHLDEEISQFAKEKGVFYMPGVMTPTELVKAMKLGHTILKL |
| | | FPGEVVGPQFVKAMKGPFPNVKFVPTGGVNLDNVAEWFKAGVLAVGVGSALVKGT |
| | | PDEVREKAKAFVEKIRGATELE |
| hMPV022 | 113 | KESYLEESCSTITEGYLSVLRTGWYTNVFTLEVGDVENLTCTDCPSLIKTELDLT |
| | | KSALRELKTVSADQLAREEQIEGGGGGGFVLGAIALGVATAAAVTAGIAIAKTIR |
| | | LESEVNAIKGALKTTNEAVSTLGNGVRVLATAVRELKEFVSKNLTSAINKNKCDI |
| | | ADLCMAVSFSQFNRRELNVVRQFSDNAGITPAISLDLMTDAELARAVSYMPTSAG |
| | | QIKLMLENRAMVRRKGFGILIGVYGSSVIYMVQLPIFGVIDTPCWIIKAAPSCSE |
| | | KDGNYACLLREDQGWYCKNAGSTVYYPNDKDCETRGDHVFCDTAAGINVAEQSRE |
| | | CNINISTTNYPCKVSTGRHPISMVALSPLGALVACYKGVSCSIGSNRVGIIKQLP |
| | | KGCSYITNQDADTVTIDNTVYQLSKVEGEQHVIKGRPVSSSFDPIAFPEDQENVA |
| | | LDQVFESIENAQAGSGGSGSGSGGSEKAAKAEEAARKMEELFKKHKIVAVLRANS |
| | | VEEAIEKAVAVFAGGVHLIEITFTVPDADTVIKALSVLKEKGAIIGAGTVTSVEQ |
| | | ARKAVESGAEFIVSPHLDEEISQFAKEKGVFYMPGVMTPTELVKAMKLGHTILKL |
| | | FPGEVVGPQFVKAMKGPFPNVKFVPTGGVNLDNVAEWFKAGVLAVGVGSALVKGT |
| | | PDEVREKAKAFVEKIRGATELE |
| hMPV023 | 114 | LKESYLEESCSTITEGYLSVLRTGWYTNVFTLEVGDVENLTCSDCPSLIKTELDL |
| | | TKSALRELKTVSADQLAREEQIEGGGGGGFVLGAIALGVATAAAVTAGVAIAKTI |
| | | RLESEVTAIKNCLKTTNECVSTLGNGVRVLATAVRELKDFVSKNLTSAINKNKCD |
| | | IDDLCMAVSFSQFNRRFLNVVRQFSDNAGITPAISLDLMTDAELARAVSNMPTSA |
| | | GQIKLMLENRAMVRRKGFGILIGVYGSSVIYMVQLPIFGVIDTPCWIIKAAPSCS |
| | | EKKGNYACLLREDQGWYCQNAGSTVYYPNEKDCETRGDHVFCDTAAGINVAEQSK |
| | | ECNINISTTNYPCKVSTGRHPISMVALSPLGALVACYKGVSCSIGSNRVGIIKQL |
| | | NKGCSYITNQDADTVTIDNTVYQLSKVEGEQHVIKGRPVSSSFDPIKFPEDQFNV |
| | | ALDQVFENIENSQAGSGGSGSGSGGSEKAAKAEEAARKMEELFKKHKIVAVLRAN |
| | | SVEEAIEKAVAVFAGGVHLIEITFTVPDADTVIKALSVLKEKGAIIGAGTVTSVE |
| | | QARKAVESGAEFIVSPHLDEEISQFAKEKGVFYMPGVMTPTELVKAMKLGHTILK |
| | | LFPGEVVGPQFVKAMKGPFPNVKFVPTGGVNLDNVAEWFKAGVLAVGVGSALVKG |
| | | TPDEVREKAKAFVEKIRGATELE |
| hMPV024 | 115 | LKESYLEESCSTITEGYLSVLRTGWYTNVFTLEVGDVENLTCADCPSLIKTELDL |
| | | TKSALRELRTVSADQLAREEQIEGGGGGGFVLGAIALGVATAAAVTAGVAIAKTI |
| | | RLESEVTAIKNCLKKTNECVSTLGNGVRVLATAVRELKDFVSKNLTRAINKNKCD |
| | | IADLCMAVSFSQFNRRFLNVVRQFSDNAGITPAISLDLMTDAELARAVSNMPTSA |
| | | GQIKLMLENRAMVRRKGFGFLIGVYGSSVIYMVQLPIFGVIDTPCWIVKAAPSCS |
| | | EKKGNYACLLREDQGWYCQNAGSTVYYPNEKDCETRGDHVFCDTAAGINVAEQSK |
| | | ECNINISTTNYPCKVSTGRHPISMVALSPLGALVACYKGVSCSIGSNRVGIIKQL |
| | | NKGCSYITNQDADTVTIDNTVYQLSKVEGEQHVIKGRPVSSSFDPVCFPEDQFNV |
| | | ALDQVFESIENCQAGSGGSGSGSGGSEKAAKAEEAARKMEELFKKHKIVAVLRAN |
| | | SVEEAIEKAVAVFAGGVHLIEITFTVPDADTVIKALSVLKEKGAIIGAGTVTSVE |
| | | QARKAVESGAEFIVSPHLDEEISQFAKEKGVFYMPGVMTPTELVKAMKLGHTILK |
| | | LFPGEVVGPQFVKAMKGPFPNVKFVPTGGVNLDNVAEWFKAGVLAVGVGSALVKG |
| | | TPDEVREKAKAFVEKIRGATELE |
| hMPV025 | 116 | LKESYLEESCSTITEGYLSVLRTGWYTNVFTLEVGDVENLTCADCPSLIKTELDL |
| | | TKSALRELRTVSADQLAREEQIEGGGGGGFVLGAIALGVATAAAVTAGVAIAKTI |
| | | RLESEVTAIKNALKKTNEAVSTLGNGVRVLATAVRELKDFVSKNLTRAINKNKCD |
| | | IADLCMAVSFSQFNRRFLNVVRQFSDNAGITPAISLDLMTDAELARAVSNMPTSA |
| | | GQIKLMLENRAMVRRKGFGFLIGVYGSSVIYMVQLPIFGVIDTPCWIVKAAPSCS |
| | | EKKGNYACLLREDQGWYCQNAGSTVYYPNEKDCETRGDHVFCDTAAGINVAEQSK |
| | | ECNINISTTNYPCKVSTGRHPISMVALSPLGALVACYKGVSCSIGSNRVGIIKQL |
| | | NKGCSYITNQDADTVTIDNTVYQLSKVEGEQHVIKGRPVSSSFDPVKFPEDQENV |
| | | ALDQVFESIENSQAGSGGSGSGSGGSEKAAKAEEAARKMEELFKKHKIVAVLRAN |
| | | SVEEAIEKAVAVFAGGVHLIEITFTVPDADTVIKALSVLKEKGAIIGAGTVTSVE |
| | | QARKAVESGAEFIVSPHLDEEISQFAKEKGVFYMPGVMTPTELVKAMKLGHTILK |
| | | LFPGEVVGPQFVKAMKGPFPNVKFVPTGGVNLDNVAEWFKAGVLAVGVGSALVKG |
| | | TPDEVREKAKAFVEKIRGATELE |
| hMPV026 | 117 | LKESYLEESCSTITEGYLSVLRTGWYTNVFTLEVGDVENLTCADGPSLIKTELDL |
| | | TKSALRELRTVSADQLAREEQIENPRRRRFVLGAIALGVATAAAVTAGVAIAKCI |
| | | RLESEVTAIKNALKKTNEAVSTLGCGVRVLATAVRELKDFVSKNLTRAINKNKCD |
| | | IPDLKMAVSFSQFNRRFLNVVRQFSDNAGITPAISKDLMTDAELARAISNMPTSA |
| | | GQIKLMLENRAMVRRKGFGILIGVYGSSVIYMVQLPIFGVIDTPCWIVKAAPSCS |
| | | EKKGNYACLLREDQGWYCQNAGSTVYYPNEKDCETRGDHVFCDTAAGINVAEQSK |
| | | ECNINISTTNYPCKVSCGRNPISMVALSPLGALVACYKGVSCSIGSNRVGIIKQL |
| | | NKGCSYITNQDADTVTIDNTVYQLSKVEGEQHVIKGRPVSSSFDPVKFPEDQENV |
| | | ALDQCFESIENSQAGSGGSGSGSGGSEKAAKAEEAARKMEELFKKHKIVAVLRAN |
| | | SVEEAIEKAVAVFAGGVHLIEITFTVPDADTVIKALSVLKEKGAIIGAGTVTSVE |
| | | QARKAVESGAEFIVSPHLDEEISQFAKEKGVFYMPGVMTPTELVKAMKLGHTILK |
| | | LFPGEVVGPQFVKAMKGPFPNVKFVPTGGVNLDNVAEWFKAGVLAVGVGSALVKG |
| | | TPDEVREKAKAFVEKIRGATELE |
| hMPV027 | 118 | LKESYLEESCSTITEGYLSVLRTGWYTNVFTLEVGDVENLTCADGPSLIKTELDL |
| | | TKSALRELRTVSADQLAREEQIENPRRRRFVLGAIALGVATAAAVTAGVAIAKCI |
| | | RLESEVTAIKNALKKTNEAVSTLGCGVRVLATAVRELKDFVSKNLTRAINKNKCD |
| | | IPDLKMAVSFSQFNRRFLNVVRQFSDNAGITPAISKDLMTDAELARAISNMPTSA |
| | | GQIKLMLENRAMVRRKGFGILIGVYGSSVIYMVQLPIFGVIDTPCWIVKAAPSCS |
| | | EKKGNYACLLREDQGWYCQNAGSTVYYPNEKDCETRGDHVFCDTAAGINVAEQSK |
| | | ECNINISTTNYPCKVSCGRNPISMVALSPLGALVACYKGVSCSIGSNRVGIIKQL |
| | | NKGCSYITNQDADTVTIDNTVYQLSKVEGEQHVIKGRPVSSSFDPVKFPEDQFNV |
| | | ALDQCFESIENSQALVDQSNRILSSAEKGNTGGSGGSGSGSGGSEKAAKAEEAAR |
| | | KMEELFKKHKIVAVLRANSVEEAIEKAVAVFAGGVHLIEITFTVPDADTVIKALS |
| | | VLKEKGAIIGAGTVTSVEQARKAVESGAEFIVSPHLDEEISQFAKEKGVFYMPGV |
| | | MTPTELVKAMKLGHTILKLFPGEVVGPQFVKAMKGPFPNVKFVPTGGVNLDNVAE |
| | | WFKAGVLAVGVGSALVKGTPDEVREKAKAFVEKIRGATELE |
| hMPV028 | 119 | KESYLEESCSTITEGYLSVLRTGWYTNVFTLEVGDVENLTCTDCPSLIKTELDLT |
| | | KSALRELKTVSADQLAREEQIEGGGGGGFVLGAIALGVATAAAVTAGIAIAKTIR |
| | | LESEVNAIKGCLKTTNECVSTLGNGVRVLATAVRELKEFVSKNLTSAINKNKCDI |
| | | ADLCMAVSFSQFNRRFLNVVRQFSDNAGITPAISLDLMTDAELARAVSYMPTSAG |
| | | QIKLMLENRAMVRRKGFGILIGVYGSSVIYMVQLPIFGVIDTPCWIIKAAPSCSE |
| | | KDGNYACLLREDQGWYCKNAGSTVYYPNDKDCETRGDHVFCDTAAGINVAEQSRE |
| | | CNINISTTNYPCKVSTGRHPISMVALSPLGALVACYKGVSCSIGSNRVGIIKQLP |
| | | KGCSYITNQDADTVTIDNTVYQLSKVEGEQHVIKGRPVSSSFDPICFPEDQENVA |
| | | LDQVFESIENCQAGSGGSGSGSGGSEKAAKAEEAARKMEELFKKHKIVAVLRANS |
| | | VEEAIEKAVAVFAGGVHLIEITFTVPDADTVIKALSVLKEKGAIIGAGTVTSVEQ |
| | | CRKAVESGAEFIVSPHLDEEISQFCKEKGVFYMPGVMTPTELVKAMKLGHTILKL |
| | | FPGEVVGPQFVKAMKGPFPNVKFVPTGGVNLDNVCEWFKAGVLAVGVGSALVKGT |
| | | PDEVREKAKAFVEKIRGCTELE |
| hMPV029 | 120 | LKESYLEESCSTITEGYLSVLRTGWYTNVFTLEVGDVENLTCADGPSLIKTELDL |
| | | TKSALRELRTVSADQLAREEQIENPRRRRFVLGAIALGVATAAAVTAGVAIAKCI |
| | | RLESEVTAIKNALKKTNEAVSTLGCGVRVLATAVRELKDFVSKNLTRAINKNKCD |
| | | IPDLKMAVSFSQFNRRFLNVVRQFSDNAGITPAISKDLMTDAELARAISNMPTSA |
| | | GQIKLMLENRAMVRRKGFGILIGVYGSSVIYMVQLPIFGVIDTPCWIVKAAPSCS |
| | | EKKGNYACLLREDQGWYCQNAGSTVYYPNEKDCETRGDHVFCDTAAGINVAEQSK |
| | | ECNINISTTNYPCKVSCGRNPISMVALSPLGALVACYKGVSCSIGSNRVGIIKQL |
| | | NKGCSYITNQDADTVTIDNTVYQLSKVEGEQHVIKGRPVSSSFDPVKFPEDQENV |
| | | ALDQCFESIENSQAGSGGSGSGSGGSEKAAKAEEAARKMEELFKKHKIVAVLRAN |
| | | SVEEAIEKAVAVFAGGVHLIEITFTVPDADTVIKALSVLKEKGAIIGAGTVTSVE |
| | | QCRKAVESGAEFIVSPHLDEEISQFCKEKGVFYMPGVMTPTELVKAMKLGHTILK |
| | | LFPGEVVGPQFVKAMKGPFPNVKFVPTGGVNLDNVCEWFKAGVLAVGVGSALVKG |
| | | TPDEVREKAKAFVEKIRGCTELE |
| hMPV030 | 121 | LKESYLEESCSTITEGYLSVLRTGWYTNVFTLEVGDVENLTCADGPSLIKTELDL |
| | | TKSALRELRTVSADQLAREEQIENPRRRRFVLGAIALGVATAAAVTAGVAIAKCI |
| | | RLESEVTAIKNALKKTNEAVSTLGCGVRVLATAVRELKDFVSKNLTRAINKNKCD |
| | | IPDLKMAVSFSQFNRRFLNVVRQFSDNAGITPAISKDLMTDAELARAISNMPTSA |
| | | GQIKLMLENRAMVRRKGFGILIGVYGSSVIYMVQLPIFGVIDTPCWIVKAAPSCS |
| | | EKKGNYACLLREDQGWYCQNAGSTVYYPNEKDCETRGDHVFCDTAAGINVAEQSK |
| | | ECNINISTTNYPCKVSCGRNPISMVALSPLGALVACYKGVSCSIGSNRVGIIKQL |
| | | NKGCSYITNQDADTVTIDNTVYQLSKVEGEQHVIKGRPVSSSFDPVKFPEDQFNV |
| | | ALDQCFESIENSQALVDQSNRILSSAEKGNTGGSGGSGSGSGGSEKAAKAEEAAR |
| | | KMEELFKKHKIVAVLRANSVEEAIEKAVAVFAGGVHLIEITFTVPDADTVIKALS |
| | | VLKEKGAIIGAGTVTSVEQCRKAVESGAEFIVSPHLDEEISQFCKEKGVFYMPGV |
| | | MTPTELVKAMKLGHTILKLFPGEVVGPQFVKAMKGPFPNVKFVPTGGVNLDNVCE |
| | | WFKAGVLAVGVGSALVKGTPDEVREKAKAFVEKIRGCTELE |
| hMPV031 | 122 | KESYLEESCSTITEGYLSVLRTGWYTNVFTLEVGDVENLTCTDGPSLIKTELDLT |
| | | KSALRELKTCSADQLAREEQIEGGGGGGFVLGAIALGVATAAAVTAGIAIAKTIR |
| | | LESEVNAIKGCLKTTNECVSTLGNGVRVLATAVRELKEFVSKNLTSAINKNKCDI |
| | | ADLKMAVSFSQFNRRFLNVVRQFSDNAGITPAISLDLMTDAELARAVSYMPTSAG |
| | | QIKLMLENRCMVRRKGFGILIGVYGSSVIYMVQLPIFGVIDTPCWIIKAAPSCSE |
| | | KDGNYACLLREDQGWYCKNAGSTVYYPNDKDCETRGDHVECDTAAGINVAEQSRE |
| | | CNINISTTNYPCKVSTGRHPISMVALSPLGALVACYKGVSCSIGSNRVGIIKQLP |
| | | KGCSYITNQDADTVTIDNTVYQLSKVEGEQHVIKGRPVSSSFDPIKFPEDQENVA |
| | | LDQVFESIENSQAGSGGSGSGSGGSEKAAKAEEAARKMEELFKKHKIVAVLRANS |
| | | VEEAIEKAVAVFAGGVHLIEITFTVPDADTVIKALSVLKEKGAIIGAGTVTSVEQ |
| | | CRKAVESGAEFIVSPHLDEEISQFCKEKGVFYMPGVMTPTELVKAMKLGHTILKL |
| | | FPGEVVGPQFVKAMKGPFPNVKFVPTGGVNLDNVCEWFKAGVLAVGVGSALVKGT |
| | | PDEVREKAKAFVEKIRGCTELE |
| hMPV033 | 123 | LKESYLEESCSTITEGYLSVLRTGWYTNVFTLEVGDVENLTCADGPSLIKTELDL |
| | | TKSALRELRTVSADQLAREEQIEGGGGGGFVLGAIALGVATAAAVTAGVAIAKCI |
| | | RLESEVTAIKNALKKTNEAVSTLGCGVRVLATAVRELKDFVSKNLTRAINKNKCD |
| | | IPDLKMAVSFSQFNRRFLNVVRQFSDNAGITPAISKDLMTDAELARAISNMPTSA |
| | | GQIKLMLENRAMVRRKGFGILIGVYGSSVIYMVQLPIFGVIDTPCWIVKAAPSCS |
| | | EKKGNYACLLREDQGWYCQNAGSTVYYPNEKDCETRGDHVFCDTAAGINVAEQSK |
| | | ECNINISTTNYPCKVSCGRNPISMVALSPLGALVACYKGVSCSIGSNRVGIIKQL |
| | | NKGCSYITNQDADTVTIDNTVYQLSKVEGEQHVIKGRPVSSSFDPVKFPEDQENV |
| | | ALDQCFESIENSQAGSGGSGSGSGGSEKAAKAEEAARKMEELFKKHKIVAVLRAN |
| | | SVEEAIEKAVAVFAGGVHLIEITFTVPDADTVIKALSVLKEKGAIIGAGTVTSVE |
| | | QARKAVESGAEFIVSPHLDEEISQFAKEKGVFYMPGVMTPTELVKAMKLGHTILK |
| | | LFPGEVVGPQFVKAMKGPFPNVKFVPTGGVNLDNVAEWFKAGVLAVGVGSALVKG |
| | | TPDEVREKAKAFVEKIRGATELE |
| hMPV034 | 124 | LKESYLEESCSTITEGYLSVLRTGWYTNVFTLEVGDVENLTCADGPSLIKTELDL |
| | | TKSALRELRTVSADQLAREEQIEGGGGGGFVLGAIALGVATAAAVTAGVAIAKCI |
| | | RLESEVTAIKNALKKTNEAVSTLGCGVRVLATAVRELKDFVSKNLTRAINKNKCD |
| | | IPDLKMAVSFSQFNRRELNVVRQFSDNAGITPAISKDLMTDAELARAISNMPTSA |
| | | GQIKLMLENRAMVRRKGFGILIGVYGSSVIYMVQLPIFGVIDTPCWIVKAAPSCS |
| | | EKKGNYACLLREDQGWYCQNAGSTVYYPNEKDCETRGDHVFCDTAAGINVAEQSK |
| | | ECNINISTTNYPCKVSCGRNPISMVALSPLGALVACYKGVSCSIGSNRVGIIKQL |
| | | NKGCSYITNQDADTVTIDNTVYQLSKVEGEQHVIKGRPVSSSFDPVKFPEDQFNV |
| | | ALDQCFESIENSQALVDQSNRILSSAEKGNTGGSGGSGSGSGGSEKAAKAEEAAR |
| | | KMEELFKKHKIVAVLRANSVEEAIEKAVAVFAGGVHLIEITFTVPDADTVIKALS |
| | | VLKEKGAIIGAGTVTSVEQARKAVESGAEFIVSPHLDEEISQFAKEKGVFYMPGV |
| | | MTPTELVKAMKLGHTILKLFPGEVVGPQFVKAMKGPFPNVKFVPTGGVNLDNVAE |
| | | WFKAGVLAVGVGSALVKGTPDEVREKAKAFVEKIRGATELE |

### Assembly of VLPs

In some embodiments, a single component self-assembles into the VLP. In some embodiments, one or more purified samples of first and second components for use in forming a VLP are mixed in an approximately equimolar molar ratio in aqueous conditions (e.g., an I53-50A/B isosahedral VLP). The first and second components (through the multimerization domains and optionally through the ectodomains) interact with one another to drive assembly of the target VLP. Successful assembly of the target VLP can be confirmed by analyzing the in vitro assembly reaction by common biochemical or biophysical methods used to assess the physical size of proteins or protein assemblies, including but not limited to size exclusion chromatography, native (non-denaturing) gel electrophoresis, dynamic light scattering, multi-angle light scattering, analytical ultracentrifugation, negative stain electron microscopy, cryo-electron microscopy, or X-ray crystallography. If necessary, the assembled VLP can be purified from other species or molecules present in the in vitro assembly reaction using preparative techniques commonly used to isolate proteins by their physical size, including but not limited to size exclusion chromatography, preparative ultracentrifugation, tangential flow filtration, or preparative gel electrophoresis. The presence of the antigenic protein in the VLP can be assessed by techniques commonly used to determine the identity of protein molecules in aqueous solutions, including but not limited to SDS-PAGE, mass spectrometry, protein sequencing, ELISA, surface plasmon resonance, biolayer interferometry, or amino acid analysis. The accessibility of the protein on the exterior of the particle, as well as its conformation or antigenicity, can be assessed by techniques commonly used to detect the presence and conformation of an antigen, including but not limited to binding by monoclonal antibodies, conformation-specific monoclonal antibodies, surface plasmon resonance, biolayer interferometry, or antisera specific to the antigen.

In various embodiments, the VLPs of the disclosure comprise two or more distinct first polypeptides bearing different antigenic proteins as genetic fusions; these VLPs co-display multiple different proteins on the same VLP. These multi-antigen VLPs are produced by performing in vitro assembly with mixtures of two or more antigens each comprising a multimerization domain. The fraction of each antigen in the mixture determines the average valency of each antigenic protein in the resulting VLPs. The presence and average valency of each antigen in a given sample can be assessed by quantitative analysis using the techniques described above for evaluating the presence of antigenic proteins in full-valency VLPs.

In various embodiments, the VLPs are between about 20 nanometers (nm) to about 40 nm in diameter, with interior lumens between about 15 nm to about 32 nm across and pore sizes in the protein shells between about 1 nm to about 14 nm in their longest dimensions.

In some embodiments, the VLPs have icosahedral symmetry. In such embodiment, the VLP may comprise 60 copies of a first component and 60 copies of a second component. In one such embodiment, the number of identical first polypeptides in each first assembly is different than the number of identical first polypeptides in each second assembly. For example, in some embodiments, the VLP comprises twelve first assemblies and twenty second assemblies; in such embodiments, each first assembly may, for example, comprise five copies of the identical first component, and each second assembly may, for example, comprise three copies of the identical second component. In other embodiments, the VLP comprises twelve first assemblies and thirty second assemblies; in such an embodiment, each first assembly may, for example, comprise five copies of the identical first component, and each second assembly may, for example, comprise two copies of the identical second component. In further embodiments, the VLP comprises twenty first assemblies and thirty second assemblies; in this embodiment, each first assembly may, for example, comprise three copies of the identical first component, and each second assembly may, for example, comprise two copies of the identical second component. All of these embodiments are capable of forming protein-based VLPs with regular icosahedral symmetry.

In various further embodiments, oligomeric states of the first and second multimerization domains are as follows:
I53-34A: trimer+I53-34B: pentamer;
I53-40A: pentamer+I53-40B: trimer;
I53-47A: trimer+I53-47B: pentamer;
I53-50A: trimer+I53-50B: pentamer;
I53-51A: trimer+I53-51B: pentamer;
I32-06A: dimer+I32-06B: trimer;
I32-19A: trimer+I32-19B: dimer;
I32-28A: trimer+I32-28B: dimer;
I52-03A: pentamer+I52-03B: dimer;
I52-32A: dimer+I52-32B: pentamer; and
I52-33A: pentamer+I52-33B: dimer

In some embodiments, the second multimerization domain of the second polypeptide comprises a sequence that shares at least 95% identity to I53-50A or a variant thereof:

I53-50A
(SEQ ID NO: 144)

| | |
|---|---|
| 1 | MEELFKKHKI VAVLRANSVE EAIEKAVAVF AGGVHLIEIT |
| 41 | FTVPDADTVI KALSVLKEKG AIIGAGTVTS VEQCRKAVES |
| 81 | GAEFIVSPHL DEEISQFCKE KGVFYMPGVM TPTELVKAMK |
| 121 | LGHTILKLEP GEVVGPQFVK AMKGPFPNVK FVPTGGVNLD |
| 161 | NVCEWFKAGV LAVGVGSALV KGTPDEVREK AKAFVEKIRG |
| 201 | CTE |

In some embodiments, the second multimerization domain shares at least 90%, at least 91%, at least 92%, at least 93%, at least 94%, at least 95%, at least 96%, at least 97%, at least 98%, at least 99%, or 100% sequence identity to SEQ ID NO: 7 or SEQ ID NO: 144, or an antigenic fragment thereof.

The I53-50A protein sequence has two intra-monomer disulfide bonds. In some embodiments, the cysteine residues are mutated to residues that do not contain a thiol group (e.g. alanine or serine). Removal of the thiol group may promote correct protein folding while not impairing multimerization. In some embodiments, the multimerization domain of the first polypeptide comprises an amino acid substitution at one or more of positions 74, 98, 163, and 201 relative to SEQ ID NO: 144, as shown here:

I53-50A
(SEQ ID NO: 144)

| | |
|---|---|
| 1 | MEELFKKHKI VAVLRANSVE EAIEKAVAVF AGGVHLIEIT |
| 41 | FTVPDADTVI KALSVLKEKG AIIGAGTVTS VEQCRKAVES |
| 81 | GAEFIVSPHL DEEISQFCKE KGVFYMPGVM TPTELVKAMK |
| 121 | LGHTILKLEP GEVVGPQFVK AMKGPFPNVK FVPTGGVNLD |
| 161 | NVCEWFKAGV LAVGVGSALV KGTPDEVREK AKAFVEKIRG |
| 201 | CTE |

In some embodiments, the multimerization domain of the first polypeptide comprises an amino acid substitution of one or more of C74A, C98A, C163A, and C201A relative to SEQ ID NO: 132. In some embodiments, the multimerization domain of the first polypeptide comprises SEQ ID NO: 132 or a variant thereof.

I53-50A-Acys (disulfide-free form)
(SEQ ID NO: 132)

| | |
|---|---|
| 1 | MEELFKKHKI VAVLRANSVE EAIEKAVAVF AGGVHLIEIT |
| 41 | FTVPDADTVI KALSVLKEKG AIIGAGTVTS VEQARKAVES |
| 81 | GAEFIVSPHL DEEISQFAKE KGVFYMPGVM TPTELVKAMK |
| 12 | LGHTILKLFP GEVVGPOFVK AMKGPFPNVK FVPTGGVNLD |
| 161 | NVAEWFKAGV LAVGVGSALV KGTPDEVREK AKAFVEKIRG |
| 201 | ATE |

In some embodiments, the multimerization domain shares at least 90%, at least 91%, at least 92%, at least 93%, at least 94%, at least 95%, at least 96%, at least 97%, at least 98%, at least 99%, or 100% sequence identity to SEQ ID NO: 144 or SEQ ID NO: 132, or an antigenic fragment thereof, and comprises one, two, three or four amino acid substitutions selected from C74A, C98A, C163A, and C201A. Alternatively, the substitution may be of C to A, T, S, L, I, or any amino acid other than C.

### Nucleic Acids

In another aspect, the present disclosure provides isolated nucleic acids encoding an antigen, a first component, and/or a second component, of the present disclosure. The isolated nucleic acid sequence may comprise RNA or DNA. As used herein, “isolated nucleic acids” are those that have been removed from their normal surrounding nucleic acid sequences in the genome or in cDNA sequences. Such isolated nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the proteins of the disclosure.

In a further aspect, the present disclosure provides recombinant expression vectors comprising the isolated nucleic acid of any embodiment or combination of embodiments of the disclosure operatively linked a suitable control sequence. “Recombinant expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type known in the art, including but not limited to plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid responsive). The construction of expression vectors for use in transfecting prokaryotic cells is also well known in the art, and thus can be accomplished via standard techniques. (See, for example, Sambrook, Fritsch, and Maniatis, in: Molecular Cloning, A Laboratory Manual, Cold Spring Harbor Laboratory Press, 1989; Gene Transfer and Expression Protocols, pp. 109-128, ed. E. J. Murray, The Humana Press Inc., Clifton, N.J.), and the Ambion 1998 Catalog (Ambion, Austin, TX). The expression vector must be replicable in the host organisms either as an episome or by integration into host chromosomal DNA. In a preferred embodiment, the expression vector comprises a plasmid. However, the disclosure is intended to include other expression vectors that serve equivalent functions, such as viral vectors.

In another aspect, the present disclosure provides host cells that have been transfected or transduced with the recombinant expression vectors disclosed herein, wherein the host cells can be either prokaryotic or eukaryotic. The cells can be transiently or stably transfected or transduced. Such transfection or transduction of expression vectors into prokaryotic and eukaryotic cells can be accomplished via any technique known in the art, including but not limited to standard bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook, et al., 1989, Cold Spring Harbor Laboratory Press; Culture of Animal Cells: A Manual of Basic Technique, 2nd Ed. (R.I. Freshney. 1987. Liss, Inc. New York, NY).

In another aspect, the disclosure provides a method of producing an antigen, component, or VLP according to the disclosure. In some embodiments, the method comprises the steps of (a) culturing a host according to this aspect of the disclosure under conditions conducive to the expression of the polypeptide, and (b) optionally, recovering the expressed polypeptide.

In some embodiments, the disclosure provides a method of manufacturing a vaccine, comprising culturing a host cell comprising a polynucleotide comprising a sequence encoding the antigen of the disclosure in a culture medium so that the host cell secretes the antigen into the culture media; optionally purifying the antigen from the culture media; mixing the antigen with a second component, wherein the second component multimerizes with the antigen to form a VLP; and optionally purifying the VLP.

In some embodiments, the disclosure provides method of manufacturing a vaccine, comprising culturing a host cell comprising one or more polynucleotides comprising sequences encoding both components of the VLP of any one of disclosure so that the host cell secretes the first component and the second component into the culture media; and optionally purifying the VLP from the culture media.

Illustrative host cells in include E. coli cells, 293 and 293F cells, HEK293 cells, Sf9 cells, Chinese hamster ovary (CHO) cells and any other cell line used in the production of recombinant proteins.

In various embodiments, the first component expresses at about 0.5 mg/mL, about 1.0 mg/mL, about 1.5 mg/mL, about 2.5 mg/mL, about 5 mg/mL, about 10 mg/mL, about 25 mg/mL, about 50 mg/mL, about 75 mg/mL, about 100 mg/mL, or greater in a method of manufacturing according to the disclosure (e.g. 293F cells grown in suspension). In various embodiments, the first component expresses at least 50%, at least 60%, at least 70%, at least 80%, at least 90%, or at least 95% of the expression level of an RSV and/or hMPV F protein (optionally the same ectodomain as in the VLP) in the same or similar expression system. In various embodiments, the first component expresses at least 105%, at least 110%, at least 115%, at least 120%, at least 125%, at least 150%, at least 175%, or at least 200% of the expression level of an RSV and/or hMPV F protein (optionally the same ectodomain as in the VLP) in the same or similar expression system.

In some embodiments, the RSV and/or hMPV F protein ectodomain of the first component is in the prefusion conformation, or a substantial fraction of the RSV and/or hMPV F protein ectodomain is in the prefusion conformation. In various embodiments, at least 50%, at least 60%, at least 70%, at least 80%, at least 90%, or at least 95% of the RSV and/or hMPV F protein ectodomain of the first component is in the prefusion conformation. In some embodiments, the RSV and/or hMPV F protein ectodomain of the VLP is in the prefusion conformation, or a substantial fraction of the RSV and/or hMPV F protein ectodomain is in the prefusion conformation. In various embodiments, at least 50%, at least 60%, at least 70%, at least 80%, at least 90%, or at least 95% of the RSV and/or hMPV F protein ectodomain of the VLP is in the prefusion conformation.

In some embodiments, the fraction of RSV and//or hMPV F protein ectodomain in the prefusion state is determined by binding to a conformation-specific antibody (e.g., D1-25 and 1112-1). In some embodiments, the fraction of hMPV F protein ectodomain in the first component in the prefusion conformation is at least 105%, at least 110%, at least 115%, at least 120%, at least 125%, at least 150%, at least 175%, or at least 200% greater than the fraction in a reference component, or than the fraction in a reference protein not linked to a multimerization domain. In some embodiments, the fraction of hMPV F protein ectodomain in the first component in the prefusion conformation is at least 105%, at least 110%, at least 115%, at least 120%, at least 125%, at least 150%, at least 175%, or at least 200% greater than the fraction in a VLP (e.g., micelle VLP) Vaccines and Administration

The disclosure also provides vaccines comprising the VLPs described herein. Such compositions can be used to raise antibodies in a mammal (e.g. a human). The vaccines compositions of the disclosure typically include a pharmaceutically acceptable carrier, and a thorough discussion of such carriers is available in Remington: The Science and Practice of Pharmacy.

The pH of the composition is usually between about 4.5 to about 11, such as between about 5 to about 11, between about 5.5 to about 11, between about 6 to about 11, between about 5 to about 10.5, between about 5.5 to about 10.5, between about 6 to about 10.5, between about 5 to about 10, between about 5.5 to about 10, between about 6 to about 10, between about 5 to about 9.5, between about 5.5 to about 9.5, between about 6 to about 9.5, between about 5 to about 9, between about 5.5 to about 9, between about 6 to about 9, between about 5 to about 8.5, between about 5.5 to about 8.5, between about 6 to about 8.5, between about 5 to about 8, between about 5.5 to about 8, between about 6 to about 8, about 4.5, about 5, about 6.5, about 6, about 6.5, about 7, about 7.5, about 8, about 8.5, about 9, about 9.5, about 10, about 10.5, about 11, etc. Stable pH may be maintained by the use of a buffer e.g. a Tris buffer, a citrate buffer, phosphate buffer, or a histidine buffer. Thus a composition will generally include a buffer.

In some embodiments, the pH of the formulation is between about pH 6.2 to about pH 8.0. In some embodiments, the pH is about 6.2, about 6.4, about 6.6, about 6.8, about 7.0, about 7.2, about 7.4, about 7.6, about 7.8, or about 8.0. Of course, the pH may also be within a range of values. Thus, in some embodiments the pH is between about 6.2 and about 8.0, between about 6.2 and 7.8, between about 6.2 and 7.6, between about 6.2 and 7.4, between about 6.2 and 7.2, between about 6.2 and 7.0, between about 6.2 and 6.8, between about 6.2 and about 6.6, or between about 6.2 and 6.4. In other embodiments, the pH is between 6.4 and about 8.0, between about 6.4 and 7.8, between about 6.4 and 7.6, between about 6.4 and 7.4, between about 6.4 and 7.2, between about 6.4 and 7.0, between about 6.4 and 6.8, or between about 6.4 and about 6.6. In still other embodiments, the pH is between about 6.6 and about 8.0, between about 6.6 and 7.8, between about 6.6 and 7.6, between about 6.6 and 7.4, between about 6.6 and 7.2, between about 6.6 and 7.0, or between about 6.6 and 6.8. In yet other embodiments, it is between about 6.8 and about 8.0, between about 6.8 and 7.8, between about 6.8 and 7.6, between about 6.8 and 7.4, between about 6.8 and 7.2, or between about 6.8 and 7.0. In still other embodiments, it is between about 7.0 and about 8.0, between about 7.0 and 7.8, between about 7.0 and 7.6, between about 7.0 and 7.4, between about 7.0 and 7.2, between about 7.2 and 8.0, between about 7.2 and 7.8, between about 7.2 and about 7.6, between about 7.2 and 7.4, between about 7.4 and about 8.0, about 7.4 and about 7.6, or between about 7.6 and about 8.0.

In some embodiments, the formulation can include one or more salts, such as sodium chloride, sodium phosphate, or a combination thereof. In general, each salt is present in the formulation at about 10 mM to about 200 mM. Thus, in some embodiments, any salt that is present is present at about 10 mM to about 200 mM, about 20 mM to about 200 mM, about 25 mM to about 200 mM, at about 30 mM to about 200 mM, at about 40 mM to about 200 mM, at about 50 mM to about 200 mM, at about 75 mM to about 200 mM, at about 100 mM to about 200 mM, at about 125 mM to about 200 mM, at about 150 mM to about 200 mM, or at about 175 mM to about 200 mM. In other embodiments, any salt that is present is present at about 10 mM to about 175 mM, about 20 mM to about 175 mM, about 25 mM to about 175 mM, at about 30 mM to about 175 mM, at about 40 mM to about 175 mM, at about 50 mM to about 175 mM, at about 75 mM to about 175 mM, at about 100 mM to about 175 mM, at about 125 mM to about 175 mM, or at about 150 mM to about 175 mM. In still other embodiments, any salt that is present is present at about 10 mM to about 150 mM, about 20 mM to about 150 mM, about 25 mM to about 150 mM, at about 30 mM to about 150 mM, at about 40 mM to about 150 mM, at about 50 mM to about 150 mM, at about 75 mM to about 150 mM, at about 100 mM to about 150 mM, or at about 125 mM to about 150 mM. In yet other embodiments, any salt that is present is present at about 10 mM to about 125 mM, about 20 mM to about 125 mM, about 25 mM to about 125 mM, at about 30 mM to about 125 mM, at about 40 mM to about 125 mM, at about 50 mM to about 125 mM, at about 75 mM to about 125 mM, or at about 100 mM to about 125 mM. In some embodiments, any salt that is present is present at about 10 mM to about 100 mM, about 20 mM to about 100 mM, about 25 mM to about 100 mM, at about 30 mM to about 100 mM, at about 40 mM to about 100 mM, at about 50 mM to about 100 mM, or at about 75 mM to about 100 mM. In yet other embodiments, any salt that is present is present at about 10 mM to about 75 mM, about 20 mM to about 75 mM, about 25 mM to about 75 mM, at about 30 mM to about 75 mM, at about 40 mM to about 75 mM, or at about 50 mM to about 75 mM. In still other embodiments, any salt that is present is present at about 10 mM to about 50 mM, about 20 mM to about 50 mM, about 25 mM to about 50 mM, at about 30 mM to about 50 mM, or at about 40 mM to about 50 mM. In other embodiments, any salt that is present is present at about 10 mM to about 40 mM, about 20 mM to about 40 mM, about 25 mM to about 40 mM, at about 30 mM to about 40 mM, at about 10 mM to about 30 mM, at about 20 mM to about 30, at about 25 mM to about 30 mM, at about 10 mM to about 25 mM, at about 20 mM to about 25 mM, or at about 10 mM to about 20 mM. In some embodiments, the sodium chloride is present in the formulation at about 100 mM. In some embodiments, the sodium phosphate is present in the formulation at about 25 mM.

A composition may be sterile and/or pyrogen free. Compositions may be isotonic with respect to humans.

Vaccine compositions may include an immunological adjuvant. Illustrative adjuvants include the following: mineral-containing compositions; oil emulsions; squalene emulsions; saponin formulations; virosomes and virus-like particles; bacterial or microbial derivatives; bioadhesives and mucoadhesives; liposomes; polyoxyethylene ether and polyoxyethylene ester formulations; polyphosphazene (pcpp); muramyl peptides; imidazoquinolone compounds; thiosemicarbazone compounds; tryptanthrin compounds; human immunomodulators; lipopeptides; benzonaphthyridines; microparticles; immunostimulatory polynucleotide (such as ma or dna; e.g., cpg-containing oligonucleotides).

For example, the composition may include an aluminum salt adjuvant, an oil in water emulsion (e.g. an oil-in-water emulsion comprising squalene, such as MF59® or AS03), a TLR7 agonist (such as imidazoquinoline or imiquimod), or a combination thereof. Suitable aluminum salts include hydroxides (e.g. oxyhydroxides), phosphates (e.g. hydroxyphosphates, orthophosphates), (e.g. see chapters 8 & 9 of Vaccine Design. (1995) eds. Powell & Newman. ISBN: 030644867X. Plenum), or mixtures thereof. The salts can take any suitable form (e.g. gel, crystalline, amorphous, etc.), with adsorption of antigen to the salt being an example. The concentration of Al⁺⁺⁺ in a composition for administration to a patient may be less than 5 mg/ml e.g. <4 mg/ml, <3 mg/ml, <2 mg/ml, <1 mg/ml, etc. A preferred range is between 0.3 and 1 mg/ml. A maximum of 0.85 mg/dose is preferred. Aluminum hydroxide and aluminium phosphate adjuvants are suitable for use with the disclosure.

Exemplary adjuvants that may be used in a pharmaceutical composition provided herein include, but are not limited to, 3M-052, Adju-Phos™, Alhydrogel™, Adjumer™ albumin-heparin microparticles, Algal Glucan, Algammulin, Alum, Antigen Formulation, AS-2 adjuvant, ASO1, ASO3, autologous dendritic cells, autologous PBMC, Avridine™, B7-2, BAK, BAY R1005, BECC TLR-4 agonists, Bupivacaine, Bupivacaine-HCl, BWZL, Calcitriol, Calcium Phosphate Gel, CCR5 peptides, CFA, Cholera holotoxin (CT) and Cholera toxin B subunit (CTB), Cholera toxin A1-subunit-Protein A D-fragment fusion protein, CpG, CPG-1018, CPG-1018 plus aluminum salt, CRL1005, Cytokine-containing Liposomes, D-Murapalmitine, DDA, DHEA, Diphtheria toxoid, DL-PGL, DMPC, DMPG, DOC/Alum Complex, Fowlpox, Freund's Complete Adjuvant, Gamma Inulin, Gerbu Adjuvant, GM-CSF, GMDP, hGM-CSF, hIL-12 (N222L), hTNF-alpha, IFA, IFN-gamma in pcDNA3, IL-12 DNA, IL-12 plasmid, IL-12/GMCSF plasmid (Sykes), IL-2 in pcDNA3, IL-2/Ig plasmid, IL-2/Ig protein, IL-4, IL-4 in pcDNA3, Imiquimod™, ImmTher™, Immunoliposomes Containing Antibodies to Costimulatory Molecules, Interferon-gamma, Interleukin-1 beta, Interleukin-12, Interleukin-2, Interleukin-7, ISCOM(s)™, Iscoprep 7.0.3™, Keyhole Limpet Hemocyanin, Lipid-based Adjuvant, Liposomes, Loxoribine, LT(R192G), LT-OA or LT Oral Adjuvant, LT-R192G, LTK63, LTK72, Matrix-M™ adjuvant, MF59, MONTANIDE ISA 51, MONTANIDE ISA 720, MPL.™., MPL-SE, MTP-PE, MTP-PE Liposomes, Murametide, Murapalmitine, NAGO, nCT native Cholera Toxin, Non-Ionic Surfactant Vesicles, non-toxic mutant E112K of Cholera Toxin mCT-E112K, p-Hydroxybenzoique acid methyl ester, pCIL-10, pCIL12, pCMVmCAT1, pCMVN, Peptomer-NP, Pleuran, PLG, PLGA, PGA, and PLA, Pluronic L121, PMMA, PODDS™, Poly rA: Poly rU, Polysorbate 80, Protein Cochleates, QS-21, Quadri A saponin, Quil-A, Rehydragel HPA, Rehydragel LV, RIBI, Ribi like adjuvant system (MPL, TMD, CWS), S-28463, SAF-1, Sclavo peptide, Sendai Proteoliposomes, Sendai-containing Lipid Matrices, Span 85, Specol, Squalane 1, Squalene 2, Stearyl Tyrosine, SWE, Tetanus toxoid (TT), Theramide™, Threonyl muramyl dipeptide (TMDP), Ty Particles, and Walter Reed Liposomes.

In preferred embodiments, the adjuvant is an aluminium hydroxide gel (e.g., Alhydrogel™). In preferred embodiments, the adjuvant is SWE. In preferred embodiments, the adjuvant is MF59.

MF59 is an oil-in-water emulsion containing squalene (4.3%) in citric acid buffer with stabilizing nonionic surfactants Tween 80 (0.5%) and Span 85 (0.5%). MF59 has been shown to be well-tolerated in humans and is used in vaccines against seasonal influenza (see Ko and Kang, Hum Vaccin Immunother. 2018; 14(12): 3041-3045; U.S. Pat. No. 6,299,884).

For example, the composition may include an aluminum salt adjuvant, an oil in water emulsion (e.g. an oil-in-water emulsion comprising squalene, such as MF59, SWE, or AS03), a TL4 agonist, a TLR9 agonist (such as CpG oligodeoxynucleotides), a TLR7 agonist (such as imidazoquinoline or imiquimod), or a combination thereof. In some embodiments, the adjuvant is a combination of an aluminum salt and CPG1018. Suitable aluminum salts include hydroxides (e.g. oxyhydroxides), phosphates (e.g. hydroxyphosphates, orthophosphates), (e.g. see chapters 8 & 9 of Vaccine Design. (1995) eds. Powell & Newman. ISBN: 030644867X. Plenum), or mixtures thereof. The salts can take any suitable form (e.g. gel, crystalline, amorphous, etc.), with adsorption of antigen to the salt being an example. The concentration of Al+++ in a composition for administration to a patient may be less than 5 mg/ml e.g. <4 mg/ml, <3 mg/ml, <2 mg/ml, <1 mg/ml, etc. A preferred range is between 0.3 and 1 mg/ml. A maximum of 0.85 mg/dose is preferred. Aluminum hydroxide and aluminium phosphate adjuvants are suitable for use with the disclosure. In a preferred embodiment, a pharmaceutical composition provided herein comprises aluminum hydroxide as an adjuvant. In some embodiment, a pharmaceutical composition provided herein comprises 500 μg aluminium hydroxide.

In one aspect, the disclosure provides a vaccine, comprising the VLP described herein, wherein the vaccine optionally comprises one or more pharmaceutically acceptable diluents, adjuvants, or excipients. In some embodiments, the vaccine is a stable emulsion. In some embodiments, the vaccine comprises one or more adjuvants. In some embodiments, the one or more adjuvants is squalene, SLA, GLA, R848, IMQ, 3M-052, CpG, saponin (QS21), or combinations thereof. In some embodiments, the adjuvant is alum. In some embodiments, the adjuvant is a squalene-based emulsion. In some embodiments, the squalene-based emulsion is MF59. Selection of an adjuvant depends on the subject to be treated. Preferably, a pharmaceutically acceptable adjuvant is used.

In some embodiments, the adjuvant is a squalene emulsion.

In some embodiments, the adjuvant is a TLR4 immunostimulant (e.g., SLA, GLA), e.g., as described in Van Hoeven at al. PLoS One. 11(2):e0149610 (2016).

In some embodiments, the adjuvant is a TLR7/8 immunostimulant (e.g., R848, IMQ, 3M-052), e.g., as described in Dowling D. ImmunoHorizons (6):185-197 (2018).

In some embodiments, the adjuvant is a TLR9 immunostimulant (CpG), e.g., as described in Bode et al. Expert Rev Vaccines. 10(4):499-511 (2011).

In some embodiments, the adjuvant is saponin (QS21), e.g., as described in Zhu et al. Nat Prod Chem Res. 3(4):e113 (2016).

In some embodiments, the vaccine comprises a combination of two or more adjuvants (e.g. squalene emulsion and alum or a TLR4 immunostimulant).

One suitable immunological adjuvant comprises a compound of Formula (I) as defined in WO2011/027222, or a pharmaceutically acceptable salt thereof, adsorbed to an aluminum salt. Many further adjuvants can be used, including any of those disclosed in Powell & Newman (1995).

Compositions may include an antimicrobial, particularly when packaged in multiple dose format. Antimicrobials such as thiomersal and 2-phenoxyethanol are commonly found in vaccines, but sometimes it may be desirable to use either a mercury-free preservative or no preservative at all.

Compositions may comprise detergent e.g. a polysorbate, such as polysorbate 80. Detergents are generally present at low levels e.g. <0.01%.

Compositions may include sodium salts (e.g. sodium chloride) to give tonicity. A concentration of 10±2 mg/ml NaCl is typical e.g. about 9 mg/ml.

In some embodiments, the buffer in the vaccine composition is a Tris buffer, a histidine buffer, a phosphate buffer, a citrate buffer or an acetate buffer. The composition may also include a lyoprotectant, e.g. sucrose, sorbitol or trehalose. In certain embodiments, the composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the composition includes a bulking agent, like glycine. In yet other embodiments, the composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The composition may also include a tonicity adjusting agent, e.g., a compound that renders the formulation substantially isotonic or isoosmotic with human blood. Illustrative tonicity adjusting agents include sucrose, sorbitol, glycine, methionine, mannitol, dextrose, inositol, sodium chloride, arginine and arginine hydrochloride. In other embodiments, the composition additionally includes a stabilizer, e.g., a molecule which substantially prevents or reduces chemical and/or physical instability of the VLP, in lyophilized or liquid form. Illustrative stabilizers include sucrose, sorbitol, glycine, inositol, sodium chloride, methionine, arginine, and arginine hydrochloride.

In some embodiments, the disclosure provides a vaccine (immunogenic composition) comprising one or more pharmaceutically acceptable excipients.

In some embodiments, the vaccine (immunogenic composition) is a stable emulsion.

In some embodiments, the disclosure provides a vaccine (immunogenic composition) comprises one or more adjuvants. In some embodiments, the one or more adjuvants comprises a TLR4 immunostimulant, e.g., Monophosphoryl Lipid A (MPL), Glucopyranosyl Lipid A (GLA), and/or Soluble Leishmania Antigen (SLA).

In another aspect, the disclosure provides a method of inducing an immune response or strengthening an existing immune response against RSV and/or hMPV, comprising administering to a subject in need thereof an immunologically effective amount of the immunogenic composition described herein, which comprises the VLP as described herein.

In certain embodiments, the immune response comprises the production of neutralizing antibodies against an infectious agent. In certain embodiments, the neutralizing antibodies are complement-independent.

The immune response can comprise a humoral immune response, a cell-mediated immune response, or both. In some embodiments an immune response is induced against each delivered antigenic protein. A cell-mediated immune response can comprise a Helper T-cell (Th) response, a CD8+ cytotoxic T-cell (CTL) response, or both. In some embodiments the immune response comprises a humoral immune response, and the antibodies are neutralizing antibodies. Neutralizing antibodies block viral infection of cells. Viruses infect epithelial cells and also fibroblast cells. In some embodiments the immune response reduces or prevents infection of both cell types. Neutralizing antibody responses can be complement-dependent or complement-independent. In some embodiments the neutralizing antibody response is complement-independent. In some embodiments the neutralizing antibody response is cross-neutralizing; i.e., an antibody generated against an administered composition neutralizes a virus of a strain other than the strain used in the composition.

A useful measure of antibody potency in the art is “50% neutralization titer.” To determine 50% neutralizing titer, serum from immunized animals is diluted to assess how dilute serum can be yet retain the ability to block entry of 50% of viruses into cells. For example, a titer of 700 means that serum retained the ability to neutralize 50% of virus after being diluted 700-fold. Thus, higher titers indicate more potent neutralizing antibody responses. In some embodiments, this titer is in a range having a lower limit of about 200, about 400, about 600, about 800, about 1000, about 1500, about 2000, about 2500, about 3000, about 3500, about 4000, about 4500, about 5000, about 5500, about 6000, about 6500, or about 7000. The 50% neutralization titer range can have an upper limit of about 400, about 600, about 800, about 1000, about 1500, about 2000, about 2500, about 3000, about 3500, about 4000, about 4500, about 5000, about 5500, about 6000, about 6500, about 7000, about 8000, about 9000, about 10000, about 11000, about 12000, about 13000, about 14000, about 15000, about 16000, about 17000, about 18000, about 19000, about 20000, about 21000, about 22000, about 23000, about 24000, about 25000, about 26000, about 27000, about 28000, about 29000, or about 30000. For example, the 50% neutralization titer can be about 3000 to about 25000. “About” means plus or minus 10% of the recited value.

In some embodiments, the virus-like particles of the disclosure generate an immune response of 0.5×10³ IU/mL, 1.0×10³ IU/mL, 1.5×10³ IU/mL, 2.0×10³ IU/mL, 3.0×10³ IU/mL, 4.0×10³ IU/mL, 5.0×10³ IU/mL, 6.0×10³ IU/mL, 7.0×10³ IU/mL, 8.0×10³ IU/mL, 9.0×10³ IU/mL, 10×10³ IU/mL, 25×10³ IU/mL, 50×10³ IU/mL, 100×10³ IU/mL or greater. In some embodiments, the virus-like particles of the disclosure generate an immune response of 0.5×10³ IU/mL or greater.

Compositions of the disclosure will generally be administered directly to a subject. Direct delivery may be accomplished by parenteral injection (e.g. subcutaneously, intraperitoneally, intravenously, intramuscularly, or to the interstitial space of a tissue), orally, intranasal, or by any other suitable route. For example, intramuscular administration may be used e.g. to the thigh or the upper arm. Injection may be via a needle (e.g. a hypodermic needle), but needle-free injection may alternatively be used. A typical intramuscular dosage volume is 0.5 ml.

Dosage can be by a single dose schedule or a multiple dose schedule. Multiple doses may be used in a primary immunization schedule and/or in a booster immunization schedule. In a multiple dose schedule the various doses may be given by the same or different routes, e.g., a parenteral prime and mucosal boost, a mucosal prime and parenteral boost, etc. Multiple doses will typically be administered at least 1 week apart (e.g., about 2 weeks, about 3 weeks, about 4 weeks, about 6 weeks, about 8 weeks, about 10 weeks, about 12 weeks, about 16 weeks, etc.). Multiple doses may be administered at least 1 month apart (e.g., about 2 months, about 3 months, about 4 months, about 6 months, about 8 months, about 10 months, about 12 months, about 16 months, etc.). A second or subsequent does may be administered over longer intervals, e.g., about 1 year, about 2 years, about 3 years, about 4 years, or about 5 years after the previous dose, such as about 3-5 years after the previous dose.

The subject may be a child (e.g., a toddler or infant), a teenager, or an adult. A vaccine intended for children may also be administered to adults, e.g., to assess safety, dosage, immunogenicity, etc.

Vaccines of the disclosure may be prophylactic (i.e. to prevent disease) or therapeutic (i.e. to reduce or eliminate the symptoms of a disease). The term prophylactic may be considered as reducing the severity of or preventing the onset of a particular condition. For the avoidance of doubt, the term prophylactic vaccine may also refer to vaccines that ameliorate the effects of a future infection, for example by reducing the severity or duration of such an infection.

Isolated and/or purified VLPs described herein can be administered alone or as either prime or boost in mixed-modality regimes, such as a RNA prime or DNA primer followed by a protein boost. For example, an adenoviral vector may be used as the prime in combination with a vaccine composition of the present disclosure. Benefits of the RNA-prime/protein-boost strategy, as compared to a protein-prime/protein-boost strategy, include, for example, increased antibody titers, a more balanced IgG1:IgG2a subtype profile, induction of TH1-type CD4+ T cell-mediated immune response that was similar to that of viral particles, and reduced production of non-neutralizing antibodies. The RNA prime can increase the immunogenicity of compositions regardless of whether they contain or do not contain an adjuvant.

In the RNA-prime/protein boost-strategy, the RNA and the protein are directed to the same target antigen. Examples of suitable modes of delivering RNAs include virus-like replicon particles (VRPs), alphavirus RNA, replicons encapsulated in lipid nanoparticles (LNPs) or formulated RNAs, such as replicons formulated with cationic nanoemulsions (CNEs). Suitable cationic oil-in-water nanoemulsions are disclosed in WO2012/006380 e.g. comprising an oil core (e.g. comprising squalene) and a cationic lipid (e.g. DOTAP, DMTAP, DSTAP, DC-cholesterol, etc.).

Alternatively, two doses of the VLP may be administered at a predetermined interval to achieve a prime-boost effect. The predetermined interval may be 1, 2, 3, 4, 6, 7, 10, or 14 days; or 3-5 days, 7-10 days, or 10-14 days, 14-21 days or the like. The predetermined interval may be 1, 2, 3, 4, or 6 weeks; or 2-3 weeks, 3-4 weeks, or 5-6 weeks or the like. The predetermined interval may be 1, 2, 3, or 4 months or 1, 2, 3, or 4 years.

In some embodiments, the RNA molecule is encapsulated in, bound to or adsorbed on a cationic lipid, a liposome, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, a polycationic peptide, a cationic nanoemulsion, or combinations thereof.

The disclosure further provides combination vaccines. The vaccines of the disclosure include vaccines comprising both an RSV and/or hMPV VLP and vaccines for one or more of: coronavirus (such as a betacoronavirus, e.g., SARS-CoV-2, respiratory syncytial virus, Rabies, Pneumococcal virus, Typhoid fever, Hepatitis A, Polio, Influenza, Hepatitis B, Yellow Fever, Japanese encephalitis, Parvovirus, Distemper, Adenovirus, Parainfluenza, Influenza, Measles, Lyme disease, Coronavirus, Vesicular stomatitis virus, Herpes simplex virus, Baculovirus, Thogotovirus, and Bornaviridae.

Also provided herein are kits for administration of nucleic acid (e.g., RNA), purified proteins, and purified VLPs described herein, and instructions for use. The disclosure also provides a delivery device pre-filled with a composition or a vaccine disclosed herein.

The pharmaceutical compositions described herein can be administered in combination with one or more additional therapeutic agents, such as an antiviral, e.g., palivizumab. The additional therapeutic agents may include, but are not limited to antibiotics or antibacterial agents, antiemetic agents, antifungal agents, anti-inflammatory agents, antiviral agents, immunomodulatory agents, cytokines, antidepressants, hormones, alkylating agents, antimetabolites, antitumour antibiotics, antimitotic agents, topoisomerase inhibitors, cytostatic agents, anti-invasion agents, antiangiogenic agents, inhibitors of growth factor function inhibitors of viral replication, viral enzyme inhibitors, anticancer agents, α-interferons, β-interferon, ribavirin, hormones, and other toll-like receptor modulators, immunoglobulins (Igs), and antibodies modulating Ig function (such as anti-IgE (omalizumab)).

In certain embodiments, the compositions disclosed herein may be used as a medicament, e.g., for use in inducing or enhancing an immune response in a subject in need thereof, such as a mammal.

In certain embodiments, the compositions disclosed herein may be used in the manufacture of a medicament for inducing or enhancing an immune response in a subject in need thereof, such as a mammal.

One way of checking efficacy of therapeutic treatment involves monitoring infection by an infectious agent after administration of the compositions or vaccines disclosed herein. One way of checking efficacy of prophylactic treatment involves monitoring immune responses, systemically (such as monitoring the level of IgG1 and IgG2a production) and/or mucosally (such as monitoring the level of IgA production), against the antigen. Typically, antigen-specific serum antibody responses are determined post-immunization but pre-challenge whereas antigen-specific mucosal antibody responses are determined post-immunization and post-challenge.

### Methods of Treatment

In one aspect, the disclosure provides a method of immunizing a subject against infection by human metapneumovirus (hMPV), the method comprising administering the vaccine described herein. In some embodiments, the subject is simultaneously immunized against infection by respiratory syncytial virus (RSV). In some embodiments, the vaccine is administered by subcutaneous injection. In some embodiments, wherein the vaccine is administered by intramuscular injection. In some embodiments, wherein the vaccine is administered by intradermal injection. In some embodiments, wherein the vaccine is administered intranasally. In one aspect, the disclosure provides a pre-filled syringe comprising the vaccine described herein. In one aspect, the disclosure provides a kit comprising the vaccine described herein or the pre-filled syringe described herein.

In another aspect, provided herein is a unit dose of the pharmaceutical composition comprising about 0.5 μg to about 1 μg, about 20 μg to about 25 μg, about 70 μg to about 75 μg, about 100 μg to about 125 μg, about 100 μg to about 150 μg, about 125 μg to about 175 μg, about 200 μg to about 250 μg, about 225 μg to about 300 μg, or about 250 μg to about 350 μg of the VLPs.

In another aspect, provided herein is a unit dose of the pharmaceutical composition comprising about 0.5 μg to about 1 μg, about 20 μg to about 25 μg, about 25 μg to about 50 μg, about 50 μg to about 70 μg, about 70 μg to about 75 μg, about 75 μg to about 100 μg, about 100 μg to about 125 μg, about 125 μg to about 150 μg, about 150 μg to about 175 μg, about 175 μg to about 200 μg, about 200 μg to about 250 μg, or about 250 μg to about 300 μg of the VLPs.

In another aspect, provided herein is a method of vaccinating a subject, comprising administering to the subject an effective amount of a pharmaceutical composition provided herein. In another aspect, provided herein is a method of generating an immune response in a subject, comprising administering to the subject an effective amount of a pharmaceutical composition provided herein. In another aspect, provided herein is a method of preventing RSV disease a subject, comprising administering to the subject an effective amount of a pharmaceutical composition provided herein. In some embodiments, the subject is at risk of severe RSV disease. In another aspect, provided herein is a method of preventing hMPV disease in a subject, comprising administering to the subject an effective amount of a pharmaceutical composition provided herein. In some embodiments, the subject is at risk of severe hMPV disease. In some embodiments, the subject is an adult of over 60 years of age. In some embodiments, the subject is a healthy adult of 18-45 years of age.

In some embodiments, a composition comprising 25 μg of a first VLP comprising a respiratory syntactical virus (RSV) F protein ectodomain or antigenic variant thereof and 125 μg of a second VLP comprising a human metapneumovirus (hMPV) F protein ectodomain or antigenic variant thereof is administered intramuscularly to a subject. In some embodiments, a composition comprising 25 μg of a first VLP comprising a respiratory syntactical virus (RSV) F protein ectodomain or antigenic variant thereof, 125 μg of a second VLP comprising a human metapneumovirus (hMPV) F protein ectodomain or antigenic variant thereof, and 9.75 mg of an oil-in-water emulsion comprising squalene, such as MF59®, is administered intramuscularly to a subject.

In some embodiments, a composition comprising 75 μg of a first VLP comprising a respiratory syntactical virus (RSV) F protein ectodomain or antigenic variant thereof and 75 μg of a second VLP comprising a human metapneumovirus (hMPV) F protein ectodomain or antigenic variant thereof is administered intramuscularly to a subject. In some embodiments, a composition comprising 75 μg of a first VLP comprising a respiratory syntactical virus (RSV) F protein ectodomain or antigenic variant thereof, 75 μg of a second VLP comprising a human metapneumovirus (hMPV) F protein ectodomain or antigenic variant thereof, and 9.75 mg of an oil-in-water emulsion comprising squalene, such as MF59®, is administered intramuscularly to a subject.

In some embodiments, a composition comprising 75 μg of a first VLP comprising a respiratory syntactical virus (RSV) F protein ectodomain or antigenic variant thereof and 150 μg of a second VLP comprising a human metapneumovirus (hMPV) F protein ectodomain or antigenic variant thereof is administered intramuscularly to a subject. In some embodiments, a composition comprising 75 μg of a first VLP comprising a respiratory syntactical virus (RSV) F protein ectodomain or antigenic variant thereof, 150 μg of a second VLP comprising a human metapneumovirus (hMPV) F protein ectodomain or antigenic variant thereof, and 9.75 mg of an oil-in-water emulsion comprising squalene, such as MF59@, is administered intramuscularly to a subject.

In some embodiments, a composition comprising 75 μg of a first VLP comprising a respiratory syntactical virus (RSV) F protein ectodomain or antigenic variant thereof and 225 μg of a second VLP comprising a human metapneumovirus (hMPV) F protein ectodomain or antigenic variant thereof is administered intramuscularly to a subject. In some embodiments, a composition comprising 75 μg of a first VLP comprising a respiratory syntactical virus (RSV) F protein ectodomain or antigenic variant thereof, 225 μg of a second VLP comprising a human metapneumovirus (hMPV) F protein ectodomain or antigenic variant thereof, and 9.75 mg of an oil-in-water emulsion comprising squalene, such as MF59®, is administered intramuscularly to a subject.

In some embodiments, a composition comprising 112.5 μg of a first VLP comprising a respiratory syntactical virus (RSV) F protein ectodomain or antigenic variant thereof and 112.5 μg of a second VLP comprising a human metapneumovirus (hMPV) F protein ectodomain or antigenic variant thereof is administered intramuscularly to a subject. In some embodiments, a composition comprising 112.5 μg of a first VLP comprising a respiratory syntactical virus (RSV) F protein ectodomain or antigenic variant thereof, 112.5 μg of a second VLP comprising a human metapneumovirus (hMPV) F protein ectodomain or antigenic variant thereof, and 9.75 mg of an oil-in-water emulsion comprising squalene, such as MF59@, is administered intramuscularly to a subject.

In some embodiments, a composition comprising 150 μg of a first VLP comprising a respiratory syntactical virus (RSV) F protein ectodomain or antigenic variant thereof and 150 μg of a second VLP comprising a human metapneumovirus (hMPV) F protein ectodomain or antigenic variant thereof is administered intramuscularly to a subject. In some embodiments, a composition comprising 150 μg of a first VLP comprising a respiratory syntactical virus (RSV) F protein ectodomain or antigenic variant thereof, 150 μg of a second VLP comprising a human metapneumovirus (hMPV) F protein ectodomain or antigenic variant thereof, and 9.75 mg of an oil-in-water emulsion comprising squalene, such as MF59®, is administered intramuscularly to a subject.

In some embodiments, a composition comprising 225 μg of a first VLP comprising a respiratory syntactical virus (RSV) F protein ectodomain or antigenic variant thereof and 150 μg of a second VLP comprising a human metapneumovirus (hMPV) F protein ectodomain or antigenic variant thereof is administered intramuscularly to a subject. In some embodiments, a composition comprising 225 μg of a first VLP comprising a respiratory syntactical virus (RSV) F protein ectodomain or antigenic variant thereof, 150 μg of a second VLP comprising a human metapneumovirus (hMPV) F protein ectodomain or antigenic variant thereof, and 9.75 mg of an oil-in-water emulsion comprising squalene, such as MF59®, is administered intramuscularly to a subject.

In some embodiments, a composition comprising 150 μg of a first VLP comprising a respiratory syntactical virus (RSV) F protein ectodomain or antigenic variant thereof and 75 μg of a second VLP comprising a human metapneumovirus (hMPV) F protein ectodomain or antigenic variant thereof is administered intramuscularly to a subject. In some embodiments, a composition comprising 150 μg of a first VLP comprising a respiratory syntactical virus (RSV) F protein ectodomain or antigenic variant thereof, 75 μg of a second VLP comprising a human metapneumovirus (hMPV) F protein ectodomain or antigenic variant thereof, and 9.75 mg of an oil-in-water emulsion comprising squalene, such as MF59®, is administered intramuscularly to a subject.

In another aspect, provided herein is a method of generating an immune response in an unborn child, the method comprising administering an effective amount of a pharmaceutical composition provided herein to the mother of said unborn child. In some embodiments, the pharmaceutical composition is administered to the mother in the last trimester of the pregnancy.

In some embodiments, a first dose of the pharmaceutical composition is administered to a female during pregnancy and a second dose of the composition is administered to the infant that is born from the pregnancy. Non-limiting examples of generating an immune response in an infant include those disclosed in Int'l Pat. Pub No. WO2012103361A1, which is incorporated herein in its entirety.

In some embodiments, an effective amount of a pharmaceutical composition comprises about 0.5 μg to about 1 μg, about 20 μg to about 25 μg, about 70 μg to about 75 μg, about 100 μg to about 125 μg, about 100 μg to about 150 μg, about 125 μg to about 175 μg, about 200 μg to about 250 μg, about 225 μg to about 300 μg, or about 250 μg to about 350 μg of the VLPs.

In some embodiments, an effective amount of a pharmaceutical composition comprises about 0.5 μg to about 1 μg, about 20 μg to about 25 μg, about 25 μg to about 50 μg, about 50 μg to about 70 μg, about 70 μg to about 75 μg, about 75 μg to about 100 μg, about 100 μg to about 125 μg, about 125 μg to about 150 μg, about 150 μg to about 175 μg, about 175 μg to about 200 μg, or about 200 μg to about 250 μg, or about 250 μg to about 300 μg of the VLP.

In some embodiments, a method provided herein further comprising administering a second dose of a pharmaceutical composition provided herein. In some embodiments, the second dose is administered within about 2, 3, 4, 5, 6, 7, 8, 9, 10, 11, or 12 weeks, about 1 month, about 2 months, about 3 months, about 4 months, about 5 months, about 6 months, about 9 months, or about 12 months of the first dose. In some embodiments, the method provided herein further comprises administering a third dose of a pharmaceutical composition provided herein. In some embodiments, the third dose is administered about 1 year, about 2 years, about 3 years, about 4 years, or about 5 years after the second dose. In some embodiments, a method provided herein further comprises administering subsequent doses at regular intervals of about 1, 2, 3, 4 or 5 years.

In some embodiments, a method provided herein limits the development of an RSV infection in a subject. In some embodiments, the method results in the production of RSV-A-specific neutralizing antibodies in the subject. In some embodiments, the method results in an increase in RSV-A-specific neutralizing antibodies in the subject of at least about 2-fold, about 3-fold, at least about 4-fold, at least about 5-fold, at least about 10-fold, at least about 15-fold, at least about 20-fold, or at least about 25-fold compared to baseline. In some embodiments, the increase in RSV-A-specific neutralizing antibodies is detectable within about one week, within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the pharmaceutical composition. In some embodiments, the method results in the production of RSV-B-specific neutralizing antibodies in the subject. In some embodiments, the method results in an increase in RSV-B-specific neutralizing antibodies in the subject of at least about 2-fold, about 3-fold, at least about 4-fold, at least about 5-fold, at least about 10-fold, at least about 15-fold, at least about 20-fold, or at least about 25-fold compared to baseline. In some embodiments, the increase in RSV-B-specific neutralizing antibodies is detectable within about one week, within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the pharmaceutical composition.

In some embodiments, the method results in the production of RSV F-protein-specific IgG antibodies in the subject. In some embodiments, the method results in an increase of RSV F-protein-specific neutralizing antibodies in the subject of at least about 2-fold, about 3-fold, at least about 4-fold, at least about 5-fold, at least about 10-fold, at least about 15-fold, at least about 20-fold, or at least about 25-fold compared to baseline. In some embodiments, the increase in RSV F-protein-specific neutralizing antibodies is detectable within about one week, within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the pharmaceutical composition.

In some embodiments, the method results in the production of Core-VLP-specific IgG antibodies in the subject. In some embodiments, the method results in an increase in Core-VLP-specific IgG antibodies in the subject of at least about 2-fold, about 3-fold, at least about 4-fold, at least about 5-fold, at least about 10-fold, at least about 15-fold, at least about 20-fold, or at least about 25-fold compared to baseline. In some embodiments, the increase in Core-VLP-specific IgG antibodies is detectable within about one week, within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the pharmaceutical composition.

In some embodiments, the method results in the production of RSV F-protein-specific memory-B-cells in the subject. In some embodiments, the method results in an increase in RSV F-protein-specific memory-B-cells in the subject of at least about 2-fold, about 3-fold, at least about 4-fold, at least about 5-fold, at least about 10-fold, at least about 15-fold, at least about 20-fold, or at least about 25-fold compared to baseline. In some embodiments, the increase in RSV F-protein-specific memory-B-cells is detectable within about one week, within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the pharmaceutical composition.

In some embodiments, the method results in the production of RSV F-protein-specific T-cells in the subject. In some embodiments, the method results in an increase in RSV F-protein-specific T-cells in the subject of at least about 2-fold, about 3-fold, at least about 4-fold, at least about 5-fold, at least about 10-fold, at least about 15-fold, at least about 20-fold, or at least about 25-fold compared to baseline. In some embodiments, the increase in RSV F-protein-specific T-cells is detectable within about one week, within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the pharmaceutical composition.

In some embodiments, the method results in the production of antibodies against human metapneumovirus in the subject. In some embodiments, the method results in an increase in antibodies against human metapneumovirus (hMPV) in the subject of at least about 2-fold, about 3-fold, at least about 4-fold, at least about 5-fold, at least about 10-fold, at least about 15-fold, at least about 20-fold, or at least about 25-fold compared to baseline. In some embodiments, the increase in antibodies against human metapneumovirus is detectable within about one week, within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the pharmaceutical composition.

In some embodiments, a method provided herein limits the development of an hMPV infection in a subject. In some embodiments, the method results in the production of hMPV-A-specific neutralizing antibodies in the subject. In some embodiments, the method results in an increase in hMPV-A-specific neutralizing antibodies in the subject of at least about 2-fold, about 3-fold, at least about 4-fold, at least about 5-fold, at least about 10-fold, at least about 15-fold, at least about 20-fold, or at least about 25-fold compared to baseline. In some embodiments, the increase in hMPV-A-specific neutralizing antibodies is detectable within about one week, within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the pharmaceutical composition. In some embodiments, the method results in the production of hMPV-B-specific neutralizing antibodies in the subject. In some embodiments, the method results in an increase in hMPV-B-specific neutralizing antibodies in the subject of at least about 2-fold, about 3-fold, at least about 4-fold, at least about 5-fold, at least about 10-fold, at least about 15-fold, at least about 20-fold, or at least about 25-fold compared to baseline. In some embodiments, the increase in hMPV-B-specific neutralizing antibodies is detectable within about one week, within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the pharmaceutical composition.

In some embodiments, the method results in the production of hMPV F-protein-specific IgG antibodies in the subject. In some embodiments, the method results in an increase of hMPV F-protein-specific neutralizing antibodies in the subject of at least about 2-fold, about 3-fold, at least about 4-fold, at least about 5-fold, at least about 10-fold, at least about 15-fold, at least about 20-fold, or at least about 25-fold compared to baseline. In some embodiments, the increase in hMPV F-protein-specific neutralizing antibodies is detectable within about one week, within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the pharmaceutical composition.

In some embodiments, the method results in the production of Core-VLP-specific IgG antibodies in the subject. In some embodiments, the method results in an increase in Core-VLP-specific IgG antibodies in the subject of at least about 2-fold, about 3-fold, at least about 4-fold, at least about 5-fold, at least about 10-fold, at least about 15-fold, at least about 20-fold, or at least about 25-fold compared to baseline. In some embodiments, the increase in Core-VLP-specific IgG antibodies is detectable within about one week, within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the pharmaceutical composition.

In some embodiments, the method results in the production of hMPV F-protein-specific memory-B-cells in the subject. In some embodiments, the method results in an increase in hMPV F-protein-specific memory-B-cells in the subject of at least about 2-fold, about 3-fold, at least about 4-fold, at least about 5-fold, at least about 10-fold, at least about 15-fold, at least about 20-fold, or at least about 25-fold compared to baseline. In some embodiments, the increase in hMPV F-protein-specific memory-B-cells is detectable within about one week, within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the pharmaceutical composition.

In some embodiments, the method results in the production of hMPV F-protein-specific T-cells in the subject. In some embodiments, the method results in an increase in hMPV F-protein-specific T-cells in the subject of at least about 2-fold, about 3-fold, at least about 4-fold, at least about 5-fold, at least about 10-fold, at least about 15-fold, at least about 20-fold, or at least about 25-fold compared to baseline. In some embodiments, the increase in hMPV F-protein-specific T-cells is detectable within about one week, within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the pharmaceutical composition. In another aspect, the disclosure provides methods to vaccinate a subject against infection with RSV (e.g., infection with RSV-A and/or RSV-B) and/or hMPV (e.g., infection with hMPV-A and/or hMPV-B), comprising administering to a subject in need thereof an amount effective to treat or limit development of the infection of the polypeptide, virus-like particle, composition, nucleic acid, pharmaceutical composition, or vaccine of any embodiment herein (referred to as the “immunogenic composition”). In some embodiments, such a method prevents disease following infection with RSV subtypes A and/or B. In some embodiments, such a method protects against the development of RSV-associated disease (e.g., severe disease), for example, pneumonia and/or acute respiratory disease. The subject may be any suitable mammalian subject, including but not limited to a human subject. In some embodiments, a subject is a human child, e.g., a child of less than 12 months of age. In some embodiments, a subject is a human toddler, e.g., of about 1 to about 3 years of age or of about 1 to about 5 years of age. In some embodiment, the subject is a human adult of more than 50 years of age or more than 60 years of age. In some embodiments, the subject is a human adult of more than 65 years of age. In particular embodiments, the subject is dependent on the help of others or with serious health concerns or risks (e.g. a frail elderly person). In some embodiments, the subject is a healthy adult of 18-60 years of age. In some embodiments, the subject is a healthy adult of 18-45 years of age. In another embodiment, the subject is a pregnant woman. In some embodiments the subject is an immunocompromised human adult. In some embodiments, the subject is a human adult suffering from chronic underlying heart and/or lung disease or from functional disability. In some embodiments, the subject is at risk of severe RSV disease (e.g., LRTI or pneumonia).

The immunogenic compositions provided herein may be used vaccinate an unborn child. The administration of certain inactivated vaccines is recommended during pregnancy to induce immunity in the unborn child, for example, the tetanus toxoid, reduced diphtheria toxoid and acellular pertussis (Tdap) vaccine and the influenza vaccine. Thus, in some embodiments, provide herein is a method of generating an immune response in an unborn child, the method comprising administering an effective amount of the immunogenic composition provided herein to the mother of said unborn child. The immunogenic composition may be administered at any suitable time point in the pregnancy, e.g., in the last trimester of the pregnancy.

An immunogenic composition provided herein may be co-administered with other treatments, such as other vaccines. Thus in some embodiments, a subject treated in accordance with a method provided herein may also be administered one or more seasonal or pandemic vaccines such as an influenza vaccine or a SARS-Cov2 vaccine. In some embodiments, a subject treated in accordance with the methods provided herein may also be administered a pneumococcal, Recombinant Zoster (Shingles), or Tdap vaccine. One, two, or more vaccines may be co-administered with an immunogenic composition provided herein. “Co-administration” includes both concurrently as well as subsequent administration. For example, the one, two, or more vaccines and an immunogenic composition provided herein may be administered on the same day. In some embodiments, the one, two, or more vaccines and an immunogenic composition provided herein are administered within 1 hour, within 2 hours, within 3 hours, within 4 hours, within 5 hours, within 6 hours, within 8 hours, within 10 hours, or within 12 hours.

In another aspect, provide herein is a method of treating a subject suffering from RSV or hMPV infection. As used herein, “treat” or “treating” includes, but is not limited to accomplishing one or more of the following: (a) reducing viral titer in the subject; (b) limiting any increase of viral titer in the subject; (c) reducing the severity of viral infection; (d) limiting or preventing development of symptoms after viral infection; (e) inhibiting worsening of symptoms of viral infection; (f) limiting or preventing recurrence of symptoms of viral infection in subjects that were previously symptomatic for viral infection; and/or (e) increasing survival. In some embodiments, a method of vaccinating decreases the subject's risk of becoming infected with a virus (e.g., RSV and/or hMPV). In some embodiments, a method of vaccinating limits the development of a viral infection. In some embodiments, a method of vaccinating decreases the severity of the symptoms of viral infection. In preferred embodiments, the infection is a lower respiratory tract infection (LRTI).

In some embodiments, the methods provided herein may be used to prevent an RSV infection or illness (e.g., pneumonia or acute respiratory disease) in a subject.

When the method comprises limiting a viral infection, the immunogenic composition is administered prophylactically to a subject that is not known to be infected but may be at risk of exposure to a virus (e.g., RSV ro hMPV). As used herein, “limiting development” includes, but is not limited to accomplishing one or more of the following: (a) generating an immune response (antibody and/or cell-based, e.g., CD4 T cells, memory B cells, and/or CD8 T cells) to a virus in the subject; (b) generating neutralizing antibodies against virus or viral proteins in the subject (b) limiting build-up of viral titer in the subject after exposure to virus; and/or (c) limiting or preventing development of symptoms after viral infection. Exemplary symptoms of viral (e.g., RSV or hMPV) infection include, but are not limited to, fever, fatigue, cough, nasal congestion, sneezing, shortness of breath, wheezing, and lower respiratory tract infections.

The methods provided herein may be used to limit development of infection with an RSV (or hMPV)-A subtype and/or an RSV (or hMPV)-B subtype. Without wishing to be bound by theory, it is believed that immunization with an F protein of one RSV (or hMPV) subtype will result in at least some immunity against the other due to the high sequence similarity between the F proteins of RSV (or hMPV)-A and RSV (or hMPV)-B.

Further, the methods provided herein may be used to limit development of infection with an original strain of RSV (or hMPV) and/or infection with a variant strain of RSV (or hMPV). Examples of variant RSV (or hMPV) strains include, without limitation, RSV (or hMPV) ONI, RSV (or hMPV) NA1, RSV (or hMPV) LBA1, RSV (or hMPV) LBA2, RSV (or hMPV) BA, RSV (or hMPV) Long, RAV A2, and others (see, e.g., Pandya et al., Pathogens 2019, 8(2), 67; and Melero and Moore, Curr Top Microbiol Immunol. 2013; 372: 59-82). The pharmaceutical composition of the present invention may be effective in limiting infection of RSV (or hMPV) strains that have not yet been described or discovered.

In some embodiments, the methods described herein generate an immune response in a subject in the subject not known to be infected with RSV (or hMPV) (e.g., RSV (or hMPV)-A and/or RSV (or hMPV)-B), wherein the immune response serves to limit development of infection and symptoms of an RSV (or hMPV) (e.g., RSV (or hMPV)-A and/or RSV (or hMPV)-B) infection. In some embodiments, the immune response comprises generation of neutralizing antibodies and/or cell-based responses against RSV (or hMPV) (e.g., RSV (or hMPV)-A and/or RSV (or hMPV)-B). In some embodiments, the immune response comprises generation of RSV (or hMPV) F protein-specific (e.g., RSV (or hMPV)-A and/or RSV (or hMPV)-B F protein-specific) responses with a mean geometric titer of at least 1×10³, at least 1×10⁴, at least 1×10⁵, at least 1×10⁶, at least 1×10⁷, at least 1×10⁸, or at least 1×10⁹. In a further embodiment, the immune response comprises generation of antibodies against multiple antigenic epitopes or RSV (or hMPV) (e.g., RSV (or hMPV)-A and/or RSV (or hMPV)-B).

In one aspect, the methods provided herein may results in an increase in antibody titers in a subject, e.g., in an increase in RSV (or hMPV)-A-specific neutralizing antibodies, RSV (or hMPV)-B-specific neutralizing antibodies, RSV (or hMPV) F-protein-specific IgG antibodies, RSV (or hMPV) F-protein-specific neutralizing antibodies, Core-VLP-specific IgG antibodies and/or antibodies against human metapneumovirus. Antibody titers may be determined using any suitable assays known in the art or described herein including, without limitation, binding enzyme-linked immunosorbent assays (ELISA), competition ELISAs, immunoprecipitation, immunoblotting, and agglutination assays.

In some embodiments, the methods provided herein result in an increase in antibodies (e.g., RSV (or hMPV)-A-specific neutralizing antibodies, RSV (or hMPV)-B-specific neutralizing antibodies, RSV (or hMPV) F-protein-specific IgG antibodies, RSV (or hMPV) F-protein-specific neutralizing antibodies, Core-VLP-specific IgG antibodies and/or antibodies against human metapneumovirus) of about 1-fold to about 2-fold, about 2-fold to about 3-fold, about 3-fold to about 4-fold, about 4-fold to about 5-fold, about 5-fold to about 6-fold, about 6-fold to about 7-fold, about 7-fold to about 8-fold, about 8-fold to about 9-fold, about 9-fold to about 10-fold, about 10-fold to about 12-fold, about 12-fold to about 15-fold, about 15-fold to about 20-fold, about 20-fold to about 25-fold, about 25-fold to about 30-fold, about 30-fold to about 40-fold, about 40-fold to about 50-fold, about 50-fold to about 60-fold, about 60-fold to about 70-fold, about 70-fold to about 80-fold, about 80-fold to about 90-fold, about 90-fold to about 100-fold, or more than about 100-fold compared to baseline. In some embodiments, the methods provided herein result in an increase in antibodies (e.g., in an increase in RSV (or hMPV)-A-specific neutralizing antibodies, RSV (or hMPV)-B-specific neutralizing antibodies, RSV (or hMPV) F-protein-specific IgG antibodies, RSV (or hMPV) F-protein-specific neutralizing antibodies, Core-VLP-specific IgG antibodies and/or antibodies against human metapneumovirus) of at least about 2-fold, about 3-fold, at least about 4-fold, at least about 5-fold, at least about 10-fold, at least about 15-fold, at least about 20-fold, or at least about 25-fold compared to baseline.

By “baseline” is meant a measurement of antibodies immediately prior to administration of the first dose of an immunogenic composition provided herein. In some embodiments, the increase in antibodies (e.g., RSV (or hMPV)-A-specific neutralizing antibodies, RSV (or hMPV)-B-specific neutralizing antibodies, RSV (or hMPV) F-protein-specific IgG antibodies, RSV (or hMPV) F-protein-specific neutralizing antibodies, Core-VLP-specific IgG antibodies and/or antibodies against human metapneumovirus) compared to baseline is detectable within about 3 days to about 7 days, about 1 week to about 2 weeks, about 2 weeks to about 3 weeks, about 3 weeks to about 4 weeks, about 4 weeks to about 5 weeks, about 5 weeks to about 6 weeks, about 6 weeks to about 7 weeks, about 7 weeks to about 8 weeks, about 8 weeks to about 9 weeks, about 9 weeks to about 10 weeks, about 10 weeks to about 11 weeks, about 11 weeks to about 12 weeks, about 3 months to about 4 months, about 4 months to about 5 months, about 5 months to about 6 months, about 6 months to about 9 months, about 9 months to about 12 months, about 12 months to about 18 months, about 18 months to about 24 months, about 2 years to about 3 years, about 3 years to about 4 years, about 4 years to about 5 years, or about 5 years to about 10 years of administration of the immunogenic composition. In some embodiments, the increase in antibodies (e.g., in an increase in RSV (or hMPV)-A-specific neutralizing antibodies, RSV (or hMPV)-B-specific neutralizing antibodies, RSV (or hMPV) F-protein-specific IgG antibodies, RSV (or hMPV) F-protein-specific neutralizing antibodies, Core-VLP-specific IgG antibodies and/or antibodies against human metapneumovirus) compared to baseline is detectable within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the immunogenic composition.

In another aspect, the methods provided herein may result in an increase in immune cells in a subject, e.g., an increase in RSV (or hMPV) F-protein-specific memory B cells and/or RSV (or hMPV) F-protein-specific T cells. The memory B cells and/or T cells may be specific to RSV (or hMPV)-A F protein or RSV (or hMPV)-B F protein, or they may be reactive to both. The number of immune cells in a subject may be determined using any suitable assay known in the art or described herein, including, without limitation, FACS and flow cytometry.

In some embodiments, the methods provided herein results in an increase in immune cells (e.g., in an increase in RSV (or hMPV) F-protein-specific memory B cells and/or RSV (or hMPV) F-protein-specific T cells) of about 1-fold to about 2-fold, about 2-fold about 3-fold, about 3-fold to about 4-fold, about 4-fold to about 5-fold, about 5-fold to about 6-fold, about 6-fold to about 7-fold, about 7-fold to about 8-fold, about 8-fold to about 9-fold, about 9-fold to about 10-fold, about 10-fold to about 12-fold, about 12-fold to about 15-fold, about 15-fold to about 20-fold, about 20-fold to about 25-fold, about 25-fold to about 30-fold, about 30-fold to about 40-fold, about 40-fold to about 50-fold, about 50-fold to about 60-fold, about 60-fold to about 70-fold, about 70-fold to about 80-fold, about 80-fold to about 90-fold, about 90-fold to about 100-fold, or more than about 100-fold compared to baseline. In some embodiments, the methods provided herein result in an increase in immune cells in a subject, e.g., an increase in RSV (or hMPV) F-protein-specific memory B cells and/or RSV (or hMPV) F-protein-specific T cells. In some embodiments, the methods provided herein results in an increase in immune cells (e.g., in an increase in RSV (or hMPV) F-protein-specific memory B cells and/or RSV (or hMPV) F-protein-specific T cells) of at least about 2-fold, about 3-fold, at least about 4-fold, at least about 5-fold, at least about 10-fold, at least about 15-fold, at least about 20-fold, or at least about 25-fold compared to baseline. The memory B cells and/or T cells may be specific to RSV (or hMPV)-A F protein or RSV (or hMPV)-B F protein, or they may be reactive to both.

In some embodiments, the increase in immune cells (e.g., in an increase in RSV (or hMPV) F-protein-specific memory B cells and/or RSV (or hMPV) F-protein-specific T cells) compared to baseline is detectable within about 3 days to about 7 days, about 1 week to about 2 weeks, about 2 weeks to about 3 weeks, about 3 weeks to about 4 weeks, about 4 weeks to about 5 weeks, about 5 weeks to about 6 weeks, about 6 weeks to about 7 weeks, about 7 weeks to about 8 weeks, about 8 weeks to about 9 weeks, about 9 weeks to about 10 weeks, about 10 weeks to about 11 weeks, about 11 weeks to about 12 weeks, about 3 months to about 4 months, about 4 months to about 5 months, about 5 months to about 6 months, about 6 months to about 9 months, about 9 months to about 12 months, about 12 months to about 18 months, about 18 months to about 24 months, about 2 years to about 3 years, about 3 years to about 4 years, about 4 years to about 5 years, or about 5 years to about 10 years of administration of the immunogenic composition. In some embodiments, the increase in increase in immune cells (e.g., in an increase in RSV (or hMPV) F-protein-specific memory B cells and/or RSV (or hMPV) F-protein-specific T cells) compared to baseline is detectable within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the immunogenic composition. The memory B cells and/or T cells may be specific to RSV (or hMPV)-A F protein or RSV (or hMPV)-B F protein, or they may be reactive to both.

The polypeptide, virus-like particle, composition, nucleic acid, pharmaceutical composition, or vaccine of any embodiment herein are typically formulated as a pharmaceutical composition, such as those disclosed above, and can be administered via any suitable route, including intranasally, sublingually, orally, parentally, by inhalation spray, rectally, or topically in dosage unit formulations containing conventional pharmaceutically acceptable carriers, adjuvants, and vehicles. The term parenteral as used herein includes, subcutaneous, intravenous, intra-arterial, intramuscular, intrasternal, intratendinous, intraspinal, intracranial, intrathoracic, infusion techniques or intraperitoneally. Polypeptide compositions may also be administered via microspheres, liposomes, immune-stimulating complexes (ISCOMs), or other microparticulate delivery systems or sustained release formulations introduced into suitable tissues (such as blood).

Dosage regimens can be adjusted to provide the optimum desired response (e.g., a therapeutic or prophylactic response). A suitable dosage range may, for instance, be 0.1 μg/kg to 0.5 μg/kg body weight, 0.5 μg/kg to 1 g body weight, 1 μg/kg to 2 μg/kg body weight, 2 g/kg to 3 μg/kg body weight, 3 μg/kg to 4 μg/kg body weight, 4 μg/kg to 5 μg/kg body weight, g/kg to 6 μg/kg body weight, 6 μg/kg to 7 μg/kg body weight, 7 μg/kg to 8 μg/kg body weight, 8 μg/kg to 9 μg/kg body weight, 9 μg/kg to 10 μg/kg body weight, 10 μg/kg to 15 μg/kg body weight, 15 μg/kg to 20 μg/kg body weight, 20 μg/kg to 25 μg/kg body weight, 25 μg/kg to 30 g/kg body weight, 30 μg/kg to 35 μg/kg body weight, 35 μg/kg to 40 μg/kg body weight, 40 g/kg to 45 μg/kg body weight, 45 μg/kg to 50 μg/kg body weight, 50 μg/kg to 55 μg/kg body weight, 55 μg/kg to 60 μg/kg body weight, 60 μg/kg to 65 μg/kg body weight, 65 μg/kg to 70 g/kg body weight, 70 μg/kg to 75 μg/kg body weight, 75 μg/kg to 80 μg/kg body weight, 80 g/kg to 85 μg/kg body weight, 85 μg/kg to 90 μg/kg body weight, 90 μg/kg to 95 μg/kg body weight, 95 μg/kg to 100 μg/kg body weight, 100 μg/kg to 150 g body weight, 150 μg/kg to 200 g body weight, 200 μg/kg to 250 μg/kg body weight, 250 μg/kg to 300 μg/kg body weight, 300 μg/kg to 350 μg/kg body weight, 350 μg/kg to 400 μg/kg body weight, 400 μg/kg to 450 g/kg body weight, 450 μg/kg to 500 g body weight, 500 μg/kg to 550 g body weight, 550 g/kg to 600 g body weight, 600 μg/kg to 650 g body weight, 650 μg/kg to 700 g body weight, 700 μg/kg to 750 μg/kg body weight, 750 μg/kg to 800 μg/kg body weight, 800 μg/kg to 850 μg/kg body weight, 850 μg/kg to 900 μg/kg body weight, 900 μg/kg to 950 μg/kg body weight, 950 μg/kg to 1 mg/kg body weight, 1 mg/kg to 2 mg/kg body weight, 2 mg/kg to 3 mg/kg body weight, 3 mg/kg to 4 mg/kg body weight, 4 mg/kg to 5 mg/kg body weight, 5 mg/kg to 6 mg/kg body weight, 6 mg/kg to 7 mg/kg body weight, 7 mg/kg to 8 mg/kg body weight, 8 mg/kg to 90 mg/kg body weight, 90 mg/kg to 100 mg/kg body weight, 100 mg/kg to 150 mg/kg body weight, 150 mg/kg to 200 mg/kg body weight, 200 mg/kg to 250 mg/kg body weight, 250 mg/kg to 300 mg/kg body weight, 300 mg/kg to 350 mg/kg body weight, 350 mg/kg to 400 mg/kg body weight, 400 mg/kg to 450 mg/kg body weight, 450 mg/kg to 500 mg/kg body weight, 500 mg/kg to 550 mg/kg body weight, 550 mg/kg to 600 mg/kg body weight, 600 mg/kg to 650 mg/kg body weight, 650 mg/kg to 700 mg/kg body weight, 700 mg/kg to 750 mg/kg body weight, 750 mg/kg to 800 mg/kg body weight, 800 mg/kg to 850 mg/kg body weight, 850 mg/kg to 900 mg/kg body weight, 900 mg/kg to 950 mg/kg body weight, or 950 mg/kg to 1 g/kg of the polypeptide or virus-like particle thereof.

The composition can be delivered in a single bolus, or may be administered more than once (e.g., 2, 3, 4, 5, or more times) as determined by attending medical personnel. In some embodiments, about 1 μg, about 2 μg, about 3 μg, about 4 μg, about 5 μg, about 10 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 100 μg, about 125 μg, about 150 μg, about 175 μg, about 200 μg, about 225 μg, about 250 μg, about 275 μg, about 300 μg, about 325 μg, bout 350 μg, about 375 μg, about 400 μg, about 425 μg, about 450 μg, about 475 μg, or about 500 μg of the polypeptide or virus-like particle are administered. In some embodiments, about 5 μg to about 10 μg, about 10 μg to about 15 μg, about 15 μg to about 20 μg, about 20 μg to about 30 μg, about 30 μg to about 40 μg, about 40 μg to about 50 μg, about 50 μg to about 60 μg, about 60 μg to about 70 μg, about 70 μg to about 80 μg, about 80 μg to about 90 μg, about 90 μg to about 100 μg, about 100 μg to about 110 μg, about 110 μg to about 120 μg, about 120 μg to about 130 μg, about 130 μg to about 140 μg, about 140 μg to about 150 μg, about 150 μg to about 200 μg, about 200 μg to about 250 μg, about 250 μg to about 300 μg, about 300 μg to about 350 μg, about 350 μg to about 400 μg, about 400 μg to about 450 μg, or about 450 μg to about 500 μg of the polypeptide or virus-like particle are administered.

In some embodiments, an immunogenic composition provided herein is administered as a booster of another RSV (or hMPV) vaccine, for example, a live-attenuated RSV (or hMPV) vaccine, an RSV (or hMPV)-A vaccine, and RSV (or hMPV)-B vaccine, or a bivalent RSV (or hMPV)-A/B vaccine. In some embodiments, the administering comprises administering a first dose and a second dose of the immunogenic composition, wherein the second dose is administered about 2 weeks to about 12 weeks, or about 4 weeks to about 12 weeks after the first dose is administered. In various further embodiments, the second dose is administered about 2, 3, 4, 5, 6, 7, 8, 9, 10, 11, or 12 weeks, about 1 month, about 2 months, about 3 months, about 4 months, about 5 months, about 6 months, about 9 months, about 12 months, about 18 months, about 2 years, about 3 years, about 4 years, or about 5 years after the first dose. In another embodiment, three doses may be administered, with a second dose administered about 1, 2, 3, 4, 5, 6, 7, 8, 9, 10, 11, or 12 weeks, about 1 month, about 2 months, about 3 months, about 4 months, about 5 months, about 6 months, about 9 months, about 12 months, about 18 months, about 2 years, about 3 years, about 4 years, or about 5 years after the first dose, and the third dose administered about 1, 2, 3, 4, 5, 6, 7, 8, 9, 10, 11, or 12 weeks, about 1 month, about 2 months, about 3 months, about 4 months, about 5 months, about 6 months, about 9 months, about 12 months, about 18 months, about 2 years, about 3 years, about 4 years, or about 5 years after the second dose. The second dose may be an RSV (or hMPV) booster dose.

In some embodiments, more than two doses of the immunogenic composition are administered. In some embodiments, the first dose and the second dose of the immunogenic composition are administered within about 2, 3, 4, 5, 6, 7, 8, 9, 10, 11, or 12 weeks, about 1 month, about 2 months, about 3 months, about 4 months, about 5 months, about 6 months, about 9 months, or about 12 months of each other, and a third dose is administered about 1 year, about 2 years, about 3 years, about 4 years, or about 5 years after the second dose. In some embodiments, the first dose and the second dose of the immunogenic composition are administered within about 2, 3, 4, 5, 6, 7, 8, 9, 10, 11, or 12 weeks, about 1 month, about 2 months, about 3 months, about 4 months, about 5 months, about 6 months, about 9 months, or about 12 months of each other, and subsequent doses are administered in regular intervals of about 1, 2, 3, 4 or 5 years.

In some embodiments, the subject has previously been infected with RSV (or hMPV) (e.g., RSV (or hMPV)-A and/or RSV (or hMPV)-B). In another embodiment of the methods, the subject is infected with RSV (or hMPV) (e.g., RSV (or hMPV)-A and/or RSV (or hMPV)-B) at the time of being administered a pharmaceutical composition provided herein, wherein the administering elicits an immune response against RSV (or hMPV) (e.g., RSV (or hMPV)-A and/or RSV (or hMPV)-B) in the subject that treats the RSV (or hMPV) infection (e.g., RSV (or hMPV)-A infection and/or RSV (or hMPV)-B infection) in the subject. When the method comprises treating an RSV (or hMPV) infection (e.g., RSV (or hMPV)-A infection and/or RSV (or hMPV)-B infection), the immunogenic compositions are administered to a subject that has already been infected with RSV (or hMPV) (e.g., RSV (or hMPV)-A and/or RSV (or hMPV)-B), and/or who is suffering from symptoms (such as described above) indicating that the subject is likely to have been infected with RSV (or hMPV) (e.g., RSV (or hMPV)-A and/or RSV (or hMPV)-B).

RSV (or hMPV) infection (e.g., RSV (or hMPV)-A infection and/or RSV (or hMPV)-B infection) may be diagnosed using any PCR-based test or antigen-based test known in the art. In some embodiments, the subject has antibodies against RSV (or hMPV). Anti-RSV (or hMPV) antibodies (e.g., RSV (or hMPV)-A antibodies and/or RSV (or hMPV)-B antibodies) may be detected using any serological test known in the art. In preferred embodiments, the compositions and methods disclosed herein prevent disease following infection with RSV (or hMPV) subtypes A and B in older adults.

In some embodiments, protein complexes and pharmaceutical compositions of the disclosure provide sustained durability (persistence) of neutralizing antibodies up to 180 days after vaccination. In some embodiments, protein complexes and pharmaceutical compositions of the disclosure provide sustained durability (persistence) of RSV-A and/or RSV-B neutralizing antibodies up to 20 days, up to 40 days, up to 60 days, up to 80 days, up to 100 days, up to 120 days, up to 140 days, up to 160 days, up to 180 days, up to 200 days, up to 250 days, up to 300 days, up to 350 days, up to 365 days, up to 400 days, up to 450 days, or up to 500 days after vaccination.

In some embodiments, protein complexes and pharmaceutical compositions of the disclosure provide sustained durability of about 40%, about 45%, about 50%, about 55%, about 60%, about 70%, about 80%, about 90%, about 95%, or about 98% at 180 days after vaccination relative to the Geometric mean titers (GMT) of RSV-A and/or RSV-B neutralizing antibodies at 28 days after vaccination.

In some embodiments, protein complexes and pharmaceutical compositions of the disclosure provide alum adjuvant-independent durability of RSV-A and/or RSV-B neutralizing antibodies.

In some embodiments, protein complexes and pharmaceutical compositions of the disclosure provide sustained durability of 3-fold, 4-fold, 5-fold, 6-fold, 7-fold, 8-fold, 9-fold, or 10-fold above baseline at 180 days after vaccination relative to the Geometric mean titers (GMT) of RSV-A and/or RSV-B neutralizing antibodies at 28 days after vaccination. In some embodiments, protein complexes and pharmaceutical compositions of the disclosure provide sustained durability of 3-fold, 4-fold, 5-fold, 6-fold, 7-fold, 8-fold, 9-fold, or 10-fold above baseline at 180 days after vaccination relative to the Geometric mean titers (GMT) of hMPV-A and/or hMPV-B neutralizing antibodies at 28 days after vaccination.

### Methods of Manufacture

In one aspect, the disclosure provides a method of manufacturing a vaccine, comprising culturing the host cell described herein in a culture medium so that the host cell secretes the antigen into the culture media; optionally purifying the antigen from the culture media; mixing the antigen with a second component, wherein the second component multimerizes with the antigen to form a virus-like particle; and optionally purifying the virus-like particle.

### Kits and Unit Doses

The disclosure further provides kits, which may be used to prepare the virus-like particles and compositions of the disclosure. In some embodiment, a kit provided herein comprises a first component and a second component as disclosed herein, and instructions for use in a method of the disclosure. In some embodiment, a kit comprises one or more unit doses as disclosed herein, and instructions for use in a method of the disclosure. In some embodiments, the kit comprises a vial comprising a single dose of a pharmaceutical composition provided herein. In some embodiments, a kit comprises a vial comprising multiple doses provided herein. In some embodiments, a kit further comprises instructions for use of the pharmaceutical composition. In some embodiments, a kit further comprises a diluent for preparing dilutions of the pharmaceutical composition prior to administration. In some embodiments, the pharmaceutical composition comprises an adjuvant. In some embodiments, a kit comprises a pharmaceutical composition and an adjuvant which must be mixed prior to administration.

Also provided herein are unit doses of the pharmaceutical composition described herein. In some embodiments, the unit dose comprises about 1 μg to about 5 μg, about 5 μg to about 10 μg, about 10 μg to about 15 μg, about 15 μg to about 20 μg, about 20 μg to about 30 μg, about 30 μg to about 40 μg, about 40 μg to about 50 μg, about 50 μg to about 60 μg, about 60 μg to about 70 μg, about 70 μg to about 80 μg, about 80 μg to about 90 μg, about 90 μg to about 100 μg, about 100 μg to about 110 μg, about 110 μg to about 120 μg, about 120 μg to about 130 μg, about 130 μg to about 140 μg, about 140 μg to about 150 μg, about 150 μg to about 175 μg, about 175 μg to about 200 μg, about 200 μg to about 250 μg, about 250 μg to about 300 μg, about 300 μg to about 350 μg, about 350 μg to about 400 μg, about 400 μg to about 450 μg, or about 450 μg to about 500 μg of protein complex. In some embodiments, the unit dose comprises about 1 μg, about 2 μg, about 5 μg, about 10 μg, about 15 μg, about 25 μg, about 50 μg, about 75 μg, about 100 μg, about 125 μg, about 150 μg, about 175 μg, about 200 μg, about 225 μg, about 250 μg, about 275 μg, about 300 μg, about 325 μg, about 350 μg, about 375 μg, about 400 μg, or about 500 μg of the VLP or of each VLP individually. In some embodiments, the unit dose comprises, 25 μg, 50 μg, 75 μg, 100 μg, 125 μg, 150 μg, 175 μg, 200 μg, 225 μg, 250 μg, 275 μg, 300 μg, 325 μg, 350 μg, 400 μg, or 500 μg of the VLP or of each VLP individually. The abbreviation “μg” may be used interchangeably with the abbreviation “mcg” to refer to micrograms of a substance.

In some embodiments, the unit dose comprises about 75 μg of the first VLP (for RSV) and about 75 μg of the second VLP (for hMPV).

In some embodiments, the unit dose comprises about 75 μg of the first VLP (for RSV) and about 100 μg of the second VLP (for hMPV).

In some embodiments, the unit dose comprises about 75 μg of the first VLP (for RSV) and about 150 μg of the second VLP (for hMPV).

In some embodiments, the unit dose comprises about 75 μg of the first VLP (for RSV) and about 200 μg of the second VLP (for hMPV).

In some embodiments, the unit dose comprises about 75 μg of the first VLP (for RSV) and about 225 μg of the second VLP (for hMPV).

In some embodiments, the unit dose comprises about 75 μg of the first VLP (for RSV) and about 250 μg of the second VLP (for hMPV).

In some embodiments, the unit dose comprises about 75 μg of the first VLP (for RSV) and about 300 μg of the second VLP (for hMPV).

In some embodiments, the unit dose comprises about 100 μg of the first VLP (for RSV) and about 75 μg of the second VLP (for hMPV).

In some embodiments, the unit dose comprises about 100 μg of the first VLP (for RSV) and about 100 μg of the second VLP (for hMPV).

In some embodiments, the unit dose comprises about 100 μg of the first VLP (for RSV) and about 150 μg of the second VLP (for hMPV).

In some embodiments, the unit dose comprises about 100 μg of the first VLP (for RSV) and about 200 μg of the second VLP (for hMPV).

In some embodiments, the unit dose comprises about 100 μg of the first VLP (for RSV) and about 225 μg of the second VLP (for hMPV).

In some embodiments, the unit dose comprises about 100 μg of the first VLP (for RSV) and about 250 μg of the second VLP (for hMPV).

In some embodiments, the unit dose comprises about 100 μg of the first VLP (for RSV) and about 300 μg of the second VLP (for hMPV).

In some embodiments, the unit dose comprises about 150 μg of the first VLP (for RSV) and about 75 μg of the second VLP (for hMPV).

In some embodiments, the unit dose comprises about 150 μg of the first VLP (for RSV) and about 100 μg of the second VLP (for hMPV).

In some embodiments, the unit dose comprises about 150 μg of the first VLP (for RSV) and about 150 μg of the second VLP (for hMPV).

In some embodiments, the unit dose comprises about 150 μg of the first VLP (for RSV) and about 200 μg of the second VLP (for hMPV).

In some embodiments, the unit dose comprises about 150 μg of the first VLP (for RSV) and about 225 μg of the second VLP (for hMPV).

In some embodiments, the unit dose comprises about 150 μg of the first VLP (for RSV) and about 250 μg of the second VLP (for hMPV).

In some embodiments, the unit dose comprises about 150 μg of the first VLP (for RSV) and about 300 μg of the second VLP (for hMPV).

In some embodiments, the unit dose comprises about 200 μg of the first VLP (for RSV) and about 75 μg of the second VLP (for hMPV).

In some embodiments, the unit dose comprises about 200 μg of the first VLP (for RSV) and about 100 μg of the second VLP (for hMPV).

In some embodiments, the unit dose comprises about 200 μg of the first VLP (for RSV) and about 150 μg of the second VLP (for hMPV).

In some embodiments, the unit dose comprises about 200 μg of the first VLP (for RSV) and about 200 μg of the second VLP (for hMPV).

In some embodiments, the unit dose comprises about 200 μg of the first VLP (for RSV) and about 225 μg of the second VLP (for hMPV).

In some embodiments, the unit dose comprises about 200 μg of the first VLP (for RSV) and about 250 μg of the second VLP (for hMPV).

In some embodiments, the unit dose comprises about 200 μg of the first VLP (for RSV) and about 300 μg of the second VLP (for hMPV).

In some embodiments, the unit dose comprises about 225 μg of the first VLP (for RSV) and about 75 μg of the second VLP (for hMPV).

In some embodiments, the unit dose comprises about 225 μg of the first VLP (for RSV) and about 100 μg of the second VLP (for hMPV).

In some embodiments, the unit dose comprises about 225 μg of the first VLP (for RSV) and about 150 μg of the second VLP (for hMPV).

In some embodiments, the unit dose comprises about 225 μg of the first VLP (for RSV) and about 200 μg of the second VLP (for hMPV).

In some embodiments, the unit dose comprises about 225 μg of the first VLP (for RSV) and about 225 μg of the second VLP (for hMPV).

In some embodiments, the unit dose comprises about 225 μg of the first VLP (for RSV) and about 250 μg of the second VLP (for hMPV).

In some embodiments, the unit dose comprises about 225 μg of the first VLP (for RSV) and about 300 μg of the second VLP (for hMPV).

In some embodiments, the unit dose comprises about 250 μg of the first VLP (for RSV) and about 75 μg of the second VLP (for hMPV).

In some embodiments, the unit dose comprises about 250 μg of the first VLP (for RSV) and about 100 μg of the second VLP (for hMPV).

In some embodiments, the unit dose comprises about 250 μg of the first VLP (for RSV) and about 150 μg of the second VLP (for hMPV).

In some embodiments, the unit dose comprises about 250 μg of the first VLP (for RSV) and about 200 μg of the second VLP (for hMPV).

In some embodiments, the unit dose comprises about 250 μg of the first VLP (for RSV) and about 225 μg of the second VLP (for hMPV).

In some embodiments, the unit dose comprises about 250 μg of the first VLP (for RSV) and about 250 μg of the second VLP (for hMPV).

In some embodiments, the unit dose comprises about 250 μg of the first VLP (for RSV) and about 300 μg of the second VLP (for hMPV).

In some embodiments, the unit dose comprises about 300 μg of the first VLP (for RSV) and about 75 μg of the second VLP (for hMPV).

In some embodiments, the unit dose comprises about 300 μg of the first VLP (for RSV) and about 100 μg of the second VLP (for hMPV).

In some embodiments, the unit dose comprises about 300 μg of the first VLP (for RSV) and about 150 μg of the second VLP (for hMPV).

In some embodiments, the unit dose comprises about 300 μg of the first VLP (for RSV) and about 200 μg of the second VLP (for hMPV).

In some embodiments, the unit dose comprises about 300 μg of the first VLP (for RSV) and about 225 μg of the second VLP (for hMPV).

In some embodiments, the unit dose comprises about 300 μg of the first VLP (for RSV) and about 250 μg of the second VLP (for hMPV).

In some embodiments, the unit dose comprises about 300 μg of the first VLP (for RSV) and about 300 μg of the second VLP (for hMPV).

In some embodiments, the unit dose comprises between about 5 μg and about 100 μg of the first VLP, between about 10 μg and about 90 μg of the first VLP, between about 20 μg and about 80 μg of the first VLP, between about 30 μg and about 70 μg of the first VLP, between about 40 μg and about 60 μg of the first VLP, or between about 50 μg and about 80 μg of the first VLP (for RSV).

In some embodiments, the unit dose comprises between about 5 μg and about 100 μg of the first VLP, between about 10 μg and about 90 μg of the first VLP, between about 20 μg and about 80 μg of the first VLP, between about 30 μg and about 70 μg of the first VLP, between about 40 μg and about 60 μg of the first VLP, and/or between about 50 μg and about 80 μg of the second VLP (for hMPV).

In some embodiments, the unit dose comprises between about 50 μg and about 180 μg of the first VLP, between about 60 μg and about 170 μg of the first VLP, between about 80 μg and about 150 μg of the first VLP, between about 70 μg and about 155 μg of the first VLP, between about 90 μg and about 160 μg of the first VLP, and/or between about 100 μg and about 170 μg of the second VLP (for hMPV).

In some embodiments, the unit dose comprises between about 130 μg and about 300 μg of the first VLP, between about 150 μg and about 280 μg of the first VLP, between about 170 μg and about 260 μg of the first VLP, between about 190 μg and about 240 μg of the first VLP, between about 200 μg and about 230 μg of the first VLP, and/or between about 210 μg and about 225 μg of the second VLP (for hMPV).

In some embodiments, the unit dose comprises between about 130 μg and about 300 μg of the first VLP, between about 150 μg and about 280 μg of the first VLP, between about 170 μg and about 260 μg of the first VLP, between about 190 μg and about 240 μg of the first VLP, between about 200 μg and about 230 μg of the first VLP, and between about 130 μg and about 300 μg of the second VLP, between about 150 μg and about 280 μg of the second VLP, between about 170 μg and about 260 μg of the second VLP, between about 190 μg and about 240 μg of the second VLP, between about or 200 μg and about 230 μg of the second VLP.

In some embodiments, the unit dose comprises about 75 μg of the first VLP (for RSV) and 75 μg of the second VLP (for hMPV). In some embodiments, the unit dose comprises about 75 μg of the first VLP (for RSV) and 225 μg of the second VLP (for hMPV). In some embodiments, the unit dose comprises about 225 μg of the first VLP (for RSV) and 75 μg of the second VLP (for hMPV). In some embodiments, the unit dose comprises about 75 μg of the first VLP (for RSV) and 150 μg of the second VLP (for hMPV). In some embodiments, the unit dose comprises about 150 μg of the first VLP (for RSV) and 75 μg of the second VLP (for hMPV). In some embodiments, the unit dose comprises about 150 μg of the first VLP (for RSV) and 150 μg of the second VLP (for hMPV).

In some embodiments, the unit dose comprises about 5 μg, about 10 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 90 μg, about 100 μg, about 110 μg, about 120 μg, about 130 μg, about 140 μg, about 150 μg, about 175 μg, or about 200 μg of the first VLP (for RSV).

In some embodiments, the unit dose comprises about 5 μg, about 10 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 90 μg, about 100 μg, about 110 μg, about 120 μg, about 130 μg, about 140 μg, about 150 μg, about 175 μg, or about 200 μg of the second VLP (for hMPV).

In some embodiments, the unit dose comprises about 5 μg, about 10 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 90 μg, about 100 μg, about 110 μg, about 120 μg, about 130 μg, about 140 μg, about 150 μg, about 175 μg, or about 200 μg of the first VLP (for RSV) and about 5 μg, about 10 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 90 μg, about 100 μg, about 110 μg, about 120 μg, about 130 μg, about 140 μg, about 150 μg, about 175 μg, or about 200 μg of the second VLP (for hMPV).

In some embodiments, the unit dose comprises about 5 μg, about 10 μg, about 15 μg, about 20 μg, about 25 μg, about 30 μg, about 35 μg, about 40 μg, about 45 μg, about 50 μg, about 55 μg, about 60 μg, about 65 μg, about 70 μg, or about 75 μg, about 100 μg, about 125 μg, about 150 μg, about 175 μg, about 200 μg, about 225 μg, about 250 μg, about 275 μg, about 300 μg of the first VLP (for RSV).

In some embodiments, the unit dose comprises about 5 μg, about 10 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 100 μg, about 125 μg, about 150 μg, about 175 μg, about 200 μg, about 225 μg, about 250 μg, about 275 μg, about 300 μg of the second VLP (for hMPV).

In some embodiments, the unit dose comprises about 5 μg, about 10 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 100 μg, about 125 μg, about 150 μg, about 175 μg, about 200 μg, about 225 μg, about 250 μg, about 275 μg, about 300 μg of the first VLP (for RSV) and about 5 μg, about 10 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 100 μg, about 125 μg, about 150 μg, about 175 μg, about 200 μg, about 225 μg, about 250 μg, about 275 μg, about 300 μg of the second VLP (for hMPV).

### EXAMPLES

### Example 1

This example illustrates production of a two-component, icosahedral VLP intended for use as a vaccine for hMPV virus. As first component A, the vaccine uses a polypeptide antigen composed of the ectodomain of hMPV F protein, C-terminally fused to the I53-50A protein (SEQ ID NO: 144). As component B, the VLP uses the I53-50B protein. It is known that I53-50A and I53-50B spontaneously assemble in 3:5 ratio to form a VLP having icosahedral (I53) symmetry, as shown in FIGS. 1A and 1B.

The expression level of hMPV F proteins fused to CompA were determined. Human codon-optimized polynucleotide sequences were made by gene synthesis and cloned into expression vectors. Each expression vector was individually expressed by transient transfection in Expi293 cells. Supernatants were collected four days after transfection.

To determine if the recombinant expressed fusion proteins were secreted into the supernatant, a Western blot using an anti-His6 monoclonal antibody was performed. Conditioned media were diluted a minimum of 10-fold in 2× Laemmli loading buffer (Bio-Rad) with 5% (v/v) β-mercaptoethanol and heated to 95° C. for 10 minutes and 10 μl run on a gel. Reference samples of recombinant His-tagged human serum albumin (HSA) were prepared by an initial dilution to 0.01 mg/mL, followed by a 2-fold dilution series to obtain a standard curve from 6.25 to 100 ng in a 10 μL loading volume in 2× Laemmli loading buffer. Samples were loaded onto a NuPAGE 4-12% Bis-Tris protein gel and run for 45 minutes at 150V. Proteins were transferred onto an Immuno-Blot PDVF membrane (Bio-Rad). Following transfer, the blot was blocked with 3% BSA (w/v) in TBST for 1 hour at room temperature with shaking. An anti-His, HRP-conjugated antibody (R&D Systems MAB050H) was diluted 1:8,000 in 3% BSA (w/v) in TBST and incubated with the membrane for 1 hour at room temperature. The membrane was then washed three times for five minutes using TBST, then His-tagged protein was detected using a luminol chemiluminescent substrate (VisiGlo, VWR) and captured using a 10-20 second exposure time on a UVP Chemstudio (Analytik Jena).

Table 4 shows the expression levels of hMPV F protein fused to CompA. Expression levels were assessed by visual inspection of Western blots and designated as no expression (X) or low expression (*) to high expression (****). The hMPV008, hMPV009, hMPV024, hMPV026, hMPV027, hMPV033, and hMPV034 CompA fusion proteins showed the highest expression levels in this system.

TABLE 4
hMPV F-CompA Fusion Proteins Tested for Expression in Expi293 cells.

| | | hMPV | CompA: | | |
|---|---|---|---|---|---|
| Construct | | strain (A | Ala or | C-term | Expression |
| Name | Mutations | or B) | Cys | Truncation | Level |
| hMPV001 | A185P, Q100R, | A | Ala | 481 | * |
| | S101R | | | | |
| hMPV002 | A185P, Q100R, | A | Ala | 472 | ** |
| | S101R | | | | |
| hMPV003 | A185P, Q100R, | A | Ala | 489 | * |
| | S101R | | | | |
| hMPV004 | Q100R, S101R | A | Ala | 472 | * |
| hMPV005 | A185P, T127C, | A | Ala | 472 | ** |
| | N153C, Q100R, | | | | |
| | S101R | | | | |
| hMPV006 | V84C, A140C, | B | Ala | 472 | ** |
| | A147C, A249C, | | | | |
| | N97G, P98G, | | | | |
| | R99G, Q100G, | | | | |
| | S101G, R102G | | | | |
| hMPV007 | V84C, A140C, | B | Ala | 488 | ** |
| | A147C, A249C, | | | | |
| | N97G,P98G, | | | | |
| | R99G, Q100G, | | | | |
| | S101G, R102G | | | | |
| hMPV008 | A63C, A140C, | B | Ala | 472 | **** |
| | A147C, K188C, | | | | |
| | K450C, S470C, | | | | |
| | N97G, P98G, | | | | |
| | R99G, Q100G, | | | | |
| | S101G, R102G | | | | |
| hMPV009 | A63C, A140C, | B | Ala | 488 | **** |
| | A147C, K188C, | | | | |
| | K450C, S470C, | | | | |
| | N97G, P98G, | | | | |
| | R99G, Q100G, | | | | |
| | S101G, R102G | | | | |
| hMPV010 | A63C, A140C, | A | Ala | 472 | X |
| | A147C, K188C, | | | | |
| | G106 deleted, | | | | |
| | N97G, P98G, | | | | |
| | R99G, Q100G, | | | | |
| | S101G, R102G | | | | |
| hMPV011 | A63C, A140C, | A | Ala | 488 | X |
| | A147C, K188C, | | | | |
| | G106 deleted, | | | | |
| | N97G, P98G, | | | | |
| | R99G, Q100G, | | | | |
| | S101G, R102G | | | | |
| hMPV012 | A113C, A120C, | B | Ala | 472 | X |
| | A339C, Q426C, | | | | |
| | T160F, I177L, | | | | |
| | Q100K, S101A | | | | |
| hMPV013 | A113C, A120C, | B | Ala | 488 | X |
| | A339C, Q426C, | | | | |
| | T160F, I177L, | | | | |
| | Q100K, S101A | | | | |
| hMPV014 | V84C, A140C, | B | Ala | 472 | ** |
| | A147C, A249C, | | | | |
| | Q100R, S101R | | | | |
| hMPV015 | A63C, A140C, | B | Ala | 472 | **** |
| | A147C, K188C, | | | | |
| | K450C, S470C, | | | | |
| | Q100R, S101R | | | | |
| hMPV016 | A63C, A140C, | A | Ala | 472 | X |
| | A147C, K188C, | | | | |
| | G106 deleted, | | | | |
| | Q100R, S101R | | | | |
| hMPV017 | A113C, A120C, | B | Ala | 472 | X |
| | A339C, Q426C, | | | | |
| | T160F, I177L, | | | | |
| | Q100R, S101R | | | | |
| hMPV018 | A185P, A113C, | A | Ala | 472 | ** |
| | A339C, Q100R, | | | | |
| | S101R | | | | |
| hMPV019 | A185P, T160F, | A | Ala | 472 | * |
| | I177L, Q100R, | | | | |
| | S101R | | | | |
| hMPV020 | A185P, A113C, | A | Ala | 472 | * |
| | A339C, T160F, | | | | |
| | I177L, Q100R, | | | | |
| | S101R | | | | |
| hMPV021 | A63C, K188C, | B | Ala | 472 | *** |
| | N97G, P98G, | | | | |
| | R99G, Q100G, | | | | |
| | S101G, R102G | | | | |
| hMPV022 | A63C, K188C, | B | Ala | 472 | *** |
| | K450A, S470A, | | | | |
| | N97G, P98G, | | | | |
| | R99G, Q100G, | | | | |
| | S101G, R102G | | | | |
| hMPV023 | A63C, A140C, | A | Ala | 472 | ** |
| | A147C, K188C, | | | | |
| | G294E, N97G, | | | | |
| | P98G, R99G, | | | | |
| | Q100G, S101G, | | | | |
| | R102G | | | | |
| hMPV024 | A63C, A140C, | A | Ala | 472 | **** |
| | A147C, K188C, | | | | |
| | K450C, S470C, | | | | |
| | N97G, P98G, | | | | |
| | R99G, Q100G, | | | | |
| | S101G, R102G, | | | | |
| | G294E | | | | |
| hMPV025 | A63C, K188C, | A | Ala | 472 | ** |
| | N97G, P98G, | | | | |
| | R99G, Q100G, | | | | |
| | S101G, R102G, | | | | |
| | G294E | | | | |
| hMPV026 | T127C, N153C, | A | Ala | 472 | **** |
| | T365C, V463C, | | | | |
| | A185P, L219K, | | | | |
| | V231I, G294E, | | | | |
| | H368N, Q100R, | | | | |
| | S101R | | | | |
| hMPV027 | T127C, N153C, | A | Ala | 490 | **** |
| | T365C, V463C, | | | | |
| | A185P, L219K, | | | | |
| | V231I, G294E, | | | | |
| | H368N, Q100R, | | | | |
| | S101R | | | | |
| hMPV028 | A63C, A140C, | B | Cys | 472 | * |
| | A147C, K188C, | | | | |
| | K450C, S470C, | | | | |
| | N97G, P98G, | | | | |
| | R99G, Q100G, | | | | |
| | S101G, R102G | | | | |
| hMPV029 | T127C, N153C, | A | Cys | 472 | * |
| | T365C, V463C, | | | | |
| | A185P, L219K, | | | | |
| | V231I, G294E, | | | | |
| | H368N, Q100R, | | | | |
| | S101R | | | | |
| hMPV030 | T127C, N153C, | A | Cys | 490 | * |
| | T365C, V463C, | | | | |
| | A185P, L219K, | | | | |
| | V231I, G294E, | | | | |
| | H368N, Q100R, | | | | |
| | S101R | | | | |
| hMPV031 | V84C, A140C, | B | Cys | 472 | * |
| | A147C, A249C, | | | | |
| | N97G, P98G, | | | | |
| | R99G, Q100G, | | | | |
| | S101G, R102G | | | | |
| hMPV033 | T127C, N153C, | A | Ala | 472 | **** |
| | T365C, V463C, | | | | |
| | A185P, L219K, | | | | |
| | V231I, G294E, | | | | |
| | N97G, P98G, | | | | |
| | R99G, Q100G, | | | | |
| | H368N, S101G, | | | | |
| | R102G | | | | |
| hMPV034 | T127C, N153C, | A | Ala | 490 | **** |
| | T365C, V463C, | | | | |
| | A185P, L219K, | | | | |
| | V231I, G294E, | | | | |
| | N97G,P98G, | | | | |
| | R99G, H368N, | | | | |
| | Q100G, S101G, | | | | |
| | R102G | | | | |

### Example 2

In this example, conditioned media containing the hMPV F-CompA fusion protein were characterized using antibody binding activity to determine the conformation of the hMPV F protein. The fusion proteins hMPV005, hMPV008, hMPV021, hMPV024, hMPV026, hMPV027, hMPV027C, and hMPV033 were evaluated (Table 5). Constructs hMPV026 and hMPV027 contain a furin cleavage site that was not cleaved in the Expi293 transient transfections. Upon co-transfection of human furin, the expressed protein was properly cleaved and termed hMPV026C or hMPV027C to represent the cleaved form.

To characterize the conformation of the recombinant expressed hMPV F protein fused to CompA, an ELISA was performed using 15 monoclonal antibodies that bind to specific forms of the hMPV F protein ectodomain.

Wells of a 96 well plate were coated with 200 ng of each antibody, or 50 ng for MPE8, in 100 μL 50 mM sodium carbonate-bicarbonate buffer, pH 9.6, at 4° C. overnight. Plates were washed 3× with 300 μl PBS with 0.05% Tween 20. After blocking with 150 μl PBS with 1% BSA for 1 hour at room temperature, the plates were emptied by tapping onto an absorbent pad. Conditioned media supernatants from the Expi293 transfections of each construct were diluted based on semi-quantitative Western blots such that antigen concentrations would bracket the expected EC₅₀ of the antigen (from Mas data). Three dilutions for each construct were plated at 100 μL per well for each antibody and the plates were incubated for 1 hour at room temperature. Plates were washed 6× with 300 μl PBS with 0.05% Tween 20. An anti-His monoclonal antibody (R&D Systems MAB050H) was diluted 1:15,000 and 100 μL was added to each well and the plate incubated for 1 hour at room temperature. After washing the plates 6× with 300 μl PBS with 0.05% Tween 20, 100 μl TMB substrate was added and the plate was developed in the dark for 10 minutes. The reaction was stopped by addition of 100 μl 0.6 N H₂SO₄ and absorbance was measured at 450 nm on a plate reader.

The postfusion specific antibodies bound hMPV021 suggesting this construct has a postfusion conformation. The other constructs did not bind the postfusion specific Abs and did bind MPE8 along with multiple Abs that recognize both prefusion and postfusion conformation. This data suggests the fusion proteins have prefusion conformation characteristics.

### Example 3

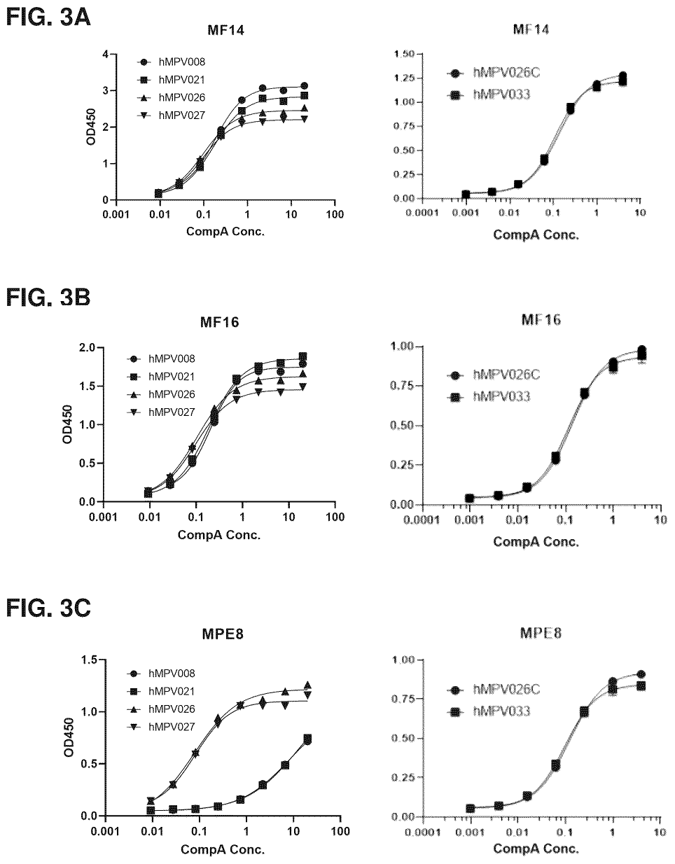

Binding activity seen with purified hMPV F-CompA fusion proteins. FIGS. 3A-3C show binding with known concentrations of purified component A (CompA) fusions—that is, hMPV F protein ectodomains N-terminally fused to I53-50A (SEQ ID NO: 144) or I53-50A ΔCys (SEQ ID NO: 145). MF14, MPE8, and MF16 recognize antigenic sites II, III, and IV respectively and these antigenic sites are the primary sites recognized by neutralizing antibodies. In addition, MPE8 recognizes preferentially the prefusion form of hMPV F protein. Wells of a 96 well plate were coated with 50 ng of MPE8 and 100 ng of MF14 and MF16 in 100 μL 50 mM sodium carbonate-bicarbonate buffer, pH 9.6, at 4° C. overnight. Plates were washed 3× with 300 μl PBS with 0.05% Tween 20. After blocking with 150 μl PBS with 1% BSA for 1 hour at room temperature, the plates were emptied by tapping onto an absorbent pad. Purified samples of each construct were diluted to 40 μg/mL (hMPV008, 021, 026, and 027) or 4 μg/mL (hMPV026C and 033), followed by a 3-fold or 4-fold dilution series respectively in PBS with 1% BSA and plated at 100 μL per well and the plates were incubated for 1 hour at room temperature. Plates were washed 6× with 300 μl PBS with 0.05% Tween 20. An anti-His monoclonal antibody (R&D Systems MAB050H) was diluted 1:15,000 and 100 μL was added to each well and the plate incubated for 1 hour at room temperature. After washing the plates 6× with 300 μl PBS with 0.05% Tween 20, 100 μl TMB substrate was added and the plate was developed in the dark for 10 minutes. The reaction was stopped by addition of 100μl 0.6 N H₂SO₄ and absorbance was measured at 450 nm on a plate reader. Binding to MF14 (site II, FIG. 3A) and MF16 (site IV, FIG. 3B) are similar but binding with MPE8 (site III, FIG. 3C) to hMPV026, hMPV027 and hMPV033 has a higher EC₅₀ than with hMPV008 and hMPV021 (Table 6). This suggests that site III, a neutralizing antibody recognition site, is more conserved in hMPV026, hMPV027, and hMPV033 and these constructs more preferentially adopt the prefusion conformation than hMPV008 and hMPV021.

TABLE 6
Neutralizing Antibody Binding EC₅₀ Values

| | EC₅₀ | | |
|---|---|---|---|
| Construct: | MPE8 | MF14 | MF16 |
| hMPV008 | 8.19E+00 | 1.78E−01 | 1.99E−01 |
| hMPV021 | 1.28E+01 | 1.66E−01 | 1.86E−01 |
| hMPV026 | 7.70E−02 | 9.57E−02 | 1.01E−01 |
| hMPV026C | 1.24E−01 | 1.36E−01 | 1.40E−01 |
| hMPV027 | 7.96E−02 | 9.37E−02 | 9.65E−02 |
| hMPV033 | 9.68E−02 | 1.08E−01 | 1.17E−01 |

### Example 4

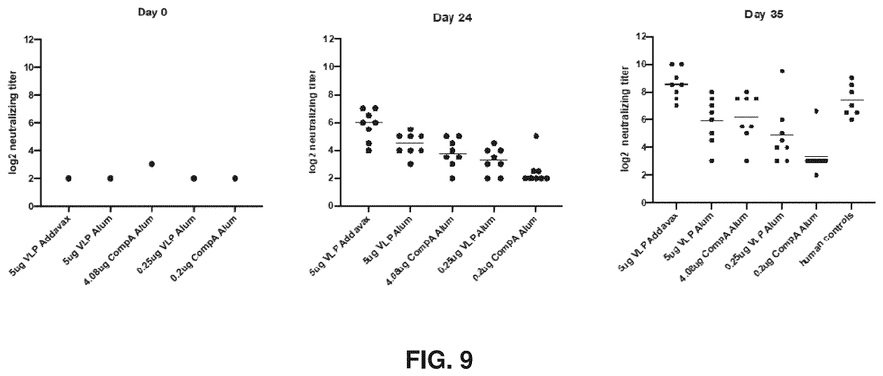

In this example, assembled hMPV008 VLPs or hMPV008 CompA alone were injected intramuscular in both hind legs of female BALB/c mice on day 0 and day 21. The quantity and formulation of each group is listed in Table 7. Blood draws were collected on days 0, 24 and 35 and processed into serum. Day 0 serum samples were pooled by group. All serum samples were evaluated in a hMPV neutralization antibody titer assay (FIG. 9). hMPV008 formulations with adjuvants Alhydrogel (Alum) or Addavax (squalene oil-in-water emulsion) induced measurable neutralizing titers at all dosages on day 24 and 35 and were comparable to titers of healthy adult human serum (FIG. 9). These results show that hMPV008 VLPs induce robust neutralizing titers comparable to healthy human control sera.

TABLE 7
Quantity and Formulations of Injected VLPs

| Antigen | Dose | Formulation |
|---|---|---|
| hMPV008 VLP | 5 μg | Addavax |
| hMPV008 VLP | 5 μg | Alhydrogel |
| hMPV008 VLP | 0.25 μg | Alhydrogel |
| hMPV008 CompA | 4.08 μg | Alhydrogel |
| hMPV008 CompA | 0.2 μg | Alhydrogel |

TABLE 5
Antibody Binding to Illustrative CompA Fusion Proteins

| | Ag | Pre/Post | Neut | | | | | hMPV026 | hMPV027 | hMPV02 | hMPV0 |
|---|---|---|---|---|---|---|---|---|---|---|---|
| Ab | site | Preference | A/B | hMPV005 | hMPV008 | hMPV021 | hMPV024 | ‡ | ‡ | 7C | 33 |
| MF1 | 6HB | post | No | low | NB | very high | low | NB | NB | NB | NB |
| MF2 | 6HB | post | No | low | NB | very high | low | NB | NB | NB | NB |
| MF3 | 6HB | post | No | low | NB | medium | NB | NB | NB | NB | NB |
| MF9 | Site | pre/post (B | A/B | very high | very high | very high | very high | very high | very high | very high | very |
| | II | preferential) | | | | | | | | | high |
| MF12 | Site | pre/post | A/B | very high | very high | very high | very high | very high | very high | very high | very |
| | II | | | | | | | | | | high |
| MF14 | Site | pre/post | A/B | very high | very high | very high | very high | very high | very high | very high | very |
| | II | | | | | | | | | | high |
| MF15 | Site | pre/post | A/B | very high | very high | very high | very high | very high | very high | very high | very |
| | II | | | | | | | | | | high |
| MF11 | Site | post | No | medium | low | very high | high | NB | NB | NB | NB |
| | IV | preferential | | | | | | | | | |
| MF16 | Site | pre/post | A/B | very high | very high | very high | high | very high | very high | very high | very |
| | IV | | | | | | | | | | high |
| MF20 | Site | pre/post (B | A/B | very high | medium | very high | medium | low | low | low | low |
| | IV | preferential) | | | | | | | | | |
| MF17 | Site | post | B | high | NB | high | low | low | low | low | low |
| | I | preferential | | | | | | | | | |
| MF18 | Site | post | B | very high | low | very high | medium | low | low | low | low |
| | I | preferential | | | | | | | | | |
| MF19 | Site | post | B | very high | medium | very high | medium | low | low | low | medium |
| | I | preferential | | | | | | | | | |
| MF10 | Site | Pre (A | A | very high | NB | NB | low | very high | very high | very high | very |
| | 0 | preferential) | | | | | | | | | high |
| MPE8 | Site | pre | A/B | high | high | high | high | high | high | high | high |
| | III | | | | | | | | | | |

* A very high response indicates OD450 > 2.0, a high response indicates an OD450 > 1.0 but less than 2.0, medium response indicates OD450 > 0.5 but < 1.0 and low response indicates OD450 > 0.1 but < 0.5 and NB (no binding) is < 0.1.
‡ Construct retains native furin cleavage site.

### Example 5

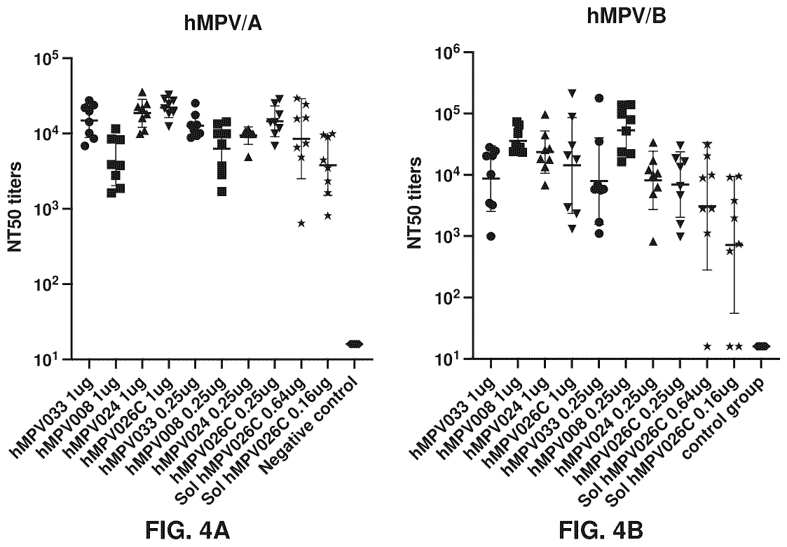

A study was undertaken in naïve BALB/c mice to explore the immunogenicity hMPV F protein mutants displayed on a 2-component virus-like particle (VLP). VLPs containing one of four different hMPV F protein mutants, hMPV008, hMPV024, hMPV026C, and hMPV033 or a soluble protein corresponding to hMPV026C were tested. Each group consisted of 8 female BALB/c mice. Mice were immunized on day 0 and day 21. All test articles were formulated with Addavax, an oil in water adjuvant. Two dosage levels (1 μg and 0.25 μg) or an equivalent antigen content of soluble protein were administered (Table 8). Neutralizing antibody titers against hMPV-A and hMPV-B were determined from day 35 serum (FIGS. 4A and 4B, respectively). The results demonstrated robust neutralizing titers were induced by all VLPs against both strains of hMPV. The soluble protein also induced titers against both hMPV strains at a lower level than the VLPs.

TABLE 8
Quantity and Formulations of Injected VLPs

| Antigen | Dose | Formulation |
|---|---|---|
| hMPV033 VLP | 1 μg | Addavax |
| hMPV008 VLP | 1 μg | Addavax |
| hMPV024 VLP | 1 μg | Addavax |
| hMPV026C VLP | 1 μg | Addavax |
| hMPV033 VLP | 0.25 μg | Addavax |
| hMPV008 VLP | 0.25 μg | Addavax |
| hMPV024 VLP | 0.25 μg | Addavax |
| hMPV026C VLP | 0.25 μg | Addavax |
| Soluble hMPV026C | 0.64 μg | Addavax |
| Soluble hMPV026C | 0.16 μg | Addavax |
| No treatment | N/A | N/A |

### Example 6

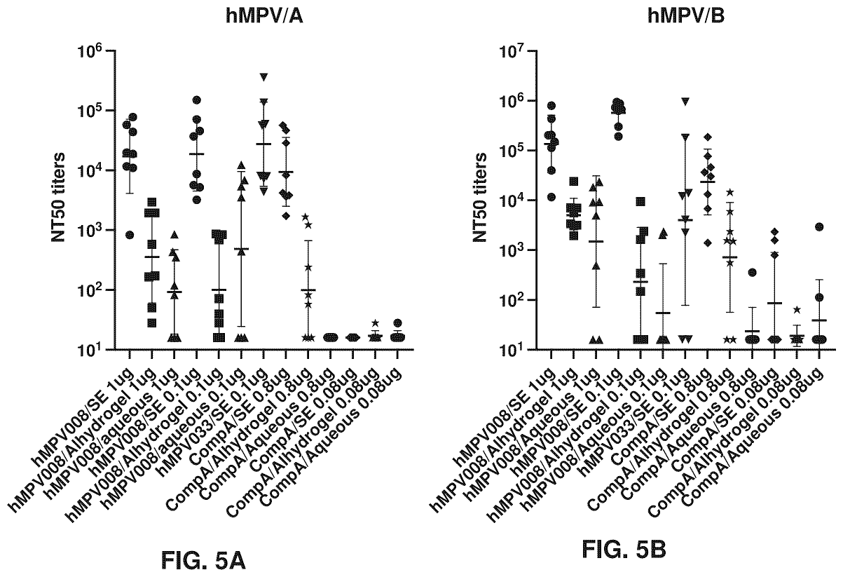

A study was undertaken in naïve mice to explore the immunogenicity of hMPV008 VLPs and the corresponding soluble protein, CompA-hMPV008. hMPV008 VLPs and CompA-hMPV008 were formulated in aqueous buffer, a squalene emulsion (SE), or Alhydrogel at two dosage levels (1 μg and 0.1 μg VLP and the equivalent antigen dose for CompA-hMPV008). In addition, one group with hMPV033 VLP (0.1 μg) formulated with SE was included (Table 9). Each group consisted of 8 female BALB/c mice. Mice were immunized on day 0 and day 21. Neutralizing antibody titers against hMPV-A and hMPV-B were determined from day 35 serum. Results demonstrated that hMPV008 VLPs formulated with SE resulted in higher titers than Alhydrogel or aqueous formulations (FIGS. 5A and 5B). VLPs induced higher neutralizing titers than the soluble protein CompA-hMPV008 with all formulations (FIGS. 5A and 5B).

TABLE 9
Quantity and Formulations of Injected VLPs

| Antigen | Dose | Formulation |
|---|---|---|
| hMPV008 VLP | 1 μg | SE |
| hMPV008 VLP | 1 μg | Alhydrogel |
| hMPV08 VLP | 1 μg | Aqueous |
| hMPV008 VLP | 0.1 μg | SE |
| hMPV008 VLP | 0.1 μg | Alhydrogel |
| hMPV008 VLP | 0.1 μg | Aqueous |
| CompA-hMPV008 | 0.81 μg | SE |
| CompA-hMPV008 | 0.81 μg | Alhydrogel |
| CompA-hMPV008 | 0.81 μg | Aqueous |
| CompA-hMPV008 | 0.081 μg | SE |
| CompA-hMPV008 | 0.081 μg | Alhydrogel |
| CompA-hMPV008 | 0.081 μg | Aqueous |
| hMPV033 | 0.1 μg | SE |

### Example 7: Bivalent Non-Interference Immunogenicity Study with hMPV F Protein (IVX-241) and RSV F Protein (IVX-121) VLPs

A study was undertaken in naïve BALB/c mice to explore the immunogenicity of VLPs displaying an hMPV F protein mutant, IVX-241 and an RSV F protein, IVX-121 when dosed as monovalent or bivalent formulations. The objective of the study was to evaluate various ratios of IVX-241 to IVX-121 and determine the neutralizing titers against RSV-A, RSV-B, hMPV-A and hMPV-B. Mice were immunized on day 0 and day 21 with IVX-121, IVX-241 or IVX-121+IVX-241 with dosage levels and formulation shown in Table 10. IVX-121 was administered at a dosage level of 1μg while IVX-241 was administered at dosage levels of 4, 2, 1, and 0.5 g. The study included a VLP displaying both the components incorporating RSV and hMPV F antigen mutants present on IVX-121 and IVX-241 (mosaic). This mosaic particle was administered at a dosage level of 2 g.

Blood samples were collected on days 0, 21, and 35 and processed to serum and the day 0 and 35 samples tested in RSV-A, RSV-B, hMPV-A, and hMPV-B specific neutralization assays. Neutralizing antibody titer data was plotted displaying geometric mean with geometric SD. Group statistical analyses were performed using nonparametric Mann-Whitney test (GraphPad Prism).

All animals were alert and responsive throughout the study with no test article or adjuvant related clinical observations.

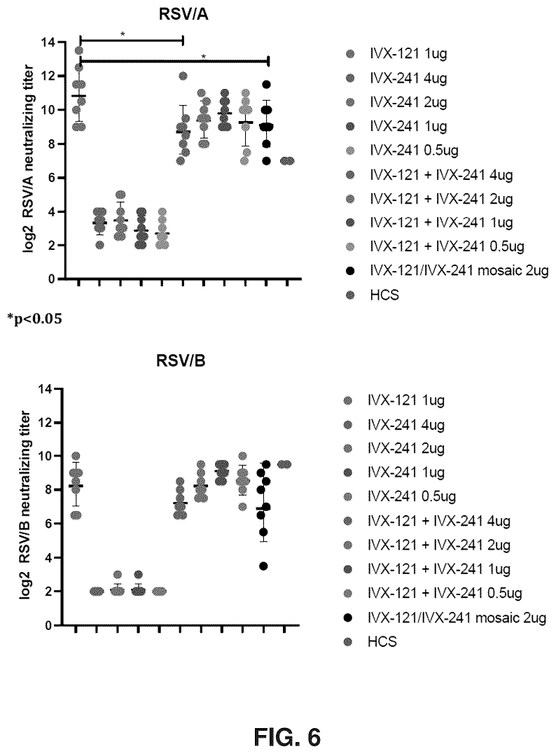

Sera from animals immunized with IVX-121 demonstrated significant increases in RSV-A and RSV-B neutralizing antibodies (FIG. 6) compared to pre-immune sera (Day 0 titers for all groups were below 2 log 2). The mean RSV-B titers were approximately 2.5 log 2 lower than RSV-A titers. Animals immunized with IVX-241 displayed minimal to no neutralizing titers against RSV-A or RSV-B (FIG. 6). The RSV-A neutralizing titers of animals administered IVX-121 and IVX-241 were slightly lower than animals dosed only with IVX-121 with statistical significance only seen between the bivalent group administered 4 μg IVX-241 and the mosaic VLP (FIG. 6). The IVX-121/IVX-241 mosaic VLP resulted in RSV-A and RSV-B titers that trended lower than IVX-121 alone but was not statistically significant. The convalescent human serum samples run in the RSV-A neutralizing titer assay displayed titers lower than the mean of the IVX-121 or IVX-121+IVX-241 immunized animals. However, the single serum run in duplicate for RSV-B had a higher titer than the IVX-121 groups.

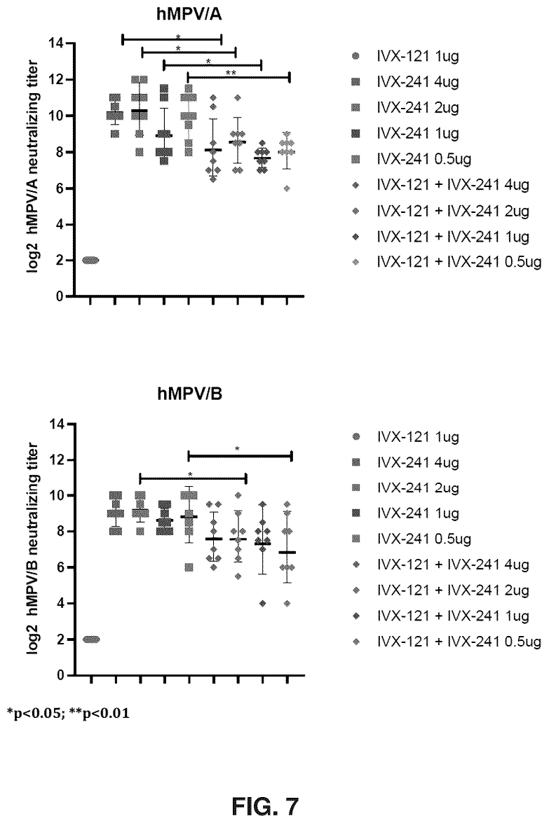

Sera from animals immunized with IVX-241 demonstrated significant increases in hMPV-A and hMPV-B neutralizing antibodies (FIG. 7) compared to pre-immune sera (Day 0 tiers for all groups were below 2 log 2). The response to all 4 doses of IVX-241 resulted in similar titers. Animals immunized with IVX-121 displayed hMPV-A and hMPV-B neutralizing titers similar to pre-immune sera (FIG. 7). The hMPV-A and hMPV-B neutralizing titers of animals administered IVX-121 and IVX-241 were approximately 1-2 log 2 lower than animals dosed only with IVX-241 (FIG. 7). The IVX-121/IVX-241 mosaic VLP resulted in hMPV-A and hMPV-B titers that trended higher than IVX-241 (1 μg) alone but was not statistically significant. The convalescent human serum samples run in the hMPV-A and hMPV-B neutralizing titer assay displayed titers equal to or lower than the mean of the IVX-241 or IVX121+IVX-241 immunized animals.

TABLE 10

| Antigen | Dose | | Formulation |
|---|---|---|---|
| IVX-121 | 1 | μg | Addavax |
| IVX-241 | 4 | μg | Addavax |
| IVX-241 | 2 | μg | Addavax |
| IVX-241 | 1 | μg | Addavax |
| IVX-241 | 0.5 | μg | Addavax |
| IVX-121 + IVX-241 | 1 + 4 | μg | Addavax |
| IVX-121 + IVX-241 | 1 + 2 | μg | Addavax |
| IVX-121 + IVX-241 | 1 + 1 | μg | Addavax |
| IVX-121 + IVX-241 | 1 + 0.5 | μg | Addavax |
| IVX-121/IVX-241 mosaic | 2 | μg | Addavax |

### Example 8: Protective Immunity Generated by Monovalent and Bivalent hMPV/RSV Combination Vaccine

A study was undertaken to assess and evaluate the ability of IVX-121, IVX-241, and IVX-121+IVX-241 candidate vaccines to protect against hMPV and RSV infection in cotton rats. The vaccine candidates were formulated with Addavax, an oil-in-water emulsion.

The study included sixty-four female cotton rats, distributed into 8 groups of 8 animals per group. Immunogens, dose levels, formulations details, and challenge virus are presented in Table 11.

Three control groups were administered saline and the groups were subsequently not challenged or challenged with RSV-Aor hMPV-A2. Groups were administered IVX-121 or IVX-241 candidate vaccines intramuscularly at a dosage level of 1.0 μg formulated in Addavax on Day 0 and Day 21 and subsequently challenged on Day 35 with RSV-A or hMPV-A2 respectively. Two groups were administered IVX-121+IVX-241 intramuscularly at a dosage level of 1.0 μg each VLP formulated in Addavax on Day 0 and Day 21 and subsequently challenged on Day 35 with RSV or hMPV respectively. One group was vaccinated with formalin inactivated (FI) hMPV and subsequently challenged with hMPV-A2 on Day 35. All animals were terminated on Day 40. Lung and nose homogenates were used for RSV-A and hMPV-A2 viral titer assessment. Serum samples were obtained at Days 0, 21 35, and 40 to measure RSV-A and hMPV-A neutralization antibody titers using a virus neutralization assay. Clinical observations were made daily, and animals were weighed weekly.

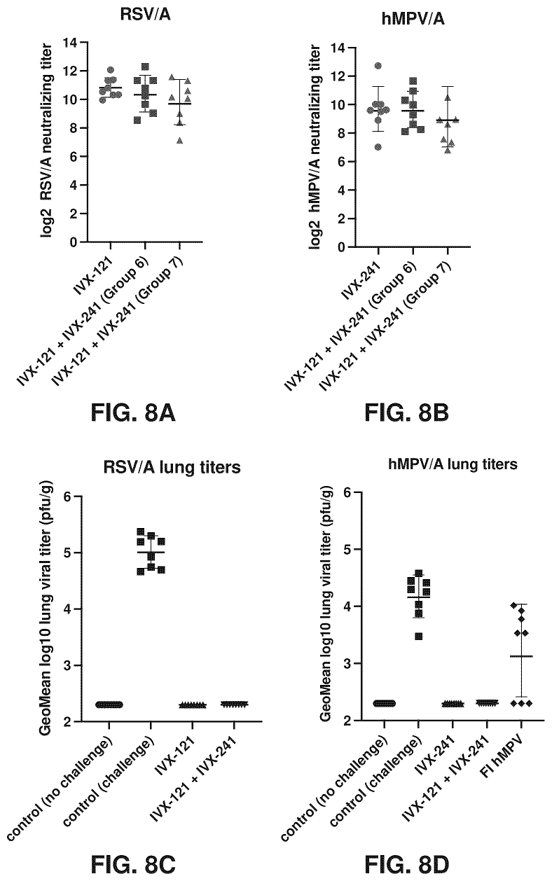

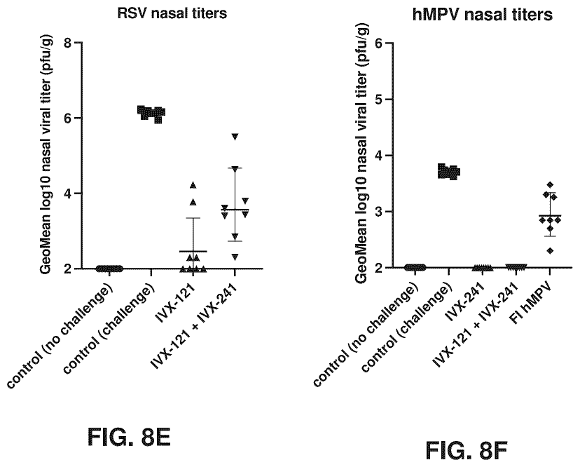

Animals vaccinated with IVX-121, IVX-241 or IVX-121+IVX-241 showed significant increases in neutralizing titers against RSV and hMPV on Day 35 compared to pre-immune sera. Titers observed with IVX-121 or IVX-241 were equivalent to the titers observed in IVX-121+IVX 241 formulations. (FIGS. 8A-8B).

The animals were challenged intranasally with 10⁵ plaque forming units (PFU) of either RSV-A2 or hMPV-A and lung tissue samples tested 5 days post challenge for viral replication. Control animals that were not challenged with RSV-A or hMPV displayed lung titers below the limit of detection (2.2 log₂). Cotton rats that were not vaccinated but challenged with RSV or hMPV resulted in substantial viral titers in the lung. Monovalent or bivalent formulations blocked viral replication in the lung of RSV and hMPV to below the lower limit of quantitation (FIGS. 8C-8D).

Control animals that were not challenged with RSV-A displayed nasal titers below the limit of detection (2.0 log₂) while unvaccinated animals challenged with RSV-A had substantial viral titers in the nose (FIG. 8E). Mice immunized with either IVX-121 or IVX-A12 and challenged with RSV-A displayed nasal viral titers that were significantly reduced demonstrating suppression of viral replication in the upper airway (FIG. 8E).

Control animals that were not challenged with hMPV-A2 displayed nasal titers below thee limit of detection (2.0 log 2) while unvaccinated animals challenged with hMPV-A2 had substantial viral titers in the nose (FIG. 8F). Mice immunized with either IVX-241 or IVX-A12 and challenged with hMPV-A displayed nasal viral titers at levels observed in the control and not challenged group (2.0 log 2) demonstrating complete suppression of viral replication (FIG. 8F). The FI hMPV vaccinated group modestly reduced nasal viral titers compared to the challenged control group (FIG. 8F).

TABLE 11

| | | | | | hMPV | RSV |
|---|---|---|---|---|---|---|
| Group | | Experimental | Antigen per | | chal- | chal- |
| No. | N | Group | Dose (μg) | Adjuvant | lenge | lenge |
| 1 | 8 | PBS | N/A | N/A | No | No |
| 2 | 8 | PBS | N/A | N/A | Yes | No |
| 3 | 8 | PBS | N/A | N/A | No | Yes |
| 4 | 8 | IVX-241 | 1 | Addavax | Yes | No |
| 5 | 8 | IVX-121 | 1 | Addavax | No | Yes |
| 6 | 8 | IVX-121 + | 1 each | Addavax | Yes | No |
| | | IVX-241 | | | | |
| 7 | 8 | IVX-121 + | 1 each | Addavax | No | Yes |
| | | IVX-241 | | | | |
| 8 | 8 | Formalin | (1:75) | N/A | Yes | No |
| | | inactivated | Dilution | | | |
| | | hMPV | | | | |
| | | Lot#071312 | | | | |

### Example 9: Overview of Clinical Evaluation of Bivalent RSV-hMPV Vaccine

These Examples describe experiments to demonstrate safety and efficacy of a RSV/hMPV combination VLP candidate (IVX-A12). The vaccine is a mixture of two monovalent protein-based virus-like particles (VLPs). Both VLPs are two-component, icosahedral VLPs displaying the ectodomain of the RSV F protein or hMPV F protein, respectively. As first component A, the vaccine uses a polypeptide antigen composed of the ectodomain of RSV or hMPV F protein, C-terminally fused to the I53-50A protein (SEQ ID NO: 144). As component B, the VLP uses the I53-50B protein. It is known that I53-50A and 153-50B spontaneously assemble in 3:5 ratio to form a VLP having icosahedral (153) symmetry, as shown in FIGS. 1A and 1B. The RSV and hMPV components are each engineered to stabilize the pre-fusion conformation of the respective F protein.

The immunogenicity of the bivalent vaccine candidate is being evaluated in a Phase 1 first-in-human (FIH) trial in healthy young and older adults by measuring the change in RSV and hMPV nAb levels compared to baseline antibody levels. Different combinations of RSV and hMPV is being assessed for potential immune interference caused by the addition of hMPV VLPs to the RSV VLP vaccine candidate. Contingent upon favorable safety results, demonstration of immunogenicity and determination of the optimal RSV-hMPV dose combination, the efficacy of our RSV-hMPV combination vaccine candidate will be assessed. The efficacy will be assessed by measuring incidence of lower respiratory infection (LRI), caused by either RSV or hMPV in patients receiving IVX-A12 compared to those receiving placebo. Different formulations of IVX-A12 may be assessed, such as with and without adjuvant.

### IVX-A12 Phase 1 Trial

A Phase 1 trial for IVX-A12 will be performed. The goal of the Phase 1 trial of IVX-A12 will be to assess safety and immunogenicity of varying doses of IVX-A12, with and without adjuvant, in older adults 60-75 years of age. IVX-A12 will be given with a fixed IVX-121 dose and one of three dose levels of IVX-241 VLP, formulated with and without adjuvant. This design will enable evaluation of the immune responses to both individual components of IVX-A12 and to see if the combination of VLPs increases the reactogenicity or leads to immune interference (i.e., imbalanced immune responses to component VLPs). All subjects in the Phase 1 trial will be evaluated for safety and antibody response for twelve months following administration of IVX-A12 or placebo. The plan is to evaluate interim data from the Phase 1 trial to determine the need for adjuvant, and to select the dose regimen for evaluation in the Phase 2 dose-confirmation trial.

### IVX-A12 Phase 2 Dose-Confirmation Trial

Following completion of the IVX-A12 Phase 1 clinical trial, a Phase 2 dose-confirmation clinical trial will be initiated in healthy older adults 60-75 years of age. The formulations and dose regimen for evaluation will be selected in the Phase 2 clinical trial based on data from the IVX-A12 Phase 1 trial. The planned Phase 2 clinical trial will evaluate different combinations of varying concentrations of hMPV and RSV VLPs in healthy older adults to assess safety and immunogenicity of varying concentrations of hMPV and RSV VLPs, and guide final dose selection for a subsequent PoC Phase 2b trial.

### IVX-A12 Phase 2 Extension Trial

Older adult subjects who complete the Phase 2 trial will be enrolled into a Phase 2 extension trial to assess duration of antibody persistence and long-term safety over multiple years.

### IVX-A12 Phase 2b Proof-of-Concept (PoC) Trial

A global Phase 2b randomized observer-blind placebo-controlled PoC efficacy trial will be conducted to evaluate the formulation of IVX-A12 selected from the Phase 2 dose-confirmation trial. The planned PoC objectives for the Phase 2b trial will include assessment of safety, immunogenicity, and efficacy against LRI caused by either RSV or hMPV. The trial population will include adults 60 years of age or older, including nested cohorts of frail and at-risk elderly, as well as healthy subjects over 85 years of age.

### Example 10: A Phase 1 Randomized, Observer-Blind, Placebo-Controlled, Multi-Center Trial to Evaluate the Safety and Immunogenicity of IVX-A12, a Respiratory Syncytial Virus and Human Metapneumovirus Bivalent Combination Virus-Like Particle Protein Subunit Vaccine, in Healthy Adults

The candidate vaccine, IVX-A12, is a bivalent combination formulation containing IVX-121 and IVX-241 virus-like particles (VLPs), computationally designed recombinant protein subunit vaccines for RSV and hMPV, respectively. Each VLP is composed of two recombinant proteins, Component A (CompA) and Component B (CompB-01), which have been designed to cooperatively assemble to form an icosahedral structure. Component A is a fusion protein specific for each vaccine candidate (either CompA-RSV-02 or CompA-hMPV-01 for RSV and hMPV, respectively) and is expressed with the prefusion F protein from the respective virus, which has been shown to induce robust neutralizing antibody responses in nonclinical models. CompB-01 is a common component across both VLPs and provides the structural element that supports the multimeric display of CompA-RSV-02 or CompA-hMPV-01. When combined, the two components self-assemble into a VLP that shows enhanced immunogenicity compared with either soluble DS-Cav1 or hMPV prefusion antigen trimer. The soluble DS-Cav1 RSV prefusion F-protein was recently tested clinically and shown to be tolerable and durably immunogenic (Ruckwardt et al., 2021).

The IVX-121 and IVX-241 VLPs display 20 copies of the respective prefusion F protein trimers on their surface. The candidate IVX-121 and IVX-241 VLPs will be manufactured independently and combined to create a bivalent formulation containing both VLPs, referred to as IVX-A12. The proposed mechanism of action of IVX-A12 is to increase the proportion of RSV- and hMPV-specific neutralizing antibodies that are associated with protection, compared to non-neutralizing antibodies (Ngwuta et al., 2015; Falloon et al., 2017). IVX-A12 is intended for active immunization of the target population of older adults who are most at risk for disease following infection with RSV and hMPV.

The candidate vaccine, IVX-A12, is expected to be a single-dose liquid formulation (0.5 mL) for intramuscular (IM) use in adults 60 to 75 years of age, the target population. The IVX-A12 candidate vaccine will be formulated and vialed as an aqueous vaccine, to be diluted 1:1 (V/V) with either oil-in-water emulsion as an adjuvant or diluent.

The proposed phase 1 clinical trial is a first-in-human (FIH) dosage-ranging evaluation of the IVX-A12 candidate vaccine in healthy older adults, 60 to 75 years of age. The purpose of this phase 1 trial is to evaluate the clinical safety and immunogenicity of one dose of the bivalent IVX-A12 of the bivalent IVX-A12 candidate vaccine, composed of a constant dosage (amount) level of IVX-121 RSV VLPs with varying concentrations (defined as low, medium, and high dosage levels) of IVX-241 hMPV VLPs with and without MF59®, compared to placebo. A placebo (diluent) will be used because there is no licensed RSV or hMPV vaccine. The three different antigen amounts that have been selected will be evaluated in a stepwise manner in three different cohorts.
low dosage level: 75 μg RSV/75 μg hMPV,
medium dosage level: 75 μg RSV/150 μg hMPV, and
high dosage level: 75 μg RSV/225 μg hMPV.

Cohort 1 will evaluate the safety and immunogenicity in healthy adults 60 to 75 years of age of one dose, administered intramuscularly, of the low dosage level (75 μg RSV/75 μg hMPV) of the unadjuvanted IVX-A12 candidate vaccine (IVX-A12a) compared to placebo.

Cohort 2 will evaluate the safety and immunogenicity in healthy adults 60 to 75 years of age of one dose, administered intramuscularly, of (i) the low dosage level (75 μg RSV/75 μg hMPV) of the IVX-A12 candidate vaccine adjuvanted with MF59® (IVX-A12d) and (ii) the medium dosage level (75 μg RSV/150 μg hMPV) of the unadjuvanted IVX-A12 candidate vaccine (IVX-A12a) compared to placebo.

Cohort 3 will evaluate the safety and immunogenicity in healthy adults 60 to 75 years of age of one dose, administered intramuscularly, of the (i) medium dosage level (75 μg RSV/150 μg hMPV) of the IVX-A12 candidate vaccine adjuvanted with MF59® (IVX-A12d) and (ii) the high dosage level (75 μg RSV/225 μg hMPV) of the unadjuvanted IVX-A12 candidate vaccine (IVX-A12a) compared to placebo. The high dosage level (75 μg RSV/225 μg hMPV) of the IVX-A12 candidate vaccine will not be evaluated with MF59® in this clinical trial.

The trial will be conducted in accordance with the protocol, International Council for Harmonisation of Technical Requirements for Pharmaceuticals for Human Use (ICH), Good Clinical Practice (GCP) Guidelines, and applicable regulatory requirements.

### Primary Objectives:

To evaluate the safety and immunogenicity of three dosage levels (low, medium, high) of the bivalent combination RSV/hMPV VLP candidate vaccine (IVX-A12), compared to placebo, when administered as a single-dose regimen in healthy older adults 60 to 75 years of age, by assessing:
Solicited local reactions and systemic AEs for 7 consecutive days (from Day 0 to Day 6), after the dose, and unsolicited AEs up to 28 days after the dose (Day 28);
RSV and hMPV neutralizing antibody (NAb) titers measured by live-virus assays, and RSV-specific and hMPV-specific prefusion F protein IgG antibody titers measured by enzyme-linked immunosorbent assay (ELISA), at Day 28;
Geometric mean fold rise (GMFR) at Day 28 versus Day 0 for RSV-A-specific and RSV-B-specific NAb titers, hMPV-A-specific and hMPV-B-specific NAb titers, and RSV and hMPV IgG prefusion F protein-specific antibody titers.

### Secondary Objectives:

To further assess the safety of IVX-A12 compared to placebo by the incidence of:
Serious adverse events (SAEs), medically-attended adverse events (MAAEs), and AEs leading to trial withdrawal throughout the trial period, up to Day 365;
AEs of special interest (AESIs) up to Day 365;
Moderate to severe LRTI (clinical event of special interest, CESI) up to Day 365;
Clinical-safety laboratory parameters up to 7 days after the dose on Day 0.
To further assess the immunogenicity of IVX-A12 compared to placebo up to Day 365 by evaluating:
RSV and hMPV NAb titers measured by live-virus assays;
RSV and hMPV prefusion F protein specific IgG antibody titers measured by ELISA;
The ratios of fold-increase in IgG titers over fold-increase in NAb titers to RSV and hMPV;
GMFR versus baseline (Day 0) for RSV-A-specific and RSV-B-specific NAb titers, hMPV-A-specific and hMPV-B-specific NAb titers, and RSV and hMPV IgG prefusion F protein-specific antibody titers;
GMFR versus baseline (Day 0) for hMPV-A-specific and hMPV-B-specific NAb titers.

### Exploratory Objectives:

To further explore the immunogenicity of IVX-A12 vaccine compared to placebo by assessing:

The epitope-specificity of IgGs to the RSV and hMPV prefusion F proteins;
VLP core-specific IgG titers.

### Clinical Trial Design:

The IVX-A12 phase 1 trial is a randomized, observer-blind, placebo-controlled, multi-center dosage- and dosage-escalation trial to assess the safety and immunogenicity of a single dose of two IVX-A12 formulations: as aqueous (unadjuvanted) and adjuvanted with MF59® (IVX-A12a and IVX-A12d, respectively). The trial will have a total of 8 treatment groups (5 active treatment groups and 3 placebo groups). The active treatment groups receiving IVX-A12 formulations will be tested at 3 dosage levels (low, medium, and high total VLP content) of IVX-A12. The low and medium dosage levels will be tested with and without MF59®. The high dosage level will be only tested without MF59®. The clinical trial will be conducted in three cohorts. The three different dosage levels (low dosage level: 75 μg RSV/75 μg hMPV, medium dosage level: 75 μg RSV/150 μg hMPV, and high dosage level: 75 μg RSV/225 μg hMPV) that have been selected will be evaluated in a stepwise manner in three different cohorts. In addition, placebo will be administered as a control. Each cohort will have a placebo treatment group. The three cohorts are given in Table 12.

TABLE 12

| Healthy Adults | | | |
|---|---|---|---|
| 60 to 75 years of | Trial | | |
| age; N = 120 | Groups | Dosage Level (1 dose regimen) | N |
| Cohort 1 | A | 75 μg RSV: 75 μg hMPV vaccine | 20 |
| | B | Placebo‡ | 4 |
| Cohort 2 | C | 75 μg RSV: 75 μg hMPV | 20 |
| | | vaccine in MF59 ® | |
| | D | 75 μg RSV: 150 μg hMPV vaccine | 20 |
| | E | Placebo | 8 |
| Cohort 3 | F | 75 μg RSV: 150 μg hMPV | 20 |
| | | vaccine in MF59 ® | |
| | G | 75 μg RSV: 225 μg hMPV vaccine | 20 |
| | H | Placebo | 8 |

Total number of placebo recipients N=20. Total amount of VLP content is 150 μg (Groups A and C), 225 μg (Groups D and F), and 300 μg (Group G). †MF59® contains 9.75 mg of squalene and surfactants. $Placebo will be aqueous diluent. For purposes of data analyses, treatment groups B, E, and H will be combined into a single placebo group.

### Trial Procedure:

Approximately 120 healthy adult subjects (60-75 years of age) will be enrolled in a stepwise dosage-escalation (from lowest to highest) manner into one of three cohorts. Each cohort will enroll adults 60 to 75 years of age. Within each cohort, subjects will be randomly allocated at a ratio 5:1 to receive investigational medicinal product (IMP; IVX-A12a or IVX-A12d) or placebo (diluent). There will be approximately 24 subjects in Cohort 1, 20 subjects in the single active group and 4 subjects in the placebo group. In Cohort 2, there will be approximately 48 subjects: 20 in each of the two active treatment group and 8 subjects in the corresponding placebo group. In Cohort 3, there will be also approximately 48 subjects: 20 in each of the two active treatment groups and 8 subjects in the corresponding placebo group. All subjects will receive one dose of a 0.5 mL intramuscular injection of IVX-A12 or placebo.

Within each cohort, vaccination will be performed in stages in order to administer the vaccine to sentinels first. The staging within each cohort is provided in Table 13.

In Cohort 1, two sentinel subjects will receive IVX-A12a and one sentinel will receive placebo (from placebo group within the corresponding cohort), with a minimum of a 36-hour observation period. If no stopping rules are triggered, the remaining 21 subjects (18 from the active group (Group A) and 3 from the corresponding placebo group of (Cohort 1) will be enrolled.

In Cohort 2 and Cohort 3, four sentinel subjects will receive IVX-A12 (two subjects from the IVX-A12d group [same dosage level tested in the previous cohort in the absence of adjuvant], and two subjects from the IVX-A12a group [next unadjuvanted dosage level]) and two sentinel subjects will receive placebo (placebo group within the corresponding cohort), with a minimum of a 36-hour observation period. If no stopping rules are triggered, the remaining 42 subjects (18 from the active IVX-A12a group, 18 from the active IVX-A12d group, and 6 from the placebo group of each dosage level cohort) will be vaccinated. The independent SMC will be responsible for the application of stopping rules, if achieved. If no stopping rules are triggered within 7 days after dosing all subjects in each dosage level cohort and IVX-A12a and IVX-A12d formulations are found to have acceptable reactogenicity as determined by the SMC, the trial will proceed to the subsequent dosage level cohort. Before proceeding to and starting the subsequent dosage level cohort, the SMC will review all available cumulative safety data through Day 7 after vaccination.

Cohort 1: Evaluation of the safety and immunogenicity of the low dosage level (75 μg RSV/75 μg hMPV) of the IVX-A12 unadjuvanted candidate vaccine (IVX-A12a) compared to placebo. Subjects (N=24) enrolled will be randomly allocated at a ratio 5:1 to receive IVX-A12a at the low dosage (Group A) or placebo (Group B). Two sentinel subjects (Stage 1) will receive IVX-A12a (Group A) one sentinel subject will receive placebo (Group B). Following the observation period, the remaining subjects of Groups A (N=18) and B (N=3) will be vaccinated (Stage 2). The SMC will review the safety data after enrollment of Cohort 1 has been completed before proceeding with enrollment of Cohort 2.

Cohort 2: Evaluation of the safety and immunogenicity of the low dosage level (75 μg RSV/75 μg hMPV) of IVX-A12 candidate vaccine with MF59® and the medium dosage level (75 μg RSV/150 μg hMPV) of the unadjuvanted IVX-A12 candidate vaccine compared to placebo. Subjects (N=48) enrolled will be randomly allocated at a ratio 5:1 to receive IVX-A12d (low dosage level) or IVX-A12a at the medium dosage level, Group C or Group D, respectively or placebo (Group E). Four sentinel subjects (Stage 3) will receive IVX-A12 (two subjects from Group C [low dosage IVX-A12d] and two subjects from Group D [medium dosage IVX-A12a]) and two sentinel subjects will receive placebo (Group E). Following the observation period, the remaining subjects of Groups C, D, and E will be vaccinated (Stage 4). The SMC will review the safety data after enrollment of Cohort 2 has been completed before proceeding with enrollment of Cohort 3.

Cohort 3: Evaluation of the safety and immunogenicity of the medium dosage level (75 μg RSV/150 μg hMPV) with MF59® and the high dosage level (75 μg RSV/225 μg hMPV) of the IVX-A12 candidate vaccine unadjuvanted (IVX-A12a) compared to placebo. Subjects (N=48) enrolled will be randomly allocated at a ratio 5:1 to receive IVX-A12d (medium dosage) or IVX-A12a at the high dosage level, Group F or Group G, respectively or placebo (Group H). Four sentinel subjects (Stage 5) will receive IVX-A12 (two subjects from Group F [medium dosage IVX-A12d] and two subjects from Group G [high dosage IVX-A12a]) and two sentinel subjects will receive placebo (Group H). Following the observation period, the remaining subjects of Groups F, G, and H will be vaccinated (Stage 6). The SMC will review the safety data after enrollment of Cohort 3 accordingly, no additional dosage escalation will be evaluated beyond that in Cohort 3. In addition, the high dosage level (75 μg RSV/225 μg hMPV) of the IVX-A12 candidate vaccine will not be evaluated with MF59® in this clinical trial.

TABLE 13

| | D 0 Vaccination | | | | | | | | |
|---|---|---|---|---|---|---|---|---|---|
| | | MF59 ®† | 60-75 years of age | | | | | | |
| Trial | VLP | (mg | Stage 1 | Stage | Stage 3 | Stage | Stage 5 | Stage | Total |
| Group | (pg) | squalene) | Sentinel | 2 | Sentinel | 4 | Sentinel | 6 | N = 120 |
| A | 75 RSV | 0 | 2 | 18 | — | — | — | — | 20 |
| | 75 hMPV | | | | | | | | |
| C | 75 RSV | 9.75 | — | — | 2 | 18 | — | — | 20 |
| | 75 hMPV | | | | | | | | |
| D | 75 RSV | 0 | — | — | 2 | 18 | — | — | 20 |
| | 150 hMPV | | | | | | | | |
| F | 75 RSV | 9.75 | — | — | — | — | 2 | 18 | 20 |
| | 150 hMPV | | | | | | | | |
| G | 75 RSV | 0 | — | — | — | — | 2 | 18 | 20 |
| | 225 hMPV | | | | | | | | |
| B, E, | 0 | 0 | 1 | 3 | 2 | 6 | 2 | 6 | 20 |
| H | (placebo)‡ | | | | | | | | |

†MF59® contains 9.75 mg of squalene and surfactants. $Placebo will be aqueous diluent. Group B corresponds to Stages 1 and 2, Group E corresponds to Stages 3 and 4; and Group H corresponds to Stages 5 and 6. For purposes of data analyses, treatments groups B, E, and H will be combined into a single placebo treatment group.

### Assessments:

There will be a total of 7 scheduled clinic visits required for each subject (at Screening, then Days 0, 7, 28, 90, 180, and 365). Blood sampling for safety will be conducted at screening and on Days 0, 7, and 28 while immunogenicity sampling will occur at Days 0, 7, 28, 180, and 365.

Safety assessments for all subjects in Cohorts 1, 2 and 3: All solicited local reactions and systemic AEs will be collected for all subjects starting from Day 0, the day of the single dose administration, to Day 6 (7 days total); unsolicited AEs through Day 28; and safety blood samples (clinical laboratory evaluation at screening, Day 0/Baseline, Day 7, and Day 28). SAEs, MAAEs, AEs leading to trial withdrawal, and AESIs will be captured from the time of randomization to the end of the trial evaluation, approximately 12 months after vaccination. During the treatment and follow-up periods, all subjects will also be actively monitored for the trial-defined CESI of moderate to severe LRTI, using the established case definition and algorithm for evaluation of acute respiratory illness (ARI) using nasopharyngeal (NP) swabs for the detection of RSV, hMPV, and other respiratory viruses. Subjects will be instructed to return to the clinic for assessment within 7 days of onset of ARI (ideally within 72 hours) to maximize detection of virus.

Immunogenicity assessments for all subjects in Cohorts 1, 2 and 3: Blood samples will be taken for serology (Day 0/Baseline, Day 7, Day 28, Day 180, and Day 365). All subjects will be monitored for persistence of antibody responses through the entire duration of the clinical trial (approximately 12 months).

In the event of local or national health related closures and limited ability for travel (e.g., as a consequence of SARS-CoV-2 circulation), the clinic may default to their institution guidelines to continue follow-up assessments with subjects as necessary.

### Stopping Rules:

Monitoring of safety signals will be performed throughout the trial by an independent SMC. Stopping rules or conditions for stopping this clinical trial would occur if there was clear evidence of harm or harmful effects. Administration of the vaccine may result in a halt to the trial and require further review and assessment if any of the following event(s) occur:

In a trial subject:
Any death occurring during the trial;
Any vaccine-related SAE during the trial;
Any life-threatening (Grade 4) vaccine-related AE during the trial, which needs medical intervention including:
   Ulceration, abscess or necrosis at the injection site;
Laryngospasm, bronchospasm or anaphylaxis within 24 hours after administration of vaccine;
   An allergic or hypersensitivity reaction such as fever >40° C. or generalized urticaria (defined as occurring at three or more body parts) within 72 hours after administration of vaccine.

If two or more of subjects in a single trial group in any of the three Cohorts experience the same severe (Grade 3) AE (Preferred Term [PT] in a given Medical Dictionary for Regulatory Activities [MedDRA] system organ class [SOC]) within the first 7 days following vaccination that persisted for at least 48 hours and cannot be clearly attributed to another cause:
Severe (Grade 3) solicited local reactions (excluding measured grades of erythema and swelling alone) or systemic AE;
Severe (Grade 3) vaccine-related unsolicited AE during the trial;
Severe (Grade 3) vaccine-related vital sign(s) abnormality;
Severe (Grade 3) vaccine-related clinical laboratory abnormality.

In the case that a pre-defined safety signal is met in any trial group, subsequent dosing will result in at least a transient halt in the trial to permit a complete evaluation of the reported event(s), and to consult the SMC. Based on the review of the data, the SMC may recommend temporary or permanent stopping, or continuation of dosing. After a temporary halt, further measures for safety may be introduced.

### Subject Population:

Healthy Subjects: Yes.

Age Range: 60 to 75 years of age.

Planned Number of Subjects: Approximately 120 total (24 in Cohort 1, 48 in Cohort 2, and 48 in Cohort 3).

Planned Number of Trial Groups: A total of 8 treatment groups (5 active treatment groups and 3 placebo groups [one placebo group for each cohort]). The active treatment groups receiving IVX-A12 formulations will be tested at 3 dosage levels (low, medium, and high total VLP content) of IVX-A12. The low and medium dosage levels will be tested with and without MF59®.The high dosage level will be only tested without MF59®.

### Key Inclusion Criteria:

1. Healthy male or non-pregnant female older adults 60 to 75 years of age at the time of first vaccination;
2. Includes subjects with stable well-controlled chronic conditions such as hypertension without clinical exacerbation of their underlying disease within the previous 12 months;
3. Subjects able to voluntarily give written informed consent and to comply with trial procedures including follow-up to approximately 12 months after first dosing;
4. Body mass index (BMI) 17 to 35 kg/m2, inclusive, at screening;
5. Screening laboratory values must be within the laboratory reference ranges or deemed not clinically significant if within Grade 1 severity on the toxicity scale.

### Key Exclusion Criteria:

1. Prior receipt of any investigational RSV or hMPV vaccine;
2. Prior receipt of another investigational medicinal product (study drug, biologic, or device) not authorized for use in the US and European Union within the past year;
3. Laboratory-confirmed severe RSV or hMPV infection within the past year prior to enrollment;
4. Currently enrolled or plan to participate in another clinical trial with an investigational agent (including licensed or unlicensed vaccine, drug, biologic, device, blood product, or medication) to be received during the trial period;
5. Presence of high-risk comorbidities for severe RSV or hMPV disease (e.g., significant cardiopulmonary disease);
6. Older adults meeting frail elderly criteria (older persons with medical, nutritional, cognitive, emotional, or activity impairments, as defined by the trial site);
7. Acute or chronic progressive, unstable or uncontrolled clinical conditions;
8. Acute illness, with or without fever at the time of planned vaccination;
9. History of hypersensitivity or serious adverse reactions to vaccines, such as anaphylaxis, Guillain-Barre, and angioedema, or any known allergies to any component of the IVX-121 and/or IVX-241 vaccine, or hypersensitivity to latex;
10. Abnormal function of the immune system resulting from clinical conditions including human immunodeficiency virus, chronic administration of systemic corticosteroids (oral/intravenous/IM at a dose equivalent of >20 mg prednisone in a period of more than 14 days), or administration of immunosuppressive chemotherapy, biologics, or radiotherapy within the past 3 months before trial randomization;
11. Refusal to maintain contraceptive practices during the trial, and (for women of childbearing potential) to be screened for pregnancy at specified times during the trial;
12. Receipt of licensed inactivated vaccines including influenza vaccine within 14 days prior to trial vaccine administration on Trial Day 0, or with live virus vaccines within 30 days of Day 0.
Receipt of licensed vaccines is permitted after completion of the trial Day 28 visit.
Receipt of licensed COVID-19 vaccines is permitted if dosing regimen completed within 21 days prior to trial vaccine administration on Day 0 or after completion of the Day 28 visit.

### Investigational Medicinal Products:

IVX-A12: The IVX-A12 formulation for testing will be composed of one constant IVX-121 RSV VLP dosage level combined with one of three different dosage levels of the IVX-241 hMPV VLP up to the maximum human dosage (amount) of combined VLP (high dosage). The final investigational IVX-A12 drug product will be administered either as an aqueous vaccine (IVX-A12a) or with MF59® to be mixed 1:1 in the clinic (IVX-A12d). Specific instructions for preparation of vaccine will be provided in the pharmacy manual. The adjuvant content will be the same for all formulations containing MF59®. The intended volume for administration of IVX-A12 is 0.5 mL for IM administration.Placebo: Sterile aqueous diluent, delivered as a 0.5 mL dose. The placebo does not contain preservatives.

Route of Administration: Intramuscular (IM) administration into the deltoid of the non-dominant arm.

### Duration Subject Participation:

The duration of subject participation in the trial will be approximately 12 months.

Criteria for Evaluation and Analyses:

### Co-Primary Endpoints (Safety):

Solicited local reactions and systemic AEs from Day 0 to Day 6 (7 days total);
Unsolicited AEs from Day 0 to Day 28.

### Co-Primary Endpoints (Immunogenicity):

RSV-A-, RSV-B-, hMPV-A-, and hMPV-B-specific NAb (live-virus assays) at Day 28.
RSV and hMPV IgG prefusion F protein-specific antibody titers (ELISAs) at Day 28;
Proportion of subjects with a ≥4-fold increase in serum anti-RSV/A-, RSV/B-, hMPV/A-, and hMPV/B-specific NAb (live-virus assays) at Day 28;
Proportion of subjects with a ≥8-fold increase in serum anti-RSV/A-, RSV/B-, hMPV/A-, and hMPV/B-specific NAb (live-virus assays) at Day 28;
GMFR at Day 28 versus Day 0 (in serum anti-RSV/A-, RSV/B-, hMPV/A-, and hMPV/B-specific NAb ([live-virus assays]) and RSV and hMPV IgG prefusion F protein-specific antibody titers ([ELISAs]).

### Secondary Endpoints (Safety):

SAEs, MAAEs, AEs leading to trial withdrawal, and AESIs from Day 0 up to the end of trial;
CESI (moderate to severe LRTI) from Day 0 up to the end of trial;
Clinical-safety laboratory parameters at screening, and after dosing, at Days 0, 7, and 28.

### Secondary Endpoints (Immunogenicity):

RSV-A-, RSV-B-, hMPV-A- and hMPV-B-specific NAbs by live-virus assays at Days 0, 7, 180, and 365;
Proportion of subjects with a ≥4-fold increase in serum anti-RSV-A-, RSV-B-, hMPV-A-, and hMPV-B-specific NAb (live-virus assays) at Days 7, 180, and 365 compared to baseline (Day 0);
Proportion of subjects with a ≥8-fold increase in serum anti-RSV-A-, RSV-B-, hMPV-A-, and hMPV-B-specific NAb (live-virus assays) at Days 7, 180, and 365 compared to baseline (Day 0);
GMFR at Day 7 versus Day 0 (in serum anti-RSV-A-, RSV-B-, hMPV-A-, and hMPV-B-specific NAb ([live-virus assays]) and RSV and hMPV IgG prefusion F protein-specific antibody titers ([ELISAs]);
GMFR at Day 180 versus Day 0 (in serum anti-RSV-A-, RSV-B-, hMPV-A-, and hMPV-B-specific NAb ([live-virus assays]) and RSV and hMPV IgG prefusion F protein-specific antibody titers ([ELISAs]);
GMFR at Day 365 versus Day 0 (in serum anti-RSV-A-, RSV-B-, hMPV-A-, and hMPV-B-specific NAb ([live-virus assays]) and RSV and hMPV IgG prefusion F protein-specific antibody titers ([ELISAs]);
Reverse cumulative distribution (RCD) of serum IgG binding antibody to RSV prefusion F protein at Days 0, 7, 28, 180, and 365;
RCD of serum IgG binding antibody to hMPV prefusion F protein at Days, 0, 7, 28, 180, and 365.

### Exploratory Endpoints (Immunogenicity):

RSV and hMPV prefusion F-protein-specific IgG titers, by competitive binding ELISA using epitope-mapped monoclonal antibodies (MAbs), at Days 0, 28, 180, and 365;
VLP core-specific IgG titers by ELISA at Days 0, 28, 180, and 365.

### Statistical Considerations:

### Analysis Sets:

Safety Set: The safety set will consist of all subjects who received at least one dose of IVX-A12 or placebo. Subjects will be analyzed as treated.

Full Analysis Set (FAS): The FAS will include all subjects who received at least one dose of IVX-A12 or placebo. Subjects will be analyzed as randomized.

Per-Protocol Set (PPS): The PPS will include all subjects in the FAS who received a dose of IMP and have no major protocol deviations.

### Analysis of Demographics and Other Baseline Characteristics

Age, gender, race, and other baseline characteristics (including baseline RSV and hMPV NAb titers) will be summarized descriptively by trial group for all randomized subjects.

### Immunogenicity Analyses:

Day 28 immunogenicity data will be summarized for each group, cohort and strain (RSV/A, RSV/B, both RSV strains and either RSV strain; hMPV/A, hMPV/B, both hMPV and either hMPV strain.

The geometric mean titers (GMTs) will be calculated for RSV/A, RSV/B, hMPV/A and hMPV/B NAb titers separately along with their 95% CI. Data displays will include boxplots and RCD curves for each trial group, and the median and interquartile range of the fold-rise in antibody titer from baseline (Day 0) to Day 28.

### Safety Analyses:

Solicited local reactions and solicited systemic AEs: Solicited local reactions and solicited systemic AEs will be collected for all subjects from Day 0 to Day 6 (7 days total).

Unsolicited AEs: Unsolicited AEs will be assessed from day of dosing (Day 0) through 28 days post-vaccination and coded according to MedDRA and summarized by SOC and preferred term (PT) for each trial group.

SAEs, MAAEs, AEs leading to withdrawal, AESIs and CESI (moderate to severe LRTI): Will be assessed throughout the trial (from randomization on Day 0) and coded using MedDRA and summarized by SOC and PT for each trial group.

### Example 11: Phase 1/1b Study of RSV Component

This Example provides interim results from a Phase 1/1b clinical trial of IVX-121, a VLP displaying a prefusion stabilized Respiratory Syncytial Virus (RSV) F antigen, in young and older adults. IVX-121 demonstrated a robust immunologic response in both young and older adult groups.

### IVX-121 Phase 1/1b Trial Design

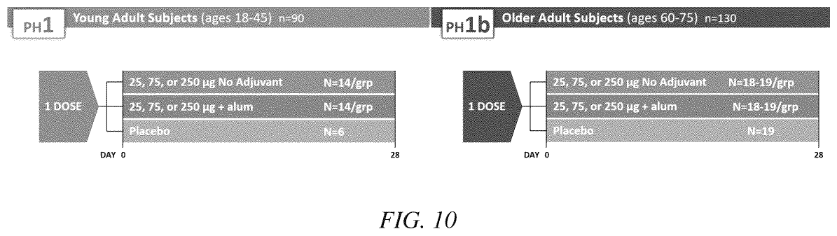

The Phase 1/1b clinical trial of IVX-121 is a randomized, observer-blinded, placebo-controlled, multi-center study designed to evaluate the safety and immunogenicity of three dose levels of IVX-121, with and without aluminum hydroxide adjuvant, in healthy young and older adults. The study design is shown in FIG. 10.

The Phase 1 part of the trial enrolled 90 healthy young adults aged 18-45 years. The Phase 1b part of the trial enrolled 130 healthy older adults aged 60-75 years. Subjects were administered a single dose of IVX-121 at one of three dose levels (25, 75, 250 μg), with or without aluminum hydroxide adjuvant, or placebo.

The primary outcomes of the study were safety and immunogenicity up to 28 days post-vaccination; neutralizing antibodies to RSV-A and RSV-B were measured in international units (IU/mL) using the WHO international reference standard.

### Topline Results

### Safety

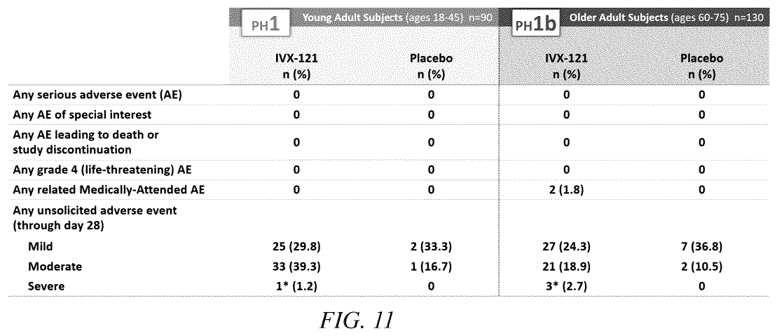

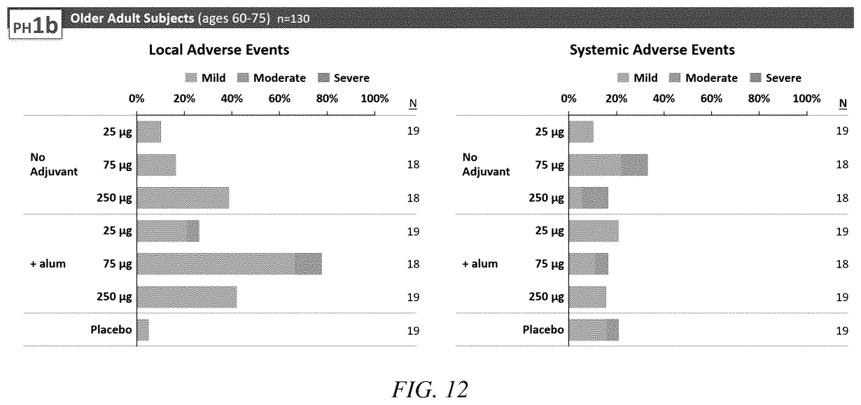

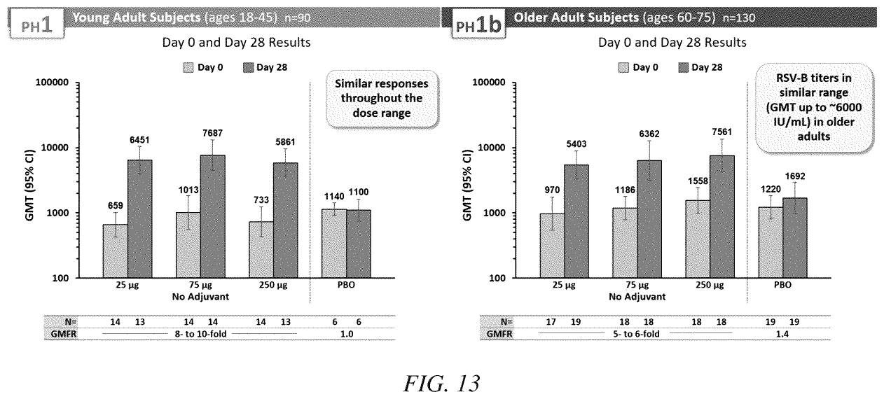

In this Phase 1/1b study, IVX-121 was generally well-tolerated across all dosage groups. Solicited local and systemic adverse events (AEs) were generally mild or moderate, without dose-limiting reactogenicity. In the older adult target population, across the six dosage groups for IVX-121 with or without adjuvant, the proportion of subjects experiencing any systemic AE within seven days was 11-33%, and similar to 21% for placebo. The most common local and systemic AEs were injection site tenderness, headache and fatigue. There were no serious AEs related to vaccine, AEs of special interest, or AEs leading to discontinuation. Data are shown in FIG. 11 and FIG. 12.

### Immunogenicity:

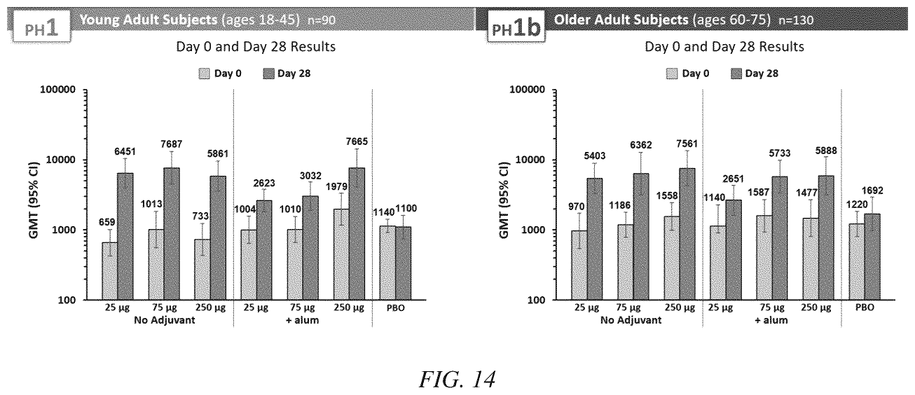

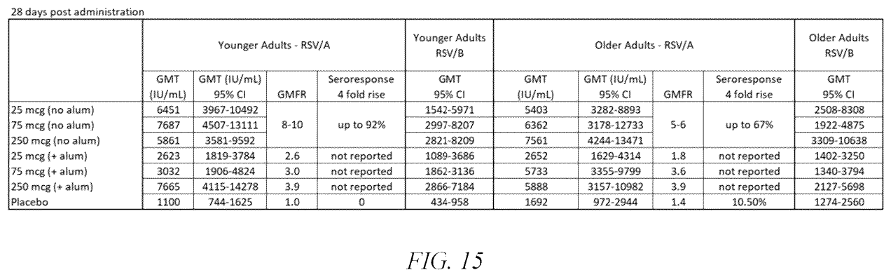

IVX-121 induced a robust immune response in both young and older adult groups. Data indicated a dose-independent response, including at the lowest non-adjuvanted dose (25 μg) (FIG. 8). No additional benefit from the aluminum hydroxide adjuvant was observed at any dosage level in either portion of the study (FIG. 14). Geometric mean titers for RSV-A and RSV-B were in comparable ranges for both groups (FIG. 15)

### Young Adults (Phase 1):

In young adults, across dosage groups, IVX-121 induced Geometric Mean Titers (GMT) in RSV-A neutralizing antibodies (nAbs) of up to 7,687 IU/mL compared to 1,100 IU/mL for placebo at Day 28. These titers corresponded to a Geometric Mean Fold Rise (GMFR) versus baseline up to 10-fold for IVX-121 at Day 28.

### Older Adults (Phase 1b):

GMT responses in IU/mL for older adults were comparable with those for young adults in the Phase 1 portion of this study. Across dosage groups, IVX-121 induced GMT in RSV-A nAbs of up to 7,561 IU/mL compared to 1,692 IU/mL for placebo at Day 28. GMFR at Day 28 was up to 6-fold, reflecting higher baseline titers in the older adults group.

### Conclusion

These data indicate that IVX-121 was generally well tolerated and elicited a strong and consistent response against RSV in healthy young and older adults. These data are particularly encouraging for the vulnerable older adult population with co-morbidities and increased risk for severe disease and hospitalization. IVX-121 exhibits immunogenicity at very low microgram dosage levels and tolerability to the highest dose level. Eligible older adults from the Phase 1b cohort will be followed out to 12 months to assess durability of response.

### Neutralizing Antibody Assay Methods

Validated RSV-A and RSV-B neutralizing antibody (NAb) assays were used to measure the presence of RSV-specific neutralizing antibodies. These assays have been validated for standard bioanalytical assay parameters to include Specificity, Accuracy, Precision, Repeatability (intra-assay precision), Intermediate Precision, Linearity, Dilutional Linearity, Range, Stability, and Robustness. Critical reagents for these assays are commercially purchased and qualified for use. In brief, human serum samples are serially diluted and incubated with a constant concentration of virus (RSV A2 (ATCC, Cat #VR-1540) and RSV B 18537 (ATCC, Manassas, VA; Cat #VR-1580)). Neutralization is measured by the inhibition of virus propagation in HEp-2 cells (ATCC, Cat #CCL23). Following an incubation period, cells are fixed and immunostained with a murine monoclonal antibody directed against RSV F protein, followed by horse radish peroxidase (HRP)-conjugated goat-anti-mouse antibody and TrueBlue (TB). The plates are scanned with an UV Analyzer and Spot counts (SC; or spot forming cells, SCF) per well at each serum/antibody concentration are quantified. The values are then analyzed to determine the dilution of serum/antibody that yield a selected reduction point (i.e., 50% or IC50).

Since there are different assay formats available for RSV neutralization assays, it is important to have a reference antiserum to standardize results. The first International Standard for antiserum to RSV-A and RSV-B (NIBSC code: 16/284) was established by the WHO Expert Committee on Biological Standardization in 2017 (McDonald et al., 2017; McDonald et al., 2018; McDonald et al., 2020). This Reference Standard was shown to be suitable for the standardization of virus neutralization methods to measure antibody levels against RSV-A and RSV-B in human sera. Due to limited quantities available of the Reference Standard, bioanalytical labs are encouraged to generate an in-house reference (IHR) standard by calibrating the IHR to the International Reference Standard. The RSV Reference Standard (16/284) with an assigned a potency of 1000 International Units (IU) anti-RSV-A or anti-RSV-B neutralizing antibodies per ampoule was used to establish and validate an IHR to be included during neutralization titer analyses. The potency of the IHR standard was determined to be 1,831 IU/mL for RSV-A and 609 IU/mL for RSV-B. These potencies were used to calculate conversion factors which can be used for converting MN titer results (AU/mL) to IU/mL. The potency of a test sample in IU/mL was calculated by multiplying the test sample MN titer by 1.0578 for RSV-A and by 0.6936 for RSV-B. The RSV-A and RSV-B NAb assays has been validated to an assay range of 9.4-180263 AU/mL and 8-195392 AU/mL, respectively. The validated assay range in IU/mL for RSV-A and RSV-B are 9.9-190682 and 5.5-135524, respectively.
McDonald et al. 2017. Report on the WHO collaborative study to establish the 1st international standard for antiserum to respiratory syncytial virus (No. WHO/BS/2017.2318). World Health Organization. https://apps.who.int/iris/handle/I0665/260488
McDonald et al. 2018. Establishment of the first WHO International Standard for antiserum to Respiratory Syncytial Virus: Report of an international collaborative study. Vaccine, 36, 7641-7649.
McDonald et al. 2020. Expansion of the 1st WHO international standard for antiserum to respiratory syncytial virus to include neutralisation titres against RSV subtype B: An international collaborative study. Vaccine, 38, 800-807.

WHO 2020. WHO International Standard 1st International Standard for Antiserum to Respiratory Syncytial Virus NIBSC code: 16/284 Instructions for use (Version 4.0, Dated 01/04/2020). https://www.nibsc.org/documents/ifu/16-284.pdf

### Example 12: Safety and Immunogencity Results of Phase 1/1b Study—Through Day 180

This Example provides Study Day 180 results from Example 11: a Phase 1/1b clinical trial of IVX-121, a VLP displaying a prefusion stabilized Respiratory Syncytial Virus (RSV) F antigen, in young and older adults. IVX-121 demonstrated a robust immunologic response in both young and older adult groups.

TABLE 14

| Study Day | | Part 1 | Part 2 |
|---|---|---|---|
| Data | | Number of subjects (N) | Number of subjects (N) |
| Analyzed | VLP (μg) | 18-45 y | 60-75 y |
| D0 | 25 | 14 | 19 |
| D28 | 25 | 13 | 19 |
| D180 | 25 | 13 | 18 |
| D0 | 75 | 14 | 17 |
| D28 | 75 | 14 | 17 |
| D180 | 75 | 14 | 15 |
| D0 | 250 | 13 | 18 |
| D28 | 250 | 12 | 18 |
| D180 | 250 | 13 | 18 |
| D0 | 0 (placebo) | 6 | 19 |
| D28 | 0 (placebo) | 6 | 19 |
| D180 | 0 (placebo) | 6 | 19 |

### IVX-121 Phase 1/1b Trial Design

The Phase 1/1b clinical trial of IVX-121 is a randomized, observer-blinded, placebo-controlled, multi-center study designed to evaluate the safety and immunogenicity of three dose levels of IVX-121, with and without aluminum hydroxide adjuvant, in healthy young and older adults.

The Phase 1 part of the trial enrolled healthy young adults aged 18-45 years. The Phase 1b part of the trial enrolled healthy older adults aged 60-75 years. Subjects were administered a single dose of IVX-121 at one of three dose levels (25, 75, 250 μg) with or without aluminum hydroxide adjuvant, or placebo.

The primary outcomes of the study were safety and immunogenicity up to 180 days post-vaccination; neutralizing antibodies to RSV-A and RSV-B were measured in international units (IU/mL) using the WHO international reference standard.

### Topline Results

### Safety

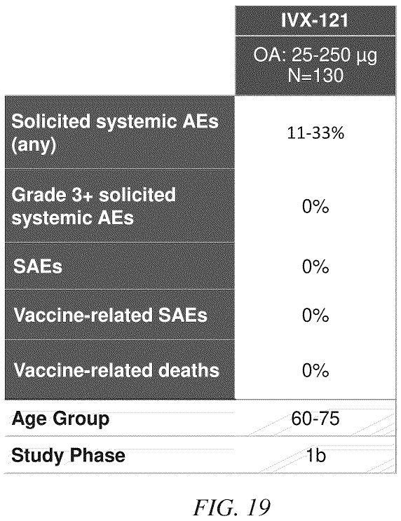

In this Phase 1/1b study, IVX-121 was generally well-tolerated across all dosage groups. Solicited local and systemic adverse events (AEs) were generally mild or moderate, without dose-limiting reactogenicity. In the older adult target population, across the six dosage groups for IVX-121, the proportion of subjects experiencing any systemic AE within seven days was 11-33%. There were no vaccine-related serious AEs (SAEs) or AEs of special interest (AESIs). There were no Grade 3+ solicited systemic AEs, serious AEs (SAEs), or AEs leading to discontinuation. Data are shown in FIG. 19.

### Immunogenicity:

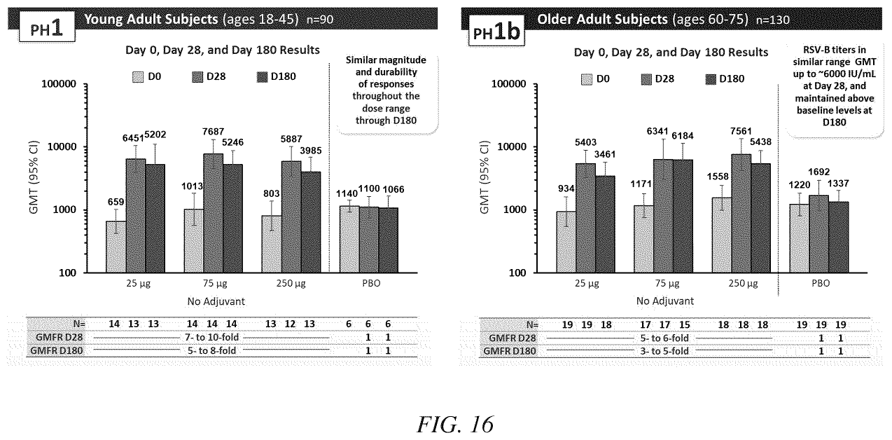

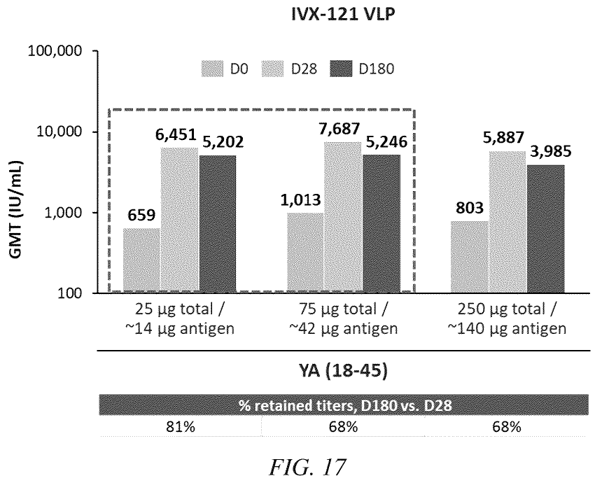

IVX-121 induced a robust immune response in both young and older adult groups and geometric mean titers (in IU/mL) were comparable in young and older adults. Data indicated a dose-independent response, including at the lowest non-adjuvanted dose (25 μg) (FIG. 16). Sustained durability (persistence) of neutralizing antibodies GMTs was observed up to 180 days after vaccination. The durability of neutralizing antibodies was observed to be dose-independent and alum adjuvant-independent in OA.

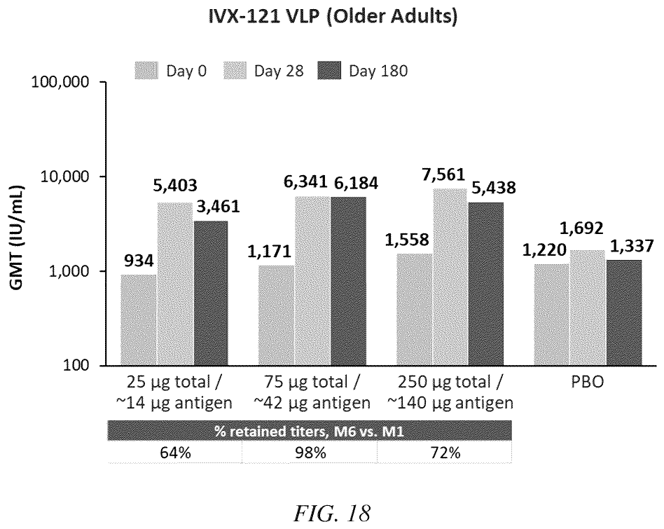

Geometric mean titers (GMT) against RSV-A through day 180 showed persistence at 64-98% of the GMTs at day 28 in older adults (FIG. 16 and FIG. 18), and remained at 3- to 8-fold above baseline.

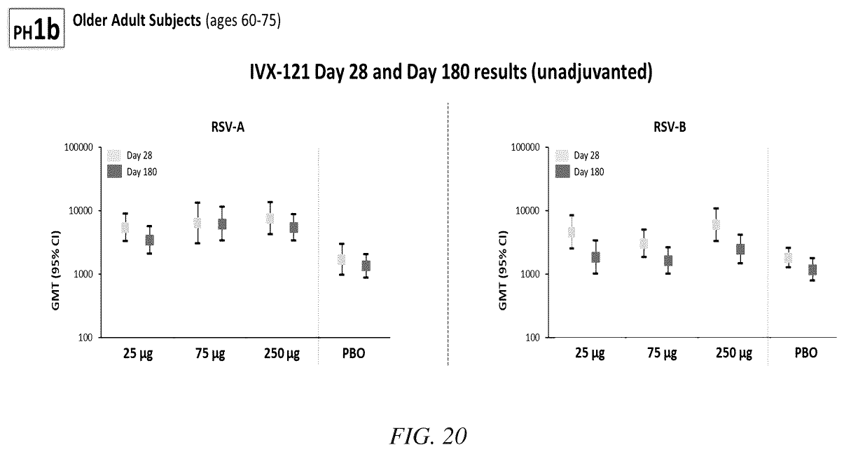

GMTs against RSV-B through day 180 showed persistence at 40-53% of the GMTs at day 28 in older adults (FIG. 20).

Placebo titers remained unchanged at baseline levels through day 180.

### Young Adults (Phase 1):

In young adults, across dosage groups, IVX-121 induced Geometric Mean Titers (GMT) of RSV-A neutralizing antibodies (nAbs) of up to 5246 IU/mL compared to 1066 IU/mL for placebo at Day 180. A consistent magnitude and durability of responses throughout the dose range was observed through Day 180.

### Older Adults (Phase 1b):

GMT responses in IU/mL for older adults were comparable with those for young adults in the Phase 1 portion of this study. Across dosage groups, IVX-121 induced GMT for RSV-A nAbs that were previously reported at up to 7,561 IU/mL at Day 28 were observed to persist at up to 6,184 IU/mL through Day 180. GMT responses for RSV-A were maintained at Day 180 within a range of 64-98% relative to the previously reported GMTs at Day 28.

GMT responses in IU/mL of RSV-B neutralizing antibodies (nAbs) for older adults in similar range of GMT response to RSV-A, GMTs observed up to ˜6000 IU/mL at Day 28 and maintained above baseline levels up to about 2,500 IU/mL at Day 180. GMTs against RSV-B showed persistence through Day 180 at 40-53% of the levels observed at Day 28.

### Conclusion

These data indicate that IVX-121 was generally well tolerated and elicited a strong and durable response against RSV in healthy young and older adults through Day 180. These data are particularly encouraging for the vulnerable older adult population with co-morbidities and increased risk for severe disease and hospitalization. IVX-121 exhibits immunogenicity at very low microgram dosage levels and tolerability to the highest dose level, as well as a sustained neutralizing antibody (nAb) response against RSV, lasting for at least six months after a single administration of IVX-121. Eligible older adults from the Phase 1b cohort will be followed out to 12 months to further assess durability of response.

### Example 13: Phase 1 Study of a Bivalent Combination Vaccine Formulation for RSV and hMPV

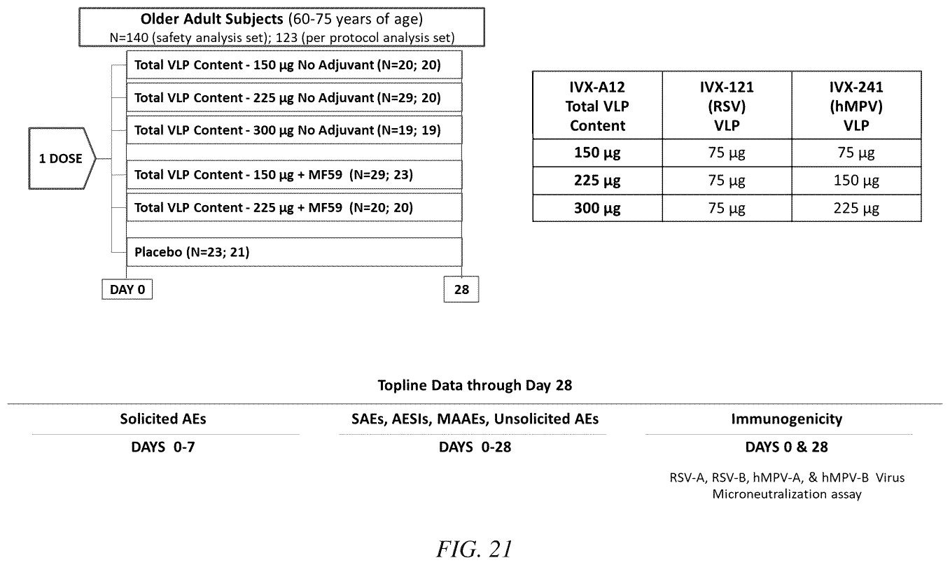

This Example provides interim results from a Phase 1 clinical trial of IVX-A12 (as described in Example 10 above), a bivalent combination formulation containing IVX-121 and IVX-241 virus-like particles (VLPs) for RSV and hMPV, respectively, in older adults. IVX-A12 demonstrated tolerability and an immunologic response in an older adult group. The study design is shown in FIG. 21.

### Clinical Trial Design:

The IVX-A12 phase 1 trial is a randomized, observer-blind, placebo-controlled, multi-center dosage- and age-escalation trial to assess the safety and immunogenicity of one dose of IVX-A12 as aqueous (unadjuvanted) and oil-in-water adjuvant emulsion-adjuvanted (MF59) formulations (IVX-A12a and IVX-A12d, respectively).

A total of 5 IVX-A12 formulations were tested: 3 dosage levels (low, medium and high total VLP content) of IVX-A12a and 2 dosage levels (low and medium) of IVX-A12d. In addition, placebo will be administered as a control.

The Phase 1 part of the trial enrolled 141 healthy older adults aged 60-75 years. 140 subjects were dosed (safety analysis set) with 17 subjects excluded for protocol violations, leaving 123 subjects in the per protocol analysis set.

The Subjects were administered a single dose of IVX-A12 at one of three dose levels (150, 225, 300 μg), or placebo. Subjects administered 150 μg or 225 μg were dosed with or without MF59 adjuvant.

The primary outcomes of the study were safety and immunogenicity up to 7 days post-vaccination; neutralizing antibodies to RSV-A, RSV-B, hMPV-A, and hMPV-B were measured in international units (IU/mL) using the WHO international reference standard.

### Topline Results

### Safety

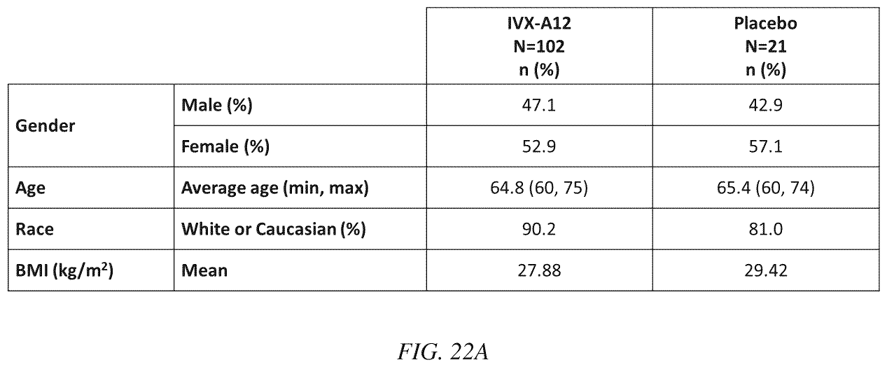

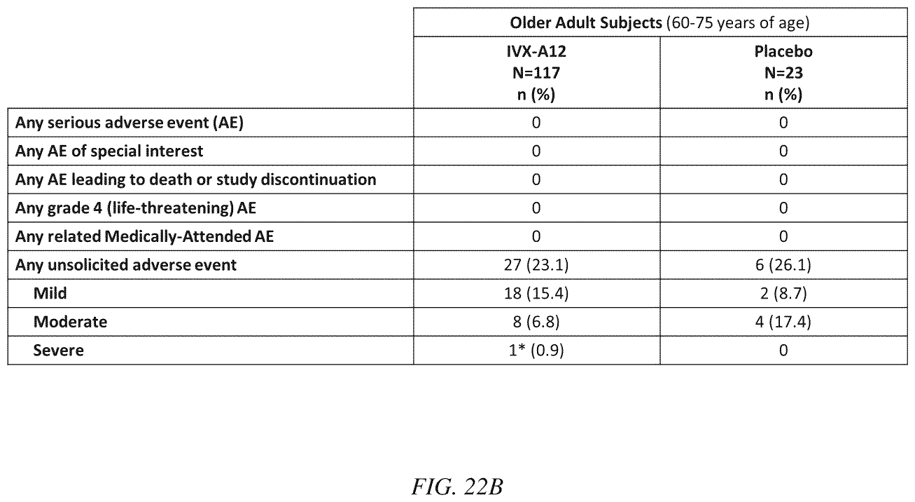

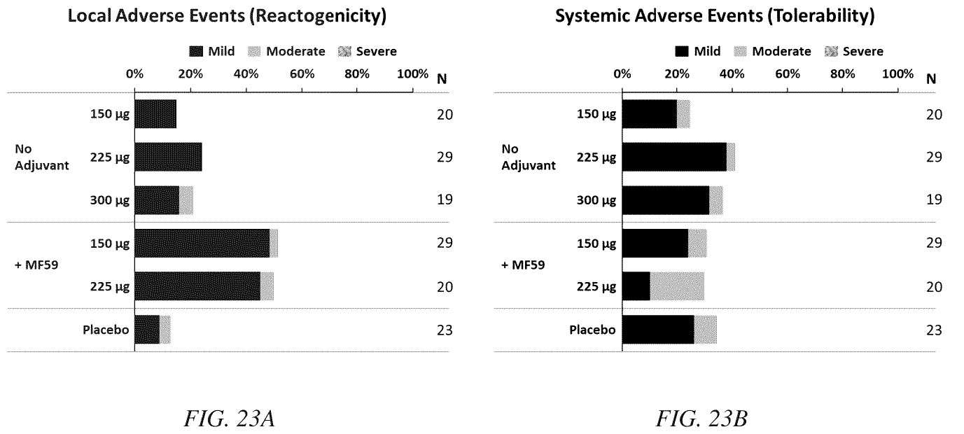

In this Phase 1 study, IVX-A12 was generally well-tolerated across all dosage groups. Solicited local and systemic adverse events (AEs) were generally mild or moderate, without dose-limiting reactogenicity. In the older adult target population, across the five dosage groups for IVX-A12 with or without adjuvant, the proportion of subjects experiencing any unsolicited AE within twenty eight days was 23.1%, and similar to 26.1% for placebo (FIG. 22). There were no serious AEs (SAEs) related to vaccine, dose-limiting adverse events, AEs of special interest (AESIs), or AEs leading to study discontinuation. Data are shown in FIGS. 23A and 23B. As shown in the data, IVX-A12 was well tolerated with mild reactogenicity across tested formulations (FIG. 23A) and with systemic AEs comparable to placebo (FIG. 23B).

### Immunogenicity:

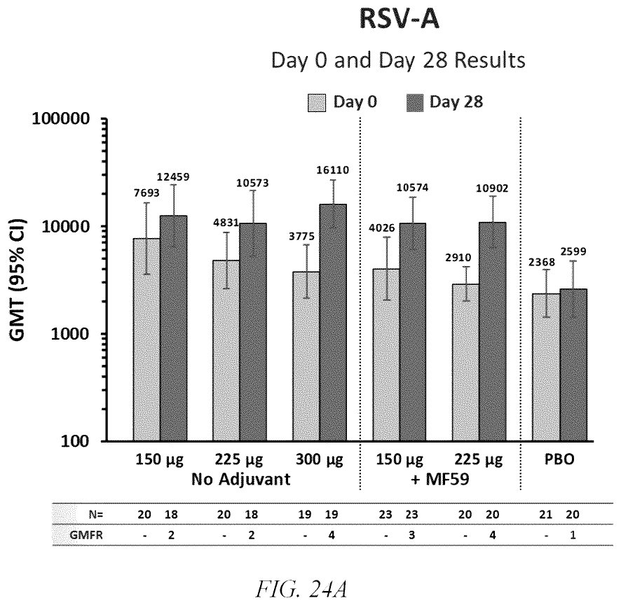

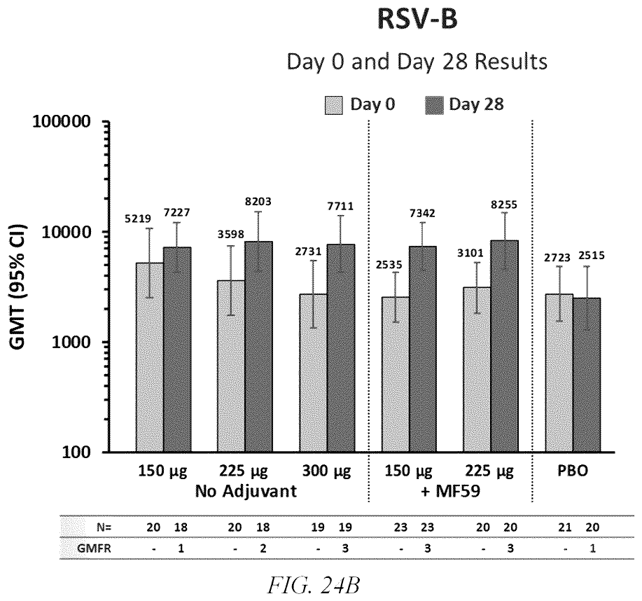

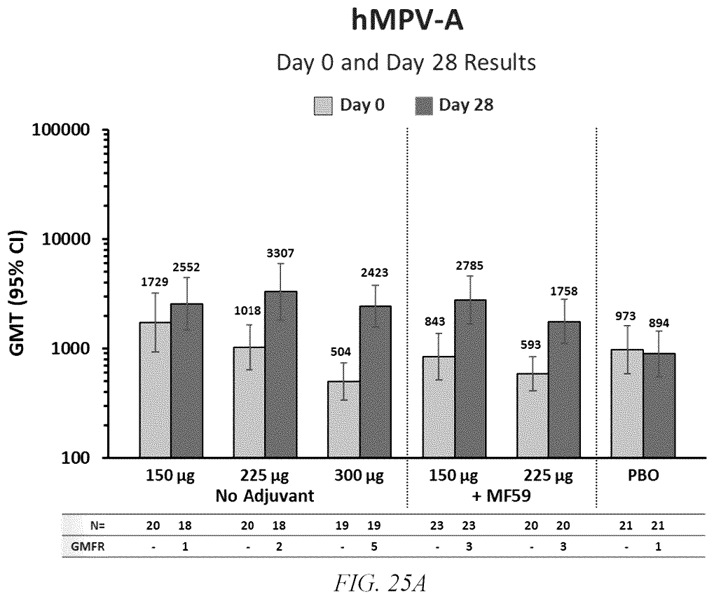

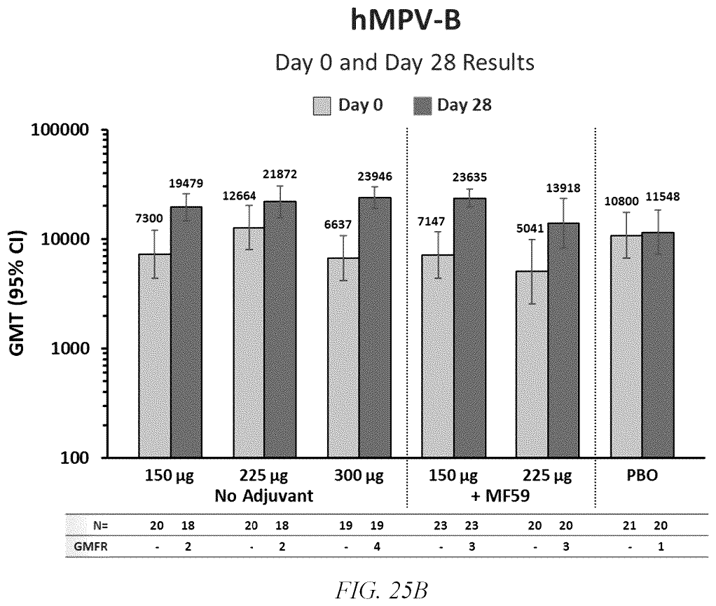

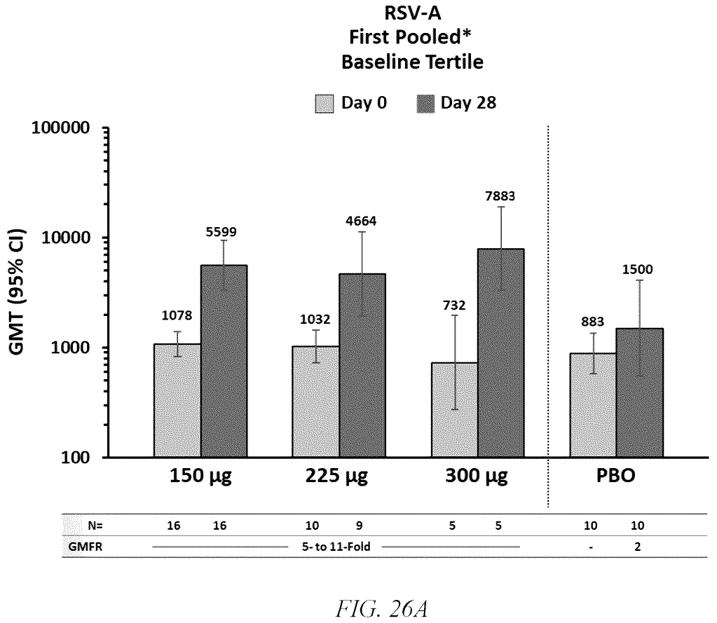

IVX-A12 induced neutralizing antibodies across both RSV and hMPV strains in the older adult group. Data indicated no interference between RSV and hMPV VLPs administered in combination and with increasing hMPV dosage levels (FIGS. 24 and 25). Data indicated evidence of immunogenicity against both RSV-A and RSV-B Neutralizing antibodies (FIGS. 24A and 24B). Geometric mean titers (GMT) for RSV-A and RSV-B were in comparable ranges (FIGS. 24A and 24B). Data indicated evidence of immunogenicity against both hMPV-A and hMPV-B Neutralizing antibodies (FIGS. 25A and 25B). Geometric mean titers (GMT) for hMPV-A and hMPV-B were in comparable ranges (FIGS. 25A and 25B).

Across dosage groups, IVX-A12 induced GMT in RSV-A nAbs of up to 16,110 IU/mL compared to 2,599 IU/mL for placebo at Day 28 (FIG. 24A). IVX-A12 induced GMT in RSV-B nAbs of up to 8,255 IU/mL compared to 2,515 IU/mL for placebo at Day 28 (FIG. 24B). IVX-A12 induced GMT in hMPV-A nAbs of up to 3,307 IU/mL compared to 973 IU/mL for placebo at Day 28 (FIG. 25A). IVX-A12 induced GMT in hMPV-B nAbs of up to 23,946 IU/mL compared to 11,548 IU/mL for placebo at Day 28 (FIG. 25B). Geometric Mean Fold Rise (GMFR) at Day 28 was up to 1- to 5-fold for hMPV-A and 2- to 4-fold for hMPV-B, reflecting high baseline titers in the older adults group (FIG. 25).

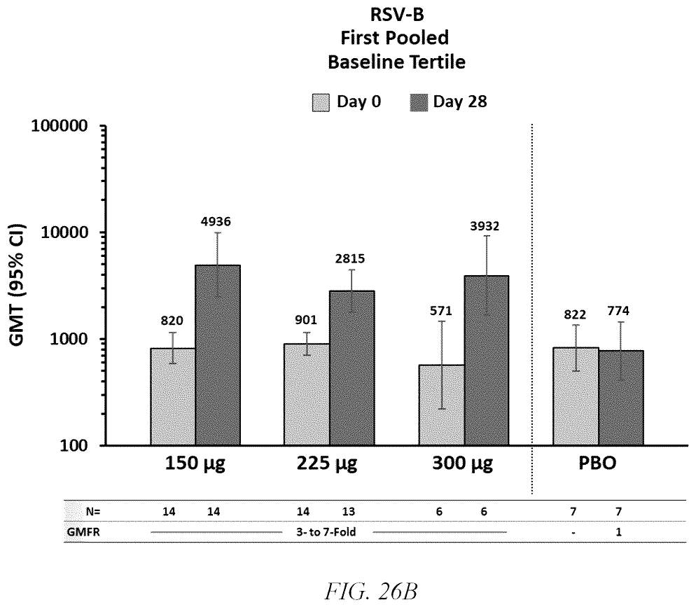

Pre-specified sub-analysis of study subjects with lowest tertile baseline neutralizing antibody titers. Data pooled across non-adjuvanted and adjuvanted groups for each relevant dose level. Data indicate up to 11- or 7-fold rise in GMFR across RSV-A (FIG. 26A), RSV-B (FIG. 26B) responses, respectively, reflecting impact of high baseline titers on full analysis set.

### Conclusion

These data indicate that IVX-A12 shows favorable safety and induced immunogenicity against RSV and hMPV with the same vaccine in healthy older adults. These data demonstrated induced antibody response against RSV and hMPV across all dose levels tested tested with and without adjuvant. IVX-A12 exhibits immunogenicity against RSV-A, RSV-B, hMPV-A and hMPV-B strains at the lowest microgram dosage levels and tolerability to the highest dose level, with no evidence of immune interference against either RSV or hMPV. Eligible older adults will be followed out to 180 days to assess durability of response.

### Neutralizing Antibody Assay Methods

Validated RSV/A, RSV/B, hMPV/A, and hMPV/B neutralizing antibody (NAb) assays were used to measure the presence of RSV- or hMPV-specific neutralizing antibodies. These assays have been validated for standard bioanalytical assay parameters to include Specificity, Accuracy, Precision, Repeatability (intra-assay precision), Intermediate Precision, Linearity, Dilutional Linearity, Range, Stability, and Robustness. Critical reagents for these assays are commercially purchased and qualified for use. In brief, human serum samples were serially diluted and incubated with a constant concentration of virus. Neutralization is measured by the inhibition of virus propagation in HEp-2 cells (ATCC, Cat #CCL23). Following an incubation period, cells were fixed and immunostained with a murine monoclonal antibody directed against RSV or hMPV F protein, followed by horse radish peroxidase (HRP)-conjugated goat-anti-mouse antibody and TrueBlue (TB). The plates were scanned with an UV Analyzer and Spot counts (SC; or spot forming cells, SCF) per well at each serum/antibody concentration were quantified. The values were then analyzed to determine the dilution of serum/antibody that yield a selected reduction point (i.e., 50% or IC50).

### Example 14: A Phase 2a Randomized, Observer-Blind, Placebo-Controlled, Dosage Optimization, Multi-Center Clinical Trial to Evaluate the Safety and Immunogenicity of IVX-A12, a Respiratory Syncytial Virus and Human Metapneumovirus Bivalent Combination Virus-Like Particle Protein Subunit Vaccine, in Adults 60 to 85 Years of Age

The candidate vaccine, IVX-A12, is a bivalent combination formulation containing IVX-121 and IVX-241 virus-like particles (VLPs), computationally designed recombinant protein subunit vaccines for RSV and hMPV, respectively. Each VLP is composed of two recombinant proteins, Component A (CompA) and Component B (CompB-01), which have been designed to cooperatively assemble to form an icosahedral structure. Component A is a fusion protein specific for each vaccine candidate (either CompA-RSV-02 or CompA-hMPV-01 for RSV and hMPV, respectively) and is expressed with the prefusion F protein from the respective virus, which has been shown to induce robust neutralizing antibody responses in nonclinical models. CompB-01 is a common component across both VLPs and provides the structural element that supports the multimeric display of CompA-RSV-02 or CompA-hMPV-01. When combined, the two components self-assemble into a VLP that shows enhanced immunogenicity compared with either soluble DS-Cav1 or hMPV prefusion antigen trimer. The soluble DS-Cav1 RSV prefusion F-protein was recently tested clinically and shown to be tolerable and durably immunogenic (Ruckwardt et al., 2021).

The IVX-121 and IVX-241 VLPs display 20 copies of the respective prefusion F protein trimers on their surface. The candidate IVX-121 and IVX-241 VLPs will be manufactured independently and combined to create a bivalent formulation containing both VLPs, referred to as IVX-A12. The proposed mechanism of action of IVX-A12 is to increase the proportion of RSV- and hMPV-specific neutralizing antibodies that are associated with protection, compared to non-neutralizing antibodies (Ngwuta et al., 2015; Falloon et al., 2017). IVX-A12 is intended for active immunization of the target population of older adults who are most at risk for disease following infection with RSV and hMPV.

The candidate vaccine, IVX-A12, is expected to be a single-dose liquid formulation (0.5 mL) for intramuscular (IM) use in adults 60 to 85 years of age, the target population. The IVX-A12 candidate vaccine will be formulated and vialed as an aqueous vaccine, to be diluted 1:1 (V/V) with diluent or MF59® an oil-in-water emulsion as an adjuvant.

The proposed Phase 2a clinical trial will evaluate the safety and immunogenicity of IVX-A12 to allow the selection of an optimal dosage level for adults 60 to 85 years of age. The safety and immunogenicity of two dosage levels of the IVX-A12 bivalent candidate vaccine, either unadjuvanted or adjuvanted with MF59®, will be evaluated compared to placebo. The investigational formulations of IVX-A12 for this Phase 2a clinical trial will be selected based on the interim safety and immunogenicity data from the ongoing IVX-A12 Phase 1 clinical trial described above. Either of two different IVX-A12 formulations or placebo will be administered as a single IM dose to older adult subjects enrolled into two age strata (adults 60 to 69 years of age and elderly adults 70 to 85 years of age). Eligible older adult subjects will be randomized to the different trial groups to ensure balance among the groups.

The trial will be conducted in accordance with the protocol, International Council for Harmonisation of Technical Requirements for Pharmaceuticals for Human Use (ICH), Good Clinical Practice (GCP) Guidelines, and applicable regulatory requirements.

### Primary Safety Objective:

To assess the safety and tolerability of IVX-A12 bivalent candidate vaccine compared to placebo up to 1 year following a single IVX-A12 vaccination.

### Primary Immunogenicity Objectives:

1. To evaluate the serum NAb response to IVX-A12 or placebo, measured by live virus serum neutralization assays (SNA), at Day 28.

2. To evaluate the serum immunoglobulin G (IgG) binding antibody response to IVX-A12 or placebo, measured by enzyme-linked immunosorbent assays (ELISA), at Day 28.

3. To evaluate the proportion of subjects with a ≥4-fold increase in serum NAb and serum IgG binding antibody titers to IVX-A12 from prevaccination/baseline (Day 0) to Day 28.

### Secondary Safety Objectives:

To assess the safety of IVX-A12 compared to placebo by the incidence of serious adverse events (SAEs), medically-attended adverse events (MAAEs), LRTI cases of any severity (mild, moderate, severe) caused by RSV and/or hMPV (clinical event of special interest [CESI]), LRTI cases of any severity (mild, moderate, severe) not caused by RSV or hMPV, and adverse events (AEs) leading to trial withdrawal up to Day 365 (end of trial).

### Secondary Immunogenicity Objectives:

1. To evaluate the serum NAb response to IVX-A12 or placebo at Day 0, Day 180, and Day 365.

2. To evaluate the serum IgG binding antibody response to IVX-A12 or placebo at Day 0, Day 180, and Day 365 after vaccination.

3. To evaluate the proportion of subjects with a ≥4-fold increase in serum NAb and serum IgG binding antibody titers at Days 180 and 365 compared to baseline (Day 0).

4. To evaluate the proportion of subjects with an ≥8-fold increase in serum NAb and serum IgG binding antibody titers at Days 28, 180 and 365 compared to baseline (Day 0).

### Exploratory Immunogenicity Objectives:

To evaluate the VLP core-specific IgG antibody titers, measured by ELISA, after vaccination.

### Exploratory Efficacy Objective:

To enumerate the RSV and/or hMPV cases of mild, moderate, or severe LRTI (CESI) and any severity LRTI by vaccine group from Day 14 post-vaccination up to Day 365.

### Clinical Trial Design:

The IVX-A12 Phase 2a clinical trial is a randomized, observer-blind, placebo-controlled, dosage optimization, multi-center trial to evaluate the safety and immunogenicity of a single IM dose of two different dosage levels (A and B) of IVX-A12 compared to placebo (diluent) in adults 60 to 85 years of age. The dosage level and formulation (either aqueous or adjuvanted with MF59®) will be determined based on the combined safety and immunogenicity data of the IA from the IVX-A12 Phase 1 clinical trial (as described in the Examples above).

This Phase 2a clinical trial will have three treatment groups: two IVX-A12 groups and one placebo group. The total amount of VLP in the two IVX-A12 groups will not exceed 300 μg. If the low or medium dosage level is selected, the total amount of VLP will be 150 μg or 225 μg, respectively, and could be either unadjuvanted (aqueous) or adjuvanted with MF59®. If the high dosage level is selected, the total amount of VLP will be 300 μg and could be either unadjuvanted (aqueous) or adjuvanted with MF59®. Dosage level selection and formulation (aqueous or adjuvanted with MF59) will be dictated by the combined safety and immunogenicity review of IA data of the Phase 1 clinical trial and a subsequent amendment of this Phase 2a clinical trial protocol will incorporate the IVX-A12 dosage levels and formulations selected for evaluation.

Approximately 500 subjects will be randomly allocated at a ratio of 2:2:1 to receive one of the two IVX-A12 antigen amounts (A or B) or placebo, respectively. Subjects will also be stratified by age group (60 to 69 years of age and 70 to 85 years of age). The stratified randomization will ensure balanced dosage level group assignment between the two age groups (60 to 69 years of age and 70 to 85 years of age, respectively). Dosing of the two IVX-A12 groups and placebo groups will start in parallel. Age-stratified randomization will balance assignment between the two age groups. Subject allocation to trial groups may be done according to one or more of Tables 15-22. The two dosage levels of the IVX-A12 bivalent candidate vaccine will be either unadjuvanted (aqueous) or adjuvanted with MF59®.The total amount of VLP in the two IVX-A12 groups will not exceed 300 μg. If the medium dosage level is selected, the total amount of VLP will be 225 μg, and could be either unadjuvanted (aqueous) or adjuvanted with MF59®. If the high dosage level is selected, the total amount of VLP will be 300 μg and could be either unadjuvanted (aqueous) or adjuvanted with MF59®.

TABLE 15
Clinical Trial Schema 1

| | | Formulation | | | |
|---|---|---|---|---|---|
| Trial | | VLP (μg) | | MF59 ® | |
| Group | IVX-A12 | RSV | hMPV | (mg squalene) | Route |
| A | Dosage level A | 25 | 125 | 0 | IM |
| B | Dosage level B | 25 | 125 | 9.75 | IM |
| C | Placebo | — | — | 0 | IM |

TABLE 16
Clinical Trial Schema 2

| | | Formulation | | | |
|---|---|---|---|---|---|
| Trial | | VLP (μg) | | MF59 ® | |
| Group | IVX-A12 | RSV | hMPV | (mg squalene) | Route |
| A | Dosage level A | 75 | 75 | 0 | IM |
| B | Dosage level B | 75 | 75 | 9.75 | IM |
| C | Placebo | — | — | 0 | IM |

TABLE 17
Clinical Trial Schema 3

| | | Formulation | | | |
|---|---|---|---|---|---|
| Trial | | VLP (μg) | | MF59 ® | |
| Group | IVX-A12 | RSV | hMPV | (mg squalene) | Route |
| A | Dosage level A | 75 | 150 | 0 | IM |
| B | Dosage level B | 75 | 150 | 9.75 | IM |
| C | Placebo | — | — | 0 | IM |

TABLE 18
Clinical Trial Schema 4

| | | Formulation | | | |
|---|---|---|---|---|---|
| Trial | | VLP (μg) | | MF59 ® | |
| Group | IVX-A12 | RSV | hMPV | (mg squalene) | Route |
| A | Dosage level A | 75 | 225 | 0 | IM |
| B | Dosage level B | 75 | 225 | 9.75 | IM |
| C | Placebo | — | — | 0 | IM |

TABLE 19
Clinical Trial Schema 5

| | | Formulation | | | |
|---|---|---|---|---|---|
| Trial | | VLP (μg) | | MF59 ® | |
| Group | IVX-A12 | RSV | hMPV | (mg squalene) | Route |
| A | Dosage level A | 112.5 | 112.5 | 0 | IM |
| B | Dosage level B | 112.5 | 112.5 | 9.75 | IM |
| C | Placebo | — | — | 0 | IM |

TABLE 20
Clinical Trial Schema 6

| | | Formulation | | | |
|---|---|---|---|---|---|
| Trial | | VLP (μg) | | MF59 ® | |
| Group | IVX-A12 | RSV | hMPV | (mg squalene) | Route |
| A | Dosage level A | 150 | 150 | 0 | IM |
| B | Dosage level B | 150 | 150 | 9.75 | IM |
| C | Placebo | — | — | 0 | IM |

TABLE 21
Clinical Trial Schema 7

| | | Formulation | | | |
|---|---|---|---|---|---|
| Trial | | VLP (μg) | | MF59 ® | |
| Group | IVX-A12 | RSV | hMPV | (mg squalene) | Route |
| A | Dosage level A | 225 | 75 | 0 | IM |
| B | Dosage level B | 225 | 75 | 9.75 | IM |
| C | Placebo | — | — | 0 | IM |

TABLE 22
Clinical Trial Schema 8

| | | Formulation | | | |
|---|---|---|---|---|---|
| Trial | | VLP (μg) | | MF59 ® | |
| Group | IVX-A12 | RSV | hMPV | (mg squalene) | Route |
| A | Dosage level A | 150 | 75 | 0 | IM |
| B | Dosage level B | 150 | 75 | 9.75 | IM |
| C | Placebo | — | — | 0 | IM |

### Trial Procedures:

Approximately 500 adult subjects (60 to 85 years of age) will be enrolled. A subject is considered to be enrolled upon having signed the informed consent form (ICF). Subjects will be randomly allocated to receive either IVX-A12 or placebo at a ratio 2:2:1, resulting in approximately 200 subjects in each IVX-A12 group and 100 in the placebo group. In addition, subjects will be stratified by age groups (60 to 69 years of age versus 70 to 85 years of age).

Randomization will occur simultaneously into all three trial groups (IVX-A12 group A, IVX-A12 group B, and placebo, group C). Initial vaccinations will be performed in sentinel subjects and then expanded to the remaining subjects in each of the three trial groups. In each IVX-A12 group, 8 sentinel subjects will receive IVX-A12 (4 subjects from age stratum 60 to 69 years of age and 4 subjects from age stratum 70 to 85 years of age). In the placebo group, 4 sentinel subjects will receive placebo (2 subjects from age stratum 60 to 69 years of age and 2 subjects from age stratum 70 to 85 years of age). Sentinels will be closely monitored for a minimum of 36 hours. If no stopping rules are triggered, the remaining subjects in the IVX-A12 and placebo groups will be enrolled.

There will be a total of seven scheduled clinic visits required for each subject (at screening, then Days 0, 7, 28, 90, 180, and 365), and five phone contacts (on Days 3, 14, 56, 135, and 270) for all subjects.

### Assessments:

### Safety Assessments:

All solicited local reactions and systemic AEs will be collected for all subjects for 7 consecutive days, starting from the day of the single-dose administration (Day 0) through Day 6 (7 days total); unsolicited AEs through Day 28; and safety blood samples (clinical laboratory evaluation at screening, Day 0/baseline, Day 7, and Day 28). AEs, SAEs, MAAEs, AEs leading to trial withdrawal, and CESI will be captured from the time of randomization to the end of the trial evaluation on Day 365, approximately 12 months after vaccination. Clinic staff (a trained healthcare provider) will contact the subject by phone call or using a digital application (e.g., SMS text) to facilitate the collection of relevant safety information such as concomitant medication use and occurrence of AEs/SAEs. Throughout the trial, all subjects will complete a respiratory symptom surveillance tool approximately twice a week (i.e., approximately twice within 7 day intervals starting on Day 0 post-vaccination) to be monitored for the trial-defined CESI of mild, moderate, or severe LRTI caused by RSV and/or hMPV, using the established case definition and algorithm for evaluation of acute respiratory illness (ARI) and using nasopharyngeal (NP) swabs for the detection of RSV, hMPV, and other respiratory viruses. Subjects will be instructed to return to the clinic for assessment within 3 7 days of onset of ARI (ideally within 3 days) to maximize detection of virus.

### Immunogenicity Assessments:

Blood sampling for immunogenicity will be taken at Days 0, 28, 180, and 365.

Serological samples will be assessed for primary or secondary immunogenicity endpoints using the following assays:
RSV-A-specific and hMPV-A-specific NAb assay;
RSV-B-specific and hMPV-B-specific NAb assay;
Anti-RSV and anti-hMPV prefusion F protein-specific IgG ELISA.

In brief, the RSV-A and RSV-B NAb, and hMPV-A and hMPV-B NAb assays have been validated to measure the presence of RSV-specific NAbs following the incubation of serially-diluted serum with a constant concentration of virus. Neutralization will be measured by the inhibition of virus propagation in Hep-2 cells (RSV) or Vero cells (hMPV). Spot counts will be quantified to determine the dilution of serum that corresponds to the selected reduction point (e.g., 50% for IC50).

Serological samples will be assessed for exploratory immunogenicity endpoints using the following assays:
Anti-VLP core IgG ELISA.

An anti-VLP core ELISA will be qualified for the quantification of binding IgG antibodies to VLP core in human serum samples.

Diagnostic samples (NP swabs) will be evaluated for the exploratory efficacy endpoint for the detection of the trial-defined CESI of mild, moderate, or severe LRTI caused by RSV and/or hMPV. Subjects will be instructed to return to the clinic for assessment within 3-7 days of onset of ARI (ideally within 3 days) to maximize detection of the etiological agent using the following assay:

A polymerase chain reaction (PCR)-based multiplexed nucleic acid assay (BioMerieux BioFire Respiratory Panel 2.1) to detect RSV and/or hMPV as well as other respiratory pathogens. RSV and/or hMPV PCR positive samples may be further analyzed by sequencing to determine the RSV and/or hMPV subgroups.

### Subject Population:

Subject Population (age range): Adults 60 to 85 years of age.

Planned Number of Subjects: Approximately 500 total; approximately 200 in each of the two IVX-A12 groups and 100 in the placebo group.

Planned Number of Groups: A total of three trial groups: two IVX-A12 groups and one placebo group. The two IVX-A12 groups will consist of different dosage levels/formulations.

### Inclusion Criteria:

1. Male or female subjects, 60 to 85 years of age, who must be in stable health based on medical history, vital signs, physical examination, and laboratory evaluation prior to vaccination, in the investigator's clinical judgment;
2. Subjects may have ongoing chronic conditions (comorbidities such as hypertension, congestive heart failure, chronic obstructive pulmonary disease, type 2 diabetes mellitus, hyperlipoproteinemia, or hypothyroidism) who are, in the investigator's opinion, medically compensated and without recent exacerbation (for example, no emergency room [ER] visits and no change to medical treatments or medications [unless change is for a more effective treatment/medication] to address the condition) within the prior 3 months;
3. Subjects able to voluntarily give written informed consent and can comply with trial procedures including follow-up for approximately 12 months after vaccination;
4. Body mass index 17 to <40 kg/m² at screening;
5. Before randomization, female subjects must be postmenopausal (postmenopausal state is defined as no menses for 12 months without alternative medical cause) and not intending to conceive by any method;
6. Subject must agree not to donate blood from the time of vaccination through 3 months after vaccination;
7. Subject must be willing to provide verifiable identification and have the means to be contacted and to contact the investigator or the site's staff during the entire clinical trial.

### Exclusion Criteria:

1. Subjects with moderate or severe liver disease, metastatic solid tumor, and acquired immunodeficiency syndrome (AIDS) are to be excluded. In addition, subjects with underlying significant illness or condition(s) or ongoing treatment that, in the opinion of the investigator, could (i) interfere with the conduct of the trial, (ii) pose an unacceptable risk to the subject in this trial, (iii) interfere with the subject's ability to comply with the trial procedures or abide by the trial restrictions, (iv) interfere with the ability to interpret safety data, or (v) prevent completion of the trial are to be excluded;
2. Older adults who meet frail elderly criteria (older persons with medical, nutritional, cognitive, emotional, or activity impairments, as defined by the Dalhousie Clinical Frailty Score ≥4 (Rockwood et al., 2005);
3. Prior receipt of any licensed or investigational RSV or hMPV vaccine within the past 12 months;
4. Prior receipt of another investigational medicinal product (IMP; trial drug, biologic, or device) not authorized for use in the USA and European Union within the past 3 months;
5. Laboratory-confirmed RSV or hMPV infection within 12 months prior to enrollment;
6. Currently enrolled or plan to participate in another clinical trial with an investigational agent (including licensed or unlicensed vaccine, drug, biologic, device, blood product, or medication) to be received during the trial period;
7. History of malignancy within 5 years before screening not in the following categories: (i) subjects with squamous and basal cell carcinomas of the skin and carcinoma in situ of the cervix may be enrolled at the discretion of the investigator, (ii) subjects with a history of malignancy within 5 years before screening, with minimal risk of recurrence per investigator's judgment, can be enrolled;
8. Per medical history, subjects with chronic active hepatitis B or hepatitis C infection, human immunodeficiency viruses type 1 or type 2 infection, acute polyneuropathy (such as Guillain-Barre syndrome), or chronic idiopathic demyelinating polyneuropathy; subjects whose hepatitis C antibody test is positive but whose viral loads are negative may be enrolled;
9. Acute illness, with or without fever at the time of planned vaccination;
10. History of hypersensitivity or serious adverse reactions to vaccines, such as anaphylaxis or angioedema, or any known allergies to any component of the IVX 121 and/or IVX-241 vaccine, or hypersensitivity to latex;
11. Abnormal function of the immune system resulting from clinical conditions including chronic administration of systemic corticosteroids (oral/intravenous/IM at a daily dose equivalent of >20 mg prednisone in a period of more than 14 days), or administration of immunosuppressive chemotherapy, biologics, or radiotherapy within the past 3 months prior to planned vaccination;
12. Subjects who have received treatment with immunoglobulins or other biologics, such as immunosuppressive therapies expected to modify immune response to vaccination (including monoclonal antibodies [MAbs] for chronic underlying conditions) in the past 3 months prior to planned vaccination;
13. Trial personnel as an immediate family or household member;
14. For licensed vaccines:
   a. Receipt of licensed inactivated vaccines (including seasonal influenza vaccine) within 14 days prior to trial vaccine administration on Day 0, or licensed replicating vaccines such as RNA or live-attenuated virus vaccines within 30 days prior to Day 0.
   b. Receipt of licensed vaccines is permitted after completion of the Day 28 visit.
   c. Receipt of any licensed COVID-19 vaccines is permitted if dosing regimen completed within 21 days prior to Day 0 or after completion of the Day 28 visit.

### Investigational Medicinal Products:

IVX-A12: The IVX-A12 formulations for testing will be composed of different IVX-121 RSV VLP dosage level combined with different dosage levels of the IVX-241 hMPV VLP. The IVX-A12 formulations may be either unadjuvanted aqueous or adjuvanted with MF59®.

Placebo: Sterile aqueous diluent, delivered as a 0.5 mL dose. The placebo does not contain preservatives.

Route of Administration: IM administration of a single IVX-A12 or placebo dose into the deltoid of the non-dominant arm.

### Duration Subject Participation:

The duration of subject participation in the trial will be approximately 12 months.

### Criteria for Evaluation and Analyses:

### Primary Safety Endpoints:

1. Solicited local reactions and systemic AEs for 7 consecutive days starting from Day 0 to Day 6;
2. Unsolicited AEs from Day 0 to Day 28.

### Primary Immunogenicity Endpoints:

1. Geometric mean titers (GMT) of RSV-A-, RSV-B-, hMPV-A-, and hMPV-B-specific NAb at Day 28;
2. GMT of RSV and hMPV prefusion F protein-specific IgG antibody titers at Day 28;
3. Proportion of subjects with a ≥4-fold increase in serum anti-RSV-A-, RSV-B-, hMPV-A-, and hMPV-B-specific NAb from prevaccination/baseline (Day 0) to Day 28 after vaccination (defined as seroresponse rate [SRR]);
4. Proportion of subjects with ≥4-fold increase (SRR) in RSV- and hMPV-specific IgG antibody titers from prevaccination/baseline (Day 0) to Day 28;
5. Geometric mean fold rise (GMFR) at Day 28 versus Day 0 in serum anti RSV-A-, RSV-B-, hMPV-A-, and hMPV-B-specific NAb and RSV and hMPV prefusion F protein specific IgG antibody titers.

### Secondary Safety Endpoints:

1. SAEs, MAAEs, and AEs leading to trial withdrawal up to Day 365 (end of trial);
2. Mild, moderate, or severe LRTI cases caused by RSV and/or hMPV (CESI) up to Day 365;
3. Clinical safety laboratory parameters at screening, and Days 0, 7, and 28;
4. Mild, moderate, or severe LRTI cases not caused by RSV or hMPV up to Day 365;

### Secondary Immunogenicity Endpoints:

1. GMT of RSV-A-, RSV-B-, hMPV-A-, and hMPV-B-specific NAb at Days 0, 180, and 365;
2. GMTs of RSV and hMPV prefusion F protein-specific IgG antibody titers at Days 0, 180, and 365;
3. Proportion of subjects with a ≥4-fold increase (SRR) in serum anti-RSV-A-, RSV-B-hMPV-A-, and hMPV-B-specific NAb from prevaccination/baseline (Day 0) to Days 180, and 365 after vaccination;
4. Proportion of subjects with an ≥8-fold increase in serum anti-RSV-A-, RSV-B-, hMPV-A-, and hMPV-B-specific NAb from prevaccination/baseline (Day 0) to Days 28, 180, and 365 after vaccination;
5. Proportion of subjects with ≥4-fold increase (SRR) in RSV- and hMPV-prefusion F protein-specific IgG antibody titers from prevaccination/baseline (Day 0) to Days 180 and 365 after vaccination;
6. GMFR at Days 180 and 365 versus Day 0 in serum anti-RSV-A-, RSV-B-, hMPV-A-, and hMPV-B-specific NAb and RSV and hMPV prefusion F protein-specific IgG antibody titers;
7. Reverse cumulative distribution (RCD) of serum NAb and IgG antibody titers at Days 0, 28, 180, and 365.

### Exploratory Immunogenicity Endpoint:

VLP core-specific IgG titers at Days 0, 28, 180, and 365.

### Exploratory Efficacy Endpoint:

RSV and/or hMPV cases of mild, moderate, or severe LRTI (CESI) and any severity LRTI by vaccine group from Day 14 post-vaccination up to Day 365.

### Statistical Considerations:

### Analysis Sets:

Safety Set: The safety set will consist of all subjects who received a dose of IVX-A12 or placebo. Subjects will be analyzed as treated.

Full Analysis Set (FAS): The FAS will include all randomized subjects who received a dose of IVX-A12 or placebo. Subjects will be analyzed as randomized.

Per-Protocol Set (PPS): The PPS will include all subjects in the FAS who received a dose of IVX-A12 or placebo and have no major protocol deviations, which may exclude subjects from the PPS.

### Analysis of Demographics and Other Baseline Characteristics

Age, gender, race, and other baseline characteristics (including baseline RSV and hMPV NAb titers) will be summarized descriptively by treatment group for all randomized subjects.

### Immunogenicity Analyses:

Immunogenicity data will be summarized for each treatment group and strain (RSV-A, RSV-B, both RSV strains and either RSV strain; hMPV-A, hMPV-B, both hMPV strains and either hMPV strain). The GMT will be calculated for RSV-A, RSV-B, hMPV-A, and hMPV-B titers separately along with their 95% confidence interval (CI). The adjusted GMT and corresponding 95% CI will be obtained from an analysis of covariance (ANCOVA) model, which will include log-transformed baseline titers, age group, and treatment group as terms. Durability will be assessed at Days 180 and 365 by comparing to Day 28 data. The geometric mean of fold changes and corresponding 95% CI will be calculated.

Proportion of subjects with ≥4 fold increase (SRR), 8 fold increase, and GMFR from prevaccination/baseline (Day 0) will be summarized for Days 28, 180, and 365 after vaccination. RCD curves will be generated.

The VLP core-specific IgG titers will be summarized at Days 0, 28, 180, and 365, with GMT and 95% CI.

### Efficacy Analysis:

RSV and/or hMPV cases of mild, moderate, or severe LRTI and any severity LRTI due to RSV and/or hMPV (CESIs), meeting the protocol-specified definitions, will be summarized by vaccine group and placebo group starting on Day 14 through the end of the trial (Day 365).

### Safety Analyses:

Solicited local reactions and solicited systemic AEs: Solicited AEs will be summarized for 7 days starting from the day of dosing (Day 0) through Day 6 (total of 7 days). The summary will be by day and overall, within 7 days. Solicited AEs by maximum severity will also be summarized.

Unsolicited AEs: Unsolicited AEs will be assessed from day of dosing (Day 0) through 28 days post-vaccination and coded according to MedDRA and summarized by SOC and PT. The summary tables will include the number and percentage of subjects reporting unsolicited AE by SOC and PT, and by SOC, PT, and maximum severity. Subjects reporting ≥Grade 3 and vaccine-related unsolicited AEs will also be summarized.

SAEs, MAAEs, AEs leading to withdrawal, CESI (mild, moderate, or severe LRTI caused by RSV and/or hMPV) and mild, moderate, or severe LRTI cases not caused by RSV or hMPV: Will be assessed throughout the trial (from randomization on Day 0) and coded using MedDRA and summarized by SOC and PT.

### List of Abbreviations

Abbreviations used in the body of the protocol.
AE adverse event
ARI acute respiratory illness
AESI adverse event of special interest
CESI clinical event of special interest
CompA component A
CompB-01componentB-01
CSR clinical study report
ELISA enzyme-linked immunosorbent assay
eCRF electronic case report form
EDC electronic data capture
FAS full analysis set
FDA Food and Drug Administration
FIH first-in-human
GCP good clinical practice
GMFR geometric mean fold rise
GMT geometric mean titer
HCG human chorionic gonadotrophin
hMPV human metapneumovirus
IB investigator's brochure
ICF informed consent form
ICH International Council for Harmonisation of Technical Requirements for Pharmaceuticals for Human Use
IEC Independent Ethics Committee
IM intramuscular
IMP investigational medicinal product
IND investigational new drug
IRB Institutional Review Board
iSRC internal safety review committee
IWRS interactive web response system
LRTI lower respiratory tract infection
MAAE medically-attended adverse events
MedDRA Medical Dictionary for Regulatory Activities
NAb neutralizing antibody
NP nasopharyngeal
PD post-dose
PPS per-protocol set
PT Preferred Term
QTL quality tolerance limit
RCD reverse cumulative distribution
RSV respiratory syncytial virus
RT-PCR reverse transcription polymerase chain reaction
SAE serious adverse event
SMC safety monitoring committee
SOC System Organ Class
SUSAR suspected unexpected serious adverse reaction
VLP virus-like particle
WHO World Health Organization
Abbreviations used without expansion:
° C. degree Centigrade
CI confidence interval
Ig immunoglobulin
mL milliliter
USA United States of America

### REFERENCES

WMA Declaration of Helsinki—Ethical Principles for Medical Research Involving Human Subjects. http://www.wma.net/en/30publications/10policies/b3 (accessed 17 Feb. 2022).
International Council for Harmonisation of Technical Requirements for Pharmaceuticals for Human Use (ICH). ICH harmonized guideline. Integrated Addendum to ICH E6 (R2): Guideline for Good Clinical Practice E6.
Falsey A R, McElhaney J E, Beran J, van Essen G A, Duval X, Esen M, et al. Respiratory syncytial virus and other respiratory viral infections in older adults with moderate to severe influenza-like illness. J Infect Dis. 2014; 209(12):1873-81.
Fleming D M, Taylor R J, Lustig R L, Schuck-Paim C, Haguinet F, Webb D J, et al. Modelling estimates of the burden of Respiratory Syncytial virus infection in adults and the elderly in the United Kingdom. BMC Infect Dis. 2015; 15(1):443.10.1186/s12879-015-1218-z.
Colosia A D, Yang J, Hillson E, Mauskopf J, Copley-Merriman C, Shinde V, Stoddard J. The Epidemiology of Medically Attended Respiratory Syncytial Virus in Older Adults in the United States: A Systematic Review. PloS One. 2017; 12(8):e0182321.
Broberg E K, Waris M, Johansen K, Snacken R, Penttinen P. Seasonality and geographical spread of respiratory syncytial virus epidemics in 15 European countries, 2010 to 2016. Euro Surveill. 2018; 23(5):pii=17-00284. https://doi.org/10.2807/1560-7917.ES.2018.23.5.17-00284.
Brandt C D, Kim H W, Arrobio J O, Jeffries B C, Wood S C, Chanock R M et al. Epidemiology of respiratory syncytial virus infection in Washington, D.C. 3. Composite analysis of eleven consecutive yearly epidemics. Am J Epidemiol. 1973; 98(5):355-64.
Weber M W, Mulholland E K, Greenwood B M. Respiratory syncytial virus infection in tropical and developing countries. Trop Med Int Health. 1998; 3(4):268-80.
Graham B S. Biological challenges and technological opportunities for respiratory syncytial virus vaccine development. Immunol Rev. 2011; 239(1):149-66.
Rima, B, Collins, P, Easton, A, Fouchier R, Kurath G, Lamb R A, et al. ICTV Virus taxonomyprofile: Pneumoviridae. J. Gen Virol, 2017; 98:1912-2913.
van den Hoogen B G, Osterhaus D M, Fouchier R A. Clinical impact and diagnosis of human metapneumovirus infection. Pediatr Infect Dis J. 2004; 23(1 Suppl):S25-32.
Skiadopoulos M H, Surman S R, St Claire M, Elkins W R, Collins P L, Murphy B R. Attenuation of the recombinant human parainfluenza virus type 3 cp45 candidate vaccine virus is augmented by importation of the respiratory syncytial virus cpts530 L polymerase mutation. Virology.1999; 260(1):125-35.
MacPhail M, Schickli J H, Tang R S, Kaur J, Robinson C, Fouchier RAM et al. Identification of small-animal and primate models for evaluation of vaccine candidates for human metapneumovirus (hMPV) and implications for hMPV vaccine design. J Gen Virol. 2004; 85(Pt 6):1655-1663.
Williams J V, Chen Z, Cseke G, Wright D W, Keefer C J, Tollefson S J et al. A recombinant human monoclonal antibody to human metapneumovirus fusion protein that neutralizes virus in vitro and is effective therapeutically in vivo. J Virol. 2007; 81(15):8315-24.
Hamelin M E, Couture C, Sackett M, Kiener P, Suzich J, Ulbrandt N. The prophylactic administration of a monoclonal antibody against human metapneumovirus attenuates viral disease and airways hyperresponsiveness in mice. Antivir Ther. 2008; 13(1):39-46.
Madhi S A, Ludewick H, Kuwanda L, van Niekerk N, Cutland C, Klugman K P. Seasonality, incidence, and repeat human metapneumovirus lower respiratory tract infections in an area with a high prevalence of human immunodeficiency virus type-1 infection. Pediatr Infect Dis J. 2007; 26(8):693-9.
Williams J V, Wang C K, Yang C F, Tollefson S J, House F S, Heck J M et al. The role of human metapneumovirus in upper respiratory tract infections in children: a 20-year experience. J Infect Dis. 2006; 193(3):387-95.
Jain B, Singh A K, Dangi T, Agarwal A, Verma A K, Dwivedi M et al. High prevalence of human metapneumovirus subtype B in cases presenting as severe acute respiratory illness: an experience at tertiary care hospital. Clin Respir J. 2014; 8(2):225-33.
Ruckwardt T J, Morabito K M, Phung E, Crank M C, Costner P J, Holman L A et al. Safety, tolerability, and immunogenicity of the respiratory syncytial virus prefusion F subunit vaccine D S-Cav1: a phase 1, randomised, open-label, dose-escalation clinical trial. Lancet Respir Med. 2021; 9(10):1111-1120.
Ngwuta J O, Chen M, Modjarrad K, Joyce M G, Kanekiyo M, Kumar A et al. Prefusion F-specific Antibodies Determine the Magnitude of RSV Neutralizing Activity in Human Sera. Sci Transl Med. 2015; 7(309):309ra162.
Falloon J, Talbot H K, Curtis C, Ervin J, Krieger D, Dubovsky F et al. Dose Selection for an Adjuvanted Respiratory Syncytial Virus F Protein Vaccine for Older Adults Based on Humoral and Cellular Immune Responses. Clin Vaccine Immunol. 2017; 24(9):e00157-17.
Pellegrini M, Nicolay U, Lindert K, Groth N, Della Cioppa G. MF59-adjuvanted versus non-adjuvanted influenza vaccines: integrated analysis from a large safety database. Vaccine. 2009; 27(49):6959-65.
Bechhofer, R. E., T. J. Santner, and D. Goldsman. 1995. Design and Analysis of Experiments for Statistical Selection, Screening and Multiple Comparisons. New York: John Wiley & Sons.
Guidance for Industry: Toxicity Grading Scale for Healthy Adult and Adolescent Volunteers Enrolled in Preventive Vaccine Clinical Trials. https://www.fda.gov/media/73679/download (accessed 17 Feb. 2022).
Kohl K S, Marcy M, Blum M, Jones M C, Dagan R, Hansen J. et al. Fever after immunization: current concepts and improved future scientific understanding. Clin Infect Dis 2004; 39:389-94.
Recommendations related to contraception and pregnancy testing in clinical trials [Clinical Trials Facilitation and Coordination Group (CFTG) Guidance]. https://legemiddelverket.no/Documents/Godkjenning/Klinisk %20utpr % C3% B8ving/2014_09_HMA_CTFG Contraception_guidance %20Version %201.1.pdf (Accessed 17 Feb. 2022).

While the invention has been described in connection with proposed specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

1. A composition or pharmaceutical composition, comprising:
a) two or more virus-like particles (VLPs), wherein
i. a first virus-like particle (VLP) comprises a first component comprising a respiratory syntactical virus (RSV) F protein ectodomain or antigenic variant thereof, and
ii. a second virus-like particle (VLP) comprises a first component comprising a human metapneumovirus (hMPV) F protein ectodomain or antigenic variant thereof, and/or
b) a virus-like particle (VLP) comprising a plurality of first components, some first components comprising a respiratory syntactical virus (RSV) F protein ectodomain or antigenic variant thereof and some first components comprising a human metapneumovirus (hMPV) F protein ectodomain or antigenic variant thereof.

2. The composition of claim 1, wherein the VLPs each independently comprise:
a) the first component, the first component comprising a first multimerization domain; and
b) a second component comprising a second multimerization domain.

3. The composition of claim 2, wherein the first multimerization domain is selected from SEQ ID NOS: 1, 4, 5, 7, 9, 18, 19, 21, 24, 25, 26, 29, 30, 31, 34, 36, 37, 39, 42, 43, 44, 45, 46, 47, 48, 49, 50, 51, 52, 53, 144, 145, or functional variants thereof.

4. The composition of claim 2, wherein the first multimerization domain shares at least 70%, at least 80%, at least 90%, at least 91%, at least 92%, at least 93%, at least 94%, at least 95%, at least 96%, at least 97%, at least 98%, at least 99%, or 100% identity to I53-50A (SEQ ID NO: 144) or I53-50A ΔCys (SEQ ID NO: 145).

5. The composition of claim 4, wherein the first multimerization domain comprises the amino acid substitutions C74A and C98A; the amino acid substitutions C163A and C201A; or the amino acid substitutions C74A, C98A, C163A, and C201A relative to SEQ ID NO: 144.

6. The composition of claim 2, wherein the second multimerization domain is selected from SEQ ID NOs: 2, 3, 6, 8, 10, 11, 12, 13, 14, 15, 16, 17, 20, 22, 23, 27, 28, 32, 33, 35, 38, 40, and 41 or functional variants and fragments thereof.

7. The composition of claim 6, wherein the second multimerization domain shares at least 70%, at least 80%, at least 90%, at least 91%, at least 92%, at least 93%, at least 94%, at least 95%, at least 96%, at least 97%, at least 98%, at least 99%, or 100% identity to I53-50B (SEQ ID NO: 8) or I53-50B.4PosT1 (SEQ ID NO: 34).

8. The composition of claim 1, wherein the RSV F protein ectodomain shares at least 70%, at least 80%, at least 90%, at least 91%, at least 92%, at least 93%, at least 94%, at least 95%, at least 96%, at least 97%, at least 98%, at least 99%, or 100% identity to RSV F DS-Cav1 (SEQ ID NO: 173):
QNITEEFYQSTCSAVSKGYLSALRTGWYTSVITIELSNIKENKCNGTDAKVKLIKQELDKYK
NAVTELQLLMQSTPATNNRARRELPRFMNYTLNNAKKTNVTLSKKRKRRFLGFLLGVGSAIA
SGVAVCKVLHLEGEVNKIKSALLSTNKAVVSLSNGVSVLTFKVLDLKNYIDKQLLPILNKQS
CSISNIETVIEFQQKNNRLLEITREFSVNAGVTTPVSTYMLTNSELLSLINDMPITNDQKKL
MSNNVQIVRQQSYSIMCIIKEEVLAYVVQLPLYGVIDTPCWKLHTSPLCTTNTKEGSNICLT
RTDRGWYCDNAGSVSFFPQAETCKVQSNRVFCDTMNSLTLPSEVNLCNVDIFNPKYDCKIMT
SKTDVSSSVITSLGAIVSCYGKTKCTASNKNRGIIKTFSNGCDYVSNKGVDTVSVGNTLYYV
NKQEGKSLYVKGEPIINFYDPLVFPSDEFDASISQVNEKINQSLAFIRKSDELL.

9. The composition of claim 8, wherein the RSV F protein ectodomain comprises amino acid substitutions S155C and S290C; and/or amino acid substitutions S190F and V207L.

10. The composition of claim 1, wherein the first component of the first VLP comprises a polypeptide that shares at least 70%, at least 80%, at least 90%, at least 91%, at least 92%, at least 93%, at least 94%, at least 95%, at least 96%, at least 97%, at least 98%, at least 99%, or 100% identity to DS-Cav1-I53-50A (SEQ ID NO: 151):
(SEQ ID NO: 151)
QNITEEFYQSTCSAVSKGYLSALRTGWYTSVITIELSNIKENKCNGTDAKVKLIKQELDKY
KNAVTELQLLMQSTPATNNRARRELPRFMNYTLNNAKKTNVTLSKKRKRRFLGFLLGVGSA
IASGVAVCKVLHLEGEVNKIKSALLSTNKAVVSLSNGVSVLTFKVLDLKNYIDKQLLPILN
KQSCSISNIETVIEFQQKNNRLLEITREFSVNAGVTTPVSTYMLTNSELLSLINDMPITND
QKKLMSNNVQIVRQQSYSIMCIIKEEVLAYVVQLPLYGVIDTPCWKLHTSPLCTTNTKEGS
NICLTRTDRGWYCDNAGSVSFFPQAETCKVQSNRVFCDTMNSLTLPSEVNLCNVDIFNPKY
DCKIMTSKTDVSSSVITSLGAIVSCYGKTKCTASNKNRGIIKTFSNGCDYVSNKGVDTVSV
GNTLYYVNKQEGKSLYVKGEPIINFYDPLVFPSDEFDASISQVNEKINQSLAFIRKSDELL
GSGGSGSGSGGSEKAAKAEEAARKMEELFKKHKIVAVLRANSVEEAIEKAVAVFAGGVHLI
EITFTVPDADTVIKALSVLKEKGAIIGAGTVTSVEQCRKAVESGAEFIVSPHLDEEISQFC
KEKGVFYMPGVMTPTELVKAMKLGHTILKLFPGEVVGPQFVKAMKGPFPNVKFVPTGGVNL
DNVCEWFKAGVLAVGVGSALVKGTPDEVREKAKAFVEKIRGCTE.

11. The composition of claim 1, wherein the hMPV F protein ectodomain shares at least 70%, at least 80%, at least 90%, at least 91%, at least 92%, at least 93%, at least 94%, at least 95%, at least 96%, at least 97%, at least 98%, at least 99%, or 100% identity to SEQ ID NO: 174 or SEQ ID NO: 175:
(SEQ ID NO: 174)
KESYLEESCSTITEGYLSVLRTGWYTNVFTLEVGDVENLTCTDCPSLIKTELDLTKSALRE
LKTVSADQLAREEQIEGGGGGGFVLGAIALGVATAAAVTAGIAIAKTIRLESEVNAIKGCL
KTTNECVSTLGNGVRVLATAVRELKEFVSKNLTSAINKNKCDIADLCMAVSFSQFNRRFLN
VVRQFSDNAGITPAISLDLMTDAELARAVSYMPTSAGQIKLMLENRAMVRRKGFGILIGVY
GSSVIYMVOLPIFGVIDTPCWIIKAAPSCSEKDGNYACLLREDQGWYCKNAGSTVYYPNDK
DCETRGDHVFCDTAAGINVAEQSRECNINISTTNYPCKVSTGRHPISMVALSPLGALVACY
KGVSCSIGSNRVGIIKQLPKGCSYITNQDADTVTIDNTVYQLSKVEGEQHVIKGRPVSSSF
DPICFPEDQFNVALDQVFESIENCQA;
(SEQ ID NO: 175)
KESYLEESCSTITEGYLSVLRTGWYTNVFTLEVGDVENLTCADGPSLIKTELDLTKSALRE
LRTVSADQLAREEQIEGGGGGGFVLGAIALGVATAAAVTAGVAIAKCIRLESEVTAIKNAL
KKTNEAVSTLGCGVRVLATAVRELKDFVSKNLTRAINKNKCDIPDLKMAVSESQFNRRFLN
VVRQFSDNAGITPAISKDLMTDAELARAISNMPTSAGQIKLMLENRAMVRRKGFGILIGVY
GSSVIYMVQLPIFGVIDTPCWIVKAAPSCSEKKGNYACLLREDQGWYCQNAGSTVYYPNEK
DCETRGDHVFCDTAAGINVAEQSKECNINISTTNYPCKVSCGRNPISMVALSPLGALVACY
KGVSCSIGSNRVGIIKQLNKGCSYITNQDADTVTIDNTVYQLSKVEGEQHVIKGRPVSSSF
DPVKFPEDQFNVALDQCFESIENSQA.

12. The composition of claim 11, wherein the hMPV F protein ectodomain comprises amino acid substitutions A63C, A140C, A147C, K188C, K450C, S470C, N97G, P98G, R99G, Q100G, S101G, and/or R102G; or wherein the hMPV F protein ectodomain comprises amino acid substitutions T127C, N153C, T365C, V463C, A185P, L219K, V231I, G294E, N97G, P98G, R99G, Q100G, H386N, S101G and/or R102G relative to relative to reference hMPV F protein sequence (SEQ ID NO: 56).

13. The composition of claim 1,
wherein the first component of the second VLP comprises a polypeptide that shares at least 70%, at least 80%, at least 90%, at least 91%, at least 92%, at least 93%, at least 94%, at least 95%, at least 96%, at least 97%, at least 98%, at least 99%, or 100% identity to SEQ ID NO: 176 or
wherein the first component of the second VLP comprises a polypeptide that shares at least 70%, at least 80%, at least 90%, at least 91%, at least 92%, at least 93%, at least 94%, at least 95%, at least 96%, at least 97%, at least 98%, at least 99%, or 100% identity to SEQ ID NO: 177.

14. (canceled)

15. The composition of claim 1, wherein the composition comprises one or more pharmaceutically acceptable diluents, adjuvants, or excipients.

16.-21. (canceled)

22. A unit dose of the composition of claim 1, wherein the unit dose comprises:
a) about 0.5 μg, about 1 μg, about 5 μg, about 10 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 100 μg, about 125 μg, about 150 μg, about 200 μg, about 225 μg, about 250 μg, about 300 μg, or about 500 μg of the first VLP; and
b) about 0.5 μg, about 1 μg, about 5 μg, about 10 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 100 μg, about 125 μg, about 150 μg, about 200 μg, about 225 μg, about 250 μg, about 300 μg, or about 500 μg of the second VLP.

23.-27. (canceled)

28. A method of vaccinating a subject, generating an immune response in a subject, preventing infection by a paramyxovirus, and/or immunizing a subject against a paramyxovirus, comprising administering to the subject an effective amount a composition according to claim 1.

29.-31. (canceled)

32. The method of claim 28, wherein the paramyxovirus is respiratory syncytial virus (RSV), human metapneumovirus (hMPV), or both RSV and hMPV.

33.-47. (canceled)

48. The method of claim 28, further comprising administering a second dose of the pharmaceutical composition, wherein the second dose is administered within about 2, 3, 4, 5, 6, 7, 8, 9, 10, 11, or 12 weeks, about 1 month, about 2 months, about 3 months, about 4 months, about 5 months, about 6 months, about 9 months, about 12 months, about 24 months, or about 36 months after the first dose.

49. (canceled)

50. The method of claim 48, further comprising administering a third dose of the pharmaceutical composition wherein the third dose is administered about 1 year, about 2 years, about 3 years, about 4 years, or about 5 years after the second dose.

51.-67. (canceled)

68. A kit, comprising one or more of:
a) a composition comprising a virus-like particle (VLP) comprising a first component comprising a respiratory syntactical virus (RSV) F protein ectodomain or antigenic variant thereof;
b) a composition comprising a second virus-like particle (VLP) comprising a first component comprising a human metapneumovirus (hMPV) F protein ectodomain or antigenic variant thereof; and
c) a composition comprising a virus-like particle (VLP) comprising a plurality of first components, some first components comprising a respiratory syntactical virus (RSV) F protein ectodomain or antigenic variant thereof and some first components comprising a human metapneumovirus (hMPV) F protein ectodomain or antigenic variant thereof.

69. (canceled)


SEQUENCE LISTING (188 sequences)

SEQ ID NO: 1 - protein, 206 aa, synthetic construct
  REGION 1..206: note = I53-34A
EGMDPLAVLAESRLLPLLTVRGGEDLAGLATVLELMGVGALEITLRTEKGLEALKALRKSGLLLGAGTVRSPKEAEAALEAGAAFLVSPGLLEEVAALAQARGVPYLPGVLTPTEVERALALGLSALKFFPAEPFQGVRVLRAYAEVFPEVRFLPTGGIKEEHLPHYAALPNLLAVGGSWLLQGDLAAVMKKVKAAKALLSPQAPG

SEQ ID NO: 2 - protein, 155 aa, synthetic construct
  REGION 1..155: note = I53-34B
TKKVGIVDTTFARVDMAEAAIRTLKALSPNIKIIRKTVPGIKDLPVACKKLLEEEGCDIVMALGMPGKAEKDKVCAHEASLGLMLAQLMTNKHIIEVFVHEDEAKDDDELDILALVRAIEHAANVYYLLFKPEYLTRMAGKGLRQGREDAGPARE

SEQ ID NO: 3 - protein, 155 aa, synthetic construct
  REGION 1..155: note = I53-40A
TKKVGIVDTTFARVDMASAAILTLKMESPNIKIIRKTVPGIKDLPVACKKLLEEEGCDIVMALGMPGKAEKDKVCAHEASLGLMLAQLMTNKHIIEVFVHEDEAKDDAELKILAARRAIEHALNVYYLLFKPEYLTRMAGKGLRQGFEDAGPARE

SEQ ID NO: 4 - protein, 208 aa, synthetic construct
  REGION 1..208: note = I53-40B
STINNQLKALKVIPVIAIDNAEDIIPLGKVLAENGLPAAEITFRSSAAVKAIMLLRSAQPEMLIGAGTILNGVQALAAKEAGATFVVSPGFNPNTVRACQIIGIDIVPGVNNPSTVEAALEMGLTTLKFFPAEASGGISMVKSLVGPYGDIRLMPTGGITPSNIDNYLAIPQVLACGGTWMVDKKLVTNGEWDEIARLTREIVEQVNP

SEQ ID NO: 5 - protein, 113 aa, synthetic construct
  REGION 1..113: note = I53-47A
PIFTLNTNIKATDVPSDFLSLTSRLVGLILSKPGSYVAVHINTDQQLSFGGSTNPAAFGTLMSIGGIEPSKNRDHSAVLFDHLNAMLGIPKNRMYIHFVNLNGDDVGWNGTTF

SEQ ID NO: 6 - protein, 156 aa, synthetic construct
  REGION 1..156: note = I53-47B
NQHSHKDYETVRIAVVRARWHADIVDACVEAFEIAMAAIGGDRFAVDVFDVPGAYEIPLHARTLAETGRYGAVLGTAFVVNGGIYRHEFVASAVIDGMMNVQLSTGVPVLSAVLTPHRYRDSAEHHRFFAAHFAVKGVEAARACIEILAAREKIAA

SEQ ID NO: 7 - protein, 202 aa, synthetic construct
  REGION 1..202: note = I53-50A
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 8 - protein, 156 aa, synthetic construct
  REGION 1..156: note = I53-50B
NQHSHKDYETVRIAVVRARWHAEIVDACVSAFEAAMADIGGDRFAVDVFDVPGAYEIPLHARTLAETGRYGAVLGTAFVVNGGIYRHEFVASAVIDGMMNVQLSTGVPVLSAVLTPHRYRDSDAHTLLFLALFAVKGMEAARACVEILAAREKIAA

SEQ ID NO: 9 - protein, 176 aa, synthetic construct
  REGION 1..176: note = I53-51A
FTKSGDDGNTNVINKRVGKDSPLVNFLGDLDELNSFIGFAISKIPWEDMKKDLERVQVELFEIGEDLSTQSSKKKIDESYVLWLLAATAIYRIESGPVKLFVIPGGSEEASVLHVTRSVARRVERNAVKYTKELPEINRMIIVYLNRLSSLLFAMALVANKRRNQSEKIYEIGKSW

SEQ ID NO: 10 - protein, 156 aa, synthetic construct
  REGION 1..156: note = I53-51B
NQHSHKDYETVRIAVVRARWHADIVDQCVRAFEEAMADAGGDRFAVDVFDVPGAYEIPLHARTLAETGRYGAVLGTAFVVNGGIYRHEFVASAVIDGMMNVQLSTGVPVLSAVLTPHRYRSSREHHEFFREHFMVKGVEAAAACITILAAREKIAA

SEQ ID NO: 11 - protein, 200 aa, synthetic construct
  REGION 1..200: note = I52-03A
GHTKGPTPQQHDGSALRIGIVHARWNKTIIMPLLIGTIAKLLECGVKASNIVVQSVPGSWELPIAVQRLYSASQLQTPSSGPSLSAGDLLGSSTTDLTALPTTTASSTGPFDALIAIGVLIKGETMHFEYIADSVSHGLMRVQLDTGVPVIFGVLTVLTDDQAKARAGVIEGSHNHGEDWGLAAVEMGVRRRDWAAGKTE

SEQ ID NO: 12 - protein, 236 aa, synthetic construct
  REGION 1..236: note = I52-03B
YEVDHADVYDLFYLGRGKDYAAEASDIADLVRSRTPEASSLLDVACGTGTHLEHFTKEFGDTAGLELSEDMLTHARKRLPDATLHQGDMRDFQLGRKFSAVVSMFSSVGYLKTVAELGAAVASFAEHLEPGGVVVVEPWWFPETFADGWVSADVVRRDGRTVARVSHSVREGNATRMEVHFTVADPGKGVRHFSDVHLITLFHQREYEAAFMAAGLRVEYLEGGPSGRGLFVGVPA

SEQ ID NO: 13 - protein, 137 aa, synthetic construct
  REGION 1..137: note = I52-32A
GMKEKFVLIITHGDFGKGLLSGAEVIIGKQENVHTVGLNLGDNIEKVAKEVMRIIIAKLAEDKEIIIVVDLFGGSPFNIALEMMKTFDVKVITGINMPMLVELLTSINVYDTTELLENISKIGKDGIKVIEKSSLKM

SEQ ID NO: 14 - protein, 153 aa, synthetic construct
  REGION 1..153: note = I52-32B
KYDGSKLRIGILHARWNLEIIAALVAGAIKRLQEFGVKAENIIIETVPGSFELPYGSKLFVEKQKRLGKPLDAIIPIGVLIKGSTMHFEYICDSTTHQLMKLNFELGIPVIFGVLTCLTDEQAEARAGLIEGKMHNHGEDWGAAAVEMATKFN

SEQ ID NO: 15 - protein, 163 aa, synthetic construct
  REGION 1..163: note = I52-33A
AVKGLGEVDQKYDGSKLRIGILHARWNRKIILALVAGAVLRLLEFGVKAENIIIETVPGSFELPYGSKLFVEKQKRLGKPLDAIIPIGVLIKGSTMHFEYICDSTTHQLMKLNFELGIPVIFGVLTCLTDEQAEARAGLIEGKMHNHGEDWGAAAVEMATKFN

SEQ ID NO: 16 - protein, 174 aa, synthetic construct
  REGION 1..174: note = I52-33B
GANWYLDNESSRLSFTSTKNADIAEVHRFLVLHGKVDPKGLAEVEVETESISTGIPLRDMLLRVLVFQVSKFPVAQINAQLDMRPINNLAPGAQLELRLPLTVSLRGKSHSYNAELLATRLDERRFQVVTLEPLVIHAQDFDMVRAFNALRLVAGLSAVSLSVPVGAVLIFTAR

SEQ ID NO: 17 - protein, 207 aa, synthetic construct
  REGION 1..207: note = I32-06A
TDYIRDGSAIKALSFAIILAEADLRHIPQDLQRLAVRVIHACGMVDVANDLAFSEGAGKAGRNALLAGAPILCDARMVAEGITRSRLPADNRVIYTLSDPSVPELAKKIGNTRSAAALDLWLPHIEGSIVAIGNAPTALFRLFELLDAGAPKPALIIGMPVGFVGAAESKDELAANSRGVPYVIVRGRRGGSAMTAAAVNALASERE

SEQ ID NO: 18 - protein, 127 aa, synthetic construct
  REGION 1..127: note = I32-06B
ITVFGLKSKLAPRREKLAEVIYSSLHLGLDIPKGKHAIRFLCLEKEDFYYPFDRSDDYTVIEINLMAGRSEETKMLLIFLLFIALERKLGIRAHDVEITIKEQPAHCWGFRGRTGDSARDLDYDIYV

SEQ ID NO: 19 - protein, 234 aa, synthetic construct
  REGION 1..234: note = I32-19A
GSDLQKLQRFSTCDISDGLLNVYNIPTGGYFPNLTAISPPQNSSIVGTAYTVLFAPIDDPRPAVNYIDSVPPNSILVLALEPHLQSQFHPFIKITQAMYGGLMSTRAQYLKSNGTVVFGRIRDVDEHRTLNHPVFAYGVGSCAPKAVVKAVGTNVQLKILTSDGVTQTICPGDYIAGDNNGIVRIPVQETDISKLVTYIEKSIEVDRLVSEAIKNGLPAKAAQTARRMVLKDYI

SEQ ID NO: 20 - protein, 161 aa, synthetic construct
  REGION 1..161: note = I32-19B
SGMRVYLGADHAGYELKQAIIAFLKMTGHEPIDCGALRYDADDDYPAFCIAAATRTVADPGSLGIVLGGSGNGEQIAANKVPGARCALAWSVQTAALAREHNNAQLIGIGGRMHTLEEALRIVKAFVTTPWSKAQRHQRRIDILAEYERTHEAPPVPGAPA

SEQ ID NO: 21 - protein, 156 aa, synthetic construct
  REGION 1..156: note = I32-28A
GDDARIAAIGDVDELNSQIGVLLAEPLPDDVRAALSAIQHDLFDLGGELCIPGHAAITEDHLLRLALWLVHYNGQLPPLEEFILPGGARGAALAHVCRTVCRRAERSIKALGASEPLNIAPAAYVNLLSDLLFVLARVLNRAAGGADVLWDRTRAH

SEQ ID NO: 22 - protein, 156 aa, synthetic construct
  REGION 1..156: note = I32-28B
ILSAEQSFTLRHPHGQAAALAFVREPAAALAGVQRLRGLDSDGEQVWGELLVRVPLLGEVDLPFRSEIVRTPQGAELRPLTLTGERAWVAVSGQATAAEGGEMAFAFQFQAHLATPEAEGEGGAAFEVMVQAAAGVTLLLVAMALPQGLAAGLPPA

SEQ ID NO: 23 - protein, 155 aa, synthetic construct
  REGION 1..155: note = I53-40A.1
TKKVGIVDTTFARVDMASAAILTLKMESPNIKIIRKTVPGIKDLPVACKKLLEEEGCDIVMALGMPGKKEKDKVCAHEASLGLMLAQLMTNKHIIEVFVHEDEAKDDAELKILAARRAIEHALNVYYLLFKPEYLTRMAGKGLRQGFEDAGPARE

SEQ ID NO: 24 - protein, 208 aa, synthetic construct
  REGION 1..208: note = I53-40B.1
DDINNQLKRLKVIPVIAIDNAEDIIPLGKVLAENGLPAAEITFRSSAAVKAIMLLRSAQPEMLIGAGTILNGVQALAAKEAGADFVVSPGFNPNTVRACQIIGIDIVPGVNNPSTVEQALEMGLTTLKFFPAEASGGISMVKSLVGPYGDIRLMPTGGITPDNIDNYLAIPQVLACGGTWMVDKKLVRNGEWDEIARLTREIVEQVNP

SEQ ID NO: 25 - protein, 113 aa, synthetic construct
  REGION 1..113: note = I53-47A.1
PIFTLNTNIKADDVPSDFLSLTSRLVGLILSKPGSYVAVHINTDQQLSFGGSTNPAAFGTLMSIGGIEPDKNRDHSAVLFDHLNAMLGIPKNRMYIHFVNLNGDDVGWNGTTF

SEQ ID NO: 26 - protein, 113 aa, synthetic construct
  REGION 1..113: note = I53-47A.1NegT2
PIFTLNTNIKADDVPSDFLSLTSRLVGLILSEPGSYVAVHINTDQQLSFGGSTNPAAFGTLMSIGGIEPDKNEDHSAVLFDHLNAMLGIPKNRMYIHFVDLDGDDVGWNGTTF

SEQ ID NO: 27 - protein, 156 aa, synthetic construct
  REGION 1..156: note = I53-47B.1
NQHSHKDHETVRIAVVRARWHADIVDACVEAFEIAMAAIGGDRFAVDVFDVPGAYEIPLHARTLAETGRYGAVLGTAFVVNGGIYRHEFVASAVIDGMMNVQLDTGVPVLSAVLTPHRYRDSDEHHRFFAAHFAVKGVEAARACIEILNAREKIAA

SEQ ID NO: 28 - protein, 156 aa, synthetic construct
  REGION 1..156: note = I53-47B.1NegT2
NQHSHKDHETVRIAVVRARWHADIVDACVEAFEIAMAAIGGDRFAVDVFDVPGAYEIPLHARTLAETGRYGAVLGTAFVVDGGIYDHEFVASAVIDGMMNVQLDTGVPVLSAVLTPHEYEDSDEDHEFFAAHFAVKGVEAARACIEILNAREKIAA

SEQ ID NO: 29 - protein, 202 aa, synthetic construct
  REGION 1..202: note = I53-50A.1
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPQFVKAMKGPFPNVKFVPTGGVNLDNVCEWFKAGVLAVGVGDALVKGDPDEVREKAKKFVEKIRGCTE

SEQ ID NO: 30 - protein, 202 aa, synthetic construct
  REGION 1..202: note = I53-50A.1NegT2
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPEFVEAMKGPFPNVKFVPTGGVDLDDVCEWFDAGVLAVGVGDALVEGDPDEVREDAKEFVEEIRGCTE

SEQ ID NO: 31 - protein, 202 aa, synthetic construct
  REGION 1..202: note = I53-50A.1PosT1
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGKALVKGKPDEVREKAKKFVKKIRGCTE

SEQ ID NO: 32 - protein, 156 aa, synthetic construct
  REGION 1..156: note = I53-50B.1
NQHSHKDHETVRIAVVRARWHAEIVDACVSAFEAAMRDIGGDRFAVDVFDVPGAYEIPLHARTLAETGRYGAVLGTAFVVNGGIYRHEFVASAVIDGMMNVQLDTGVPVLSAVLTPHRYRDSDAHTLLFLALFAVKGMEAARACVEILAAREKIAA

SEQ ID NO: 33 - protein, 156 aa, synthetic construct
  REGION 1..156: note = I53-50B.1NegT2
NQHSHKDHETVRIAVVRARWHAEIVDACVSAFEAAMRDIGGDRFAVDVFDVPGAYEIPLHARTLAETGRYGAVLGTAFVVDGGIYDHEFVASAVIDGMMNVQLDTGVPVLSAVLTPHEYEDSDADTLLFLALFAVKGMEAARACVEILAAREKIAA

SEQ ID NO: 34 - protein, 156 aa, synthetic construct
  REGION 1..156: note = I53-50B.4PosT1
NQHSHKDHETVRIAVVRARWHAEIVDACVSAFEAAMRDIGGDRFAVDVFDVPGAYEIPLHARTLAETGRYGAVLGTAFVVNGGIYRHEFVASAVINGMMNVQLNTGVPVLSAVLTPHNYDKSKAHTLLFLALFAVKGMEAARACVEILAAREKIAA

SEQ ID NO: 35 - protein, 155 aa, synthetic construct
  REGION 1..155: note = I53-40A genus
  VARIANT 69: note = Xaa is A or K
TKKVGIVDTTFARVDMASAAILTLKMESPNIKIIRKTVPGIKDLPVACKKLLEEEGCDIVMALGMPGKXEKDKVCAHEASLGLMLAQLMTNKHIIEVFVHEDEAKDDAELKILAARRAIEHALNVYYLLFKPEYLTRMAGKGLRQGFEDAGPARE

SEQ ID NO: 36 - protein, 208 aa, synthetic construct
  REGION 1..208: note = I53-40B genus
  VARIANT 1: note = Xaa is S or D
  VARIANT 2: note = Xaa is T or D
  VARIANT 9: note = Xaa is A or R
  VARIANT 84: note = Xaa is T or D
  VARIANT 118: note = Xaa is A or Q
  VARIANT 162: note = Xaa is S or D
  VARIANT 188: note = Xaa is T or R
XXINNQLKXLKVIPVIAIDNAEDIIPLGKVLAENGLPAAEITFRSSAAVKAIMLLRSAQPEMLIGAGTILNGVQALAAKEAGAXFVVSPGFNPNTVRACQIIGIDIVPGVNNPSTVEXALEMGLTTLKFFPAEASGGISMVKSLVGPYGDIRLMPTGGITPXNIDNYLAIPQVLACGGTWMVDKKLVXNGEWDEIARLTREIVEQVNP

SEQ ID NO: 37 - protein, 113 aa, synthetic construct
  REGION 1..113: note = I53-47A genus
  VARIANT 12: note = Xaa is T or D
  VARIANT 32: note = Xaa is K or E
  VARIANT 70: note = Xaa is S or D
  VARIANT 73: note = Xaa is R or E
  VARIANT 100: note = Xaa is N or D
  VARIANT 102: note = Xaa is N or D
PIFTLNTNIKAXDVPSDFLSLTSRLVGLILSXPGSYVAVHINTDQQLSFGGSTNPAAFGTLMSIGGIEPXKNXDHSAVLFDHLNAMLGIPKNRMYIHFVXLXGDDVGWNGTTF

SEQ ID NO: 38 - protein, 156 aa, synthetic construct
  REGION 1..156: note = I53-47B genus
  VARIANT 8: note = Xaa is Y or H
  VARIANT 81: note = Xaa is N or D
  VARIANT 86: note = Xaa is R or D
  VARIANT 104: note = Xaa is S or D
  VARIANT 118: note = Xaa is R or E
  VARIANT 120: note = Xaa is R or E
  VARIANT 123: note = Xaa is A or D
  VARIANT 125: note = Xaa is H or D
  VARIANT 127: note = Xaa is R or E
  VARIANT 149: note = Xaa is A or N
NQHSHKDXETVRIAVVRARWHADIVDACVEAFEIAMAAIGGDRFAVDVFDVPGAYEIPLHARTLAETGRYGAVLGTAFVVXGGIYXHEFVASAVIDGMMNVQLXTGVPVLSAVLTPHXYXDSXEXHXFFAAHFAVKGVEAARACIEILXAREKIAA

SEQ ID NO: 39 - protein, 202 aa, synthetic construct
  REGION 1..202: note = I53-50A genus
  VARIANT 123: note = Xaa is T or D
  VARIANT 136: note = Xaa is Q or E
  VARIANT 139: note = Xaa is K or E
  VARIANT 157: note = Xaa is N or D
  VARIANT 160: note = Xaa is N or D
  VARIANT 163: note = Xaa is E or K
  VARIANT 166: note = Xaa is D or K
  VARIANT 176: note = Xaa is S, D or K
  VARIANT 180: note = Xaa is K or E
  VARIANT 182: note = Xaa is T, D, or K
  VARIANT 189: note = Xaa is D or K
  VARIANT 192: note = Xaa is A, E,or K
  VARIANT 195: note = Xaa is E or K
  VARIANT 196: note = Xaa is E or K
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHXILKLFPGEVVGPXFVXAMKGPFPNVKFVPTGGVXLDXVCXWFXAGVLAVGVGXALVXGXPDEVREXAKXFVXXIRGCTE

SEQ ID NO: 40 - protein, 156 aa, synthetic construct
  REGION 1..156: note = I53-50B genus
  VARIANT 8: note = Xaa is Y or H
  VARIANT 37: note = Xaa is A or R
  VARIANT 81: note = Xaa is N or D
  VARIANT 86: note = Xaa is R or D
  VARIANT 96: note = Xaa is N or D
  VARIANT 104: note = Xaa is S, N, or D
  VARIANT 118: note = Xaa is R, E, or N
  VARIANT 120: note = Xaa is R, E, or D
  VARIANT 121: note = Xaa is K or D
  VARIANT 123: note = Xaa is K or D
  VARIANT 125: note = Xaa is H or D
NQHSHKDXETVRIAVVRARWHAEIVDACVSAFEAAMXDIGGDRFAVDVFDVPGAYEIPLHARTLAETGRYGAVLGTAFVVXGGIYXHEFVASAVIXGMMNVQLXTGVPVLSAVLTPHXYXXSXAXTLLFLALFAVKGMEAARACVEILAAREKIAA

SEQ ID NO: 41 - protein, 158 aa, synthetic construct
  REGION 1..158: note = T32-28A
GEVPIGDPKELNGMEIAAVYLQPIEMEPRGIDLAASLADIHLEADIHALKNNPNGFPEGFWMPYLTIAYALANADTGAIKTGTLMPMVADDGPHYGANIAMEKDKKGGFGVGTYALTFLISNPEKQGFGRHVDEETGVGKWFEPFVVTYFFKYTGTPK

SEQ ID NO: 42 - protein, 183 aa, synthetic construct
  REGION 1..183: note = T32-28B
SQAIGILELTSIAKGMELGDAMLKSANVDLLVSKTISPGKFLLMLGGDIGAIQQAIETGTSQAGEMLVDSLVLANIHPSVLPAISGLNSVDKRQAVGIVETWSVAACISAADLAVKGSNVTLVRVHMAFGIGGKCYMVVAGDVLDVAAAVATASLAAGAKGLLVYASIIPRPHEAMWRQMVEG

SEQ ID NO: 43 - protein, 102 aa, synthetic construct
  REGION 1..102: note = T33-09A
EEVVLITVPSALVAVKIAHALVEERLAACVNIVPGLTSIYRWQGSVVSDHELLLLVKTTTHAFPKLKERVKALHPYTVPEIVALPIAEGNREYLDWLRENTG

SEQ ID NO: 44 - protein, 121 aa, synthetic construct
  REGION 1..121: note = T33-09B
VRGIRGAITVEEDTPAAILAATIELLLKMLEANGIQSYEELAAVIFTVTEDLTSAFPAEAARLIGMHRVPLLSAREVPVPGSLPRVIRVLALWNTDTPQDRVRHVYLNEAVRLRPDLESAQ

SEQ ID NO: 45 - protein, 176 aa, synthetic construct
  REGION 1..176: note = T33-15A
SKAKIGIVTVSDRASAGITADISGKAIILALNLYLTSEWEPIYQVIPDEQDVIETTLIKMADEQDCCLIVTTGGTGPAKRDVTPEATEAVCDRMMPGFGELMRAESLKEVPTAILSRQTAGLRGDSLIVNLPGDPASISDCLLAVFPAIPYCIDLMEGPYLECNEAMIKPFRPKAK

SEQ ID NO: 46 - protein, 121 aa, synthetic construct
  REGION 1..121: note = T33-15B
VRGIRGAITVNSDTPTSIIIATILLLEKMLEANGIQSYEELAAVIFTVTEDLTSAFPAEAARQIGMHRVPLLSAREVPVPGSLPRVIRVLALWNTDTPQDRVRHVYLSEAVRLRPDLESAQ

SEQ ID NO: 47 - protein, 171 aa, synthetic construct
  REGION 1..171: note = T33-21A
RITTKVGDKGSTRLFGGEEVWKDSPIIEANGTLDELTSFIGEAKHYVDEEMKGILEEIQNDIYKIMGEIGSKGKIEGISEERIAWLLKLILRYMEMVNLKSFVLPGGTLESAKLDVCRTIARRALRKVLTVTREFGIGAEAAAYLLALSDLLFLLARVIEIEKNKLKEVRS

SEQ ID NO: 48 - protein, 122 aa, synthetic construct
  REGION 1..122: note = T33-21B
PHLVIEATANLRLETSPGELLEQANKALFASGQFGEADIKSRFVTLEAYRQGTAAVERAYLHACLSILDGRDIATRTLLGASLCAVLAEAVAGGGEEGVQVSVEVREMERLSYAKRVVARQR

SEQ ID NO: 49 - protein, 157 aa, synthetic construct
  REGION 1..157: note = T33-28A
ESVNTSFLSPSLVTIRDFDNGQFAVLRIGRTGFPADKGDIDLCLDKMIGVRAAQIFLGDDTEDGFKGPHIRIRCVDIDDKHTYNAMVYVDLIVGTGASEVERETAEEEAKLALRVALQVDIADEHSCVTQFEMKLREELLSSDSFHPDKDEYYKDFL

SEQ ID NO: 50 - protein, 112 aa, synthetic construct
  REGION 1..112: note = T33-28B
PVIQTFVSTPLDHHKRLLLAIIYRIVTRVVLGKPEDLVMMTFHDSTPMHFFGSTDPVACVRVEALGGYGPSEPEKVTSIVTAAITAVCGIVADRIFVLYFSPLHCGWNGTNF

SEQ ID NO: 51 - protein, 102 aa, synthetic construct
  REGION 1..102: note = T33-31A
EEVVLITVPSALVAVKIAHALVEERLAACVNIVPGLTSIYREEGSVVSDHELLLLVKTTTDAFPKLKERVKELHPYEVPEIVALPIAEGNREYLDWLRENTG

SEQ ID NO: 52 - skipped

SEQ ID NO: 53 - protein, 202 aa, synthetic construct
  REGION 1..202: note = I53-50A
EELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 54 - protein, 4 aa, synthetic construct
  REGION 1..4: note = furin cleavage native sequence
RQSR

SEQ ID NO: 55 - protein, 4 aa, synthetic construct
  REGION 1..4: note = furin cleavage mutated sequence
RRRR

SEQ ID NO: 56 - protein, 539 aa, Homo sapiens
MSWKVVIIFSLLITPQHGLKESYLEESCSTITEGYLSVLRTGWYTNVFTLEVGDVENLTCSDGPSLIKTELDLTKSALRELKTVSADQLAREEQIENPRQSRFVLGAIALGVATAAAVTAGVAIAKTIRLESEVTAIKNALKTTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSGKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTGFIIVIILIAVLGSSMILVSIFIIIKKTKKPTGAPPELSGVTNNGFIPHS

SEQ ID NO: 57 - protein, 472 aa, Homo sapiens
KESYLEESCSTITEGYLSVLRTGWYTNVFTLEVGDVENLTCSDGPSLIKTELDLTKSALRELKTVSADQLAREEQIENPRQSRFVLGAIALGVATAAAVTAGVAIAKTIRLESEVTAIKNALKTTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSGKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTGF

SEQ ID NO: 58 - protein, 462 aa, synthetic construct
  REGION 1..462: note = A185P, Q100R, S101R
KESYLEESCSTITEGYLSVLRTGWYTNVFTLEVGDVENLTCADGPSLIKTELDLTKSALRELRTVSADQLAREEQIENPRRRRFVLGAIALGVATAAAVTAGVAIAKTIRLESEVTAIKNALKKTNEAVSTLGNGVRVLATAVRELKDFVSKNLTRAINKNKCDIPDLKMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCSGKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRIL

SEQ ID NO: 59 - protein, 453 aa, synthetic construct
  REGION 1..453: note = A185P, Q100R, S101R
KESYLEESCSTITEGYLSVLRTGWYTNVFTLEVGDVENLTCADGPSLIKTELDLTKSALRELRTVSADQLAREEQIENPRRRRFVLGAIALGVATAAAVTAGVAIAKTIRLESEVTAIKNALKKTNEAVSTLGNGVRVLATAVRELKDFVSKNLTRAINKNKCDIPDLKMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCSGKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQA

SEQ ID NO: 60 - protein, 470 aa, synthetic construct
  REGION 1..470: note = A185P, Q100R, S101R
KESYLEESCSTITEGYLSVLRTGWYTNVFTLEVGDVENLTCADGPSLIKTELDLTKSALRELRTVSADQLAREEQIENPRRRRFVLGAIALGVATAAAVTAGVAIAKTIRLESEVTAIKNALKKTNEAVSTLGNGVRVLATAVRELKDFVSKNLTRAINKNKCDIPDLKMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCSGKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNT

SEQ ID NO: 61 - protein, 453 aa, synthetic construct
  REGION 1..453: note = Q100R, S101R
KESYLEESCSTITEGYLSVLRTGWYTNVFTLEVGDVENLTCADGPSLIKTELDLTKSALRELRTVSADQLAREEQIENPRRRRFVLGAIALGVATAAAVTAGVAIAKTIRLESEVTAIKNALKKTNEAVSTLGNGVRVLATAVRELKDFVSKNLTRAINKNKCDIADLKMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCSGKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQA

SEQ ID NO: 62 - protein, 453 aa, synthetic construct
  REGION 1..453: note = A185P, T127C, N153C, Q100R, S101R
KESYLEESCSTITEGYLSVLRTGWYTNVFTLEVGDVENLTCADGPSLIKTELDLTKSALRELRTVSADQLAREEQIENPRRRRFVLGAIALGVATAAAVTAGVAIAKCIRLESEVTAIKNALKKTNEAVSTLGCGVRVLATAVRELKDFVSKNLTRAINKNKCDIPDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSGKKGNYACLLREDQGWYCQNAGSTVYYPNEKDCETRGDHVFCDTAAGINVAEQSKECNINISTTNYPCKVSTGRNPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQA

SEQ ID NO: 63 - protein, 453 aa, synthetic construct
  REGION 1..453: note = V84C, A140C, A147C, A249C, N97G, P98G, R99G, Q100G, S101G, R102G
KESYLEESCSTITEGYLSVLRTGWYTNVFTLEVGDVENLTCTDGPSLIKTELDLTKSALRELKTCSADQLAREEQIEGGGGGGFVLGAIALGVATAAAVTAGIAIAKTIRLESEVNAIKGCLKTTNECVSTLGNGVRVLATAVRELKEFVSKNLTSAINKNKCDIADLKMAVSFSQFNRRFLNVVRQFSDNAGITPAISLDLMTDAELARAVSYMPTSAGQIKLMLENRCMVRRKGFGILIGVYGSSVIYMVQLPIFGVIDTPCWIIKAAPSCSEKDGNYACLLREDQGWYCKNAGSTVYYPNDKDCETRGDHVFCDTAAGINVAEQSRECNINISTTNYPCKVSTGRHPISMVALSPLGALVACYKGVSCSIGSNRVGIIKQLPKGCSYITNQDADTVTIDNTVYQLSKVEGEQHVIKGRPVSSSFDPIKFPEDQFNVALDQVFESIENSQA

SEQ ID NO: 64 - protein, 469 aa, synthetic construct
  REGION 1..469: note = V84C, A140C, A147C, A249C, N97G, P98G, R99G, Q100G, S101G, R102G
KESYLEESCSTITEGYLSVLRTGWYTNVFTLEVGDVENLTCTDGPSLIKTELDLTKSALRELKTCSADQLAREEQIEGGGGGGFVLGAIALGVATAAAVTAGIAIAKTIRLESEVNAIKGCLKTTNECVSTLGNGVRVLATAVRELKEFVSKNLTSAINKNKCDIADLKMAVSFSQFNRRFLNVVRQFSDNAGITPAISLDLMTDAELARAVSYMPTSAGQIKLMLENRCMVRRKGFGILIGVYGSSVIYMVQLPIFGVIDTPCWIIKAAPSCSEKDGNYACLLREDQGWYCKNAGSTVYYPNDKDCETRGDHVFCDTAAGINVAEQSRECNINISTTNYPCKVSTGRHPISMVALSPLGALVACYKGVSCSIGSNRVGIIKQLPKGCSYITNQDADTVTIDNTVYQLSKVEGEQHVIKGRPVSSSFDPIKFPEDQFNVALDQVFESIENSQALVDQSNKILNSAESAI

SEQ ID NO: 65 - protein, 453 aa, synthetic construct
  REGION 1..453: note = A63C, A140C, A147C, K188C, K450C, S470C, N97G, P98G, R99G, Q100G,S101G, R102G
KESYLEESCSTITEGYLSVLRTGWYTNVFTLEVGDVENLTCTDCPSLIKTELDLTKSALRELKTVSADQLAREEQIEGGGGGGFVLGAIALGVATAAAVTAGIAIAKTIRLESEVNAIKGCLKTTNECVSTLGNGVRVLATAVRELKEFVSKNLTSAINKNKCDIADLCMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKDGNYACLLREDQGWYCKNAGSTVYYPNDKDCETRGDHVFCDTAAGINVAEQSRECNINISTTNYPCKVSTGRHPISMVALSPLGALVACYKGVSCSIGSNRVGIIKQLPKGCSYITNQDADTVTIDNTVYQLSKVEGEQHVIKGRPVSSSFDPICFPEDQFNVALDQVFESIENCQA

SEQ ID NO: 66 - protein, 469 aa, synthetic construct
  REGION 1..469: note = A63C, A140C, A147C, K188C, K450C, S470C, N97G, P98G, R99G, Q100G,S101G, R102G
KESYLEESCSTITEGYLSVLRTGWYTNVFTLEVGDVENLTCTDCPSLIKTELDLTKSALRELKTVSADQLAREEQIEGGGGGGFVLGAIALGVATAAAVTAGIAIAKTIRLESEVNAIKGCLKTTNECVSTLGNGVRVLATAVRELKEFVSKNLTSAINKNKCDIADLCMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKDGNYACLLREDQGWYCKNAGSTVYYPNDKDCETRGDHVFCDTAAGINVAEQSRECNINISTTNYPCKVSTGRHPISMVALSPLGALVACYKGVSCSIGSNRVGIIKQLPKGCSYITNQDADTVTIDNTVYQLSKVEGEQHVIKGRPVSSSFDPICFPEDQFNVALDQVFESIENCQALVDQSNKILNSAESAI

SEQ ID NO: 67 - protein, 452 aa, synthetic construct
  REGION 1..452: note = A63C, A140C, A147C, K188C, G106deleted, N97G, P98G, R99G,Q100G, S101G, R102G
KESYLEESCSTITEGYLSVLRTGWYTNVFTLEVGDVENLTCSDCPSLIKTELDLTKSALRELKTVSADQLAREEQIEGGGGGGFVLAIALGVATAAAVTAGVAIAKTIRLESEVTAIKNCLKTTNECVSTLGNGVRVLATAVRELKDFVSKNLTSAINKNKCDIDDLCMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIIKAAPSCSGKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQA

SEQ ID NO: 68 - protein, 468 aa, synthetic construct
  REGION 1..468: note = A63C, A140C, A147C, K188C, G106deleted, N97G, P98G, R99G,Q100G, S101G, R102G
KESYLEESCSTITEGYLSVLRTGWYTNVFTLEVGDVENLTCSDCPSLIKTELDLTKSALRELKTVSADQLAREEQIEGGGGGGFVLAIALGVATAAAVTAGVAIAKTIRLESEVTAIKNCLKTTNECVSTLGNGVRVLATAVRELKDFVSKNLTSAINKNKCDIDDLCMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIIKAAPSCSGKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAESAI

SEQ ID NO: 69 - protein, 453 aa, synthetic construct
  REGION 1..453: note = A113C, A120C, A339C, Q426C, T160F, I17L, Q100K, S101A
KESYLEESCSTITEGYLSVLRTGWYTNVFTLEVGDVENLTCTDGPSLIKTELDLTKSALRELKTVSADQLAREEQIENPRKARFVLGAIALGVCTAAAVTCGIAIAKTIRLESEVNAIKGALKTTNEAVSTLGNGVRVLAFAVRELKEFVSKNLTSALNKNKCDIADLKMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKDGNYACLLREDQGWYCKNAGSTVYYPNDKDCETRGDHVFCDTACGINVAEQSRECNINISTTNYPCKVSTGRHPISMVALSPLGALVACYKGVSCSIGSNRVGIIKQLPKGCSYITNQDADTVTIDNTVYCLSKVEGEQHVIKGRPVSSSFDPIKFPEDQFNVALDQVFESIENSQA

SEQ ID NO: 70 - protein, 469 aa, synthetic construct
  REGION 1..469: note = A113C, A120C, A339C, Q426C, T160F, I177L, Q100K, S101A
KESYLEESCSTITEGYLSVLRTGWYTNVFTLEVGDVENLTCTDGPSLIKTELDLTKSALRELKTVSADQLAREEQIENPRKARFVLGAIALGVCTAAAVTCGIAIAKTIRLESEVNAIKGALKTTNEAVSTLGNGVRVLAFAVRELKEFVSKNLTSALNKNKCDIADLKMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKDGNYACLLREDQGWYCKNAGSTVYYPNDKDCETRGDHVFCDTACGINVAEQSRECNINISTTNYPCKVSTGRHPISMVALSPLGALVACYKGVSCSIGSNRVGIIKQLPKGCSYITNQDADTVTIDNTVYCLSKVEGEQHVIKGRPVSSSFDPIKFPEDQFNVALDQVFESIENSQALVDQSNKILNSAESAI

SEQ ID NO: 71 - protein, 472 aa, synthetic construct
  REGION 1..472: note = V84C, A140C, A147C, A249C, Q100R, S101R
MSWKVMIIISLLITPQHGLKESYLEESCSTITEGYLSVLRTGWYTNVFTLEVGDVENLTCTDGPSLIKTELDLTKSALRELKTCSADQLAREEQIENPRRRRFVLGAIALGVATAAAVTAGIAIAKTIRLESEVNAIKGCLKTTNECVSTLGNGVRVLATAVRELKEFVSKNLTSAINKNKCDIADLKMAVSFSQFNRRFLNVVRQFSDNAGITPAISLDLMTDAELARAVSYMPTSAGQIKLMLENRCMVRRKGFGILIGVYGSSVIYMVQLPIFGVIDTPCWIIKAAPSCSEKDGNYACLLREDQGWYCKNAGSTVYYPNDKDCETRGDHVFCDTAAGINVAEQSRECNINISTTNYPCKVSTGRHPISMVALSPLGALVACYKGVSCSIGSNRVGIIKQLPKGCSYITNQDADTVTIDNTVYQLSKVEGEQHVIKGRPVSSSFDPIKFPEDQFNVALDQVFESIENSQA

SEQ ID NO: 72 - protein, 453 aa, synthetic construct
  REGION 1..453: note = A63C, A140C, A147C, K188C, K450C, S470C, Q100R, S101R
KESYLEESCSTITEGYLSVLRTGWYTNVFTLEVGDVENLTCTDCPSLIKTELDLTKSALRELKTVSADQLAREEQIENPRRRRFVLGAIALGVATAAAVTAGIAIAKTIRLESEVNAIKGCLKTTNECVSTLGNGVRVLATAVRELKEFVSKNLTSAINKNKCDIADLCMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKDGNYACLLREDQGWYCKNAGSTVYYPNDKDCETRGDHVFCDTAAGINVAEQSRECNINISTTNYPCKVSTGRHPISMVALSPLGALVACYKGVSCSIGSNRVGIIKQLPKGCSYITNQDADTVTIDNTVYQLSKVEGEQHVIKGRPVSSSFDPICFPEDQFNVALDQVFESIENCQA

SEQ ID NO: 73 - protein, 452 aa, synthetic construct
  REGION 1..452: note = A63C, A140C, A147C, K188C, G106deleted, Q100R, S101R
KESYLEESCSTITEGYLSVLRTGWYTNVFTLEVGDVENLTCSDCPSLIKTELDLTKSALRELKTVSADQLAREEQIENPRRRRFVLAIALGVATAAAVTAGVAIAKTIRLESEVTAIKNCLKTTNECVSTLGNGVRVLATAVRELKDFVSKNLTSAINKNKCDIDDLCMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIIKAAPSCSGKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQA

SEQ ID NO: 74 - protein, 453 aa, synthetic construct
  REGION 1..453: note = A113C, A120C, A339C, Q426C, T160F, I177L, Q100R, S101R
KESYLEESCSTITEGYLSVLRTGWYTNVFTLEVGDVENLTCTDGPSLIKTELDLTKSALRELKTVSADQLAREEQIENPRRRRFVLGAIALGVCTAAAVTCGIAIAKTIRLESEVNAIKGALKTTNEAVSTLGNGVRVLAFAVRELKEFVSKNLTSALNKNKCDIADLKMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKDGNYACLLREDQGWYCKNAGSTVYYPNDKDCETRGDHVFCDTACGINVAEQSRECNINISTTNYPCKVSTGRHPISMVALSPLGALVACYKGVSCSIGSNRVGIIKQLPKGCSYITNQDADTVTIDNTVYCLSKVEGEQHVIKGRPVSSSFDPIKFPEDQFNVALDQVFESIENSQA

SEQ ID NO: 75 - protein, 453 aa, synthetic construct
  REGION 1..453: note = A185P, A113C, A339C, Q100R, S101R
KESYLEESCSTITEGYLSVLRTGWYTNVFTLEVGDVENLTCADGPSLIKTELDLTKSALRELRTVSADQLAREEQIENPRRRRFVLGAIALGVCTAAAVTAGVAIAKTIRLESEVTAIKNALKKTNEAVSTLGNGVRVLATAVRELKDFVSKNLTRAINKNKCDIPDLKMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCSGKKGNYACLLREDQGWYCQNAGSTVYYPNEKDCETRGDHVFCDTACGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQA

SEQ ID NO: 76 - protein, 453 aa, synthetic construct
  REGION 1..453: note = A185P, T160F, I177L, Q100R, S101R
KESYLEESCSTITEGYLSVLRTGWYTNVFTLEVGDVENLTCADGPSLIKTELDLTKSALRELRTVSADQLAREEQIENPRRRRFVLGAIALGVATAAAVTAGVAIAKTIRLESEVTAIKNALKKTNEAVSTLGNGVRVLAFAVRELKDFVSKNLTRALNKNKCDIPDLKMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCSGKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQA

SEQ ID NO: 77 - protein, 453 aa, synthetic construct
  REGION 1..453: note = A185P, A113C, A339C, T160F, I177L, Q100R, S101R
KESYLEESCSTITEGYLSVLRTGWYTNVFTLEVGDVENLTCADGPSLIKTELDLTKSALRELRTVSADQLAREEQIENPRRRRFVLGAIALGVCTAAAVTAGVAIAKTIRLESEVTAIKNALKKTNEAVSTLGNGVRVLAFAVRELKDFVSKNLTRALNKNKCDIPDLKMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCSGKKGNYACLLREDQGWYCQNAGSTVYYPNEKDCETRGDHVFCDTACGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQA

SEQ ID NO: 78 - protein, 453 aa, synthetic construct
  REGION 1..453: note = A63C, K188C, N97G, P98G, R99G, Q100G, S101G, R102G
KESYLEESCSTITEGYLSVLRTGWYTNVFTLEVGDVENLTCTDCPSLIKTELDLTKSALRELKTVSADQLAREEQIEGGGGGGFVLGAIALGVATAAAVTAGIAIAKTIRLESEVNAIKGALKTTNEAVSTLGNGVRVLATAVRELKEFVSKNLTSAINKNKCDIADLCMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKDGNYACLLREDQGWYCKNAGSTVYYPNDKDCETRGDHVFCDTAAGINVAEQSRECNINISTTNYPCKVSTGRHPISMVALSPLGALVACYKGVSCSIGSNRVGIIKQLPKGCSYITNQDADTVTIDNTVYQLSKVEGEQHVIKGRPVSSSFDPIKFPEDQFNVALDQVFESIENSQA

SEQ ID NO: 79 - protein, 453 aa, synthetic construct
  REGION 1..453: note = A63C, K188C, K450A, S470A, N97G, P98G, R99G, Q100G, S101G, R102G
KESYLEESCSTITEGYLSVLRTGWYTNVFTLEVGDVENLTCTDCPSLIKTELDLTKSALRELKTVSADQLAREEQIEGGGGGGFVLGAIALGVATAAAVTAGIAIAKTIRLESEVNAIKGALKTTNEAVSTLGNGVRVLATAVRELKEFVSKNLTSAINKNKCDIADLCMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKDGNYACLLREDQGWYCKNAGSTVYYPNDKDCETRGDHVFCDTAAGINVAEQSRECNINISTTNYPCKVSTGRHPISMVALSPLGALVACYKGVSCSIGSNRVGIIKQLPKGCSYITNQDADTVTIDNTVYQLSKVEGEQHVIKGRPVSSSFDPIAFPEDQFNVALDQVFESIENAQA

SEQ ID NO: 80 - protein, 453 aa, synthetic construct
  REGION 1..453: note = A63C, A140C, A147C, K188C, G294E, N97G, P98G, R99G, Q100G, S101G,R102G
KESYLEESCSTITEGYLSVLRTGWYTNVFTLEVGDVENLTCSDCPSLIKTELDLTKSALRELKTVSADQLAREEQIEGGGGGGFVLGAIALGVATAAAVTAGVAIAKTIRLESEVTAIKNCLKTTNECVSTLGNGVRVLATAVRELKDFVSKNLTSAINKNKCDIDDLCMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIIKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQA

SEQ ID NO: 81 - protein, 453 aa, synthetic construct
  REGION 1..453: note = A63C, A140C, A147C, K188C, K450C, S470C, N97G, P98G, R99G, Q100G,S101G, R102G, G294E
KESYLEESCSTITEGYLSVLRTGWYTNVFTLEVGDVENLTCADCPSLIKTELDLTKSALRELRTVSADQLAREEQIEGGGGGGFVLGAIALGVATAAAVTAGVAIAKTIRLESEVTAIKNCLKKTNECVSTLGNGVRVLATAVRELKDFVSKNLTRAINKNKCDIADLCMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVCFPEDQFNVALDQVFESIENCQA

SEQ ID NO: 82 - protein, 453 aa, synthetic construct
  REGION 1..453: note = A63C, K188C, N97G, P98G, R99G, Q100G, S101G, R102G, G294E
KESYLEESCSTITEGYLSVLRTGWYTNVFTLEVGDVENLTCADCPSLIKTELDLTKSALRELRTVSADQLAREEQIEGGGGGGFVLGAIALGVATAAAVTAGVAIAKTIRLESEVTAIKNALKKTNEAVSTLGNGVRVLATAVRELKDFVSKNLTRAINKNKCDIADLCMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQA

SEQ ID NO: 83 - protein, 453 aa, synthetic construct
  REGION 1..453: note = T127C, N153C, T365C, V463C, A185P, L219K, V231I, G294E, H368N,Q100R, S101R
KESYLEESCSTITEGYLSVLRTGWYTNVFTLEVGDVENLTCADGPSLIKTELDLTKSALRELRTVSADQLAREEQIENPRRRRFVLGAIALGVATAAAVTAGVAIAKCIRLESEVTAIKNALKKTNEAVSTLGCGVRVLATAVRELKDFVSKNLTRAINKNKCDIPDLKMAVSFSQFNRRFLNVVRQFSDNAGITPAISKDLMTDAELARAISNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSCGRNPISMVALSPLGALVACYKGVSCSIGSNRVGIIKQLNKGCSYITNQDADTVTIDNTVYQLSKVEGEQHVIKGRPVSSSFDPVKFPEDQFNVALDQCFESIENSQA

SEQ ID NO: 84 - protein, 471 aa, synthetic construct
  REGION 1..471: note = T127C, N153C, T365C, V463C, A185P, L219K, V231I, G294E, H368N,Q100R, S101R
KESYLEESCSTITEGYLSVLRTGWYTNVFTLEVGDVENLTCADGPSLIKTELDLTKSALRELRTVSADQLAREEQIENPRRRRFVLGAIALGVATAAAVTAGVAIAKCIRLESEVTAIKNALKKTNEAVSTLGCGVRVLATAVRELKDFVSKNLTRAINKNKCDIPDLKMAVSFSQFNRRFLNVVRQFSDNAGITPAISKDLMTDAELARAISNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSCGRNPISMVALSPLGALVACYKGVSCSIGSNRVGIIKQLNKGCSYITNQDADTVTIDNTVYQLSKVEGEQHVIKGRPVSSSFDPVKFPEDQFNVALDQCFESIENSQALVDQSNRILSSAEKGNTG

SEQ ID NO: 85 - protein, 453 aa, synthetic construct; identical to SEQ ID NO: 65
  REGION 1..453: note = A63C, A140C, A147C, K188C, K450C, S470C, N97G, P98G, R99G, Q100G,S101G, R102G

SEQ ID NO: 86 - protein, 453 aa, synthetic construct; identical to SEQ ID NO: 83
  REGION 1..453: note = T127C, N153C, T365C, V463C, A185P, L219K, V231I, G294E, Q100R,S101R

SEQ ID NO: 87 - protein, 471 aa, synthetic construct; identical to SEQ ID NO: 84
  REGION 1..471: note = T127C, N153C, T365C, V463C, A185P, L219K, V231I, G294E, Q100R,S101R

SEQ ID NO: 88 - protein, 453 aa, synthetic construct; identical to SEQ ID NO: 63
  REGION 1..453: note = V84C, A140C, A147C, A249C, N97G, P98G, R99G, Q100G, S101G, R102G

SEQ ID NO: 89 - protein, 453 aa, synthetic construct
  REGION 1..453: note = T127C, N153C, T365C, V463C, A185P, L219K, V231I, G294E, N97G,P98G, R99G, Q100G, S101G, H368N, R102G
KESYLEESCSTITEGYLSVLRTGWYTNVFTLEVGDVENLTCADGPSLIKTELDLTKSALRELRTVSADQLAREEQIEGGGGGGFVLGAIALGVATAAAVTAGVAIAKCIRLESEVTAIKNALKKTNEAVSTLGCGVRVLATAVRELKDFVSKNLTRAINKNKCDIPDLKMAVSFSQFNRRFLNVVRQFSDNAGITPAISKDLMTDAELARAISNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSCGRNPISMVALSPLGALVACYKGVSCSIGSNRVGIIKQLNKGCSYITNQDADTVTIDNTVYQLSKVEGEQHVIKGRPVSSSFDPVKFPEDQFNVALDQCFESIENSQA

SEQ ID NO: 90 - protein, 471 aa, synthetic construct
  REGION 1..471: note = T127C, N153C, T365C, V463C, A185P, L219K, V231I, G294E, N97G,P98G, R99G, Q100G, S101G,H368N, R102G
KESYLEESCSTITEGYLSVLRTGWYTNVFTLEVGDVENLTCADGPSLIKTELDLTKSALRELRTVSADQLAREEQIEGGGGGGFVLGAIALGVATAAAVTAGVAIAKCIRLESEVTAIKNALKKTNEAVSTLGCGVRVLATAVRELKDFVSKNLTRAINKNKCDIPDLKMAVSFSQFNRRFLNVVRQFSDNAGITPAISKDLMTDAELARAISNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSCGRNPISMVALSPLGALVACYKGVSCSIGSNRVGIIKQLNKGCSYITNQDADTVTIDNTVYQLSKVEGEQHVIKGRPVSSSFDPVKFPEDQFNVALDQCFESIENSQALVDQSNRILSSAEKGNTG

SEQ ID NO: 91 - protein, 27 aa, synthetic construct
  REGION 1..27: note = the foldon domain of T4 fibritin
GYIPEAPRDGQAYVRKDGEWVLLSTFL

SEQ ID NO: 92 - protein, 691 aa, synthetic construct
  REGION 1..691: note = hMPV001
KESYLEESCSTITEGYLSVLRTGWYTNVFTLEVGDVENLTCADGPSLIKTELDLTKSALRELRTVSADQLAREEQIENPRRRRFVLGAIALGVATAAAVTAGVAIAKTIRLESEVTAIKNALKKTNEAVSTLGNGVRVLATAVRELKDFVSKNLTRAINKNKCDIPDLKMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCSGKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELE

SEQ ID NO: 93 - protein, 683 aa, synthetic construct
  REGION 1..683: note = hMPV002
LKESYLEESCSTITEGYLSVLRTGWYTNVFTLEVGDVENLTCADGPSLIKTELDLTKSALRELRTVSADQLAREEQIENPRRRRFVLGAIALGVATAAAVTAGVAIAKTIRLESEVTAIKNALKKTNEAVSTLGNGVRVLATAVRELKDFVSKNLTRAINKNKCDIPDLKMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCSGKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQAGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELE

SEQ ID NO: 94 - protein, 701 aa, synthetic construct
  REGION 1..701: note = hMPV003
LKESYLEESCSTITEGYLSVLRTGWYTNVFTLEVGDVENLTCADGPSLIKTELDLTKSALRELRTVSADQLAREEQIENPRRRRFVLGAIALGVATAAAVTAGVAIAKTIRLESEVTAIKNALKKTNEAVSTLGNGVRVLATAVRELKDFVSKNLTRAINKNKCDIPDLKMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCSGKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTGGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELE

SEQ ID NO: 95 - protein, 683 aa, synthetic construct
  REGION 1..683: note = hMPV004
LKESYLEESCSTITEGYLSVLRTGWYTNVFTLEVGDVENLTCADGPSLIKTELDLTKSALRELRTVSADQLAREEQIENPRRRRFVLGAIALGVATAAAVTAGVAIAKTIRLESEVTAIKNALKKTNEAVSTLGNGVRVLATAVRELKDFVSKNLTRAINKNKCDIADLKMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCSGKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQAGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELE

SEQ ID NO: 96 - protein, 683 aa, synthetic construct
  REGION 1..683: note = hMPV005
LKESYLEESCSTITEGYLSVLRTGWYTNVFTLEVGDVENLTCADGPSLIKTELDLTKSALRELRTVSADQLAREEQIENPRRRRFVLGAIALGVATAAAVTAGVAIAKCIRLESEVTAIKNALKKTNEAVSTLGCGVRVLATAVRELKDFVSKNLTRAINKNKCDIPDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSGKKGNYACLLREDQGWYCQNAGSTVYYPNEKDCETRGDHVFCDTAAGINVAEQSKECNINISTTNYPCKVSTGRNPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQAGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELE

SEQ ID NO: 97 - protein, 682 aa, synthetic construct
  REGION 1..682: note = hMPV006
KESYLEESCSTITEGYLSVLRTGWYTNVFTLEVGDVENLTCTDGPSLIKTELDLTKSALRELKTCSADQLAREEQIEGGGGGGFVLGAIALGVATAAAVTAGIAIAKTIRLESEVNAIKGCLKTTNECVSTLGNGVRVLATAVRELKEFVSKNLTSAINKNKCDIADLKMAVSFSQFNRRFLNVVRQFSDNAGITPAISLDLMTDAELARAVSYMPTSAGQIKLMLENRCMVRRKGFGILIGVYGSSVIYMVQLPIFGVIDTPCWIIKAAPSCSEKDGNYACLLREDQGWYCKNAGSTVYYPNDKDCETRGDHVFCDTAAGINVAEQSRECNINISTTNYPCKVSTGRHPISMVALSPLGALVACYKGVSCSIGSNRVGIIKQLPKGCSYITNQDADTVTIDNTVYQLSKVEGEQHVIKGRPVSSSFDPIKFPEDQFNVALDQVFESIENSQAGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELE

SEQ ID NO: 98 - protein, 698 aa, synthetic construct
  REGION 1..698: note = hMPV007
KESYLEESCSTITEGYLSVLRTGWYTNVFTLEVGDVENLTCTDGPSLIKTELDLTKSALRELKTCSADQLAREEQIEGGGGGGFVLGAIALGVATAAAVTAGIAIAKTIRLESEVNAIKGCLKTTNECVSTLGNGVRVLATAVRELKEFVSKNLTSAINKNKCDIADLKMAVSFSQFNRRFLNVVRQFSDNAGITPAISLDLMTDAELARAVSYMPTSAGQIKLMLENRCMVRRKGFGILIGVYGSSVIYMVQLPIFGVIDTPCWIIKAAPSCSEKDGNYACLLREDQGWYCKNAGSTVYYPNDKDCETRGDHVFCDTAAGINVAEQSRECNINISTTNYPCKVSTGRHPISMVALSPLGALVACYKGVSCSIGSNRVGIIKQLPKGCSYITNQDADTVTIDNTVYQLSKVEGEQHVIKGRPVSSSFDPIKFPEDQFNVALDQVFESIENSQALVDQSNKILNSAESAIGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELE

SEQ ID NO: 99 - protein, 682 aa, synthetic construct
  REGION 1..682: note = hMPV008
KESYLEESCSTITEGYLSVLRTGWYTNVFTLEVGDVENLTCTDCPSLIKTELDLTKSALRELKTVSADQLAREEQIEGGGGGGFVLGAIALGVATAAAVTAGIAIAKTIRLESEVNAIKGCLKTTNECVSTLGNGVRVLATAVRELKEFVSKNLTSAINKNKCDIADLCMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKDGNYACLLREDQGWYCKNAGSTVYYPNDKDCETRGDHVFCDTAAGINVAEQSRECNINISTTNYPCKVSTGRHPISMVALSPLGALVACYKGVSCSIGSNRVGIIKQLPKGCSYITNQDADTVTIDNTVYQLSKVEGEQHVIKGRPVSSSFDPICFPEDQFNVALDQVFESIENCQAGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELE

SEQ ID NO: 100 - protein, 698 aa, synthetic construct
  REGION 1..698: note = hMPV009
KESYLEESCSTITEGYLSVLRTGWYTNVFTLEVGDVENLTCTDCPSLIKTELDLTKSALRELKTVSADQLAREEQIEGGGGGGFVLGAIALGVATAAAVTAGIAIAKTIRLESEVNAIKGCLKTTNECVSTLGNGVRVLATAVRELKEFVSKNLTSAINKNKCDIADLCMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKDGNYACLLREDQGWYCKNAGSTVYYPNDKDCETRGDHVFCDTAAGINVAEQSRECNINISTTNYPCKVSTGRHPISMVALSPLGALVACYKGVSCSIGSNRVGIIKQLPKGCSYITNQDADTVTIDNTVYQLSKVEGEQHVIKGRPVSSSFDPICFPEDQFNVALDQVFESIENCQALVDQSNKILNSAESAIGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELE

SEQ ID NO: 101 - protein, 682 aa, synthetic construct
  REGION 1..682: note = hMPV010
LKESYLEESCSTITEGYLSVLRTGWYTNVFTLEVGDVENLTCSDCPSLIKTELDLTKSALRELKTVSADQLAREEQIEGGGGGGFVLAIALGVATAAAVTAGVAIAKTIRLESEVTAIKNCLKTTNECVSTLGNGVRVLATAVRELKDFVSKNLTSAINKNKCDIDDLCMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIIKAAPSCSGKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQAGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELE

SEQ ID NO: 102 - protein, 698 aa, synthetic construct
  REGION 1..698: note = hMPV011
LKESYLEESCSTITEGYLSVLRTGWYTNVFTLEVGDVENLTCSDCPSLIKTELDLTKSALRELKTVSADQLAREEQIEGGGGGGFVLAIALGVATAAAVTAGVAIAKTIRLESEVTAIKNCLKTTNECVSTLGNGVRVLATAVRELKDFVSKNLTSAINKNKCDIDDLCMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIIKAAPSCSGKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAESAIGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELE

SEQ ID NO: 103 - protein, 682 aa, synthetic construct
  REGION 1..682: note = hMPV012
KESYLEESCSTITEGYLSVLRTGWYTNVFTLEVGDVENLTCTDGPSLIKTELDLTKSALRELKTVSADQLAREEQIENPRKARFVLGAIALGVCTAAAVTCGIAIAKTIRLESEVNAIKGALKTTNEAVSTLGNGVRVLAFAVRELKEFVSKNLTSALNKNKCDIADLKMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKDGNYACLLREDQGWYCKNAGSTVYYPNDKDCETRGDHVFCDTACGINVAEQSRECNINISTTNYPCKVSTGRHPISMVALSPLGALVACYKGVSCSIGSNRVGIIKQLPKGCSYITNQDADTVTIDNTVYCLSKVEGEQHVIKGRPVSSSFDPIKFPEDQFNVALDQVFESIENSQAGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELE

SEQ ID NO: 104 - protein, 698 aa, synthetic construct
  REGION 1..698: note = hMPV013
KESYLEESCSTITEGYLSVLRTGWYTNVFTLEVGDVENLTCTDGPSLIKTELDLTKSALRELKTVSADQLAREEQIENPRKARFVLGAIALGVCTAAAVTCGIAIAKTIRLESEVNAIKGALKTTNEAVSTLGNGVRVLAFAVRELKEFVSKNLTSALNKNKCDIADLKMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKDGNYACLLREDQGWYCKNAGSTVYYPNDKDCETRGDHVFCDTACGINVAEQSRECNINISTTNYPCKVSTGRHPISMVALSPLGALVACYKGVSCSIGSNRVGIIKQLPKGCSYITNQDADTVTIDNTVYCLSKVEGEQHVIKGRPVSSSFDPIKFPEDQFNVALDQVFESIENSQALVDQSNKILNSAESAIGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELE

SEQ ID NO: 105 - protein, 682 aa, synthetic construct
  REGION 1..682: note = hMPV014
KESYLEESCSTITEGYLSVLRTGWYTNVFTLEVGDVENLTCTDGPSLIKTELDLTKSALRELKTCSADQLAREEQIENPRRRRFVLGAIALGVATAAAVTAGIAIAKTIRLESEVNAIKGCLKTTNECVSTLGNGVRVLATAVRELKEFVSKNLTSAINKNKCDIADLKMAVSFSQFNRRFLNVVRQFSDNAGITPAISLDLMTDAELARAVSYMPTSAGQIKLMLENRCMVRRKGFGILIGVYGSSVIYMVQLPIFGVIDTPCWIIKAAPSCSEKDGNYACLLREDQGWYCKNAGSTVYYPNDKDCETRGDHVFCDTAAGINVAEQSRECNINISTTNYPCKVSTGRHPISMVALSPLGALVACYKGVSCSIGSNRVGIIKQLPKGCSYITNQDADTVTIDNTVYQLSKVEGEQHVIKGRPVSSSFDPIKFPEDQFNVALDQVFESIENSQAGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELE

SEQ ID NO: 106 - protein, 682 aa, synthetic construct
  REGION 1..682: note = hMPV015
KESYLEESCSTITEGYLSVLRTGWYTNVFTLEVGDVENLTCTDCPSLIKTELDLTKSALRELKTVSADQLAREEQIENPRRRRFVLGAIALGVATAAAVTAGIAIAKTIRLESEVNAIKGCLKTTNECVSTLGNGVRVLATAVRELKEFVSKNLTSAINKNKCDIADLCMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKDGNYACLLREDQGWYCKNAGSTVYYPNDKDCETRGDHVFCDTAAGINVAEQSRECNINISTTNYPCKVSTGRHPISMVALSPLGALVACYKGVSCSIGSNRVGIIKQLPKGCSYITNQDADTVTIDNTVYQLSKVEGEQHVIKGRPVSSSFDPICFPEDQFNVALDQVFESIENCQAGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELE

SEQ ID NO: 107 - protein, 682 aa, synthetic construct
  REGION 1..682: note = hMPV016
LKESYLEESCSTITEGYLSVLRTGWYTNVFTLEVGDVENLTCSDCPSLIKTELDLTKSALRELKTVSADQLAREEQIENPRRRRFVLAIALGVATAAAVTAGVAIAKTIRLESEVTAIKNCLKTTNECVSTLGNGVRVLATAVRELKDFVSKNLTSAINKNKCDIDDLCMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIIKAAPSCSGKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQAGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELE

SEQ ID NO: 108 - protein, 682 aa, synthetic construct
  REGION 1..682: note = hMPV017
KESYLEESCSTITEGYLSVLRTGWYTNVFTLEVGDVENLTCTDGPSLIKTELDLTKSALRELKTVSADQLAREEQIENPRRRRFVLGAIALGVCTAAAVTCGIAIAKTIRLESEVNAIKGALKTTNEAVSTLGNGVRVLAFAVRELKEFVSKNLTSALNKNKCDIADLKMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKDGNYACLLREDQGWYCKNAGSTVYYPNDKDCETRGDHVFCDTACGINVAEQSRECNINISTTNYPCKVSTGRHPISMVALSPLGALVACYKGVSCSIGSNRVGIIKQLPKGCSYITNQDADTVTIDNTVYCLSKVEGEQHVIKGRPVSSSFDPIKFPEDQFNVALDQVFESIENSQAGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELE

SEQ ID NO: 109 - protein, 683 aa, synthetic construct
  REGION 1..683: note = hMPV018
LKESYLEESCSTITEGYLSVLRTGWYTNVFTLEVGDVENLTCADGPSLIKTELDLTKSALRELRTVSADQLAREEQIENPRRRRFVLGAIALGVCTAAAVTAGVAIAKTIRLESEVTAIKNALKKTNEAVSTLGNGVRVLATAVRELKDFVSKNLTRAINKNKCDIPDLKMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCSGKKGNYACLLREDQGWYCQNAGSTVYYPNEKDCETRGDHVFCDTACGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQAGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELE

SEQ ID NO: 110 - protein, 683 aa, synthetic construct
  REGION 1..683: note = hMPV019
LKESYLEESCSTITEGYLSVLRTGWYTNVFTLEVGDVENLTCADGPSLIKTELDLTKSALRELRTVSADQLAREEQIENPRRRRFVLGAIALGVATAAAVTAGVAIAKTIRLESEVTAIKNALKKTNEAVSTLGNGVRVLAFAVRELKDFVSKNLTRALNKNKCDIPDLKMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCSGKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQAGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELE

SEQ ID NO: 111 - protein, 683 aa, synthetic construct
  REGION 1..683: note = hMPV020
LKESYLEESCSTITEGYLSVLRTGWYTNVFTLEVGDVENLTCADGPSLIKTELDLTKSALRELRTVSADQLAREEQIENPRRRRFVLGAIALGVCTAAAVTAGVAIAKTIRLESEVTAIKNALKKTNEAVSTLGNGVRVLAFAVRELKDFVSKNLTRALNKNKCDIPDLKMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCSGKKGNYACLLREDQGWYCQNAGSTVYYPNEKDCETRGDHVFCDTACGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQAGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELE

SEQ ID NO: 112 - protein, 682 aa, synthetic construct
  REGION 1..682: note = hMPV021
KESYLEESCSTITEGYLSVLRTGWYTNVFTLEVGDVENLTCTDCPSLIKTELDLTKSALRELKTVSADQLAREEQIEGGGGGGFVLGAIALGVATAAAVTAGIAIAKTIRLESEVNAIKGALKTTNEAVSTLGNGVRVLATAVRELKEFVSKNLTSAINKNKCDIADLCMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKDGNYACLLREDQGWYCKNAGSTVYYPNDKDCETRGDHVFCDTAAGINVAEQSRECNINISTTNYPCKVSTGRHPISMVALSPLGALVACYKGVSCSIGSNRVGIIKQLPKGCSYITNQDADTVTIDNTVYQLSKVEGEQHVIKGRPVSSSFDPIKFPEDQFNVALDQVFESIENSQAGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELE

SEQ ID NO: 113 - protein, 682 aa, synthetic construct
  REGION 1..682: note = hMPV022
KESYLEESCSTITEGYLSVLRTGWYTNVFTLEVGDVENLTCTDCPSLIKTELDLTKSALRELKTVSADQLAREEQIEGGGGGGFVLGAIALGVATAAAVTAGIAIAKTIRLESEVNAIKGALKTTNEAVSTLGNGVRVLATAVRELKEFVSKNLTSAINKNKCDIADLCMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKDGNYACLLREDQGWYCKNAGSTVYYPNDKDCETRGDHVFCDTAAGINVAEQSRECNINISTTNYPCKVSTGRHPISMVALSPLGALVACYKGVSCSIGSNRVGIIKQLPKGCSYITNQDADTVTIDNTVYQLSKVEGEQHVIKGRPVSSSFDPIAFPEDQFNVALDQVFESIENAQAGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELE

SEQ ID NO: 114 - protein, 683 aa, synthetic construct
  REGION 1..683: note = hMPV023
LKESYLEESCSTITEGYLSVLRTGWYTNVFTLEVGDVENLTCSDCPSLIKTELDLTKSALRELKTVSADQLAREEQIEGGGGGGFVLGAIALGVATAAAVTAGVAIAKTIRLESEVTAIKNCLKTTNECVSTLGNGVRVLATAVRELKDFVSKNLTSAINKNKCDIDDLCMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIIKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQAGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELE

SEQ ID NO: 115 - protein, 683 aa, synthetic construct
  REGION 1..683: note = hMPV024
LKESYLEESCSTITEGYLSVLRTGWYTNVFTLEVGDVENLTCADCPSLIKTELDLTKSALRELRTVSADQLAREEQIEGGGGGGFVLGAIALGVATAAAVTAGVAIAKTIRLESEVTAIKNCLKKTNECVSTLGNGVRVLATAVRELKDFVSKNLTRAINKNKCDIADLCMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVCFPEDQFNVALDQVFESIENCQAGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELE

SEQ ID NO: 116 - protein, 683 aa, synthetic construct
  REGION 1..683: note = hMPV025
LKESYLEESCSTITEGYLSVLRTGWYTNVFTLEVGDVENLTCADCPSLIKTELDLTKSALRELRTVSADQLAREEQIEGGGGGGFVLGAIALGVATAAAVTAGVAIAKTIRLESEVTAIKNALKKTNEAVSTLGNGVRVLATAVRELKDFVSKNLTRAINKNKCDIADLCMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQAGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELE

SEQ ID NO: 117 - protein, 683 aa, synthetic construct
  REGION 1..683: note = hMPV026
LKESYLEESCSTITEGYLSVLRTGWYTNVFTLEVGDVENLTCADGPSLIKTELDLTKSALRELRTVSADQLAREEQIENPRRRRFVLGAIALGVATAAAVTAGVAIAKCIRLESEVTAIKNALKKTNEAVSTLGCGVRVLATAVRELKDFVSKNLTRAINKNKCDIPDLKMAVSFSQFNRRFLNVVRQFSDNAGITPAISKDLMTDAELARAISNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSCGRNPISMVALSPLGALVACYKGVSCSIGSNRVGIIKQLNKGCSYITNQDADTVTIDNTVYQLSKVEGEQHVIKGRPVSSSFDPVKFPEDQFNVALDQCFESIENSQAGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELE

SEQ ID NO: 118 - protein, 701 aa, synthetic construct
  REGION 1..701: note = hMPV027
LKESYLEESCSTITEGYLSVLRTGWYTNVFTLEVGDVENLTCADGPSLIKTELDLTKSALRELRTVSADQLAREEQIENPRRRRFVLGAIALGVATAAAVTAGVAIAKCIRLESEVTAIKNALKKTNEAVSTLGCGVRVLATAVRELKDFVSKNLTRAINKNKCDIPDLKMAVSFSQFNRRFLNVVRQFSDNAGITPAISKDLMTDAELARAISNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSCGRNPISMVALSPLGALVACYKGVSCSIGSNRVGIIKQLNKGCSYITNQDADTVTIDNTVYQLSKVEGEQHVIKGRPVSSSFDPVKFPEDQFNVALDQCFESIENSQALVDQSNRILSSAEKGNTGGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELE

SEQ ID NO: 119 - protein, 682 aa, synthetic construct
  REGION 1..682: note = hMPV028
KESYLEESCSTITEGYLSVLRTGWYTNVFTLEVGDVENLTCTDCPSLIKTELDLTKSALRELKTVSADQLAREEQIEGGGGGGFVLGAIALGVATAAAVTAGIAIAKTIRLESEVNAIKGCLKTTNECVSTLGNGVRVLATAVRELKEFVSKNLTSAINKNKCDIADLCMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKDGNYACLLREDQGWYCKNAGSTVYYPNDKDCETRGDHVFCDTAAGINVAEQSRECNINISTTNYPCKVSTGRHPISMVALSPLGALVACYKGVSCSIGSNRVGIIKQLPKGCSYITNQDADTVTIDNTVYQLSKVEGEQHVIKGRPVSSSFDPICFPEDQFNVALDQVFESIENCQAGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTELE

SEQ ID NO: 120 - protein, 683 aa, synthetic construct
  REGION 1..683: note = hMPV029
LKESYLEESCSTITEGYLSVLRTGWYTNVFTLEVGDVENLTCADGPSLIKTELDLTKSALRELRTVSADQLAREEQIENPRRRRFVLGAIALGVATAAAVTAGVAIAKCIRLESEVTAIKNALKKTNEAVSTLGCGVRVLATAVRELKDFVSKNLTRAINKNKCDIPDLKMAVSFSQFNRRFLNVVRQFSDNAGITPAISKDLMTDAELARAISNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSCGRNPISMVALSPLGALVACYKGVSCSIGSNRVGIIKQLNKGCSYITNQDADTVTIDNTVYQLSKVEGEQHVIKGRPVSSSFDPVKFPEDQFNVALDQCFESIENSQAGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTELE

SEQ ID NO: 121 - protein, 701 aa, synthetic construct
  REGION 1..701: note = hMPV030
LKESYLEESCSTITEGYLSVLRTGWYTNVFTLEVGDVENLTCADGPSLIKTELDLTKSALRELRTVSADQLAREEQIENPRRRRFVLGAIALGVATAAAVTAGVAIAKCIRLESEVTAIKNALKKTNEAVSTLGCGVRVLATAVRELKDFVSKNLTRAINKNKCDIPDLKMAVSFSQFNRRFLNVVRQFSDNAGITPAISKDLMTDAELARAISNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSCGRNPISMVALSPLGALVACYKGVSCSIGSNRVGIIKQLNKGCSYITNQDADTVTIDNTVYQLSKVEGEQHVIKGRPVSSSFDPVKFPEDQFNVALDQCFESIENSQALVDQSNRILSSAEKGNTGGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTELE

SEQ ID NO: 122 - protein, 682 aa, synthetic construct
  REGION 1..682: note = hMPV031
KESYLEESCSTITEGYLSVLRTGWYTNVFTLEVGDVENLTCTDGPSLIKTELDLTKSALRELKTCSADQLAREEQIEGGGGGGFVLGAIALGVATAAAVTAGIAIAKTIRLESEVNAIKGCLKTTNECVSTLGNGVRVLATAVRELKEFVSKNLTSAINKNKCDIADLKMAVSFSQFNRRFLNVVRQFSDNAGITPAISLDLMTDAELARAVSYMPTSAGQIKLMLENRCMVRRKGFGILIGVYGSSVIYMVQLPIFGVIDTPCWIIKAAPSCSEKDGNYACLLREDQGWYCKNAGSTVYYPNDKDCETRGDHVFCDTAAGINVAEQSRECNINISTTNYPCKVSTGRHPISMVALSPLGALVACYKGVSCSIGSNRVGIIKQLPKGCSYITNQDADTVTIDNTVYQLSKVEGEQHVIKGRPVSSSFDPIKFPEDQFNVALDQVFESIENSQAGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTELE

SEQ ID NO: 123 - protein, 683 aa, synthetic construct
  REGION 1..683: note = hMPV033
LKESYLEESCSTITEGYLSVLRTGWYTNVFTLEVGDVENLTCADGPSLIKTELDLTKSALRELRTVSADQLAREEQIEGGGGGGFVLGAIALGVATAAAVTAGVAIAKCIRLESEVTAIKNALKKTNEAVSTLGCGVRVLATAVRELKDFVSKNLTRAINKNKCDIPDLKMAVSFSQFNRRFLNVVRQFSDNAGITPAISKDLMTDAELARAISNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSCGRNPISMVALSPLGALVACYKGVSCSIGSNRVGIIKQLNKGCSYITNQDADTVTIDNTVYQLSKVEGEQHVIKGRPVSSSFDPVKFPEDQFNVALDQCFESIENSQAGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELE

SEQ ID NO: 124 - protein, 701 aa, synthetic construct
  REGION 1..701: note = hMPV034
LKESYLEESCSTITEGYLSVLRTGWYTNVFTLEVGDVENLTCADGPSLIKTELDLTKSALRELRTVSADQLAREEQIEGGGGGGFVLGAIALGVATAAAVTAGVAIAKCIRLESEVTAIKNALKKTNEAVSTLGCGVRVLATAVRELKDFVSKNLTRAINKNKCDIPDLKMAVSFSQFNRRFLNVVRQFSDNAGITPAISKDLMTDAELARAISNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSCGRNPISMVALSPLGALVACYKGVSCSIGSNRVGIIKQLNKGCSYITNQDADTVTIDNTVYQLSKVEGEQHVIKGRPVSSSFDPVKFPEDQFNVALDQCFESIENSQALVDQSNRILSSAEKGNTGGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELE

SEQ ID NO: 125 - protein, 12 aa, synthetic construct
  REGION 1..12: note = foldon sequence
EKAAKAEEAARK

SEQ ID NO: 126 - protein, 12 aa, synthetic construct
  REGION 1..12: note = Gly-Ser linker
GSGGSGSGSGGS

SEQ ID NO: 127 - protein, 15 aa, synthetic construct
  REGION 1..15: note = Gly-Ser linker
GSGGSGSGSGGSGSG

SEQ ID NO: 128 - protein, 8 aa, synthetic construct
  REGION 1..8: note = Gly-Ser linker
GGSGGSGS

SEQ ID NO: 129 - protein, 8 aa, synthetic construct
  REGION 1..8: note = Gly-Ser linker
GSGGSGSG

SEQ ID NO: 130 - protein, 7 aa, synthetic construct
  REGION 1..7: note = Gly-Ser linker
GSGSGSG

SEQ ID NO: 131 - protein, 15 aa, synthetic construct
  REGION 1..15: note = Gly-Ser linker
GSGSGSGSGSGSGSG

SEQ ID NO: 132 - protein, 203 aa, synthetic construct
  REGION 1..203: note = multimerization domain of the first polypeptide
MEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 133 - protein, 19 aa, synthetic construct
  REGION 1..19: note = ectodomain N-terminal signal peptide
MSWKVVIIFSLLITPQHGL

SEQ ID NO: 134 - protein, 19 aa, synthetic construct
  REGION 1..19: note = ectodomain N-terminal signal peptide
MSWKVMIIISLLITPQHGL

SEQ ID NO: 135 - protein, 119 aa, synthetic construct
  REGION 1..119: note = T33_dn2A
NLAEKMYKAGNAMYRKGQYTIAIIAYTLALLKDPNNAEAWYNLGNAAYKKGEYDEAIEAYQKALELDPNNAEAWYNLGNAYYKQGDYDEAIEYYKKALRLDPRNVDAIENLIEAEEKQG

SEQ ID NO: 136 - protein, 119 aa, synthetic construct
  REGION 1..119: note = T33_dn2B
EEAELAYLLGELAYKLGEYRIAIRAYRIALKRDPNNAEAWYNLGNAYYKQGDYREAIRYYLRALKLDPENAEAWYNLGNALYKQGKYDLAIIAYQAALEEDPNNAEAKQNLGNAKQKQG

SEQ ID NO: 137 - protein, 119 aa, synthetic construct
  REGION 1..119: note = T33_dn5A
NSAEAMYKMGNAAYKQGDYILAIIAYLLALEKDPNNAEAWYNLGNAAYKQGDYDEAIEYYQKALELDPNNAEAWYNLGNAYYKQGDYDEAIEYYEKALELDPNNAEALKNLLEAIAEQD

SEQ ID NO: 138 - protein, 187 aa, synthetic construct
  REGION 1..187: note = T33_dn5A
TDPLAVILYIAILKAEKSIARAKAAEALGKIGDERAVEPLIKALKDEDALVRAAAADALGQIGDERAVEPLIKALKDEEGLVRASAAIALGQIGDERAVQPLIKALTDERDLVRVAAAVALGRIGDEKAVRPLIIVLKDEEGEVREAAAIALGSIGGERVRAAMEKLAERGTGFARKVAVNYLETHK

SEQ ID NO: 139 - protein, 119 aa, synthetic construct
  REGION 1..119: note = T33_dn10A
EEAELAYLLGELAYKLGEYRIAIRAYRIALKRDPNNAEAWYNLGNAYYKQGDYDEAIEYYQKALELDPNNAEAWYNLGNAYYKQGDYDEAIEYYEKALELDPENLEALQNLLNAMDKQG

SEQ ID NO: 140 - protein, 279 aa, synthetic construct
  REGION 1..279: note = T33_dn10B
IEEVVAEMIDILAESSKKSIEELARAADNKTTEKAVAEAIEEIARLATAAIQLIEALAKNLASEEFMARAISAIAELAKKAIEAIYRLADNHTTDTFMARAIAAIANLAVTAILAIAALASNHTTEEFMARAISAIAELAKKAIEAIYRLADNHTTDKFMAAAIEAIALLATLAILAIALLASNHTTEKFMARAIMAIAILAAKAIEAIYRLADNHTSPTYIEKAIEAIEKIARKAIKAIEMLAKNITTEEYKEKAKKIIDIIRKLAKMAIKKLEDNRT

SEQ ID NO: 141 - protein, 153 aa, synthetic construct
  REGION 1..153: note = I53_dn5A
KYDGSKLRIGILHARWNAEIILALVLGALKRLQEFGVKRENIIIETVPGSFELPYGSKLFVEKQKRLGKPLDAIIPIGVLIKGSTMHFEYICDSTTHQLMKLNFELGIPVIFGVLTCLTDEQAEARAGLIEGKMHNHGEDWGAAAVEMATKFN

SEQ ID NO: 142 - protein, 119 aa, synthetic construct
  REGION 1..119: note = I53_dn5B
EEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 143 - protein, 6 aa, synthetic construct
  REGION 1..6: note = poly-His tag
HHHHHH

SEQ ID NO: 144 - protein, 203 aa, synthetic construct
  REGION 1..203: note = I53-50A
MEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 145 - protein, 203 aa, synthetic construct; identical to SEQ ID NO: 132
  REGION 1..203: note = I53-50A deltaCys

SEQ ID NO: 146 - protein, 613 aa, synthetic construct
  REGION 1..613: note = DS-Cav1-T33-15B
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLGGSMVRGIRGAITVNSDTPTSIIIATILLLEKMLEANGIQSYEELAAVIFTVTEDLTSAFPAEAARQIGMHRVPLLSAREVPVPGSLPRVIRVLALWNTDTPQDRVRHVYLSEAVRLRPDLESAQ

SEQ ID NO: 147 - protein, 744 aa, synthetic construct
  REGION 1..744: note = DS-Cav1-foldon-I53-50A
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRGYIPEAPRDGQAYVRKDGEWVLLSTFLGSGSHHHHHHHHGGSGGSGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 148 - protein, 705 aa, synthetic construct
  REGION 1..705: note = DS-Cav1-I53-50A
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRGGSGGSGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 149 - protein, 651 aa, synthetic construct
  REGION 1..651: note = DS-Cav1-I32-28A
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLGGSGGSGSDDARIAAIGDVDELNSQIGVLLAEPLPDDVRAALSAIQHDLFDLGGELCIPGHAAITEDHLLRLALWLVHYNGQLPPLEEFILPGGARGAALAHVCRTVCRRAERSIKALGASEPLNIAPAAYVNLLSDLLFVLARVLNRAAGGADVLWDRTRAH

SEQ ID NO: 150 - protein, 739 aa, synthetic construct
  REGION 1..739: note = DS-Cav1-foldon-15GS-HelExt-I53-50A (F14)
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRGYIPEAPRDGQAYVRKDGEWVLLSTFLGSGGSGSGSGGSGSGEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 151 - protein, 715 aa, synthetic construct
  REGION 1..715: note = CompA-RSV-02
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 152 - protein, 515 aa, synthetic construct
  REGION 1..515: note = DS-Cav1-foldon
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLGYIPEAPRDGQAYVRKDGEWVLLSTFL

SEQ ID NO: 153 - protein, 621 aa, synthetic construct
  REGION 1..621: note = DS-Cav1-foldon-T33-31A
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLGYIPEAPRDGQAYVRKDGEWVLLSTFLGGSMEEVVLITVPSALVAVKIAHALVEERLAACVNIVPGLTSIYREEGSVVSDHELLLLVKTTTDAFPKLKERVKELHPYEVPEIVALPIAEGNREYLDWLRENTG

SEQ ID NO: 154 - protein, 594 aa, synthetic construct
  REGION 1..594: note = DS-Cav1-T33-31A
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLGGSMEEVVLITVPSALVAVKIAHALVEERLAACVNIVPGLTSIYREEGSVVSDHELLLLVKTTTDAFPKLKERVKELHPYEVPEIVALPIAEGNREYLDWLRENTG

SEQ ID NO: 155 - protein, 640 aa, synthetic construct
  REGION 1..640: note = DS-Cav1-foldon-T33-15B
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLGYIPEAPRDGQAYVRKDGEWVLLSTFLGGSMVRGIRGAITVNSDTPTSIIIATILLLEKMLEANGIQSYEELAAVIFTVTEDLTSAFPAEAARQIGMHRVPLLSAREVPVPGSLPRVIRVLALWNTDTPQDRVRHVYLSEAVRLRPDLESAQ

SEQ ID NO: 156 - protein, 119 aa, synthetic construct; identical to SEQ ID NO: 142
  REGION 1..119: note = I53_dn5B

SEQ ID NO: 157 - protein, 25 aa, synthetic construct
  REGION 1..25: note = Signal sequence
MELLILKANAITTILTAVTFCFASG

SEQ ID NO: 158 - protein, 25 aa, synthetic construct
  REGION 1..25: note = Signal sequence
MELLILKANVIATILTAVTFCFASS

SEQ ID NO: 159 - protein, 18 aa, synthetic construct
  REGION 1..18: note = Signal sequence
MSWKVVIIFSLLITPQHG

SEQ ID NO: 160 - protein, 488 aa, synthetic construct
  REGION 1..488: note = RSV F protein DS-Cav1
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELL

SEQ ID NO: 161 - protein, 488 aa, synthetic construct
  REGION 1..488: note = RSV-B F protein
QNITEEFYQSTCSAVSRGYFSALRTGWYTSVITIELSNITETKCNGTDTKVKLIKQELDKYKNAVTELQLLMQNTPAANNRARREAPQHMNYTINTTKNLNVSISKKRKRRFLGFLLGVGSAIASGIAVSKVLHLEGEVNKIKNALLSTNKAVVSLSNGVSVLTSKVLDLKNYINNQLLPIVNQQSCRIFNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRKSDELL

SEQ ID NO: 162 - protein, 488 aa, synthetic construct
  REGION 1..488: note = RSV-B F protein (stabilized)
QNITEEFYQSTCSAVSRGYFSALRTGWYTSVITIELSNITETKCNGTDTKVKLIKQELDKYKNAVTELQLLMQNTPAANNRARREAPQHMNYTINTTKNLNVSISKKRKRRFLGFLLGVGSAIASGIAVCKVLHLEGEVNKIKNALLSTNKAVVSLSNGVSVLTFKVLDLKNYINNQLLPILNQQSCRIFNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRKSDELL

SEQ ID NO: 163 - protein, 11 aa, synthetic construct
  REGION 1..11: note = Extension
EKAAKAEEAAR

SEQ ID NO: 164 - protein, 157 aa, synthetic construct
  REGION 1..157: note = I53-50B
MNQHSHKDYETVRIAVVRARWHAEIVDACVSAFEAAMADIGGDRFAVDVFDVPGAYEIPLHARTLAETGRYGAVLGTAFVVNGGIYRHEFVASAVIDGMMNVQLSTGVPVLSAVLTPHRYRDSDAHTLLFLALFAVKGMEAARACVEILAAREKIAA

SEQ ID NO: 165 - protein, 157 aa, synthetic construct
  REGION 1..157: note = I53-50B.1
MNQHSHKDHETVRIAVVRARWHAEIVDACVSAFEAAMRDIGGDRFAVDVFDVPGAYEIPLHARTLAETGRYGAVLGTAFVVNGGIYRHEFVASAVIDGMMNVQLDTGVPVLSAVLTPHRYRDSDAHTLLFLALFAVKGMEAARACVEILAAREKIAA

SEQ ID NO: 166 - protein, 203 aa, synthetic construct; identical to SEQ ID NO: 144
  REGION 1..203: note = I53-50A

SEQ ID NO: 167 - protein, 156 aa, synthetic construct; identical to SEQ ID NO: 33
  REGION 1..156: note = I53-50B.1NegT2

SEQ ID NO: 168 - protein, 157 aa, synthetic construct
  REGION 1..157: note = I53-50B.4PosT1
MNQHSHKDHETVRIAVVRARWHAEIVDACVSAFEAAMRDIGGDRFAVDVFDVPGAYEIPLHARTLAETGRYGAVLGTAFVVNGGIYRHEFVASAVINGMMNVQLNTGVPVLSAVLTPHNYDKSKAHTLLFLALFAVKGMEAARACVEILAAREKIAA

SEQ ID NO: 169 - protein, 122 aa, synthetic construct
  REGION 1..122: note = T33-15B
MVRGIRGAITVNSDTPTSIIIATILLLEKMLEANGIQSYEELAAVIFTVTEDLTSAFPAEAARQIGMHRVPLLSAREVPVPGSLPRVIRVLALWNTDTPQDRVRHVYLSEAVRLRPDLESAQ

SEQ ID NO: 170 - protein, 157 aa, synthetic construct
  REGION 1..157: note = I32-28A
MGDDARIAAIGDVDELNSQIGVLLAEPLPDDVRAALSAIQHDLFDLGGELCIPGHAAITEDHLLRLALWLVHYNGQLPPLEEFILPGGARGAALAHVCRTVCRRAERSIKALGASEPLNIAPAAYVNLLSDLLFVLARVLNRAAGGADVLWDRTRAH

SEQ ID NO: 171 - protein, 103 aa, synthetic construct
  REGION 1..103: note = T33-31A
MEEVVLITVPSALVAVKIAHALVEERLAACVNIVPGLTSIYREEGSVVSDHELLLLVKTTTDAFPKLKERVKELHPYEVPEIVALPIAEGNREYLDWLRENTG

SEQ ID NO: 172 - protein, 153 aa, synthetic construct; identical to SEQ ID NO: 141
  REGION 1..153: note = I53_dn5A

SEQ ID NO: 173 - protein, 488 aa, synthetic construct; identical to SEQ ID NO: 160
  REGION 1..488: note = RSV F DS-Cav1

SEQ ID NO: 174 - protein, 453 aa, synthetic construct; identical to SEQ ID NO: 65
  REGION 1..453: note = hMPV F protein ectodomain hMPV008

SEQ ID NO: 175 - protein, 453 aa, synthetic construct; identical to SEQ ID NO: 89
  REGION 1..453: note = hMPV F protein ectodomain hMPV033

SEQ ID NO: 176 - protein, 680 aa, synthetic construct
  REGION 1..680: note = hMPV008 VLP
KESYLEESCSTITEGYLSVLRTGWYTNVFTLEVGDVENLTCTDCPSLIKTELDLTKSALRELKTVSADQLAREEQIEGGGGGGFVLGAIALGVATAAAVTAGIAIAKTIRLESEVNAIKGCLKTTNECVSTLGNGVRVLATAVRELKEFVSKNLTSAINKNKCDIADLCMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKDGNYACLLREDQGWYCKNAGSTVYYPNDKDCETRGDHVFCDTAAGINVAEQSRECNINISTTNYPCKVSTGRHPISMVALSPLGALVACYKGVSCSIGSNRVGIIKQLPKGCSYITNQDADTVTIDNTVYQLSKVEGEQHVIKGRPVSSSFDPICFPEDQFNVALDQVFESIENCQAGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 177 - protein, 680 aa, synthetic construct
  REGION 1..680: note = hMPV033 VLP
KESYLEESCSTITEGYLSVLRTGWYTNVFTLEVGDVENLTCADGPSLIKTELDLTKSALRELRTVSADQLAREEQIEGGGGGGFVLGAIALGVATAAAVTAGVAIAKCIRLESEVTAIKNALKKTNEAVSTLGCGVRVLATAVRELKDFVSKNLTRAINKNKCDIPDLKMAVSFSQFNRRFLNVVRQFSDNAGITPAISKDLMTDAELARAISNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSCGRNPISMVALSPLGALVACYKGVSCSIGSNRVGIIKQLNKGCSYITNQDADTVTIDNTVYQLSKVEGEQHVIKGRPVSSSFDPVKFPEDQFNVALDQCFESIENSQAGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 178 - protein, 488 aa, synthetic construct
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELL

SEQ ID NO: 179 - protein, 613 aa, synthetic construct
QNITEEFYQSTCSAVSRGYFSALRTGWYTSVITIELSNITETKCNGTDTKVKLIKQELDKYKNAVTELQLLMQNTPAANNRARREAPQHMNYTINTTKNLNVSISKKRKRRFLGFLLGVGSAIASGIAVCKVLHLEGEVNKIKNALLSTNKAVVSLSNGVSVLTFKVLDLKNYINNQLLPILNQQSCRIFNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRKSDELLGGSMVRGIRGAITVNSDTPTSIIIATILLLEKMLEANGIQSYEELAAVIFTVTEDLTSAFPAEAARQIGMHRVPLLSAREVPVPGSLPRVIRVLALWNTDTPQDRVRHVYLSEAVRLRPDLESAQ

SEQ ID NO: 180 - protein, 744 aa, synthetic construct
QNITEEFYQSTCSAVSRGYFSALRTGWYTSVITIELSNITETKCNGTDTKVKLIKQELDKYKNAVTELQLLMQNTPAANNRARREAPQHMNYTINTTKNLNVSISKKRKRRFLGFLLGVGSAIASGIAVCKVLHLEGEVNKIKNALLSTNKAVVSLSNGVSVLTFKVLDLKNYINNQLLPILNQQSCRIFNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRGYIPEAPRDGQAYVRKDGEWVLLSTFLGSGSHHHHHHHHGGSGGSGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 181 - protein, 705 aa, synthetic construct
QNITEEFYQSTCSAVSRGYFSALRTGWYTSVITIELSNITETKCNGTDTKVKLIKQELDKYKNAVTELQLLMQNTPAANNRARREAPQHMNYTINTTKNLNVSISKKRKRRFLGFLLGVGSAIASGIAVCKVLHLEGEVNKIKNALLSTNKAVVSLSNGVSVLTFKVLDLKNYINNQLLPILNQQSCRIFNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRGGSGGSGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 182 - protein, 651 aa, synthetic construct
QNITEEFYQSTCSAVSRGYFSALRTGWYTSVITIELSNITETKCNGTDTKVKLIKQELDKYKNAVTELQLLMQNTPAANNRARREAPQHMNYTINTTKNLNVSISKKRKRRFLGFLLGVGSAIASGIAVCKVLHLEGEVNKIKNALLSTNKAVVSLSNGVSVLTFKVLDLKNYINNQLLPILNQQSCRIFNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRKSDELLGGSGGSGSDDARIAAIGDVDELNSQIGVLLAEPLPDDVRAALSAIQHDLFDLGGELCIPGHAAITEDHLLRLALWLVHYNGQLPPLEEFILPGGARGAALAHVCRTVCRRAERSIKALGASEPLNIAPAAYVNLLSDLLFVLARVLNRAAGGADVLWDRTRAH

SEQ ID NO: 183 - protein, 739 aa, synthetic construct
QNITEEFYQSTCSAVSRGYFSALRTGWYTSVITIELSNITETKCNGTDTKVKLIKQELDKYKNAVTELQLLMQNTPAANNRARREAPQHMNYTINTTKNLNVSISKKRKRRFLGFLLGVGSAIASGIAVCKVLHLEGEVNKIKNALLSTNKAVVSLSNGVSVLTFKVLDLKNYINNQLLPILNQQSCRIFNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRGYIPEAPRDGQAYVRKDGEWVLLSTFLGSGGSGSGSGGSGSGEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 184 - protein, 715 aa, synthetic construct
QNITEEFYQSTCSAVSRGYFSALRTGWYTSVITIELSNITETKCNGTDTKVKLIKQELDKYKNAVTELQLLMQNTPAANNRARREAPQHMNYTINTTKNLNVSISKKRKRRFLGFLLGVGSAIASGIAVCKVLHLEGEVNKIKNALLSTNKAVVSLSNGVSVLTFKVLDLKNYINNQLLPILNQQSCRIFNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRKSDELLGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 185 - protein, 515 aa, synthetic construct
QNITEEFYQSTCSAVSRGYFSALRTGWYTSVITIELSNITETKCNGTDTKVKLIKQELDKYKNAVTELQLLMQNTPAANNRARREAPQHMNYTINTTKNLNVSISKKRKRRFLGFLLGVGSAIASGIAVCKVLHLEGEVNKIKNALLSTNKAVVSLSNGVSVLTFKVLDLKNYINNQLLPILNQQSCRIFNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRKSDELLGYIPEAPRDGQAYVRKDGEWVLLSTFL

SEQ ID NO: 186 - protein, 621 aa, synthetic construct
QNITEEFYQSTCSAVSRGYFSALRTGWYTSVITIELSNITETKCNGTDTKVKLIKQELDKYKNAVTELQLLMQNTPAANNRARREAPQHMNYTINTTKNLNVSISKKRKRRFLGFLLGVGSAIASGIAVCKVLHLEGEVNKIKNALLSTNKAVVSLSNGVSVLTFKVLDLKNYINNQLLPILNQQSCRIFNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRKSDELLGYIPEAPRDGQAYVRKDGEWVLLSTFLGGSMEEVVLITVPSALVAVKIAHALVEERLAACVNIVPGLTSIYREEGSVVSDHELLLLVKTTTDAFPKLKERVKELHPYEVPEIVALPIAEGNREYLDWLRENTG

SEQ ID NO: 187 - protein, 594 aa, synthetic construct
QNITEEFYQSTCSAVSRGYFSALRTGWYTSVITIELSNITETKCNGTDTKVKLIKQELDKYKNAVTELQLLMQNTPAANNRARREAPQHMNYTINTTKNLNVSISKKRKRRFLGFLLGVGSAIASGIAVCKVLHLEGEVNKIKNALLSTNKAVVSLSNGVSVLTFKVLDLKNYINNQLLPILNQQSCRIFNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRKSDELLGGSMEEVVLITVPSALVAVKIAHALVEERLAACVNIVPGLTSIYREEGSVVSDHELLLLVKTTTDAFPKLKERVKELHPYEVPEIVALPIAEGNREYLDWLRENTG

SEQ ID NO: 188 - protein, 640 aa, synthetic construct
QNITEEFYQSTCSAVSRGYFSALRTGWYTSVITIELSNITETKCNGTDTKVKLIKQELDKYKNAVTELQLLMQNTPAANNRARREAPQHMNYTINTTKNLNVSISKKRKRRFLGFLLGVGSAIASGIAVCKVLHLEGEVNKIKNALLSTNKAVVSLSNGVSVLTFKVLDLKNYINNQLLPILNQQSCRIFNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRKSDELLGYIPEAPRDGQAYVRKDGEWVLLSTFLGGSMVRGIRGAITVNSDTPTSIIIATILLLEKMLEANGIQSYEELAAVIFTVTEDLTSAFPAEAARQIGMHRVPLLSAREVPVPGSLPRVIRVLALWNTDTPQDRVRHVYLSEAVRLRPDLESAQ